SEQUENCE LISTING (202 sequences)

SEQ ID NO: 1 - DNA, 28 bp, Artificial Sequence
  Synthetic Polynucleotide
gcaagattttataccatgttcaccaact

SEQ ID NO: 2 - DNA, 25 bp, Artificial Sequence
  Synthetic Polynucleotide
cccacctactaaatgaacacacaga

SEQ ID NO: 3 - DNA, 19 bp, Artificial Sequence
  Synthetic Polynucleotide
ctcaccttgttgtaaatgt

SEQ ID NO: 4 - DNA, 22 bp, Artificial Sequence
  Synthetic Polynucleotide
cacagcttaggcgtgaagaaaa

SEQ ID NO: 5 - DNA, 25 bp, Artificial Sequence
  Synthetic Polynucleotide
gctgtcttcataaggatgcactttt

SEQ ID NO: 6 - DNA, 18 bp, Artificial Sequence
  Synthetic Polynucleotide
aaggaaatggaaactttg

SEQ ID NO: 7 - DNA, 22 bp, Artificial Sequence
  Synthetic Polynucleotide
cacagcttaggcgtgaagaaaa

SEQ ID NO: 8 - DNA, 27 bp, Artificial Sequence
  Synthetic Polynucleotide
ccactcaatgaagttgtcacaataagg

SEQ ID NO: 9 - DNA, 22 bp, Artificial Sequence
  Synthetic Polynucleotide
caatggagatagcaaaggtatt

SEQ ID NO: 10 - protein, 400 aa, Homo Sapien
PYVHKILVVIEPLLIDEDYYARVEGREIISNLAKAAGLATMISTMRPDIDNMDEYVRNTTARAFAVVASALGIPSLLPFLKAVCKSKKSWQARHTGIKIVQQIAILMGCAILPHLRSLVEIIEHGLVDEQQKVRTISALAIAALAEAATPYGIESFDSVLKPLWKGIRQHRGKGLAAFLKAIGYLIPLMDAEYANYYTREVMLILIREFQSPDEEMKKIVLKVVKQCCGTDGVEANYIKTEILPPFFKHFWQHRMALDRRNYRQLVDTTVELANKVGAAEIISRIVDDLKDEAEQYRKMVMETIEKIMGNLGAADIDHKLEEQLIDGILYAFQEQTTEDSVMLNGFGTVVNALGKRVKPYLPQICGTVLWRLNNKSAKVRQQAADLISRTAVVMKTCQEE

SEQ ID NO: 11 - protein, 400 aa, Mus musculus; identical to SEQ ID NO: 10

SEQ ID NO: 12 - protein, 400 aa, Danio rerio; identical to SEQ ID NO: 10

SEQ ID NO: 13 - protein, 400 aa, c.elegans
PYVHKILVVIEPLLIDEDYYARVEGREIISNLAKAAGLATMISTMRPDIDNVDEYVRNTTARAFAVVASALGIPALLPFLKAVCKSKKSWQARHTGIKIVQQMAILMGCAVLPHLKALVDIVESGLDDEQQKVRTITALCLAALAEASSPYGIEAFDSVLKPLWKGIRMHRGKGLAAFLKAIGYLIPLMDAEYASYYTREVMLILIREFASPDEEMKKIVLKVVKQCCATDGVEASYIRDEVLPSFFKAFWNQRMAMDRRNYRQLVDTTVEIAQKVGCVEMIARIVDDLKDENEQYRKMVMETIENIVALQGATDIDARLEEQLIDGLLYAFQEQTQEDSVMLDGFGTICSSLGRRAKAYIPQICGTILWRLNNKSAKVRQQAADLIARIAPVMHMCEEE

SEQ ID NO: 14 - protein, 400 aa, s.pombe
PFTHKILVVIEPLLIDEDYYARAEGREIISNLAKASGLAHMIATMRPDIDHVDEYVRNTTARAFSVVASALGVPALLPFLKAVCRSKKSWQARHTGVRIIQQIALLLGCSILPHLKNLVDCIGHGLEDEQQKVRIMTALSLSALAEAATPYGIEAFDSVLKPLWSGVQRHRGKSLAAFLKATGFIIPLMEPEYASHFTRRIMKILLREFNSPDEEMKKIVLKVVSQCASTDGVTPEYLRTDVLPEFFHCFWSRRMASDRRSYKQVVETTVVLAQQVGSRQIVERVVNNFKDESEPYRKMTAETVDKVIGSLGVSEIDERLEELLLDGVLFAFQEQSVEEKVILTCFSTVVNALGTRCKPYLPQIVSTILYRLNNKSANVREQAADLVSSITIVLKACGEE

SEQ ID NO: 15 - protein, 400 aa, Homo sapiens
LQEWLKMFQSWSGPEKLLALDELIDSCEPTQVKHMMQVIEPQFQRDFISLLPKELALYVLSFLEPKDLLQAAQTCRYWRILAEDNLLWREKCKEEGIDEPLHIKRRKVIKPGFIHSPWKSAYIRQHRIDTNWRRGELKSPKVLKGHDDHVITCLQFCGNRIVSGSDDNTLKVWSAVTGKCLRTLVGHTGGVWSSQMRDNIIISGSTDRTLKVWNAETGECIHTLYGHTSTVRCMHLHEKRVVSGSRDATLRVWDIETGQCLHVLMGHVAAVRCVQYDGRRVVSGAYDFMVKVWDPETETCLHTLQGHTNRVYSLQFDGIHVVSGSLDTSIRVWDVETGNCIHTLTGHQSLTSGMELKDNILVSGNADSTVKIWDIKTGQCLQTLQGPNKHQSAVTCLQFN

SEQ ID NO: 16 - protein, 400 aa, Mus musculus
LQEWLKMFQSWSGPEKLLALDELIDSCEPTQVKHMMQVIEPQFQRDFISLLPKELALYVLSFLEPKDLLQAAQTCRYWRILAEDNLLWREKCKEEGIDEPLHIKRRKIIKPGFIHSPWKSAYIRQHRIDTNWRRGELKSPKVLKGHDDHVITCLQFCGNRIVSGSDDNTLKVWSAVTGKCLRTLVGHTGGVWSSQMRDNIIISGSTDRTLKVWNAETGECIHTLYGHTSTVRCMHLHEKRVVSGSRDATLRVWDIETGQCLHVLMGHVAAVRCVQYDGRRVVSGAYDFMVKVWDPETETCLHTLQGHTNRVYSLQFDGIHVVSGSLDTSIRVWDVETGNCIHTLTGHQSLTSGMELKDNILVSGNADSTVKIWDIKTGQCLQTLQGPNKHQSAVTCLQFN

SEQ ID NO: 17 - protein, 400 aa, Danio rerio
LQEWLRTFQSWSGPEKLLALDELIDSCEPTQVKHMMQVIEPQFQRDFISLLPRELALHVLSFLEPKDLLQAAQTCRYWRILAEDNLLWKEKCKEEGIDEPLHIKRRKVIKPGFTHSPWKSAYIRQHRIDTNWRRGDLKSPKVLKGHDDHVITCLQFCGNRIVSGSDDNTLKVWSAVTGKCLRTLVGHTGGVWSSQMRDNIIISGSTDRTLKVWNAETGECIHTLYGHTSTVRCMHLHEKRVVSGSRDATLRVWDIETGQCLHVLMGHVAAVRCVQYDGRRVVSGAYDFMVKVWDPETETCLHTLQGHTNRVYSLQFDGIHVVSGSLDTSIRVWDVETGNCIHTLTGHQSLTSGMELKDNILVSGNADSTVKIWDIKTGQCLQTLQGPNKHQSAVTCLQFN

SEQ ID NO: 18 - protein, 480 aa, Homo sapien
MSHVAVENALGLDQQFAGLDLNSSDNQSGGSTASKGRYIPPHLRNREATKGFYDKDSSGWSSSKDKDAYSSFGSRSDSRGKSSFFSDRGSGSRGRFDDRGRSDYDGIGSRGDRSGFGKFERGGNSRWCDKSDEDDWSKPLPPSERLEQELFSGGNTGINFEKYDDIPVEATGNNCPPHIESFSDVEMGEIIMGNIELTRYTRPTPVQKHAIPIIKEKRDLMACAQTGSGKTAAFLLPILSQIYSDGPGEALRAMKENGRYGRRKQYPISLVLAPTRELAVQIYEEARKFSYRSRVRPCVVYGGADIGQQIRDLERGCHLLVATPGRLVDMMERGKIGLDFCKYLVLDEADRMLDMGFEPQIRRIVEQDTMPPKGVRHTMMFSATFPKEIQMLARDFLDEYIFLAVGRVGSTSENITQKVVWVEESDKRSFLLDLLNATGKDSLTLVFVETKKGADSLEDFLYHEGYACTSIHGDRSQRDR

SEQ ID NO: 19 - protein, 480 aa, Mus musculus; identical to SEQ ID NO: 18

SEQ ID NO: 20 - protein, 209 aa, Homo sapien
PSNLLLNTTCDLKICDFGLARVADPDHDHTGFLTEYVATRWYRAPEIMLNSKGYTKSIDIWSVGCILAEMLSNRPIFPGKHYLDQLNHILGILGSPSQEDLNCIINLKARNYLLSLPHKNKVPWNRLFPNADSKALDLLDKMLTFNPHKRIEVEQALAHPYLEQYYDPSDEPIAEAPFKFDMELDDLPKEKLKELIFEETARFQPGYRS

SEQ ID NO: 21 - protein, 209 aa, Mus musculus; identical to SEQ ID NO: 20

SEQ ID NO: 22 - protein, 209 aa, Danio rerio
PSNLLLNTTCDLKICDFGLARVADPDHDHTGFLTEYVATRWYRAPEIMLNSKGYTKSIDIWSVGCILAEMLSNRPIFPGKHYLDQLNHILGILGSPSQEDLNCIINLKARNYLLSLPHKNKVPWNRLFPNADSKALDLLDKMLTFNPHKRIEVEQALAHPYLEQYYDPTDEPVAEAPFKFDMELDDLPKETLKELIFEETARFQPGYRP

SEQ ID NO: 23 - protein, 53 aa, Homo sapien
QVPEHPFLTPSPESPDQWSSSSPHSNVSDWSEGVSSPPTSMQSQIARIPEAFK

SEQ ID NO: 24 - protein, 53 aa, Mus musculus
QVPEHPFLTPSPESPDQWSSSSPHSNISDWSEGISSPPTTMPSQITHIPEAFK

SEQ ID NO: 25 - protein, 51 aa, Danio rerio
QVPDHPFLTPSAGSPDQWSSSSPHSNLSDWSEGISSPPTSMQMNHIPEAFK

SEQ ID NO: 26 - protein, 53 aa, Drosophila
QVPDHPFLTPSPESPDQWSSSSPHSNVSDWSEGISSPPTSMQSQMGHIPEAFK

SEQ ID NO: 27 - protein, 79 aa, Homo sapien
MDPSDFPSPFDPLTLPEKPLAGDLPVDMEFGEDLLESQTAPTRGWAPPGPSPSSGALDLLDTPAGLEKDPGVLDGATEL

SEQ ID NO: 28 - protein, 80 aa, Mus musculus
MDPSDFPSPFDPLTLPEKPLAGDLPVDMEFGEDLLESQTAPSRGWAPPGPSPSSGALDLLDTPSGLEKDPGGVLDGATEL

SEQ ID NO: 29 - protein, 21 aa, Danio rerio
MSTEDFIGGTAESGKMDTTET

SEQ ID NO: 30 - protein, 78 aa, Homo sapien
RPIPQSGDPADATRCSICQKTGEVLHEVSNGSVVHRLCSDSCFSKFRANKGLKTNCCDQCGAYIYTKTGSPGPELLFH

SEQ ID NO: 31 - protein, 78 aa, Mus musculus
RPIPQSGDPADATRCSICQKTGEVLHEVSNGSVVHRLCSDSCFSKFRANKGLKTNCCDQCGAYIYARPGGLGPELLFH

SEQ ID NO: 32 - protein, 64 aa, Danio rerio
KCSVCQKAGTTFTHKVNLMDSVHILCSDDCFNQFRTSNKLNGNSCMNCGGICYGTDAPCQSLQI

SEQ ID NO: 33 - protein, 152 aa, Homo sapien
MFFNTKFFGLQTAEEHMQLSFTNVVRQSRKCTTPRGTTKVVSIRYYAPVRQRKGRDTGPGKRKREDEAPILEQRENRMNPLRCPVKFYEFYLSKCPESLRTRNDVFYLQPERSCIAESPLWYSVIPMDRSMLESMLNRILAVREIYEELGRP

SEQ ID NO: 34 - protein, 151 aa, Mus musculus
MFFNTKFFGLQTAEEHMQLSFTNVVRQSRKCTTPRGTTKVVSIRYYAPVRQRKGRDTGPGKRKREDETILEQRENRMNPLRCPVKFYEFYLSKCPESLRTRNDVFYLQPERSCIAESPLWYSVIPMDRSMLESMLNRILAVREIYEELGRP

SEQ ID NO: 35 - protein, 152 aa, Danio rerio
IYFFTKYFNYRTAEQHRLLSFGHIVRCSRSKGNTKVACLRFYPPKDEADGVPAKRRKEEGEEEDETVYEIKENSDNPLRCPVRLYEFYLSKCSPSVRQRTTDFYLSPERSCVPNSPMWFSISALSDEALNSMLTRILTVRELHLDTEKTPAD

SEQ ID NO: 36 - protein, 75 aa, Homo sapien
KKNRLRRKSSTRHIHALELVVSRNLSPPNCTELQIDSCSSSEEIKKKKYNQMPVRHSRNLQLMEGKEPATGAKKS

SEQ ID NO: 37 - protein, 73 aa, Mus musculus
KKNRLRRKSSIRCALPLEPISRNPSPPTCAELQIDSCGSSEETKKNHSNQQPAGHLREPQLIEDTEPAADAKK

SEQ ID NO: 38 - protein, 76 aa, Gallus gallus domesticus
KNACSTAKGCRHSTRTRCAIHLVDRNPGSFDLAEPLINSYPSNEEPSKADCERRQVRRSRRLQLLSEEITKETGKM

SEQ ID NO: 39 - protein, 80 aa, Homo sapien
FGQNSGWLFLDSSTSMFINARARVYHLPDAKMSKKEKISEKMEIKEGEETKKELVLESNPKWEALTEVLKEIEAENKESE

SEQ ID NO: 40 - protein, 80 aa, Mus musculus
FGQNSGWLFLDASTSMFVNARARVYRVPDVKLNKKAKTSEKTSSPEVQETKKELVLESNPKWEALTDVLKEIEAENKESE

SEQ ID NO: 41 - protein, 80 aa, Gallus gallus domesticus
FGENSGWLFLDSSTSMFVNARARVYRTADEKLNQKGKVSENRDVKKENELKRELVLESNPKWEALREVLNEIENENKNSE

SEQ ID NO: 42 - protein, 78 aa, Danio rerio
FGSNSGWLFLDSSMSMFVNARSRVYRIQESKKKLKVGETEQKQNLAPPKRELVLEKNPKWEALTEVLQEIEKENSKSE

SEQ ID NO: 43 - protein, 80 aa, Homo sapien
TINMRLDCVQELLQDEELFFGLQSVISRFLDTEQLLSVLVQIPEQDTVNAAESKITNLIYLKHTLELVDPLKIAMKNCNT

SEQ ID NO: 44 - protein, 80 aa, Mus musculus
TISMRLDCVQELLQDEELFFGLQSVISRFLDTEQLLSVLVQIPKQDTVNAAESKITNLIYLKHTLELVEPLKVTLKNCST

SEQ ID NO: 45 - protein, 80 aa, Danio rerio
TIKSRQDTIQELLQNEELFFSLKNAIAHFLDIDELLSALVQIPKQETIAVAEAKITQVIQLKHIVELVPSLKVMLQHSTT

SEQ ID NO: 46 - protein, 80 aa, Homo sapien
KSEVNDKNHEMEEIRKKLGGANKEMTHLQKEVTAIETKLEQKRSDRHNLLQACKMQDIKLPLSKGTMDDISQEEGSSQGE

SEQ ID NO: 47 - protein, 80 aa, Mus musculus; identical to SEQ ID NO: 46

SEQ ID NO: 48 - protein, 80 aa, Danio rerio
KGEVNDKNREMEEIRKKLGSANKELTQLQKEVTAIETKLEQKRSDRHNLLQACKMQDIKLPLKSGTMDDISQGEGGSQVE

SEQ ID NO: 49 - protein, 71 aa, Homo sapien
VVKAPPETRLEVPDSIESLQIHLASTQGPIEVYLCPEETETHSPMKTNNQDHNGNIPKPASKDLASTNSGH

SEQ ID NO: 50 - protein, 71 aa, Mus musculus
VVKAPPETRLEVPDSIESLQIHLASTQGPIEVYLCPEETETHRPMKTNNQDHNGNIPKPTSKDLASNNSGH

SEQ ID NO: 51 - protein, 79 aa, Danio rerio
AIKAPSETKLEVPDPKESLQVHLSSSKGPIDVFLCTDGGDSGSPLQNGLDVNGNHPAFLKVSQEAASDGPADNVKMNGN

SEQ ID NO: 52 - protein, 70 aa, Homo sapien
MATLIYVDKENGEPGTRVVAKDGLKLGSGPSIKALDGRSQVSTPRFGKTFDAPPALPKATRKALGTVNRA

SEQ ID NO: 53 - protein, 67 aa, Mus musculus
MATLIFVDKDNEEPGRRLASKDGLKLGTGVKALDGKLQVSTPRVGKVFNAPAVPKASRKALGTVNRV

SEQ ID NO: 54 - protein, 75 aa, Gallus gallus domesticus
MARGVLINPEMTTLIFVDKENDEVGATKNQLRRLSVPSKVLSERAQVNTPLPKKPIRTPATSCSVRKALGNVNRT

SEQ ID NO: 55 - protein, 61 aa, Danio rerio
METMIYMDQENGRLMTPAIKSRQNRLHSAPDQCLRTPLNGKAHLGAPLQSSRKALGVINKI

SEQ ID NO: 56 - protein, 80 aa, Homo sapien
EINILNVLKCDINIPIAYHFLRRYARCIHTNMKTLTLSRYICEMTLQEYHYVQEKASKLAAASLLLALYMKKLGYWVPFL

SEQ ID NO: 57 - protein, 80 aa, Mus musculus
ESSILQTLNFDINIPTAYNFLRRYASCIHASMKTLTLSRFICEMTLQEYEYIEERPSKLAAASFILALYMRNLSNCVPTL

SEQ ID NO: 58 - protein, 80 aa, Gallus gallus domesticus
ETSILRTLNFDINIPIPYRFLRRFAKCARASMETLTLARFVCEMTLQEYDYARERPSKLAASSLLLALTMKNLGGWTPTL

SEQ ID NO: 59 - protein, 80 aa, Danio rerio
EISILQALNFDTNIPVPYRFLRRYAKCVNAGMDTLTLARFICELSLLEMEFVPVRASLLASACLLIALVTKDLGGWTQCL

SEQ ID NO: 60 - DNA, 33 bp, Homo sapien
tgttttttcttgttttagttttggcctttgctc

SEQ ID NO: 61 - DNA, 37 bp, Mus musculus
tatttccttactttgtttgtagttttggcctttgctc

SEQ ID NO: 62 - DNA, 32 bp, Gallus gallus domesticus
tactcatctttttctagttttggcttttggcc

SEQ ID NO: 63 - DNA, 41 bp, Danio rerio
tatccacattggatcgttgtgtgcagttctagcgttcagtg

SEQ ID NO: 64 - protein, 75 aa, Homo sapien
SINGVENQDQQEPEPYSDDDEINGVTQGDRLRALKSRRQSKTNAIPLTVILQSSVQSCKTSEPNISGSAGITKRT

SEQ ID NO: 65 - protein, 75 aa, Mus musculus
SLNGLENQDNQEPEPYSDDDEVSGMTQGDRLRALKSRRQPKASAIPLTVILQSSVQSCKTPEPNLSGSAGITKRT

SEQ ID NO: 66 - protein, 76 aa, Danio rerio
SINGTNKSTSQRKTASQNHTEEEEEDCAGLTQGDKLRALKSKRQSRASTGSISLEENTVHTKSTSKSLSSEKRKRT

SEQ ID NO: 67 - protein, 76 aa, Homo sapien
HVIRALVGERGSSSGLLSPQRALCLLELTLEHCRRFCWSRHHDKAISAVEKAHSYLRNTNLAPSLQLCQLGVKLLQ

SEQ ID NO: 68 - protein, 76 aa, Mus musculus
HLIRVLVGEGGSSPGPLSPQRALCLLEITLEHCRRLCWNHHHRQAARAVERARNHLEKTSVAPSLQLCQMGVELLE

SEQ ID NO: 69 - protein, 74 aa, Danio rerio
LFSGAFINPQDSASTSSLLAVWCEVAFKVSKLLCKSSFSPEAVELLRGTLKEVNGHVGLRSALGLADRAVQMQC

SEQ ID NO: 70 - protein, 77 aa, Homo sapien
RHEKKKKVRKYWDVPPPGFEHITPMQYKAMQAAGQIPATALLPTMTPDGLAVTPTPVPVVGSQMTRQARRLYVGNIP

SEQ ID NO: 71 - protein, 77 aa, Mus musculus; identical to SEQ ID NO: 70

SEQ ID NO: 72 - protein, 71 aa, Danio rerio
RHHRNSRRKPSLYWDVPPPGFEHITPMQYKAMQASGQIPASVVPDTPQTAVPVVGSTITRQARRLYVGNIP

SEQ ID NO: 73 - protein, 80 aa, Homo sapien
DLLVLGLHRASEMAGPPQMPNDFLSFQDIATEAAHPIRLFCRYIDRIHIFFRFTADEARDLIQRYLTEHPDPNNENIVGY

SEQ ID NO: 74 - protein, 80 aa, Mus musculus; identical to SEQ ID NO: 73

SEQ ID NO: 75 - protein, 80 aa, Gallus gallus domesticus
DLLVLGLHRASEMAGPPQMPNDFLSFQDTATESAHPIRLYCRYIDRIHIFFRFSADEARDLIQRYLTEHPDPNNENIVGY

SEQ ID NO: 76 - protein, 80 aa, Danio rerio
DLLVLGLHRSSEMAGPPQMPNDFLTFQDTVTETAHPIRLYCRYVDRIHLFFRFSAEEARDLIQRYLTEHPDPNNENIVGY

SEQ ID NO: 77 - protein, 80 aa, Homo sapien
PEAQQLLEDWVARLMAQAFESCQLDSMVTAFLVVRQAALEGPSAFLSYADWFKASFGSTRGYHGCSKKALVFLFTFLSEL

SEQ ID NO: 78 - protein, 80 aa, Mus musculus
PEAQQLVKGWVASLMARAFESYHLDSMVTAFLIVRQATLEGPYVFPSYADWFKESFGSSHGYHSCSKKTLVFLFKFLSDL

SEQ ID NO: 79 - protein, 80 aa, Homo sapien
EAFTAKISDFGLARASEKFAQTVMTSRIVGTTAYMAPEALRGEITPKSDIYSFGVVLLEIITGLPAVDEHREPQLLLDIK

SEQ ID NO: 80 - protein, 80 aa, Mus musculus
KDFTAKISDFGLARASARLAQTVMTSRIVGTTAYMAPEALRGEITPKSDIYSFGVVLLELITGLAAVDENREPQLLLDIK

SEQ ID NO: 81 - protein, 80 aa, Homo sapien
KENVDLWSVGCIMGEMVCHKILFPGRDYIDQWNKVIEQLGTPCPEFMKKLQPTVRTYVENRPKYAGYSFEKLFPDVLFPA

SEQ ID NO: 82 - protein, 80 aa, Mus musculus; identical to SEQ ID NO: 81

SEQ ID NO: 83 - protein, 80 aa, Danio rerio
KENVDIWSVGCIMGEMVRHKILFPGRDYIDQWNKVIEQLGTPSPEFMKKLQPTVRNYVENRPKYAGLTFPKLFPDCLFPA

SEQ ID NO: 84 - protein, 79 aa, Homo sapien
TSLRKINAAWFKNMPHLKVLDLEFNYLVGEIASGAFLTMLPRLEILDLSFNYIKGSYPQHINISRNFSKLLSLRALHLR

SEQ ID NO: 85 - protein, 79 aa, Mus musculus
TSLRTIPSTWFENLSNLKELHLEFNYLVQEIASGAFLTKLPSLQILDLSFNFQYKEYLQFINISSNFSKLRSLKKLHLR

SEQ ID NO: 86 - protein, 79 aa, Danio rerio
NSLQSINSMWFQNLTNLKYLYLSFNSLISEFESGQFFSVLPQVEVVDISYNNPSERIYPRLKLSEGFSRLESLQTLHLE

SEQ ID NO: 87 - protein, 64 aa, Homo sapien
APQPDELPEVDNLTLDGNPFLVPGTALPHEGSMNSGVVPACARSTLSVGVSGTLVLLQGARGFA

SEQ ID NO: 88 - protein, 61 aa, Mus musculus
NPSPDELPQVGNLSLKGNPFLDSESHSEKFNSGVVTAGAPSSQAVALSGTLALLLGDRLFV

SEQ ID NO: 89 - protein, 80 aa, Gallus gallus domesticus
SLQALFLSRNALPAAPSIRGCPALHTLHLDNNLIAELPRDEALLLEHLQDVAVAGNPFNCTCGGAGAVQALAAVGCLRQG

SEQ ID NO: 90 - DNA, 31 bp, Homo sapien
tgtgtttccctgcagctcttcaagtccaaaa

SEQ ID NO: 91 - DNA, 29 bp, Mus musculus
ctgcttccttcagctcctccagtccaaaa

SEQ ID NO: 92 - DNA, 31 bp, Gallus gallus domesticus
tttttttctctgcagcactccgagtccaaaa

SEQ ID NO: 93 - protein, 78 aa, Homo sapien
AYNYMEIGGTSSSLLDSTNTNFKEEPAAKYQAIFDNTTSLTDKHLDPIRENLGKHWKNCARKLGFTQSQIDEIDHDYE

SEQ ID NO: 94 - protein, 74 aa, Mus musculus
NHNYMDVGLNSQPPNNTCKEESTSRHQAIFDNTTSLTDEHLNPIRENLGRQWKNCARKLGFTESQIDEIDHDYE

SEQ ID NO: 95 - protein, 78 aa, Gallus gallus domesticus
SHNHMKIEEQNQHISTSSVAKDASYSYYESTGIFDNNTVLTKKQLSLVRENLGKQWKHCARELGFSNSVIEEIDHDYE

SEQ ID NO: 96 - protein, 78 aa, Danio rerio
SYNTMNLRISDSSPYSSLSTTGSTTSRYKELLLMYESHTVTESHLELVRQNVGANWKQVARKLGLSEIDVETIEHDYD

SEQ ID NO: 97 - protein, 80 aa, Homo sapien
MAVMEMACPGAPGSAVGQQKELPKAKEKTPPLGKKQSSVYKLEAVEKSPVFCGKWEILNDVITKGTAKEGSEAGPAAISI

SEQ ID NO: 98 - protein, 80 aa, Mus musculus
MAVMEVACPGTPGSAVGQQKELAKAKEKTQSLGKKQSCIFKLEAVEKSPVFCGKWEILNDVITKGTAKDGSEGGPPAISI

SEQ ID NO: 99 - protein, 79 aa, Gallus gallus domesticus
MAVMGIACHGAPAPPAKQQQELSATKGLHEAASEKQSSVCKVEDSKKAIFHSQWKILNDVITKGTAKEETEGGCASISI

SEQ ID NO: 100 - protein, 79 aa, Homo sapien
PKMEKHNAQGQGNGLRYGLSSMQGWRVEMEDAHTAVIGLPSGLESWSFFAVYDGHAGSQVAKYCCEHLLDHITNNQDFK

SEQ ID NO: 101 - protein, 79 aa, Mus musculus
PKMEKHNAQGQGNGLRYGLSSMQGWRVEMEDAHTAVIGLPSGLETWSFFAVYDGHAGSQVAKYCCEHLLDHITNNQDFR

SEQ ID NO: 102 - protein, 79 aa, Gallus gallus domesticus
PKMEKHNAQGQGNGLRYGLSSMQGWRVEMEDAHTAVIGLPNGLDGWSFFAVYDGHAGSQVAKYCCEHLLDHITSNQDFK

SEQ ID NO: 103 - protein, 80 aa, Danio rerio
PKTEKHNAHGAGNGLNFGLSSMQGWRVEMEDAHTAVVGLPHGLDDWSFFAVYDGHAGSRVANYCSKHLLEHIITSSEDFR

SEQ ID NO: 104 - protein, 80 aa, Homo sapien
RSRSRSRDRRFRGRYRSPYSGPKFNSAIRGKIGLPHSIKLSRRRSRSKSPFRKDKSPVREPIDNLTPEERDARTVFCMQL

SEQ ID NO: 105 - protein, 80 aa, Mus musculus; identical to SEQ ID NO: 104

SEQ ID NO: 106 - protein, 80 aa, Gallus gallus domesticus; identical to SEQ ID NO: 104

SEQ ID NO: 107 - protein, 78 aa, Danio rerio
RSRSRSRERGGRYRAPFSGLKFNGGPRGKTGPPPAIKLSRRRSRSRSPFKKDRSPVRQPIDNLTPEERDARTVFCMQL

SEQ ID NO: 108 - protein, 80 aa, Homo sapien
GRVLELAQLLDQIWRTKDAGLGVYSLALLNNVSYNVVEFSKSQVEWMSDKLMRCFEDKRNNPFQFRHLSLCHGLSDLARV

SEQ ID NO: 109 - protein, 80 aa, Mus musculus; identical to SEQ ID NO: 108

SEQ ID NO: 110 - protein, 80 aa, Gallus gallus domesticus
GRVLELAQLLDQIWRTKDAGLGVYSLALLNNVSYNVVEFSKSQVEWMSDKLMRCFEDKRNNPFQFRHLSLCHSLSDLARV

SEQ ID NO: 111 - protein, 80 aa, Danio rerio; identical to SEQ ID NO: 110

SEQ ID NO: 112 - protein, 81 aa, Homo sapien
KFLKTVVNKLFEFMHETHDGVQDMACDTFIKIAQKCRRHFVQVQVGEVMPFIDEILNNINTIICDLQPQQVHTFYEAVGYM

SEQ ID NO: 113 - protein, 81 aa, Mus musculus; identical to SEQ ID NO: 112

SEQ ID NO: 114 - protein, 81 aa, Gallus gallus domesticus; identical to SEQ ID NO: 112

SEQ ID NO: 115 - protein, 81 aa, Danio rerio; identical to SEQ ID NO: 112

SEQ ID NO: 116 - protein, 80 aa, Homo sapien
KDYLSLYLLLVSCPKSEVRAKFKFSILNAKGEETKAMESQRAYRFVQGKDWGFKKFIRRDFLLDEANGLLPDDKLTLFCE

SEQ ID NO: 117 - protein, 80 aa, Mus musculus; identical to SEQ ID NO: 116

SEQ ID NO: 118 - protein, 80 aa, Gallus gallus domesticus; identical to SEQ ID NO: 116

SEQ ID NO: 119 - protein, 80 aa, Danio rerio; identical to SEQ ID NO: 116

SEQ ID NO: 120 - protein, 74 aa, Homo sapien
MFQAAERPQEWAMEGPRDGLKKERLLDDRHDSGLDSMKDEEYEQMVKELQEIRLEPQEVPRGSEPWKQQLTEDG

SEQ ID NO: 121 - protein, 74 aa, Mus musculus
MFQPAGHGQDWAMEGPRDGLKKERLVDDRHDSGLDSMKDEEYEQMVKELREIRLQPQEAPLAAEPWKQQLTEDG

SEQ ID NO: 122 - protein, 80 aa, Danio rerio
MDLHRAAMLNFVDCNVDEMDTKNRKTQQCEDRVDSGVDSLKEEEYETREISEEMERLTIQTASLKETICEPWAKVVTEDG

SEQ ID NO: 123 - protein, 80 aa, Homo sapien
MALTFYLLVALVVLSYKSFSSLGCDLPQTHSLGNRRALILLAQMRRISPFSCLKDRHDFEFPQEEFDDKQFQKAQAISVL

SEQ ID NO: 124 - protein, 80 aa, Mus musculus
MARLCAFLMVLVVLSYWSTCSLGCDLPQTHNLRNKKALTLLVQMRRLSPLSCLKDRKDFGFPLEKVDAQQIQEAQAIPVL

SEQ ID NO: 125 - protein, 80 aa, Homo sapien
LQHLVIQQQHQQFLEKHKQQFQQQQLQMNKIIPKPSEPARQPESHPEETEEELREHQALLDEPYLDRLPGQKEAHAQAGV

SEQ ID NO: 126 - protein, 79 aa, Mus musculus
LQHLVIQQQHQQFLEKHKQQFQQQQLHLSKIISKPSEPPRQPESHPEETEEELREHQALLDEPYLDRLPGQKEPSLAGV

SEQ ID NO: 127 - protein, 68 aa, Danio rerio
LQQLVVQQQHQQFLEKHKQQFQQQQLHISKMMVKPSEPNRQHESHPEETEEELREHQGLGDPADPLPH

SEQ ID NO: 128 - protein, 80 aa, Homo sapien
DDSLLYKTLIDFKSNHRLLITGTPLQNSLKELWSLLHFIMPEKFEFWEDFEEDHGKGRENGYQSLHKVLEPFLLRRVKKD

SEQ ID NO: 129 - protein, 80 aa, Mus musculus; identical to SEQ ID NO: 128

SEQ ID NO: 130 - protein, 80 aa, Danio rerio
DDSLLYKTLIDFRSNHRLLITGTPLQNSLKELWSLLHFLMSDKFESWEDFEDEHGKGRDNGYQSLHKVLEPFLLRRVKKD

SEQ ID NO: 131 - protein, 80 aa, Homo sapien
SLIRSSGKLILLDKLLTRLRERGNRVLIFSQMVRMLDILAEYLTIKHYPFQRLDGSIKGEIRKQALDHFNADGSEDFCFL

SEQ ID NO: 132 - protein, 80 aa, Mus musculus; identical to SEQ ID NO: 131

SEQ ID NO: 133 - protein, 80 aa, Danio rerio
SLVRGGGKLVLLDKLLTRLKDRGNRVLIFSQMVRMLDILADYLSMKRYQFQRLDGSIKGELRKQALDHFNAEGSEDFCFL

SEQ ID NO: 134 - protein, 80 aa, Homo sapien
NFATMEDEEELEERPHKDWDEIIPEEQRKKVEEEERQKELEEIYMLPRIRSSTKKAQTNDSDSDTESKRQAQRSSASESE

SEQ ID NO: 135 - protein, 80 aa, Mus musculus; identical to SEQ ID NO: 134

SEQ ID NO: 136 - protein, 79 aa, Danio rerio
NFTMDESTPDLDEKPGRDWDEIIPEEQRRKVEEEQKQKEMEDIYMLPRSRSSNKKAQANDSDSDVGSKLKHRSSGSDSE

SEQ ID NO: 137 - protein, 160 aa, Homo sapien
NKAIIASNIMYIVGQYPRFLRAHWKFLKTVVNKLFEFMHETHDGVQDMACDTFIKIAQKCRRHFVQVQVGEVMPFIDEILNNINTIICDLQPQQVHTFYEAVGYMIGAQTDQTVQEHLIEKYMLLPNQVWDSIIQQATKNVDILKDPETVKQLGSILKTN

SEQ ID NO: 138 - protein, 160 aa, Mus musculus; identical to SEQ ID NO: 137

SEQ ID NO: 139 - protein, 160 aa, Danio rerio
NKAIIASNIMYIVGQYPRFLRAHWKFLKTVVNKLFEFMHETHDGVQDMACDTFIKIAQKCRRHFVQVQVGEVMPFIDEILNNINTIICDLQPQQVHTFYEAVGYMIGAQTDQAVQEHLIEKYMLLPNQVWDSIIQQATKNVDILKDPETVKQLGSILKTN

SEQ ID NO: 140 - protein, 160 aa, C. elegans
NKAVIASNIMYVVGQYPRFLRAHWKFLKTVINKLFEFMHETHEGVQDMACDTFIKISIKCKRHFVIVQPAENKPFVEEMLENLTGIICDLSHAQVHVFYEAVGHIISAQIDGNLQEDLIMKLMDIPNRTWNDIIAAASTNDSVLEEPEMVKSVLNILKTN

SEQ ID NO: 141 - protein, 160 aa, S.pombe
NKAVVASNIMYVVGQYPRFLKAHWKFLKTVVNKLFEFMHEYHEGVQDMACDTFIKIAQKCRRHFVAQQLGETEPFINEIIRNLAKTTEDLTPQQTHTFYEACGYMISAQPQKHLQERLIFDLMALPNQAWENIVAQAAQNAQVLGDPQTVKILANVLKTN

SEQ ID NO: 142 - protein, 80 aa, Homo sapien
MSLVPATNYIYTPLNQLKGGTIVNVYGVVKFFKPPYLSKGTDYCSVVTIVDQTNVKLTCLLFSGNYEALPIIYKNGDIVR

SEQ ID NO: 143 - protein, 80 aa, Mus musculus
MSLVSTAPYTYTPLNLLKEGTIANVYGVVKFFKPPYVSKGTDYCSVVTIVDQTNVKLTCMLFSGNYEALPIIYKVGDIVR

SEQ ID NO: 144 - protein, 80 aa, Homo sapien
PRTSSKYFNFTTEDHKMVEALRVWASTHMSPSWTLLKLCDVQPMQYFDLTCQLLGKAEVDGASFLLKVWDGTRTPFPSWR

SEQ ID NO: 145 - protein, 80 aa, Mus musculus
ARTSSKVFSFTPQDQKMVEALRVWASKHISASSTLVQLCDAQPMQYYDLTCQLLGKAQVDSTAFLLKVWDGTQTVLPSWR

SEQ ID NO: 146 - protein, 80 aa, Homo sapien
MSETAPAAPAAPAPAEKTPVKKKARKSAGAAKRKASGPPVSELITKAVAASKERSGVSLAALKKALAAAGYDVEKNNSRI

SEQ ID NO: 147 - protein, 80 aa, Mus musculus
MSETAPAAPAAPAPAEKTPVKKKARKAAGGAKRKTSGPPVSELITKAVAASKERSGVSLAALKKALAAAGYDVEKNNSRI

SEQ ID NO: 148 - protein, 59 aa, Homo sapien
PAAAAGAKKAKSPKKAKAAKPKKAPKSPAKAKAVKPKAAKPKTAKPKAAKPKKAAAKKK

SEQ ID NO: 149 - protein, 59 aa, Mus musculus
PAAAAGAKKAKSPKKAKATKAKKAPKSPAKAKTVKPKAAKPKTSKPKAAKPKKTAAKKK

SEQ ID NO: 150 - protein, 80 aa, Homo sapien
MTEYKLVVVGAGGVGKSALTIQLIQNHFVDEYDPTIEDSYRKQVVIDGETCLLDILDTAGQEEYSAMRDQYMRTGEGFLC

SEQ ID NO: 151 - protein, 80 aa, Mus musculus; identical to SEQ ID NO: 150

SEQ ID NO: 152 - protein, 80 aa, Danio rerio; identical to SEQ ID NO: 150

SEQ ID NO: 153 - protein, 78 aa, Homo sapien
GLRVPGNIVYSSLCGLGSEKGREAATSTLGGLGFSSERNPEMQFKPNTPETVEASAVSGKPPNGFSAIYKTPPGIQKS

SEQ ID NO: 154 - protein, 78 aa, Mus musculus
GLRVPGNIVYSGLCGLGSEKGREATPSSLSGLGFSSERNPEMQFKPNTPETVEASAVSGKPPNGFSAIYKTPPGIQKS

SEQ ID NO: 155 - protein, 62 aa, Danio rerio
SLRVHGGMVYPPGIQTLQSEKGYVGDRIPNLLYKPDVSLDSRKTANSFVGLYKSSPPGIQKP

SEQ ID NO: 156 - protein, 72 aa, Homo sapien
AVATAEALGLDRPASDKQSPLNINGASYLRLPWVNPYMEGATPAIYPFLDSPNKYSLNMYKALLPQQSYSLA

SEQ ID NO: 157 - protein, 72 aa, Mus musculus
AVATAESLGLDRPASDKQSPLNINGASYLRLPWVNPYMEGATPAIYPFLDSPNKYSLNMYKALLPQQSYGLA

SEQ ID NO: 158 - protein, 77 aa, Danio rerio
LIVPGAGADTLGLDRRVVPGDKPPELSLNGGSSYLRVPSWMSPYHETGMYPFLDSSKYAALNMYKASILSQPPPYLP

SEQ ID NO: 159 - protein, 79 aa, Homo sapien
TAVQDRKDGSSPPLLEKQTVTKDVTDKPLDLSSKVVDVDASKADHMKKMAPTVLVHSRAGSGLVLSGSEIPKETLSPPG

SEQ ID NO: 160 - protein, 79 aa, Mus musculus
TTVQDRKDGGSPPLLEKQTVTKDVTDKPLDLSSKVVDADASKGDHMKKMAPTVLVHSRAASGLVLSGSEIPKETLSPPG

SEQ ID NO: 161 - protein, 79 aa, Danio rerio
GSSSPVKTSSDKSPQQGPITKDPADKPLDLSAKIMEFEGHTNGYPSKLEALAKLGYSPAARYGLPPNRELLKETLSPSS

SEQ ID NO: 162 - protein, 75 aa, Homo sapien
NSEKPSGKRLCKTKHLIPQESRRGLPLTGEYYVENADGKVTVRRFRKRPEPSSDYDLSPAKQEPKPFDRLQQLLP

SEQ ID NO: 163 - protein, 76 aa, Mus musculus
NIEKPSGKRLCKTKHLIPQESRRSLQITGDYYVENTDTKMTVRRFRKRPEPSSDYDLSPPAKQEPKPFDRLQQLLP

SEQ ID NO: 164 - protein, 79 aa, Danio rerio
PEKPKGKRQCKTKHLSQQERRMLLQSTNQECPELSPAHQHKVVKCSAQKRPALLSNYESSPIKPHPPSPAYQPAASPTP

SEQ ID NO: 165 - protein, 79 aa, Homo sapien; identical to SEQ ID NO: 27

SEQ ID NO: 166 - protein, 80 aa, Mus musculus; identical to SEQ ID NO: 28

SEQ ID NO: 167 - protein, 21 aa, Danio rerio
MSTEDFIGGTAESGKMDTTET

SEQ ID NO: 168 - protein, 78 aa, Homo sapien; identical to SEQ ID NO: 30

SEQ ID NO: 169 - protein, 78 aa, Mus musculus; identical to SEQ ID NO: 31

SEQ ID NO: 170 - protein, 64 aa, Danio rerio; identical to SEQ ID NO: 32

SEQ ID NO: 171 - protein, 75 aa, Homo sapien
MFFNTKFFGLQTAEEHMQLSFTNVVRQSRKCTTPRGTTKVVSIRYYAPVRQRKGRDTGPGKRKREDEAPILEQRE

SEQ ID NO: 172 - protein, 74 aa, Mus musculus
MFFNTKFFGLQTAEEHMQLSFTNVVRQSRKCTTPRGTTKVVSIRYYAPVRQRKGRDTGPGKRKREDETILEQRE

SEQ ID NO: 173 - protein, 72 aa, Danio rerio
IYFFTKYFNYRTAEQHRLLSFGHIVRCSRSKGNTKVACLRFYPPKDEADGVPAKRRKEEGEEEDETVYEIKE

SEQ ID NO: 174 - protein, 77 aa, Homo sapien
NRMNPLRCPVKFYEFYLSKCPESLRTRNDVFYLQPERSCIAESPLWYSVIPMDRSMLESMLNRILAVREIYEELGRP

SEQ ID NO: 175 - protein, 77 aa, Mus musculus; identical to SEQ ID NO: 174

SEQ ID NO: 176 - protein, 80 aa, Danio rerio
NSDNPLRCPVRLYEFYLSKCSPSVRQRTTDFYLSPERSCVPNSPMWFSISALSDEALNSMLTRILTVRELHLDTEKTPAD

SEQ ID NO: 177 - protein, 72 aa, Homo sapien
QSLQLDCVAVPSSRSNSATEQPGSLHSSQGLGMGPVEESWFAPSLEHPQEENEPSLQSKLQDEANYHLYGSR

SEQ ID NO: 178 - protein, 70 aa, Mus musculus
FSLQHDCVPLPPSRSNSEQPGSLHSSQGLQMGPVEESWFSSSPEYPQDENDRSVQAKLQEEASYHAFGIF

SEQ ID NO: 179 - protein, 76 aa, Danio rerio
KLLNTNALIPEDPSLCSRDSPTPLRSSDIGPVEASIEDLSFRSCEDSVLEADATPSCPNLELKLEQEYNYHKFGSR

SEQ ID NO: 180 - protein, 74 aa, Homo sapien
MDRQTKQQPRQNVAYNREEERRRRVSHDPFAQQRPYENFQNTEGKGTAYSSAASHGNAVHQPSGLTSQPQVLYQ

SEQ ID NO: 181 - protein, 71 aa, Mus musculus
AEKQTKPQPRQNEAYNREEERKRRVSHDPFAQQRARENIKSAGARGHSDPSTTSRGIAVQQLSWPATQTVW

SEQ ID NO: 182 - protein, 69 aa, Danio rerio
IVDQSDGSMWSPAQQPASSTDASSQMSSVKSWTKPSAPSTEEELYPRAAASFDSLHRPELRSQFSVPQS

SEQ ID NO: 183 - protein, 80 aa, Homo sapien
NYMEIGGTSSSLLDSTNTNFKEEPAAKYQAIFDNTTSLTDKHLDPIRENLGKHWKNCARKLGFTQSQIDEIDHDYERDGL

SEQ ID NO: 184 - protein, 76 aa, Mus musculus
NYMDVGLNSQPPNNTCKEESTSRHQAIFDNTTSLTDEHLNPIRENLGRQWKNCARKLGFTESQIDEIDHDYERDGL

SEQ ID NO: 185 - protein, 80 aa, Danio rerio
NTMNLRISDSSPYSSLSTTGSTTSRYKELLLMYESHTVTESHLELVRQNVGANWKQVARKLGLSEIDVETIEHDYDRDGL

SEQ ID NO: 186 - protein, 229 aa, Homo sapien
GHGPFSHMFDGRFIPLARPEVKWTHEQGSVMMFEHLINSNGIKPVMEQYGLIPEEDICFIKEQIVGPLESPVEDSLWPYKGRPENKSFLYEIVSNKRNGIDVDKWDYFARDCHHLGIQNNFDYKRFIKFARVCEVDNELRICARDKEVGNLYDMFHTRNSLHRRAYQHKVGNIIDTMITDAFLKADDYIEITGAGGKKYRISTAIDDMEAYTKLTDNIFLEILYSTDPK

SEQ ID NO: 187 - protein, 240 aa, Mus musculus
GHGPFSHMFDGRFIPRARPEKKWKHEQGSIEMFEHLVNSNELKLVMKNYGLVPEEDITFIKEQIMGPPITPVKDSLWPYKGRPATKSFLYEIVSNKRNGIDVDKWDYFARDCHHLGIQNNFDYKRFIKFARICEVEYKVKEDKTYIRKVKHICSREKEVGNLYDMFHTRNCLHRRAYQHKISNLIDIMITDAFLKADPYVEITGTAGKKFRISTAIDDMEAFTKLTDNIFLEVLHSTDPQ

SEQ ID NO: 188 - protein, 80 aa, Homo sapien; identical to SEQ ID NO: 150

SEQ ID NO: 189 - protein, 80 aa, Mus musculus; identical to SEQ ID NO: 150

SEQ ID NO: 190 - protein, 80 aa, Danio rerio; identical to SEQ ID NO: 150

SEQ ID NO: 191 - protein, 80 aa, Homo sapien
MAAFGILSYEHRPLKRPRLGPPDVYPQDPKQKEDELTALNVKQGFNNQPAVSGDEHGSAKNVSFNPAKISSNFSSIIAEK

SEQ ID NO: 192 - protein, 80 aa, Mus musculus
MAAFGILSYEHRPLKRLRLGPPDVYPQDPKQKEDELTALNVKQGFNNQPAVSGDEHGSAKNVNFNPAKISSNFSSIIAEK

SEQ ID NO: 193 - protein, 80 aa, Danio rerio
MAAFGVLSYEHRPLKRPRLGPPDVYPQDPKQKEDELTALNVKQGFNNQPAVSGDEHGSAKNVNFNSSKISSNFSSIIAEK

SEQ ID NO: 194 - protein, 80 aa, Homo sapien
QAQSSAIQAPRSPRLGRARSPSPCPFRSSSQPPGRVLVQGARSEERRTKSWGEQCPETSGTDSGRKGGPSLCSSQVKKGM

SEQ ID NO: 195 - protein, 80 aa, Mus musculus
QAQSSAIQAPRSPRLGRARSPSPCPFRSSSQPPERVLAPCSPSEERRTKSWGEQCTETPDTNSGRRSRLSTHPSKDKEGV

SEQ ID NO: 196 - protein, 77 aa, Homo sapien
KPFLRKACSPSNIPAVIITDMGTQEDGALEETQGSPRGNLPLRKLSSSSASSTGFSSSYEDSEEDISSDPERTLDPN

SEQ ID NO: 197 - protein, 80 aa, Mus musculus
KASLKEACSPSNIPAIPAVIITDMGAQEDGGLEEIQGSPRGPLPLRKLSSSSASSTGFSSSYDDSEEDISSDPERTLDPN

SEQ ID NO: 198 - protein, 480 aa, Homo sapien; identical to SEQ ID NO: 18

SEQ ID NO: 199 - protein, 480 aa, Mus musculus; identical to SEQ ID NO: 18

SEQ ID NO: 200 - protein, 80 aa, Homo sapien
RPILRPRKYPNRPSKTPVHERPYPCPAEGCDRRFSRSDELTRHIRIHTGHKPFQCRICMRNFSRSDHLTTHIRTHTGEKP

SEQ ID NO: 201 - protein, 80 aa, Mus musculus; identical to SEQ ID NO: 200

SEQ ID NO: 202 - protein, 80 aa, Danio rerio; identical to SEQ ID NO: 200

(19) United States
(12) Patent Application Publication
(10) Pub. No.: US 2014/0364439 A1
(43) Pub. Date: Dec. 11, 2014

(54) MARKERS ASSOCIATED WITH CHRONIC LYMPHOCYTIC LEUKEMIA PROGNOSIS AND PROGRESSION

(71) Applicants: The Broad Institute, Inc., Cambridge, MA (US); Dana-Farber Cancer Institute, Inc., Boston, MA (US)

(72) Inventors: Catherine J Wu, Brookline, MA (US); Gad Getz, Belmont, MA (US)

(73) Assignees: The Broad Institute, Inc., Cambridge, MA (US); Dana-Farber Cancer Institute, Inc., Boston, MA (US)

(21) Appl. No.: 14/362,648

(22) PCT Filed: Dec. 7, 2012
(86) PCT No.: PCT/US2012/068633

§ 371 Date: Jun. 4, 2014

Related U.S. Application Data
(60) Provisional application No. 61/567,941, filed on Dec. 7, 2011.

(51) Int. Cl. C12Q 1/68

(52) U.S. Cl. CPC C12Q 1/6886; C12Q 2600/106; C12Q 2600/156
USPC 514/254.1; 435/6.11; 514/387; 514/450

(57) ABSTRACT

The present invention provides methods and devices related to markers (or biomarkers) associated with chronic lymphocytic leukemia (CLL). Examples of these markers include drivers of CLL progression. The invention contemplates, inter alia, detecting the clonal, including subclonal, profile of CLL in a subject and the presence (or absence) of subclonal driver mutations, and utilizing this information in predicting disease progression, need, timing and/or nature of treatment regimen, and likelihood and frequency of relapse.

### RELATED APPLICATIONS

This application claims the benefit under 35 U.S.C. §119(e) of U.S. Provisional Application No. 61/567,941, filed Dec. 7, 2011, the entire contents of which are incorporated by reference herein.

### FEDERALLY SPONSORED RESEARCH

This invention was made with U.S. Government support under grant number 1RO1HL103532-01 from the NHLBI and grant number 1RO1CA155010-01A1 from the NCI. Accordingly, the U.S. Government has certain rights in this invention.

### FIELD OF THE INVENTION

The present invention provides methods and devices for prognosing chronic lymphocytic leukemia (CLL) using one or more markers, as well methods of treating CLL using for example a modulator of SF3B1 activity.

### BACKGROUND OF THE INVENTION

Chronic lymphocytic leukemia (CLL) remains incurable and displays vast clinical heterogeneity despite a common diagnostic immunophenotype (surface expression of CD19+CD20+_{dim}CD5+CD23+ and sIgM_{dim}). While some patients experience an indolent disease course, approximately half have steadily progressive disease leading to significant morbidity and mortality (Zenz, Nat Rev Cancer, 2010, 10:37-50). Our ability to predict a more aggressive disease course has improved through the use of biologic markers (such as presence of somatic hypermutation of the immunoglobulin heavy chain variable region [IGHV status] and ZAP70 expression), and detection of cytogenetic abnormalities (such as deletions in chromosomes 11q, 13q, and 17p and trisomy 12) (Rassenti, N Engl J Med, 2004, 351:893-901; Dohner, N Engl J Med, 2000, 343:1910-6). Still, prediction of disease course is not highly reliable. Accordingly a need exists for the identification of biomarkers that can predict aggressive disease progression in patients with CLL.

### SUMMARY OF THE INVENTION

The invention provides, inter alia, prognostic factors for chronic lymphocytic leukemia (CLL). An example of such a prognostic factor is SF3B1. According to some aspect of the invention, it has been found unexpectedly that the presence of a SF3B1 mutation in a CLL sample indicates a poor prognosis. Detection of SF3B1 mutations may dictate, in some instances, an altered treatment, including but not limited to an aggressive treatment. The invention contemplates integrating SF3B1 mutation status into predictive and prognostic algorithms that currently use other markers, given the now recognized value of SF3B1 as an independent prognostic factor. SF3B1 mutation status can be used together with other factors, such as ZAP70 expression status and mutated IGVH status, to more accurately determine disease progression and likelihood of response to treatment, among other things. Other such prognostic factors include HIST1H1E, NRAS, BCOR, RIPK1, SAMHD1, KRAS, MED12, ITPKB, and EGR2.

In one aspect, the invention provides methods of determining a treatment regimen for a subject having CLL by identifying a mutation in the SF3B1 gene in a subject sample. The presence of one or more mutations in the SF3B1 gene may indicate that the subject should receive an alternative treatment regimen (compared to a prior treatment regimen administered to the patient). In some embodiments, the presence of one or more mutations in the SF3B1 gene indicates that the subject should receive an aggressive treatment regimen (for example a treatment that is more aggressive than a prior treatment administered to the patient). In some embodiments, the presence of one or more mutations in the SF3B1 gene indicates that the subject should receive a treatment that acts through a different mechanism than a prior treatment or a modality that is different from a prior treatment.

In another aspect, the invention provides methods of determining whether a subject having CLL would derive a clinical benefit of early treatment by identifying a mutation in the SF3B1 gene in a subject sample. The presence of one or more mutations in the SF3B1 gene indicates that the subject would derive a clinical benefit of early treatment.

In a further aspect, the invention provides methods predicting survivability of a subject having CLL by identifying a mutation in the SF3B1 gene in a subject sample. The presence of one or more mutations in the SF3B1 gene indicates the subject is less likely to survive or has a poor clinical prognosis.

Also included in the invention is method of identifying a candidate subject for a clinical trial for a treatment protocol for CLL by identifying a mutation in the SF3B1 gene in a subject sample. The presence of one or more mutations in the SF3B1 gene indicates that the subject is a candidate for the clinical trial.

In some embodiments, the mutation is a missense mutation. In some embodiments, the mutation is a R625L, a N626H, a K700E, a G740E, a K741N or a Q903R mutation in the SF3B1 polypeptide. In some embodiments, the mutation is a E622D, a R625G, a Q659R, a K666Q, a K666E, and a G742D mutation in the SF3B1 polypeptide. It is to be understood that the invention contemplates detection of nucleic acid mutations that correspond to the various amino acid mutations recited herein. In some embodiments, the mutation in the SF3B1 gene is within exons 14-17 of the SF3B1 gene.

In some embodiments, the method further comprises detecting at least one other CLL-associated marker. In some embodiments, the at least one other CLL-associated marker is mutated IGVH status or ZAP70 expression status.

In some embodiments, the method further comprises detecting (or identifying) at least one CLL-associated chromosomal abnormality. In some embodiments, the at least one CLL-associated chromosomal abnormality is selected from the group consisting of 8p deletion, 11q deletion, 13q deletion, 17p deletion, trisomy 12, monosomy 13, and rearrangements of chromosome 14.

The invention further contemplates methods related to those recited above but wherein mutations in one or more of HIST1H1E, NRAS, BCOR, RIPK1, SAMHD1, KRAS, MED12, ITPKB, and EGR2 genes are analyzed.

Any of the foregoing methods may further comprise analyzing genomic DNA for the presence of mutations in one or more of TP53, ATM, MYD88, NOTCH1, DDX3X, ZMYM3, FBXW7, XPO1, CHD2, and POT1.

In yet another aspect the invention provides methods of treating or alleviating a symptom of CLL by administering to a subject a compound that modulates SF3B1. Such a compound may inhibit or activate SF3B1 activity or may alter SF3B1 expression. The compound may be, for example, spliceostatin, E7107, or pladienolide.

In another aspect, the invention provides a kit comprising (i) a first reagent that detects a mutation in a SF3B1 gene; (ii) optionally, a second reagent that detects at least one other CLL-associated marker; (iii) optionally, a third reagent that detects at least one CLL-associated chromosomal abnormality; and (iv) instructions for their use. The mutations in (i), (ii), and (iii) may be any of the foregoing recited mutations. The invention further provides other related kits in which the first reagent detects mutations in a risk allele selected from the group consisting of HIST1H1E, NRAS, BCOR, RIPK1, SAMHD1, KRAS, MED12, ITPKB, and EGR2. The second reagent may be a reagent that detects mutations in TP53, ATM, MYD88, NOTCH1, DDX3X, ZMYM3, FBXW7, XPO1, CHD2, or POT1. The third reagent may be a reagent that detects a 8p deletion, 11q deletion, 13q deletion, 17p deletion, trisomy 12, monosomy 13, or a rearrangement of chromosome 14. The kit may comprise one or more first reagents (specific for the same or different risk alleles), one or more second reagents (specific for the same or different risk alleles), and one or more third reagents (specific for the same or different risk alleles).

In some embodiments, the first, second and third reagents are polynucleotides that are capable of hybridizing to the genes or chromosomes of (i), (ii) and/or (iii), wherein said polynucleotides are optionally linked to a detection label. The binding pattern of these polynucleotides denotes the presence or absence of the above-noted mutations.

The invention is further premised in part on the discovery that the clonal (including subclonal) profile of a CLL has independent prognostic value. It has been found that the presence of particular mutations, referred to herein as drivers, in CLL subclones is indicative of more rapid disease progression, greater likelihood of relapse, and shorter remission times. The ability to analyze a CLL sample for the presence of subclonal populations and more importantly drivers in the subclonal populations informs the subject and the medical practitioner about the likely disease course, and thereby influences decisions relating to whether to treat a subject or to delay treatment of the subject, the nature of the treatment (e.g., relative to prior treatment), and the timing and frequency of the treatment.

Some aspects of this disclosure therefore relate to the surprising discovery that the clonal heterogeneity of CLL in a subject is prognostic of the course of the disease, and informs decisions regarding treatment. In some aspects, the disclosure provides novel, independent prognostic markers of CLL. The invention provides methods and apparati for detection of one or more of these independent prognostic factors. In some aspects, the presence of one or more of these independent prognostic markers in a CLL sample, and particularly in a subclonal population, alone or in combination with other CLL prognostic markers whether or not in subclonal populations, indicates the severity or aggressiveness of the disease, and informs the type, timing, and degree of treatment to be prescribed for a patient.

These independent prognostic factors include mutations in a risk allele selected from the group consisting of SF3B1, HIST1H1E, NRAS, BCOR, RIPK1, SAMHD1, KRAS, MED12, ITPKB, EGR2, DDX3X, ZMYM3, FBXW7, ATM, TP53, MYD88, NOTCH1, XPO1, CHD2, and POT1, and mutations that are selected from the group consisting of del(8p), del(13q), del(11q), del(17p), and trisomy 12. Any combination of two or more of these mutations may be used, in some methods of the invention. In some embodiments where two or more mutations are analyzed, at least one of those mutations is selected from the group consisting of HIST1H1E, NRAS, BCOR, RIPK1, SAMHD1, KRAS, MED12, ITPKB, and EGR2, and optionally also including SF3B1.

In some embodiments, the independent prognostic factors include subclonal mutations in any one of HIST1H1E, NRAS, BCOR, RIPK1, SAMHD1, KRAS, MED12, ITPKB, EGR2, DDX3X, ZMYM3, FBXW7, NOTCH1, XPO1, CHD2, POT1, del(8p), del(11q), and del(17p). Additional independent prognostic factors include subclonal mutations in SF3B1, MYD88, and TP53 and subclonal del(13q) and subclonal trisomy 12.

In another aspect, the invention provides a method comprising (a) analyzing genomic DNA in a sample obtained from a subject having or suspected of having CLL for the presence of mutation in a risk allele, (b) determining whether the mutation is clonal or subclonal (i.e., whether the mutation is present in a clonal population of CLL cells or a subclonal population of CLL cells), and optionally (c) identifying the subject as a subject at elevated risk of having CLL with rapid disease progression if the mutation is a driver event and subclonal.

In some embodiments, the risk allele is selected from SF3B1, HIST1H1E, NRAS, BCOR, RIPK1, SAMHD1, KRAS, MED12, ITPKB, EGR2, TP53, ATM, MYD88, NOTCH1, DDX3X, ZMYM3, FBXW7, XPO1, CHD2, and POT1. In some embodiments, the risk allele is selected from SF3B1, HIST1H1E, NRAS, BCOR, RIPK1, SAMHD1, KRAS, MED12, ITPKB, EGR2, TP53, MYD88, NOTCH1, DDX3X, ZMYM3, FBXW7, XPO1, CHD2, and POT1. In some embodiments, the risk allele is selected from HIST1H1E, NRAS, BCOR, RIPK1, SAMHD1, KRAS, MED12, ITPKB, EGR2, NOTCH1, DDX3X, ZMYM3, FBXW7, XPO1, CHD2, and POT1. In some embodiments, the risk allele is selected from HIST1H1E, NRAS, BCOR, RIPK1, SAMHD1, KRAS, MED12, ITPKB, and EGR2.

In some embodiments, the risk allele is selected from del(8p), del(13q), del(11q), del(17p), and trisomy 12. In some embodiments, the risk allele is selected from del(8p), del(11q), and del(17p).

In some embodiments, the method comprises analyzing genomic DNA for (a) a mutation in one or more risk alleles selected from the group consisting of SF3B1, HIST1H1E, NRAS, BCOR, RIPK1, SAMHD1, KRAS, MED12, ITPKB, EGR2, DDX3X, ZMYM3, FBXW7, ATM, TP53, MYD88, NOTCH1, XPO1, CHD2, and POT1, and/or (b) a mutation that is selected from the group consisting of del(8p), del(13q), del(11q), del(17p), and trisomy 12.

In some embodiments, the method comprises analyzing genomic DNA for (a) a mutation in one or more risk alleles selected from the group consisting of SF3B1, HIST1H1E, NRAS, BCOR, RIPK1, SAMHD1, KRAS, MED12, ITPKB, EGR2, DDX3X, ZMYM3, FBXW7, TP53, MYD88, NOTCH1, XPO1, CHD2, and POT1, and/or (b) a mutation that is selected from the group consisting of del(8p), del(13q), del(11q), del(17p), and trisomy 12.

In some embodiments, the method comprises analyzing genomic DNA for (a) a mutation in one or more risk alleles selected from the group consisting of HIST1H1E, NRAS, BCOR, RIPK1, SAMHD1, KRAS, MED12, ITPKB, EGR2, DDX3X, ZMYM3, FBXW7, NOTCH1, XPO1, CHD2, and POT1, and/or (b) a mutation that is selected from the group consisting of del(8p), del(11q), and del(17p).

In some embodiments, the method comprises analyzing genomic DNA for a mutation in one or more risk alleles selected from the group consisting of HIST1H1E, NRAS, BCOR, RIPK1, SAMHD1, KRAS, MED12, ITPKB, and EGR2.

In some embodiments, the method comprises analyzing genomic DNA for the presence of a mutation in one or more of at least 2 risk alleles chosen from the group consisting of SF3B1, HIST1H1E, NRAS, BCOR, RIPK1, SAMHD1, KRAS, MED12, ITPKB, EGR2, DDX3X, ZMYM3, FBXW7, ATM, TP53, MYD88, NOTCH1, XPO1, CHD2, POT1, del(8p), del(13q), del(11q), del(17p), and trisomy 12.

In some embodiments, the method comprises analyzing genomic DNA for the presence of a mutation in one or more of at least 2 risk alleles chosen from the group consisting of SF3B1, HIST1H1E, NRAS, BCOR, RIPK1, SAMHD1, KRAS, MED12, ITPKB, EGR2, DDX3X, ZMYM3, FBXW7, TP53, MYD88, NOTCH1, XPO1, CHD2, POT1, del(8p), del(13q), del(11q), del(17p), and trisomy 12.

In some embodiments, the method comprises analyzing genomic DNA for the presence of a mutation in one or more of at least 2 risk alleles chosen from the group consisting of HIST1H1E, NRAS, BCOR, RIPK1, SAMHD1, KRAS, MED12, ITPKB, EGR2, DDX3X, ZMYM3, FBXW7, NOTCH1, XPO1, CHD2, POT1, del(8p), del(11q), and del(17p).

In some embodiments, the method comprises analyzing genomic DNA for the presence of a mutation in one or more of at least 2 risk alleles chosen from the group consisting of HIST1H1E, NRAS, BCOR, RIPK1, SAMHD1, KRAS, MED12, ITPKB, and EGR2.

At least 2 intends and embraces at least 3, at least 4, at least 5, at least 6, at least 7, at least 8, at least 9, or at least 10. In some embodiments, the at least 2, at least 3, at least 4, at least 5, at least 6, at least 7, at least 8, or at least 9 of the risk alleles analyzed are selected from the group consisting of HIST1H1E, NRAS, BCOR, RIPK1, SAMHD1, KRAS, MED12, ITPKB, and EGR2.

In another aspect, the invention provides a method comprising (a) detecting a mutation in genomic DNA from a sample obtained from a subject having or suspected of having CLL, (b) detecting clonal and/or subclonal populations of cells carrying the mutation, and optionally (c) identifying the subject as a subject at elevated risk of having CLL with rapid disease progression if the mutation is a driver event present in a subclonal population of cells.

In another aspect, the invention provides a method comprising detecting, in genomic DNA of a sample from a subject having or suspected of having CLL, presence or absence of a mutation in a risk allele selected from the group consisting of SF3B1, HIST1H1E, NRAS, BCOR, RIPK1, SAMHD1, KRAS, MED12, ITPKB, EGR2, DDX3X, ZMYM3, FBXW7, ATM, TP53, MYD88, NOTCH1, XPO1, CHD2, and POT1 and/or a mutation that is selected from the group consisting of del(8p), del(13q), del(11q), del(17p), and trisomy 12, and determining if the mutation, if present, is in a subclonal population of the CLL sample. In some embodiments, the mutation is in a risk allele selected from the group consisting of SF3B1, HIST1H1E, NRAS, BCOR, RIPK1, SAMHD1, KRAS, MED12, ITPKB, EGR2, DDX3X, ZMYM3, FBXW7, TP53, MYD88, NOTCH1, XPO1, CHD2, and POT1. In some embodiments, the mutation is in a risk allele selected from the group consisting of HIST1H1E, NRAS, BCOR, RIPK1, SAMHD1, KRAS, MED12, ITPKB, EGR2, DDX3X, ZMYM3, FBXW7, NOTCH1, XPO1, CHD2, and POT1. In some embodiments, the mutation is in a risk allele selected from the group consisting of HIST1H1E, NRAS, BCOR, RIPK1, SAMHD1, KRAS, MED12, ITPKB, and EGR2. In some embodiments, the mutation is selected from the group consisting of del(8p), del(11q), and del(17p).

Various embodiments apply equally to the foregoing methods and these are recited now for brevity.

The methods of the invention are typically performed on a sample obtained from a subject and are in vitro methods. In some embodiments, the sample is obtained from peripheral blood, bone marrow, or lymph node tissue. In some embodiments, the genomic DNA is analyzed using whole genome sequencing (WGS), whole exome sequencing (WES), single nucleotide polymorphism (SNP) analysis, or deep sequencing, targeted gene sequencing, or any combination thereof. These techniques may be used in whole or in part to detect the mutations and the subclonal nature of the mutations.

In some embodiments, the methods further comprise treating a subject identified as a subject at elevated risk of having CLL with rapid disease progression. In some embodiments, the methods further comprise delaying treatment of the subject for a specified or unspecified period of time (e.g., months or years). In some embodiments, the methods are performed before and after treatment. In some embodiments, the methods are repeated every 6 months or if there is a change in clinical status. In some embodiments, genomic DNA is analyzed for mutations in more than one risk allele.

In some embodiments, the method analyzes genomic DNA for mutations in two or more of the HIST1H1E, NRAS, BCOR, RIPK1, SAMHD1, KRAS, MED12, ITPKB, and EGR2 genes, including three or more, four or more, five or more, six or more, seven or more, eight or more, or all nine of the genes.

Any of the foregoing subclonal driver methods may be combined with detection of mutations in other genes (or gene loci or chromosomal regions) regardless of whether these latter mutations are clonal or subclonal. For example, the methods may comprise detection of mutations in one or more of TP53, ATM, MYD88, SF3B1, NOTCH1, DDX3X, ZMYM3, FBXW7, XPO1, CHD2, POT1, del(8p), del(13q), del(11q), del(17p), and trisomy 12, without determining the clonal or subclonal nature of such mutations.

In another aspect, the invention provides a kit comprising reagents for detecting (1) mutations in one or more risk alleles selected from the group consisting of SF3B1, HIST1H1E, NRAS, BCOR, RIPK1, SAMHD1, KRAS, MED12, ITPKB, EGR2, DDX3X, ZMYM3, FBXW7, XPO1, CHD2, POT1, TP53, MYD88, NOTCH1, and ATM, and/or (2) mutations selected from the group consisting of del(8p), del(13q), del(11q), del(17p), or trisomy 12, in a sample obtained from a patient.

In another aspect, the invention provides a kit comprising reagents for detecting (1) mutations in one or more risk alleles selected from the group consisting of SF3B1, HIST1H1E, NRAS, BCOR, RIPK1, SAMHD1, KRAS, MED12, ITPKB, EGR2, DDX3X, ZMYM3, FBXW7, XPO1, CHD2, POT1, TP53, MYD88, and NOTCH1, and/or (2) mutations selected from the group consisting of del(8p), del(13q), del(11q), del(17p), or trisomy 12, in a sample obtained from a patient.

In another aspect, the invention provides a kit comprising reagents for detecting (1) mutations in one or more risk alleles selected from the group consisting of HIST1H1E, NRAS, BCOR, RIPK1, SAMHD1, KRAS, MED12, ITPKB, EGR2, DDX3X, ZMYM3, FBXW7, XPO1, CHD2, POT1, and NOTCH1, and/or (2) mutations selected from the group consisting of del(8p), del(11q), and del(17p), in a sample obtained from a patient.

The kit may comprise reagents for detecting on mutations in (1) or only mutations in (2), or any combination thereof. In some embodiments, the kit comprises reagents for detecting mutations in at least one, two, three, four, five, six, seven, eight, or nine risk alleles selected from the group consisting of HIST1H1E, NRAS, BCOR, RIPK1, SAMHD1, KRAS, MED12, ITPKB, and EGR2. In some embodiments, the kit is used to determine whether the mutation is a subclonal mutation. In some embodiments, the kit comprises instructions for determining whether the mutation is a subclonal mutation. In some embodiments, the subclonal mutation is at least one, two, three, four, five, six, seven, eight, nine or ten risk alleles selected from the group consisting of SF3B1, HIST1H1E, NRAS, BCOR, RIPK1, SAMHD1, KRAS, MED12, ITPKB, TP53, MYD88, NOTCH1, DDX3x, ZMYM3, FBXW7, XPO1, CHD2, POT1, and EGR2. In some embodiments, the kit comprises instructions for the prognosis of the patient based on presence or absence of subclonal mutations, wherein the presence of a subclonal mutation indicates the patient has an elevated risk of rapid CLL disease progression. The kits are therefore useful in determining prognosis of a patient with CLL.

Unless otherwise defined, all technical and scientific terms used herein have the same meaning as commonly understood by one of ordinary skill in the art to which this invention pertains. Although methods and materials similar or equivalent to those described herein can be used in the practice of the present invention, suitable methods and materials are described below. All publications, patent applications, patents, and other references mentioned herein are expressly incorporated by reference in their entirety. In cases of conflict, the present specification, including definitions, will control. In addition, the materials, methods, and examples described herein are illustrative only and are not intended to be limiting.

Other features and advantages of the invention will be apparent from and encompassed by the following detailed description and claims.

### BRIEF DESCRIPTION OF THE DRAWINGS

FIG. 1 shows significantly mutated genes in CLL. The 9 significantly mutated genes across 91 CLL samples are summarized. n—number of mutations per gene detected in 91 CLL samples. (%)—percent patients harboring the mutated gene. N—total territory in base pairs with sufficient sequencing coverage across 91 sequenced tumor/normal pairs. p- and q-values were calculated by comparing the probability of seeing the observed constellation of mutations to the background mutation rates calculated across the dataset.

FIG. 2 shows core signaling pathways in CLL. Genes in which mutations were identified are depicted within their respective core signaling pathways. The significantly mutated genes are indicated in dark grey, while mutations in other genes within a pathway are indicated in light. A list of the additional mutated pathway-associated genes is provided in Table 7.

FIG. 3 shows associations between gene mutations and clinical characteristics. The 91 CLL samples were sorted based on the Dohner hierarchy for FISH cytogenetics (Dohner, N Engl J Med, 2000, 343:1910-6) and were scored for presence or absence of mutations in the 9 significantly mutated genes as well as additional pathway-associated genes (scored in lighter shade), and for IGHV status (darker shade—mutated; white-unmutated; hatched-unknown). A list of the additional mutated pathway-associated genes is provided in Table 7. Associations between gene mutation status and FISH cytogenetics or IGHV status were calculated using the Fisher exact test, and corrected for multiple hypothesis testing (q<=0.1 for all comparisons shown).

FIG. 4 shows mutation in SF3B1 is associated with altered splicing in CLL. (A) Cox multivariable regression model analysis of significant factors contributing to earlier TTFT from the 91 genome/exome sequenced CLL samples. HR-hazards ratio. CI-confidence interval. (B) The relative amounts of spliced and unspliced spliceosome target mRNAs BRD2 and RIOK3 in normal CD19+ B (n=6) and CLL-B cells with wildtype (WT, n=17) or mutated SF3B1 (mut, n=13) were measured by quantitative PCR. The ratios of unspliced to spliced mRNAs were normalized to the percentage of leukemia cells per sample, and comparisons were calculated using the Wilcoxon rank sum test. Analysis of the 30 CLL samples based on presence or absence of del(11q) further revealed this result to be independent of del(11q) (see FIG. 10B).

FIG. 5 shows mutation rate is unrelated to treatment status in CLL patients. (A) Clinical summary of the 91 patients sequenced. (B) Mutation rate is similar between 61 chemotherapy-naïve and 30 chemo-treated CLL samples.

FIGS. 6A-F show mutations in SF3B1, FBXW7, DDX3X, NOTCH1 and ZMYM3 occur in evolutionarily conserved regions. For SF3B1, of the 14 novel mutations discovered in 91 CLL samples, all were localized to conserved regions of genes. Where available, alignments of gene sequences around each mutation are shown for human, mouse, zebrafish, C. elegans and S. pombe genes using sequences available at the USCS Genomic Bioinformatics website. A similar analysis was performed in the other significantly mutated genes.

FIG. 7 shows mutation types and locations in the 9 significantly mutated genes. (A-I) Type (missense, splice-site, nonsense) and location of mutations in the 9 significantly mutated genes discovered among the 91 CLL samples (top) compared to previously reported mutations in literature or in the COSMIC database (v76) (bottom). Dashed boxes in (B), (C) and (F) indicate mutations localizing to a discrete gene territory.

FIG. 8 shows mutations in genes that are pathway related to driver mutations occur in evolutionarily conserved locations. Where available, alignments of gene sequences around each mutation are shown for human, mouse, chicken and zebrafish, genes. These nucleotide sequences can be found at the USCS Genomic Bioinformatics website.

FIG. 9 shows mutation in SF3B1 is associated with earlier TTFT. (A) Percent samples harboring the SF3B1-K700E, MYD88-L265P or NOTCH1-P2514fs mutations, within the 78 exomes with known IGHV mutation status (U-unmutated; M-mutated), and the 82 extension set CLL samples with known IGHV mutation status. Mutations were detected by exome sequencing for the 78 samples in the discovery set and by Mass Sequenom genotyping for the 82 samples analyzed in the extension set. (B) Kaplan-Meier curves of the probability of time-to-first-therapy for 91 patients included in our discovery set (left), and for 101 patient samples that underwent genotyping of the SF3B1-K700E mutation in the extension set (right). Samples were categorized based on the presence or absence of del(11q) and the presence or absence of SF3B1 mutations. Patients with either del(11q) or SF3B1 mutation or both demonstrate significantly shorter time to first therapy as compared to all others (log-rank test).

FIG. 10 shows altered splicing in CLL is associated with mutation in SF3B1 but not del(11q). (A) Treatment with E7107, which targets the SF3b complex generates increased ratio of unspliced to spliced RIOK3 and BRD2 mRNA. Hela cells, normal CD19+ B cells and CLL cells were treated with E7107 for 4 hours. Unspliced (U) and spliced (S) BRD2 and RIOK3 were amplified by reverse transcription PCR and analyzed by agarose gel electrophoresis. (B) The relative amounts of spliced and unspliced BRD2 and RIOK3 mRNAs, measured by quantitative PCR, based on presence or absence of del(11q) and WT or mut SF3B1 are shown. The ratios of unspliced to spliced mRNAs were normalized to the percentage of leukemia cells per sample, and comparisons were calculated using the Wilcoxon rank sum test.

FIG. 11 shows the distribution of allelic fraction of 2348 coding mutations (535 synonymous, 1813 non-synonymous) detected from 91 sequenced CLL samples.

FIGS. 12A and B show significantly mutated genes and associated gene pathways in 160 CLL samples. (A) Mutation significance analysis, using the MutSig2.0 and GISTIC2.0 algorithms identifies recurrently mutated genes and recurrent sCNAs in CLL, respectively. Bold—significantly mutated genes identified in the previous CLL analysis discussed above (Wang et al., 2011). *—additional novel CLL genes identified in this experiment (also see FIG. 19). ‘n’—number of samples out of 160 CLLs harboring a mutation in a specific gene; ‘n_cosmic’—number of samples harboring a mutation in a specific gene at a site previously observed in the COSMIC database. (B) The significantly mutated genes fall into seven core signaling pathways, in which the genes play roles in DNA damage repair and cell-cycle control, Notch signaling, inflammatory pathways, Wnt signaling, RNA splicing and processing, B cell receptor signaling and chromatin modification. Darker shade—genes with significant mutation frequencies; lighter shade—additional pathway genes with mutations.

FIGS. 13A-D show that subclonal and clonal somatic single nucleotide variants (sSNVs) are detected in CLL in varying quantities based on age at diagnosis, IGHV mutation status, and treatment status (also see FIG. 20). (A) The analysis workflow. Whole-exome sequencing (WES) and SNP array data were collected from matched germline and tumor DNA and processed to identify recurrent driver events using MutSig2.0 and GISTIC2.0 (‘CLL driver events’, in darker shaded box). For the 149 samples that had matched WES and copy number data, the algorithm ABSOLUTE (Carter et al., 2012) was applied to provide estimates of cancer cell fraction (CCF). Mutations were classified as subclonal or clonal, as indicated, based on the probability that their CCF is greater than 0.95 (clonal). Inset—Histogram of the probability of being clonal for the entire set of sSNVs across 149 CLL samples. (B) A representative example of the transformations generated by ABSOLUTE (for sample CLL088). First, probability density distributions of allelic fractions for each mutation are plotted (representative peaks for sSNVs a, b and c shown in this example). Second, these data are converted to CCF (right panel), incorporating purity and local copy number information. The probability of the event being clonal (i.e., affecting >0.95 of cells) is represented by the shade of the event: lighter shade—high probability; darker shade—low probability. *—marks the allelic fraction of a clonal mutation at multiplicity of 1 (for example, a heterozygous mutation in a diploid region). (C) Comparison of the number of subclonal and clonal sSNVs per sample based on patient age at diagnosis and IGHV mutation status. (D) Comparison of the number of subclonal and clonal sSNVs per sample based on treatment status at time of sample collection (top panel). Cumulative distribution of the sSNVs by CCF is shown for samples from treated and untreated patients for all sSNVs (middle panel) and only driver sSNVs (bottom panel).

FIGS. 14A and B show the identification of earlier and later CLL driver mutations (also see FIG. 21). (A) Distribution of estimated cancer cell fraction (CCF) (bottom panel) and percent of the mutations classified as clonal (top panel-orange) or subclonal (top-blue) for each of the defined CLL drivers; *—drivers with q-values<0.1 for a higher proportion of clonal mutations compared with the entire CLL drivers set (Fisher exact test and FWER with the Bonferroni method). Het—heterozygous deletion; Hom—homozygous deletion. The analysis includes all recurrently mutated genes (see also FIG. 12A) with 3 or more events in the 149 samples, excluding sSNVs affecting the X chromosome currently not analyzable by ABSOLUTE, and also excluding indels in genes other than in NOTCH1. (B) All CLL samples with the early drivers MYD88 (left) or trisomy 12 (right) and at least 1 additional defined CLL driver (i.e. 9 of 12 samples with mutated MYD88; 14 of 16 tumors with trisomy 12) are depicted. Each dot denotes a separate individual CLL sample.

FIGS. 15A and B show the results of a longitudinal analysis of subclonal evolution in CLL and its relation to therapy (also see FIG. 22). Joint distributions of cancer cell fraction (CCF) values across two timepoints were estimated using clustering analysis. *—denotes a mutation that had an increase in CCF of greater than 0.2 (with probability>0.5). The dotted diagonal line represents y=x, or where identical CCF values across the two timepoints fall; the dotted parallel lines denote the 0.2 CCF interval on either side. Likely driver mutations were labeled. Six CLLs with no intervening treatment (A) and 12 CLLs with intervening treatment (B) were classified according to clonal evolution status, based on the presence of mutations with an increase of CCF>0.2. (C) Hypothesized sequence of evolution, inferred from the patients' WBC counts, treatment dates, and changes in CCF for 3 representative examples.

FIG. 16 shows genetic evolution and clonal heterogeneity results in altered clinical outcome. (A) Schema of the main clinical outcome measures that were analyzed: failure free survival from time of sample (FFS_Sample) and from initiation of first treatment after sampling (FFS_Rx). Within the longitudinally followed CLLs that received intervening treatment (12 of 18), shorter FFS_Rx was observed in CLL samples that (B) had evidence of genetic evolution (n=10) compared to samples with absent or minimal evolution (n=2; Fisher exact test), and that (C) harbored a detectable subclonal driver in the pretreatment sample (n=8) compared to samples with absent subclonal driver (n=4).

FIGS. 17A-D show that the presence of subclonal drivers mutations adversely impacts clinical outcome. (A) Analysis of genetic evolution and clonal heterogeneity in 149 CLL samples. The top panel—the total number of mutations (lighter shade) and the number of subclonal mutations (darker shade) per sample. Bottom panel—co-occurring driver mutations (y-axis) are marked per individual CLL sample (x-axis). Rows—CLL or cancer drivers (sSNVs in highly conserved sites in Cancer Gene Census genes) detected in the 149 samples. Greyscale spectrum (near white to black) corresponds to estimated cancer cell fraction (CCF); white boxes—not detected; patterned—CCF not estimated (genes on the X chromosome and indels other than in NOTCH1). (B-C) Subclonal drivers are associated with adverse clinical outcome. (B) CLL samples containing a detectable subclonal driver (n=68) exhibited shorter FFS_Sample compared to samples with absent subclonal drivers (n=81) (also see FIG. 23). (C) Subclonal drivers were associated with shorter FFS_Rx in 67 samples which were treated after sampling. (D) A Cox multivariable regression model designed to test for prognostic factors contributing to shorter FFS_Rx showed that presence of a subclonal driver was an independent predictor of outcome.

FIG. 18 shows a model for the stepwise transformation of CLL. The data provided herein indicate distinct periods in the life history of CLL. An increase in clonal mutations was observed in older patients and in the IGHV mutated subtype, likely corresponding to pre-transformation mutagenesis (A). Earlier and later mutations in CLL were identified, consistent with B cell-specific (B) and ubiquitous cancer events (C-D), respectively. Finally, clonal evolution and treatment show a complex relationship. Most untreated CLLs and a minority of treated CLLs maintain stable clonal equilibrium over years (C). However, in the presence of a subclone containing a strong driver, treatment may disrupt inter-clonal equilibrium and hasten clonal evolution (D).

FIGS. 19A-S show significantly mutated genes in 160 CLL samples, related to FIG. 12. (A-S) Type (missense, splice-site, nonsense) and location of mutations in the significantly mutated genes discovered among the 160 CLL samples (top) compared to previously reported mutations in literature or in the COSMIC database (v76) (bottom). Dashed boxes in A, C, D, J, O and P indicate mutations localizing to a discrete gene territory. Please refer to previous publication for mutation information for FBXW7 (Wang et al., 2011)

FIG. 20 shows mutation sites in 14 significantly mutated genes are localized to conserved regions of genes. Where available, alignments of gene sequences around each mutation are shown for human, mouse, zebrafish, C. elegans and S. pombe genes. The nucleotide sequences can be found at the website of USCS Genomic Bioinformatics.

FIG. 21 shows the results of whole exome sequencing allelic fraction estimates. Estimates are consistent with deep sequencing and RNA sequencing measurements, related to FIG. 13. (A) Comparison of ploidy estimates by ABSOLUTE with flow analyses for DNA content of 7 CLL samples and one normal B cell control (not analyzed by ABSOLUTE). Vertical lines indicate 95% confidence intervals of ploidy measurements by FACS. (B) Comparison of measurements of allelic fraction of 256 gene mutations detected by WES compared to detection using Fluidigm-based amplification following by deep sequencing (average 4200× coverage) using a MiSeq instrument. Significantly different estimates were assigned open circles. (C) Comparison of allelic fraction measured for 74 validated sites from 16 CLL samples by WES or RNA sequencing. (D) Comparison of mutational spectrum between subclonal and clonal sSNVs (detected in 149 CLLs). Rates were calculated as the fraction of the total number of sSNVs in the set with a particular mutation variant.

FIG. 22 shows graphs depicting the co-occurrence of mutations, related to FIG. 14. The commonly occurring mutations, sorted in the order of decreasing frequency of affected. The top panel—the total number of mutations (lighter shade) and the number of subclonal mutations (darker shade) per sample. Bottom panel—co-occurring CLL driver events (y-axis) are marked per individual CLL sample (x-axis). Greyscale spectrum (near white to black) corresponds to CCF; white boxes—no driver mutation identified; patterned—mutations whose CCF was not estimated (i.e., mutations involving the X chromosome and indels other than in NOTCH1, currently not evaluated with ABSOLUTE).

FIGS. 23A and B show the characterization of CLL clonal evolution through analysis of subclonal mutations at two timepoints in 18 patients, related to FIG. 15. (A-B) Unclustered results for 18 longitudinally studied CLLs, comparing CCF at two timepoints, * denotes a mutation with an increase in CCF greater than 0.2 (with probability>0.5). Six CLLs with no interval treatment (A) and 12 CLLs with intervening treatment (B) were classified as non-evolvers or evolvers, based on the presence of mutations with a statistically significant increase in CCF. (C) Deep sequencing validation of 6 of the 18 CLLs. For each set of samples, allelic frequency (AF) by WES (red) (with 95% CI by binofit shown by cross bars) is shown on the left and AF by deep sequencing (blue) (with 95% CI by binofit shown by cross bars) is shown on the right. Deep sequencing was performed to an average coverage of 4200×. (D) RNA pyrosequencing demonstrates a change in mRNA transcript levels that are consistent with changes in DNA allelic 4 frequencies. (E) Genetic changes correlate with transcript level of pre-defined gene sets expected to be altered as a result of the genetic lesion. These include change in expression level in the nonsense-mediated mRNA decay (NMD) pathway gene set, expected to be increased in association with splicing abnormalities such as SF3B1 mutations (data not shown). In addition, changes in expression level of the NRASQ61 gene set (data not shown) accompany the shift in allelic frequency for the NRAS mutations.

FIG. 24 shows a series of graphs demonstrating that the presence of a subclonal driver is associated with shorter FFS_Sample when added to known clinical high risk indicators (related to FIG. 17). FFS_Sample plots of the patient groups based on presence or absence of a subclonal driver (‘+/− SC driver’) and their (A)IGHV mutation status; (B) exposure to prior therapy; (C) presence or absence of del(11q) and (D) presence or absence of del(17p).

### DETAILED DESCRIPTION OF THE INVENTION

The invention is based, in part, upon the surprising discovery that patients with chronic lymphocytic leukemia (CLL) who harbor mutations in the SF3B1 gene and certain other genes demonstrate a significantly shorter time to first therapy, signifying a more aggressive disease course. This is particularly the case if such mutations are subclonal. Furthermore, a Cox multivariable regression model for clinical factors contributing to an earlier time to first therapy in a series of 91 CLL samples revealed that SF3B1 mutation was predictive of shorter time to requiring treatment, independent of other established predictive markers such as IGHV mutation, presence of del(17p) or ATM mutation. Accordingly, mutations in the SF3B1 and certain other genes are prognostic markers of disease aggressiveness in CLL patients.

Ninety-one CLL samples, consisting of 88 exomes and 3 genomes, representing the broad clinical spectrum of CLL were analyzed. Nine driver genes in six distinct pathways involved in pathogenesis of this disease were identified. These driver genes were identified as TP53, ATM, MYD88, SF3B1, NOTCH1, DDX3X, ZMYM3, and FBXW7. Moreover, novel associations with prognostic markers that shed light on the biology underlying this clinically heterogeneous disease were discovered.

These data led to several general conclusions. First, similar to other hematologic malignancies (Ley, Nature 2008; 456:66-72), the somatic mutation rate is lower in CLL than in most solid tumors (Fabbri, J Exp Med, 2011; Puente, Nature, 2011). Second, the rate of non-synonymous mutation was not strongly affected by therapy. Third, in addition to expected mutations in cell cycle and DNA repair pathways, genetic alterations were found in Notch signaling, inflammatory pathways and RNA splicing and processing. Fourth, driver mutations showed striking associations with standard prognostic markers in CLL, suggesting that particular combinations of genetic alterations may cooperate to drive malignancy.

A surprise was the finding that a core spliceosome component, SF3B1, is mutated in about 15% of CLL patients. Further analysis demonstrated that CLL samples with SF3B1 mutations displayed enhanced intron retention within two specific transcripts previously shown to be affected by compounds that disrupt SF3b spliceosome function (Kotake, Nat Chem Biol, 2007, 3:570-5; Kaida, Nat Chem Biol, 2007, 3:576-83). Studies of these compounds have suggested that rather than inducing a global change in splicing, SF3b inhibitors alter the splicing of a narrow spectrum of transcripts derived from genes involved in cancer-related processes, including cell-cycle control (p27, CCA2, STK6, MDM2) (Kaida, Nat Chem Biol, 2007, 3:576-83; Corrionero, Genes Dev 2011, 25:445-59; Fan, ACS Chem Biol, 2011), angiogenesis, and apoptosis (Massiello, FASEB J, 2006, 20:1680-2). These results suggest that SF3B1 mutations induce mistakes in splicing of these and other specific transcripts that affect CLL pathogenesis. Since mutations in SF3B1 are highly enriched in patients with del(11q), SF3B1 mutations may synergize with loss of ATM, a possibility further supported by the observation of 2 patients with point mutations in both ATM and SF3B1 without del(11q).

The invention is further premised, in part, on the discovery of additional novel CLL drivers. These drivers include mutations in risk alleles HIST1H1E, NRAS, BCOR, RIPK1, SAMHD1, KRAS, MED12, ITPKB, and EGR2.

The invention is further based, in part, on the discovery of the significance and impact of subclonal mutations, and particularly subclonal driver mutations such as subclonal SFB1 mutation, including SF3B1, in CLL on disease progression. As shown in the Examples, presence of a subclonal driver mutation (or event) was predictive of the clinical course of CLL from first diagnosis and then following therapy. In both instances, patients with subclonal driver mutations (otherwise referred to herein as subclonal drivers for brevity) had poorer clinical course as compared to patients without subclonal drivers. This discovery indicates that CLL disease course and treatment regimens can be informed by an analysis of subclonal mutation at the time of first presentation but also throughout the disease progression including before and after treatment or simply at staged intervals even in the absence of treatment. Significantly, the data show and the invention contemplates that the impact of certain mutations will vary depending on whether the mutation is present in a clonal population of the CLL or a subclonal population. Certain mutations, when present in subclonal populations, were found to be better predictors of clinical course and outcome than if they were present in clonal populations. Prior to these findings, the effect of any given mutation, when present subclonally, on disease progression was not recognized. Thus, the invention allows subclonal mutation profiles in a subject to be determined, thereby resulting in a more targeted, personalized therapy.

The invention contemplates that subclonal analysis can inform disease management and treatment including decisions such as whether to treat a subject (e.g., if a subclonal driver mutation is found), or whether to delay treatment and monitor the subject instead (e.g., if no subclonal driver mutation is found), when to treat a subject, how to treat a subject, and when to monitor a subject post-treatment for expected relapse. Prior to this disclosure, the impact of the frequency, identity and evolution of subclonal genetic alterations on clinical course was unknown.

Subclonal mutations in one or more of SF3B1, HIST1H1E, NRAS, BCOR, RIPK1, SAMHD1, KRAS, MED12, ITPKB, EGR2, TP53, ATM, MYD88, NOTCH1, DDX3X, ZMYM3, FBXW7, XPO1, CHD2, POT1, del(8p), del(13q), del(11q), del(17p), and trisomy 12 are of interest in some embodiments. Analysis of a genomic DNA sample for the presence (or absence) of mutation in any one, any two, any three, any four, any five, any six, any seven, any eight, any nine, any ten, any eleven, or more of these genes is contemplated by the invention, in any combination.

As described in the Examples in greater detail, Briefly, analysis of 160 matched CLL and germline DNA samples (including 82 of the 91 samples described above) was performed. These patients represented the broad spectrum of CLL clinical heterogeneity, and included patients with both low- and high-risk features based on established prognostic risk factors (ZAP70 expression, the degree of somatic hypermutation in the variable region of the immunoglobulin heavy chain (IGHV) gene, and presence of specific cytogenetic abnormalities). Somatic single nucleotide variations (sSNVs) present in as few as 10% of cancer cells were detected, and in total, 2,444 nonsynonymous and 837 synonymous mutations in protein-coding sequences were identified, corresponding to a mean (±SD) somatic mutation rate of 0.6±0.28 per megabase (range, 0.03 to 2.3), and an average of 15.3 nonsynonymous mutations per patient (range, 2 to 53).

Expansion of the sample cohort provided the sensitivity to detect 20 putative CLL cancer genes (q<0.1). These included 8 of the 9 genes identified in the 91 CLL sample cohort described above (TP53, ATM, MYD88, SF3B1, NOTCH1, DDX3X, ZMYM3, FBXW7). The 12 newly identified genes were mutated at lower frequencies, and hence were not detected in the subset of the 91 sequenced samples. Three of the 12 additional candidate driver genes were recently identified (XPO1, CHD2, and POT1) (Fabbri et al., J Exp Med. 208, 1389-1401 (2011); Puente et al., Nature. 475, 101-105. (2011)). The 9 remaining genes, NRAS, KRAS, BCOR, EGR2, MED12, RIPK1, SAMHD1, ITPKB, and HIST1H1E, represent additional novel candidate CLL drivers. Together, the 20 candidate CLL driver genes appear to fall into 7 core signaling pathways. Two new pathways were implicated by the analysis: B cell receptor signaling and chromatin modification.

Because recurrent chromosomal abnormalities have defined roles in CLL biology (Darner et al., N Engl J. Med. 343, 1910-1916 (2000); Klein et al., Cancer Cell. 17, 28-40 (2010)), loci that were significantly amplified or deleted were searched by analyzing somatic copy-number alterations (sCNAs). Analysis of 111 matched tumor and normal samples identified deletions in chromosome 8p, 13q, 11q, and 17p and trisomy of chromosome 12 as significantly recurrent events. Thus, based on sSNV and sCNA analysis, 20 mutated genes and 5 cytogenetic alterations were identified as CLL driver events.

Methods described herein were also used to determine whether the CLL driver events were clonal or subclonal. Overall, 1,543 clonal mutations (54% of all detected mutations, average of 10.3±5.5 mutations per sample) were identified, and a total of 1,266 subclonal sSNVs were detected in 146 of 149 samples (46%; average of 8.5±5.8 subclonal mutations per sample). Further analysis revealed that age and mutated IGHV status are associated with an increased number of clonal somatic mutations, subclonal mutations are increased with treatment, and the presence of subclonal driver mutations adversely impacts clinical outcome.

### CLL Disease Progression and Management

While generally considered incurable, CLL progresses slowly in most cases. Many people with CLL lead normal and active lives for many years—in some cases for decades. Because of its slow onset, early-stage CLL is, in general, not treated since it is believed that early CLL intervention does not improve survival time or quality of life. Instead, the condition is monitored over time to detect any change in the disease pattern.

Traditionally, the decision to start CLL treatment is taken when the patient's clinical symptoms or blood counts indicate that the disease has progressed to a point where it may affect the patient's quality of life.

Clinical “staging systems” such as the Rai 4-stage system and the Binet classification can help to determine when and how to treat the patient (Dohner, N Engl J Med, 2000, 343:1910-6).

Determining when to start treatment and by what means is often difficult; studies have shown there is no survival advantage to treating the disease too early. The invention provided herein is useful in determining whether and when to start treatment.

Accordingly, the invention provides methods of determining the aggressiveness of the disease course in subjects having or suspected of having CLL by identifying one or more mutations in the group consisting of SF3B1, NRAS, KRAS, BCOR, EGR2, MED12, RIPK1, SAMHD1, ITPKB, and HIST1H1E in a subject. Mutations in such genes are considered to be drivers (referred to interchangeably as CLL drivers), intending that they play a central role in the survival and continued growth of CLL cells in a subject. In some aspects, the disclosure provides methods for determining the aggressiveness of the disease course in subjects having or suspected of having CLL by determining whether a CLL driver is clonal or subclonal.

These methods are also useful for monitoring subjects undergoing treatments and therapies for CLL and for selecting therapies and treatments that would be efficacious in subjects having CLL, wherein selection and use of such treatments and therapies slow the progression of the cancer. More specifically, the invention provides methods of determining whether a patient with CLL will derive a clinical benefit of early treatment. Also included in the invention are methods of treating CLL by administering a compound that modulates the expression or activity of SF3B1, including compounds that activate or inhibit expression or activity of SF3B1.

### DEFINITIONS

“Accuracy” refers to the degree of conformity of a measured or calculated quantity (a test reported value) to its actual (or true) value. Clinical accuracy relates to the proportion of true outcomes (true positives (TP) or true negatives (TN) versus misclassified outcomes (false positives (FP) or false negatives (FN)), and may be stated as a sensitivity, specificity, positive predictive values (PPV) or negative predictive values (NPV), or as a likelihood, odds ratio, among other measures.

“Biomarker” in the context of the present invention encompasses, without limitation, proteins, nucleic acids, and metabolites, together with their polymorphisms, mutations, variants, modifications, subunits, fragments, protein-ligand complexes, and degradation products, protein-ligand complexes, elements, related metabolites, and other analytes or sample-derived measures. Biomarkers can also include mutated proteins or mutated nucleic acids. Biomarkers also encompass non-blood borne factors or non-analyte physiological markers of health status, such as “clinical parameters” defined herein, as well as “traditional laboratory risk factors”, also defined herein. Biomarkers also include any calculated indices created mathematically or combinations of any one or more of the foregoing measurements, including temporal trends and differences. Where available, and unless otherwise described herein, biomarkers which are gene products are identified based on the official letter abbreviation or gene symbol assigned by the international Human Genome Organization Naming Committee (HGNC) and listed at the date of this filing at the US National Center for Biotechnology Information (NCBI) web site.

A “CLL driver” is any mutation, chromosomal abnormality, or altered gene expression, that contributes to the etiology, progression, severity, aggressiveness, or prognosis of CLL. In some aspects, a CLL driver is a mutation that provides a selectable fitness advantage to a CLL cell and facilitates its clonal expansion in the population. CLL driver may be used interchangeably with CLL driver event and CLL driver mutation. CLL driver mutations occur in genes, genetic loci, or chromosomal regions which may be referred to herein interchangeably as CLL risk alleles, CLL alleles, CLL risk genes, CLL genes, CLL-associated genes and the like.

The disclosure also refers to CLL-associated markers. Such markers may be those known in the art including for example ZAP expression status and IGHV mutation status. Such markers may also include those newly discovered and described herein. Accordingly, CLL-associated markers include CLL drivers, including subclonal CLL drivers, of the invention. Some CLL-associated markers have prognostic value and may be referred to as CLL prognostic markers. Some prognostic markers are referred to as independent prognostic markers intending that they can be used individually to assess prognosis of a patient.

A “clinical indicator” is any physiological datum used alone or in conjunction with other data in evaluating the physiological condition of a collection of cells or of an organism. This term includes pre-clinical indicators.

“Clinical parameters” encompasses all non-sample or non-analyte biomarkers of subject health status or other characteristics, such as, without limitation, age (Age), ethnicity (RACE), gender (Sex), or family history (FamHX).

“FN” is false negative, which for a disease state test means classifying a disease subject incorrectly as non-disease or normal.

“FP” is false positive, which for a disease state test means classifying a normal subject incorrectly as having disease.

A “formula,” “algorithm,” or “model” is any mathematical equation, algorithmic, analytical or programmed process, or statistical technique that takes one or more continuous or categorical inputs (herein called “parameters”) and calculates an output value, sometimes referred to as an “index” or “index value.” Non-limiting examples of “formulas” include sums, ratios, and regression operators, such as coefficients or exponents, biomarker value transformations and normalizations (including, without limitation, those normalization schemes based on clinical parameters, such as gender, age, or ethnicity), rules and guidelines, statistical classification models, and neural networks trained on historical populations. Of particular use in combining biomarkers are linear and non-linear equations and statistical classification analyses to determine the relationship between biomarkers detected in a subject sample and the subject's responsiveness to chemotherapy. In panel and combination construction, of particular interest are structural and synactic statistical classification algorithms, and methods of risk index construction, utilizing pattern recognition features, including established techniques such as cross-correlation, Principal Components Analysis (PCA), factor rotation, Logistic Regression (LogReg), Linear Discriminant Analysis (LDA), Eigengene Linear Discriminant Analysis (ELDA), Support Vector Machines (SVM), Random Forest (RF), Recursive Partitioning Tree (RPART), as well as other related decision tree classification techniques, Shrunken Centroids (SC), StepAIC, Kth-Nearest Neighbor, Boosting, Decision Trees, Neural Networks, Bayesian Networks, Support Vector Machines, and Hidden Markov Models, among others. Other techniques may be used in survival and time to event hazard analysis, including Cox, Weibull, Kaplan-Meier and Greenwood models well known to those of skill in the art. Many of these techniques are useful as forward selection, backwards selection, or stepwise selection, complete enumeration of all potential panels of a given size, genetic algorithms, or they may themselves include biomarker selection methodologies in their own technique. These may be coupled with information criteria, such as Akaike's Information Criterion (AIC) or Bayes Information Criterion (BIC), in order to quantify the tradeoff between additional biomarkers and model improvement, and to aid in minimizing overfit. The resulting predictive models may be validated in other studies, or cross-validated in the study they were originally trained in, using such techniques as Bootstrap, Leave-One-Out (LOO) and 10-Fold cross-validation (10-Fold CV). At various steps, false discovery rates may be estimated by value permutation according to techniques known in the art. A “health economic utility function” is a formula that is derived from a combination of the expected probability of a range of clinical outcomes in an idealized applicable patient population, both before and after the introduction of a diagnostic or therapeutic intervention into the standard of care. It encompasses estimates of the accuracy, effectiveness and performance characteristics of such intervention, and a cost and/or value measurement (a utility) associated with each outcome, which may be derived from actual health system costs of care (services, supplies, devices and drugs, etc.) and/or as an estimated acceptable value per quality adjusted life year (QALY) resulting in each outcome. The sum, across all predicted outcomes, of the product of the predicted population size for an outcome multiplied by the respective outcome's expected utility is the total health economic utility of a given standard of care. The difference between (i) the total health economic utility calculated for the standard of care with the intervention versus (ii) the total health economic utility for the standard of care without the intervention results in an overall measure of the health economic cost or value of the intervention. This may itself be divided amongst the entire patient group being analyzed (or solely amongst the intervention group) to arrive at a cost per unit intervention, and to guide such decisions as market positioning, pricing, and assumptions of health system acceptance. Such health economic utility functions are commonly used to compare the cost-effectiveness of the intervention, but may also be transformed to estimate the acceptable value per QALY the health care system is willing to pay, or the acceptable cost-effective clinical performance characteristics required of a new intervention.

For diagnostic (or prognostic) interventions of the invention, as each outcome (which in a disease classifying diagnostic test may be a TP, FP, TN, or FN) bears a different cost, a health economic utility function may preferentially favor sensitivity over specificity, or PPV over NPV based on the clinical situation and individual outcome costs and value, and thus provides another measure of health economic performance and value which may be different from more direct clinical or analytical performance measures. These different measurements and relative trade-offs generally will converge only in the case of a perfect test, with zero error rate (a.k.a., zero predicted subject outcome misclassifications or FP and FN), which all performance measures will favor over imperfection, but to differing degrees.

“Measuring” or “measurement,” or alternatively “detecting” or “detection,” means assessing the presence, absence, quantity or amount (which can be an effective amount) of either a given substance within a clinical or subject-derived sample, including the derivation of qualitative or quantitative concentration levels of such substances, or otherwise evaluating the values or categorization of a subject's non-analyte clinical parameters. It is to be understood, as will be described in greater detail herein, that the analyzing and detecting steps of the invention are typically carried out using sequencing techniques including but not limited to nucleic acid arrays. Accordingly, analysis or detection, as referred to in the invention, generally depends upon the use of a device or a machine that transforms a nucleic acid into a visible rendering of its nucleic acid sequence in whole or in part. Such rendering may take the form of a computer read-out or output. In order for nucleic acid mutations to be detected, as provided herein, such nucleic acids must be extracted from their natural source and manipulated by devices or machines.

“Mutation” encompasses any change in a DNA, RNA, or protein sequence from the wild type sequence or some other reference, including without limitation point mutations, transitions, insertions, transversions, translocations, deletions, inversions, duplications, recombinations, or combinations thereof. A “clonal mutation” is a mutation present in the majority of CLL cells in a CLL tumor or CLL sample. In some preferred embodiments, “clonal mutation” is a mutation likely present in more than 0.95 (95%) of the cancer cells of a CLL sample, i.e. the cancer cell fraction of the mutation (CCF)>0.95. In other words, there is a probability of greater than 50% that the mutation is present in more than 95% of the cancer cells. A “subclonal mutation” is a mutation present in a single cell or a minority of cells in a CLL tumor or CLL sample. In some preferred aspects, a “subclonal mutation” is a mutation that is unlikely to be present in more than 0.95 (95%) of the cancer cells of a CLL sample (i.e., there is a probability of greater than 50% that the mutation is present in less than 95% of the cancer cells). As will be appreciated, a “clonal mutation” exists in the vast majority of cancer cells and while a “sub-clonal mutation” is only in a fraction of the cancer cells.

“Negative predictive value” or “NPV” is calculated by TN/(TN+FN) or the true negative fraction of all negative test results. It also is inherently impacted by the prevalence of the disease and pre-test probability of the population intended to be tested. See, e.g., O'Marcaigh A S, Jacobson R M, “Estimating The Predictive Value Of A Diagnostic Test, How To Prevent Misleading Or Confusing Results,” Clin. Ped. 1993, 32(8): 485-491, which discusses specificity, sensitivity, and positive and negative predictive values of a test, e.g., a clinical diagnostic test. Often, for binary disease state classification approaches using a continuous diagnostic test measurement, the sensitivity and specificity is summarized by Receiver Operating Characteristics (ROC) curves according to Pepe et al., “Limitations of the Odds Ratio in Gauging the Performance of a Diagnostic, Prognostic, or Screening Marker,” Am. J. Epidemiol 2004, 159 (9): 882-890, and summarized by the Area Under the Curve (AUC) or c-statistic, an indicator that allows representation of the sensitivity and specificity of a test, assay, or method over the entire range of test (or assay) cut points with just a single value. See also, e.g., Shultz, “Clinical Interpretation Of Laboratory Procedures,” chapter 14 in Teitz, Fundamentals of Clinical Chemistry, Burtis and Ashwood (eds.), 4th edition 1996, W.B. Saunders Company, pages 192-199; and Zweig et al., “ROC Curve Analysis: An Example Showing The Relationships Among Serum Lipid And Apolipoprotein Concentrations In Identifying Subjects With Coronory Artery Disease,” Clin. Chem., 1992, 38(8): 1425-1428. An alternative approach using likelihood functions, odds ratios, information theory, predictive values, calibration (including goodness-of-fit), and reclassification measurements is summarized according to Cook, “Use and Misuse of the Receiver Operating Characteristic Curve in Risk Prediction,” Circulation 2007, 115: 928-935.

Finally, hazard ratios and absolute and relative risk ratios within subject cohorts defined by a test are a further measurement of clinical accuracy and utility. Multiple methods are frequently used to defining abnormal or disease values, including reference limits, discrimination limits, and risk thresholds.

“Analytical accuracy” refers to the reproducibility and predictability of the measurement process itself, and may be summarized in such measurements as coefficients of variation, and tests of concordance and calibration of the same samples or controls with different times, users, equipment and/or reagents. These and other considerations in evaluating new biomarkers are also summarized in Vasan, 2006.

“Performance” is a term that relates to the overall usefulness and quality of a diagnostic or prognostic test, including, among others, clinical and analytical accuracy, other analytical and process characteristics, such as use characteristics (e.g., stability, ease of use), health economic value, and relative costs of components of the test. Any of these factors may be the source of superior performance and thus usefulness of the test, and may be measured by appropriate “performance metrics,” such as AUC, time to result, shelf life, etc. as relevant.

“Positive predictive value” or “PPV” is calculated by TP/(TP+FP) or the true positive fraction of all positive test results. It is inherently impacted by the prevalence of the disease and pre-test probability of the population intended to be tested.

“Risk” in the context of the present invention, relates to the probability that an event will occur over a specific time period, as in the responsiveness to treatment, cancer recurrence or survival and can mean a subject's “absolute” risk or “relative” risk. Absolute risk can be measured with reference to either actual observation post-measurement for the relevant time cohort, or with reference to index values developed from statistically valid historical cohorts that have been followed for the relevant time period. Relative risk refers to the ratio of absolute risks of a subject compared either to the absolute risks of low risk cohorts or an average population risk, which can vary by how clinical risk factors are assessed. Odds ratios, the proportion of positive events to negative events for a given test result, are also commonly used (odds are according to the formula p/(1−p) where p is the probability of event and (1−p) is the probability of no event) to no-conversion.

“Elevated risk” relates to an increased probability than an event will occur compared to another population. In the context of the present disclosure, “a subject at elevated risk of having CLL with rapid disease progression” refers to a CLL subject having an increased probability of rapid disease progression due to the presence of one or more mutations, including subclonal mutations, in a CLL risk allele, as compared to a CLL subject not having such mutation(s).

“Risk evaluation” or “evaluation of risk” in the context of the present invention encompasses making a prediction of the probability, odds, or likelihood that an event or disease state may occur, the rate of occurrence of the event or conversion from one disease state. Risk evaluation can also comprise prediction of future clinical parameters, traditional laboratory risk factor values, or other indices of cancer, either in absolute or relative terms in reference to a previously measured population. The methods of the present invention may be used to make continuous or categorical measurements of the responsiveness to treatment thus diagnosing and defining the risk spectrum of a category of subjects defined as being responders or non-responders. In the categorical scenario, the invention can be used to discriminate between normal and other subject cohorts at higher risk for responding. Such differing use may require different biomarker combinations and individualized panels, mathematical algorithms, and/or cut-off points, but be subject to the same aforementioned measurements of accuracy and performance for the respective intended use.

A “sample” in the context of the present invention is a biological sample isolated from a subject and can include, by way of example and not limitation, tissue biopies, lymph node tissue, whole blood, serum, plasma, blood cells, endothelial cells, lymphatic fluid, ascites fluid, interstitial fluid (also known as “extracellular fluid” and encompasses the fluid found in spaces between cells, including, inter alia, gingival crevicular fluid), bone marrow, cerebrospinal fluid (CSF), saliva, mucous, sputum, sweat, urine, or any other secretion, excretion, or other bodily fluids. A “sample” may include a single cell or multiple cells or fragments of cells. The sample is also a tissue sample. The sample is or contains a circulating endothelial cell or a circulating tumor cell. The sample includes a primary tumor cell, primary tumor, a recurrent tumor cell, or a metastatic tumor cell.

“CLL sample” refers to a sample taken from a subject having or suspected of having CLL, wherein the sample is believed to contain CLL cells if such cells are present in the subject. The CLL sample preferably contains white blood cells from the subject.

“Sensitivity” is calculated by TP/(TP+FN) or the true positive fraction of disease subjects.

“Specificity”, as it relates to some aspects of the invention, is calculated by TN/(TN+FP) or the true negative fraction of non-disease or normal subjects.

By “statistically significant”, it is meant that the alteration is greater than what might be expected to happen by chance alone (which could be a “false positive”). Statistical significance can be determined by any method known in the art. Commonly used measures of significance include the p-value, which presents the probability of obtaining a result at least as extreme as a given data point, assuming the data point was the result of chance alone. A result is considered highly significant at a p-value of 0.05 or less. Preferably, the p-value is 0.04, 0.03, 0.02, 0.01, 0.005, 0.001 or less.

A “subject” in the context of the present invention is preferably a mammal. The mammal can be a human, non-human primate, mouse, rat, dog, cat, horse, or cow, but are not limited to these examples. Mammals other than humans can be advantageously used as subjects that represent animal models of cancer. A subject can be male or female. In some aspects, a subject is a mammal having or suspected of having CLL. Human subjects may be referred to herein as patients.

“TN” is true negative, which for a disease state test means classifying a non-disease or normal subject correctly.

“TP” is true positive, which for a disease state test means correctly classifying a disease subject.

“Traditional laboratory risk factors” correspond to biomarkers isolated or derived from subject samples and which are currently evaluated in the clinical laboratory and used in traditional global risk assessment algorithms. Traditional laboratory risk factors for tumor recurrence include for example Proliferative index, tumor infiltrating lymphocytes. Other traditional laboratory risk factors for tumor recurrence known to those skilled in the art.

### Methods and Uses of the Invention

The methods disclosed herein are used with subjects undergoing treatment and/or therapies for CLL, subjects who are at risk for developing a reoccurrence of CLL, and subjects who have been diagnosed with CLL. The methods of the present invention are to be used to monitor or select a treatment regimen for a subject who has CLL, and to evaluate the predicted survivability and/or survival time of a CLL-diagnosed subject.

Aggressiveness of the disease course of CLL is determined by detecting a mutation in one or more of the driver genes provided herein, such as for example the SF3B1 gene, in a test sample (e.g., a subject-derived sample). Optionally, the mutation in the SF3B1 gene occurs at nucleotides that provide coding sequence for the amino acid region between amino acids 550 to 1050 of a SF3B1 polypeptide. The mutation associated with an aggressive disease course includes for example one or more somatic mutations in the SF3B1 gene leading to an amino acid substitution at positions 622, 625, 626, 659, 666, 700, 740, 741, 742 and 903 of the SF3B1 polypeptide. Specifically these mutations results in: glutamic acid to aspartic acid at 622 (E622D); an arginine to leucine or arginine to glycine at position 625 (R625L, R625G); an asparagine to histidine at position 626 (N626H); a glutamine to arginine at 656 (Q659R); a lysine to glutamine or lysine to glutamic acid at 666 (K666Q, K666E); a lysine to glutamic acid at position 700 (K700E); a glycine to glutamic acid at position 740 (G740E); a lysine to asparagine at position 741 (K741N); a glycine to aspartic acid at 742 (G742D); and/or a glutamine to arginine at position 903 (Q903R). These mutations associated with aggressiveness of disease course are referred to herein as the CLL/SF3B1 mutations. In analyzing 160 CLL samples, the K700E SF3B1 mutation was identified in 9 samples, the G742D mutation in four samples, and the following mutations were identified in one CLL sample: E622D, R625G, R625L, Q659R, K666E, G740E, K741N, and Q903R. See Table 1.1 for further details regarding the specific mutations identified in the cohort of 160 CLL samples. The presence of a CLL/SF3B1 mutation indicates a more aggressive disease course. Other mutations in the SF3B1 gene are also contemplated by the invention.

TABLE 1.1

| | Entrez | | | | | | | | |
|---|---|---|---|---|---|---|---|---|---|
| | Gene | | | | Genome | Annotation | cDNA | Protein | |
| Hugo_ID | ID | Chr | Position | Variant | Change | Transcript | Change | Change | Pt_ID |
| SF3B1 | 23451 | 2 | 197973694 | Mis | g.chr2: 197973694T > C | uc002uue.1 | c.2708A > G | p.Q903R | CLL040 |
| SF3B1 | 23451 | 2 | 197974856 | Mis | g.chr2: 197974856C > T | uc002uue.1 | c.2225G > A | p.G742D | CLL007 |
| SF3B1 | 23451 | 2 | 197974856 | Mis | g.chr2: 197974856C > T | uc002uue.1 | c.2225G > A | p.G742D | CLL051 |
| SF3B1 | 23451 | 2 | 197974856 | Mis | g.chr2: 197974856C > T | uc002uue.1 | c.2225G > A | p.G742D | CLL096 |
| SF3B1 | 23451 | 2 | 197974856 | Mis | g.chr2: 197974856C > T | uc002uue.1 | c.2225G > A | p.G742D | CLL165 |
| SF3B1 | 23451 | 2 | 197974954 | Mis | g.chr2: 197974954C > A | uc002uue.1 | c.2223G > T | p.K741N | CLL084 |
| SF3B1 | 23451 | 2 | 197974958 | Mis | g.chr2: 197974958C > T | uc002uue.1 | c.2219G > A | p.G740E | CLL058 |
| SF3B1 | 23451 | 2 | 197975079 | Mis | g.chr2: 197975079T > C | uc002uue.1 | c.2098A > G | p.K700E | CLL032 |
| SF3B1 | 23451 | 2 | 197975079 | Mis | g.chr2: 197975079T > C | uc002uue.1 | c.2098A > G | p.K700E | CLL037 |
| SF3B1 | 23451 | 2 | 197975079 | Mis | g.chr2: 197975079T > C | uc002uue.1 | c.2098A > G | p.K700E | CLL043 |
| SF3B1 | 23451 | 2 | 197975079 | Mis | g.chr2: 197975079T > C | uc002uue.1 | c.2098A > G | p.K700E | CLL059 |
| SF3B1 | 23451 | 2 | 197975079 | Mis | g.chr2: 197975079T > C | uc002uue.1 | c.2098A > G | p.K700E | CLL061 |
| SF3B1 | 23451 | 2 | 197975079 | Mis | g.chr2: 197975079T > C | uc002uue.1 | c.2098A > G | p.K700E | CLL085 |
| SF3B1 | 23451 | 2 | 197975079 | Mis | g.chr2: 197975079T > C | uc002uue.1 | c.2098A > G | p.K700E | CLL101 |
| SF3B1 | 23451 | 2 | 197975079 | Mis | g.chr2: 197975079T > C | uc002uue.1 | c.2098A > G | p.K700E | CLL107 |
| SF3B1 | 23451 | 2 | 197975079 | Mis | g.chr2: 197975079T > C | uc002uue.1 | c.2098A > G | p.K700E | CLL115 |
| SF3B1 | 23451 | 2 | 197975606 | Mis | g.chr2: 197975606T > C | uc002uue.1 | c.1996A > G | p.K666E | CLL102 |
| SF3B1 | 23451 | 2 | 197975606 | Mis | g.chr2: 197975606T > G | uc002uue.1 | c.1996A > C | p.K666Q | CLL109 |
| SF3B1 | 23451 | 2 | 197975626 | Mis | g.chr2: 197975626T > C | uc002uue.1 | c.1976A > G | p.Q659R | CLL013 |
| SF3B1 | 23451 | 2 | 197975728 | Mis | g.chr2: 197975728C > A | uc002uue.1 | c.1874G > T | p.R625L | CLL060 |
| SF3B1 | 23451 | 2 | 197975729 | Mis | g.chr2: 197975729G > C | uc002uue.1 | c.1873C > G | p.R625G | CLL127 |
| SF3B1 | 23451 | 2 | 197975736 | Mis | g.chr2: 197975736C > G | uc002uue.1 | c.1866G > C | p.E622D | CLL169 |

In some aspects, aggressiveness of the CLL disease course, or identifying a subject as a subject at elevated risk of having CLL with rapid disease progression, is determined by detecting a mutation in a test sample (e.g., a subject-derived sample) in one or more genes selected from the group consisting of SF3B1, HIST1H1E, NRAS, BCOR, RIPK1, SAMHD1, KRAS, MED12, ITPKB, EGR2, DDX3X, ZMYM3, FBXW7, ATM, TP53, MYD88, NOTCH1, XPO1, CHD2, and POT1, whether alone or in some combination with each other or with other mutations. In some important embodiments of the invention these driver events are subclonal.

In some embodiments, the mutation in HIST1H1E is DV72del, R79H, A167V, P196S, and/or K202E. In some embodiments, the mutation in NRAS is Q61R, and/or Q61K. In some embodiments, the mutation in BCOR is a frame shift mutation at V132, T200, and/or P463, and/or a nonsense mutation at E1382. In some embodiments, the mutation in RIPK1 is A448V, K599R, R603S, and/or a nonsense mutation at Q375. In some embodiments, the mutation in SAMHD1 is M254I, R339S, I386S, and/or a frame shift mutation at R290. In some aspects, the mutation in KRAS is G13D, and/or Q61H. In some embodiments, the mutation in MED12 is E33K, G44S, and/or A59P. In some embodiments, the mutation in ITPKB is a frame shift mutation at E207, and/or E584, and/or the mutation T626S. In some embodiments, the mutation in EGR2 is H384N. In some embodiments, the mutation in DDX3X is a nonsense mutation at S24, and/or a splicing mutation at K342, and/or a frame shift mutation at S410. In some embodiments, the mutation in ZMYM3 is Y1113del, F1302S, and/or a frame shift mutation at S53, and/or a nonsense mutation at Q399. In some embodiments, the mutation in FBXW7 is F280L, R465H, R505C, and/or G597E. In some embodiments, the mutation in ATM is L120R, H2038R, E2164Q, Y2437S, Q2522H, Y2954C, A3006T, and/or a frame shift mutation at K468, L546, and/or L2135, and/or a splicing mutation at C1726, and/or a nonsense mutation at Y2817. In some embodiments, the mutation in TP53 occurs in the DNA binding domain (DBD) of TP53. In some embodiments the mutation in TP53 is L111R, N131del, R175H, H193P, I195T, H214R, 1232F, C238S, C242F, R248Q, I255F, G266V, R267Q, R273C, R273H, R267Q, C275Y, D281N, and/or a splicing mutation at G187. In some embodiments, the mutation in MYD88 occurs in the Toll/Interleukin-1 receptor (TIR) domain of MYD88. In some embodiments, the mutation in MYD88 is M219T, and or L252P. In some embodiments, the mutation in NOTCH1 occurs in the glutamic acid/serine/threonine (PEST) domain of NOTCH1. In some embodiments, the mutation in NOTCH1 is a nonsense mutation at Q2409, and/or a frame shift mutation at P2514. In some embodiments, the mutation in XPO1 is E571K, E571A, and/or D624G. In some embodiments, the mutation in CHD2 is T645M, K702R, R836P, and/or a nonsense mutation at R1072, and/or a splicing mutation at I1427 and/or I1471. In some embodiments, the mutation in POT1 is Y36H, D77G, R137C, and/or a nonsense mutation at Y73 and/or W194. These mutations associated with aggressiveness of disease course are referred to herein as CLL mutations and/or CLL drivers. In some embodiments, the presence of a CLL mutation indicates a more aggressive disease course, or identifies a subject as a subject at elevated risk of having CLL with rapid disease progression.

In some aspects, methods are provided for determining the aggressiveness of the disease course, or identifying a subject as a subject at elevated risk of having CLL with rapid disease progression, by detecting in a test sample (e.g., a subject-derived sample) one or more chromosomal abnormalities including deletions in chromosome 8p, 13q, 11q, and 17p, and trisomy of chromosome 12, whether alone or in some combination with each other or with other mutations. In some important embodiments of the invention these driver events are subclonal. These chromosomal abnormalities are also referred to herein as CLL mutations and/or CLL drivers, and are associated with aggressiveness of disease course. In some embodiments, the presence of a CLL mutation such as a chromosomal abnormality indicates a more aggressive disease course, or identifies a subject as a subject at elevated risk of having CLL with rapid disease progression.

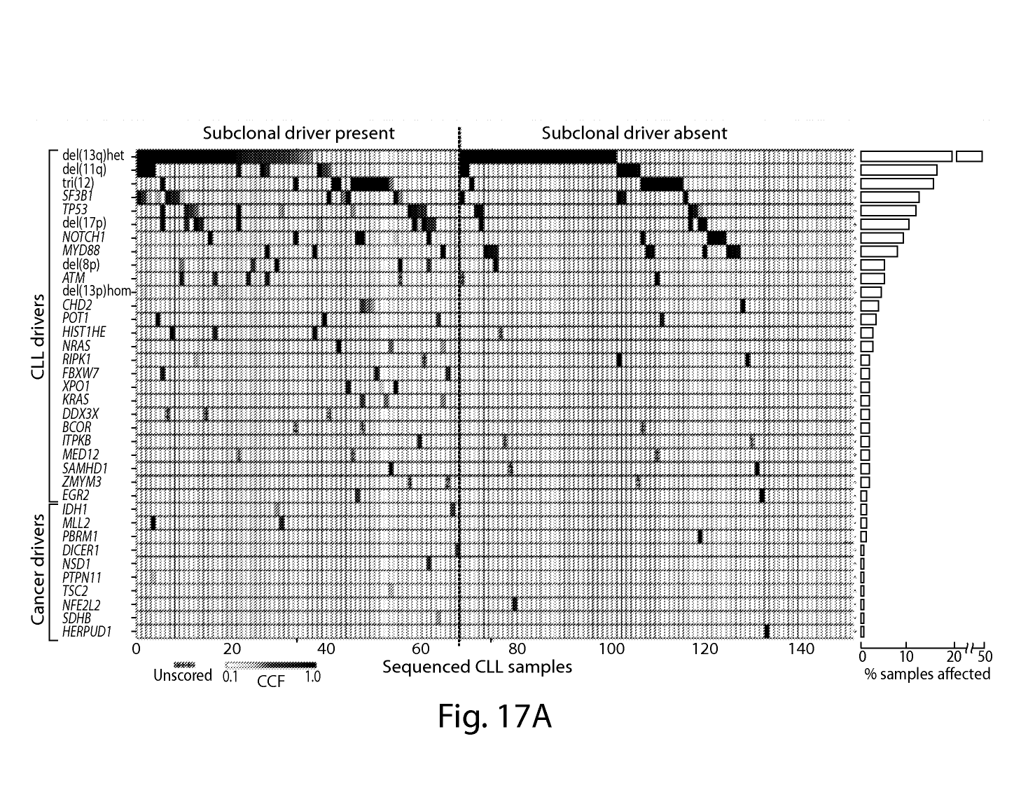

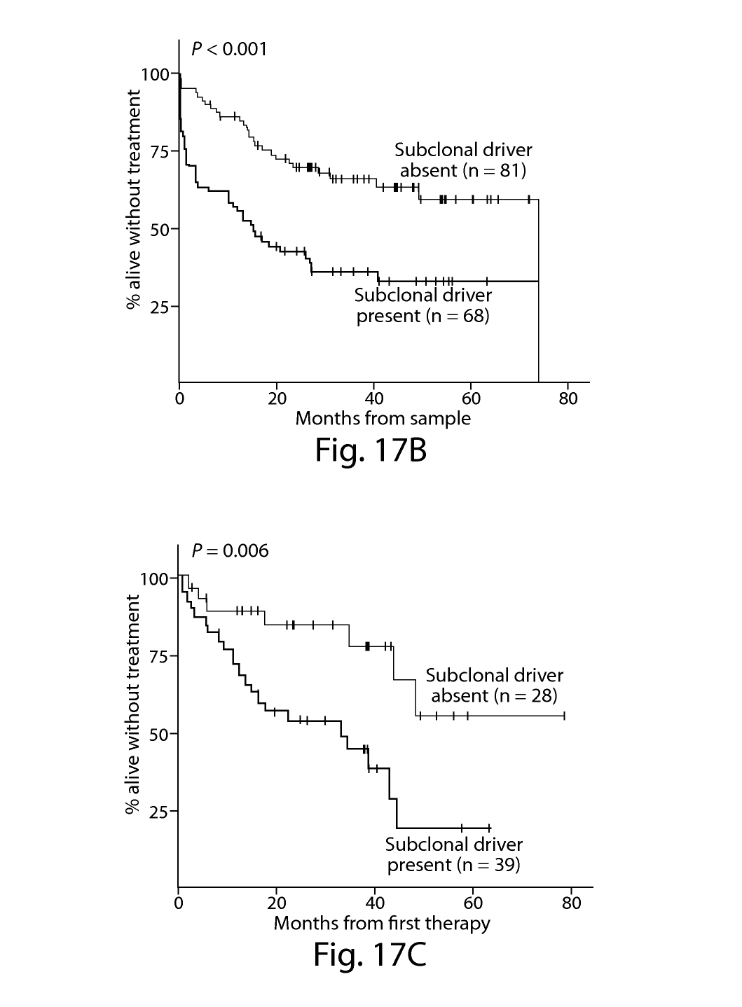

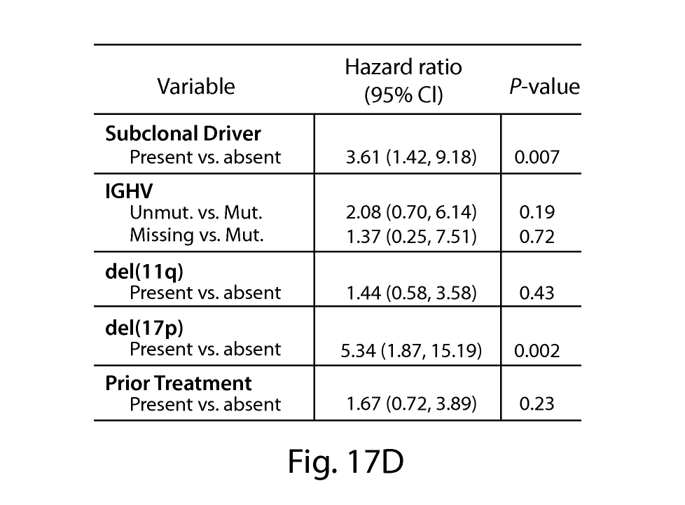

In some aspects, the disclosure provides methods for determining the aggressiveness of the disease course, or identifying a subject as a subject at elevated risk of having CLL with rapid disease progression, in subjects having or suspected of having CLL by determining whether a mutation or a chromosomal abnormality in a CLL driver is clonal or subclonal. In some embodiments, the detection of a subclonal CLL mutation or chromosomal abnormality indicates a more aggressive disease course, or identifies a subject as a subject at elevated risk of having CLL with rapid disease progression. In some embodiments, individual or combined subclonal CLL mutations are independent prognostic markers of CLL, and are used to determine a treatment regimen. For example, as shown in FIG. 17B, at 60 months post-sample, less than ˜35% of subjects identified as having a subclonal CLL mutation were alive without treatment, whereas greater than ˜60% of subjects identified as not having a subclonal CLL mutation were alive without treatment. Further, as shown in FIG. 17C, at 60 months following first therapy, less than ˜20% of subjects identified as having a subclonal CLL mutation were alive without retreatment, whereas greater than ˜55% of subjects identified as not having a subclonal CLL mutation were alive without retreatment. Thus the detection of a subclonal CLL mutation indicates a more rapid, or aggressive disease course, and informs decisions regarding treatment.

In some aspects, the detection of a subclonal CLL driver mutation in a subject-derived sample identifies the subject as a subject requiring immediate treatment. In some aspects, the presence of a subclonal CLL mutation in a subject-derived sample identifies the subject as a subject requiring aggressive treatment. In some aspects, the detection of a CLL mutation, including a subclonal CLL mutation, in a subject-derived sample identifies the subject as a subject requiring alternative therapy. By an alternative therapy it is meant that the subject should be treated with a different or altered dose of a medicament, different combinations of medicaments, medicaments that work through varied mechanisms (including a mechanism that is different from that of a previous treatment), or the timing of treatment should be adjusted depending on the identification of a CLL mutation, including subclonal CLL mutations, and/or other clinical indicators. In some examples, alternative therapies are to be considered for subjects identified as having a CLL mutation, including subclonal CLL mutations, wherein the subject had previously been treated for CLL.

In some aspects, methods are methods for determining the aggressiveness of the disease course, or identifying a subject as a subject at elevated risk of having cancer with rapid disease progression, by detecting mutations, and particularly subclonal mutations, in one or more (including two or more) risk alleles selected from the group consisting of SF3B1, HIST1H1E, NRAS, BCOR, RIPK1, SAMHD1, KRAS, MED12, ITPKB, EGR2, DDX3X, ZMYM3, FBXW7, TP53, MYD88, NOTCH1, XPO1, CHD2, POT1, del(8p), del(13q), del(11q), del(17p), and trisomy 12. The presence of a mutations, and particularly subclonal mutations, in two or more risk alleles indicates a more aggressive disease course. The presence of two or more subclonal driver mutations indicates a more aggressive disease course, or identifies a subject as a subject at elevated risk of having CLL with rapid disease progression.

In some aspects, methods are provided for determining the aggressiveness of the disease course, or identifying a subject as a subject at elevated risk of having cancer with rapid disease progression, by (i) detecting a mutation in one or more (including two or more) risk alleles group consisting of SF3B1, HIST1H1E, NRAS, BCOR, RIPK1, SAMHD1, KRAS, MED12, ITPKB, EGR2, DDX3X, ZMYM3, and FBXW7; and (ii) detecting a mutation in one or more CLL drivers TP53, MYD88, NOTCH1, XPO1, CHD2, POT1, del(8p), del(13q), del(11q), del(17p), or trisomy 12. In some aspects, the method further comprises determining whether the mutations in the risk alleles in (i) and (ii) are clonal or subclonal. In some aspects, the presence of two or more subclonal driver mutations indicates a more aggressive disease course, or identifies a subject as a subject at elevated risk of having CLL with rapid disease progression.

In some aspects, methods are provided for determining the aggressiveness of the disease course, or identifying a subject as a subject at elevated risk of having cancer with rapid disease progression, by detecting a mutation in a CLL sample in one or more risk alleles selected from the group consisting SF3B1, HIST1H1E, NRAS, BCOR, RIPK1, SAMHD1, KRAS, MED12, ITPKB, EGR2, DDX3X, ZMYM3, FBXW7, ATM, TP53, MYD88, NOTCH1, XPO1, CHD2, POT1, del(8p), del(13q), del(11q), del(17p), and trisomy 12, wherein mutations are detected in at least 2, at least 3, at least 4, at least 5, at least 6, at least 7, at least 8, at least 9 or at least 10 risk alleles selected from the group consisting of HIST1H1E, NRAS, BCOR, RIPK1, SAMHD1, KRAS, MED12, ITPKB, and EGR2, and optionally SF3B1. In some aspects the method further comprises determining whether the mutation is clonal or subclonal, and identifying the subject as a subject at elevated risk of having CLL with rapid disease progression if the mutation is a driver event and subclonal.

The cell is for example a cancer cell. In all preferred embodiments, the cancer is leukemia such as chronic lymphocytic leukemia (CLL).

By a more aggressive disease course it is meant that the subject having CLL will need treatment earlier than in a CLL subject that does not have the mutation. The methods of the present invention are useful to treat, alleviate the symptoms of, monitor the progression of or delay the onset of cancer.

Preferably, the methods of the present invention are used to identify and/or diagnose subjects who are asymptomatic for a cancer recurrence. “Asymptomatic” means not exhibiting the traditional symptoms.

The methods of the present invention are also useful to identify and/or diagnose subjects already at higher risk of developing a CLL.

Identification of one or more mutations in the SF3B1 gene and other CLL drivers identified herein allows for the determination of whether a subject will derive a benefit from a particular course of treatment, e.g. choice of treatment (i.e., more aggressive) or timing of treatment (e.g., earlier treatment). In this method, a biological sample is provided from a subject before undergoing treatment. Alternately, the sample is provides after a subject has undergone treatment. By “derive a benefit” it is meant that the subject will respond to the course of treatment. By responding it is meant that the treatment decreases in size, prevalence, a cancer in a subject. When treatment is applied prophylactically, “responding” means that the treatment retards or prevents a cancer recurrence from forming or retards, prevents, or alleviates a symptom. Assessments of cancers are made using standard clinical protocols.

The invention also provides method of treating CLL by administering to the subject a compound that modulates (e.g., inhibits or activates) the expression or activity of SF3B1 in which patients harboring mutated SF3B1 may be more sensitive to this compound. The methods are useful to alleviate the symptoms of cancer. Any cancer containing a SF3B1 mutation described herein is amenable to treatment by the methods of the invention. In some aspects the subject is suffering from CLL.

Treatment is efficacious if the treatment leads to clinical benefit such as, a decrease in size, prevalence, or metastatic potential of the tumor in the subject. When treatment is applied prophylactically, “efficacious” means that the treatment retards or prevents tumors from forming or prevents or alleviates a symptom of clinical symptom of the tumor. Efficaciousness is determined in association with any known method for diagnosing or treating the particular tumor type.

In some aspects, methods of treating a subject are provided. In some examples, a method of treatment comprises administering to a subject a therapy (including a therapeutic agent (or medicament), radiation, or other procedures such as transplantation), wherein the subject is identified as having an unfavorable CLL prognosis based upon the detection of one or more CLL mutations, including subclonal mutations.

Treatments or therapeutic agents contemplated by the present disclosure include but are not limited to immunotherapy, chemotherapy, bone marrow and stem cell transplantation, and others known in the art. In some examples, a subject-derived sample wherein a CLL mutation, including a subclonal CLL mutation, is detected, identifies the subject as requiring chemotherapy, wherein one or more of the following non-limiting chemotherapy regimens is administered to the subject: FC (fludarabine with cyclophosphamide), FR (fludarabine with rituximab), FCR (fludarabine, cyclophosphamide, and rituximab), and CHOP (cyclophosphamide, doxorubicin, vincristine and prednisolone). In some examples, combination chemotherapy regimens are administered to a subject identified according to the methods described herein, in both newly-diagnosed and relapsed CLL. In some aspects, combinations of fludarabine with alkylating agents (cyclophosphamide) produce higher response rates and a longer progression-free survival than single agents. Alkylating agents include bendamustine and cyclophosphamide.

In some examples, a subject-derived sample wherein a CLL mutation, including a subclonal CLL mutation, is detected, identifies the subject as requiring immunotherapy, wherein one or more of the following non-limiting immunotherapeutic agents is administered: alemtuzumab (Campath, MabCampath or Campath-1H), rituximab (Rituxan, MabThera) and ofatumumab (Arzerra, HuMax-CD20).

In some examples, a subject-derived sample harboring a CLL mutation, including a subclonal CLL mutation, identifies the subject as requiring bone marrow and/or stem cell transplantation. In some examples, a subject is identified according to the methods provided herein and is indicated as requiring more aggressive therapies, including lenalidomide, flavopiridol, and bone marrow and/or stem cell transplantation.

In some aspects, an aggressive treatment may comprise administering any therapeutic agent described herein or known in the art, either alone or in combination, and will depend upon individual patient characteristics and clinical indicators, as well the identification of prognostic markers as herein described.

Other therapies contemplated include compounds that decrease expression or activity of SF3B1. A decrease in SF3B1 expression or activity can be defined by a reduction of a biological function of SF3B1. A reduction of a biological function of SF3B1 includes a decrease in splicing of a gene or a set of genes. Altered splicing of genes can be measured by detecting a certain gene or subset of genes that are known to be spliced by SF3b spliceosome complex, or SF3B1 in particular, by methods known in the art and described herein. For example, the genes are ROIK3 or BRD2. SF3B1 is measured by detecting by methods known in the art.

SF3B1 modulators, including inhibitors, are known in the art or are identified using methods described herein. The SF3B1 inhibitor is for example splicostatin, E71707 or pladienolide. SF3B1 inhibitors alter splicing activity, for example, reduce, decrease or inhibit splicing. The invention further contemplates targeting of splice variants generated from mutated SF3B1, as a therapeutic target. For example, the impact of these splice variants may be reduced by targeting through inhibitory nucleic acid technologies such as siRNA and antisense.

The present invention can also be used to screen patient or subject populations in any number of settings. For example, a health maintenance organization, public health entity or school health program can screen a group of subjects to identify those requiring interventions, as described above, or for the collection of epidemiological data. Insurance companies (e.g., health, life or disability) may screen applicants in the process of determining coverage or pricing, or existing clients for possible intervention. Data collected in such population screens, particularly when tied to any clinical progression to conditions like cancer, will be of value in the operations of, for example, health maintenance organizations, public health programs and insurance companies. Such data arrays or collections can be stored in machine-readable media and used in any number of health-related data management systems to provide improved healthcare services, cost effective healthcare, improved insurance operation, etc. See, for example, U.S. Patent Application No. 2002/0038227; U.S. Patent Application No. US 2004/0122296; U.S. Patent Application No. US 2004/0122297; and U.S. Pat. No. 5,018,067. Such systems can access the data directly from internal data storage or remotely from one or more data storage sites as further detailed herein.

Each program can be implemented in a high level procedural or object oriented programming language to communicate with a computer system. However, the programs can be implemented in assembly or machine language, if desired. The language can be a compiled or interpreted language. Each such computer program can be stored on a storage media or device (e.g., ROM or magnetic diskette or others as defined elsewhere in this disclosure) readable by a general or special purpose programmable computer, for configuring and operating the computer when the storage media or device is read by the computer to perform the procedures described herein. The health-related data management system of the invention may also be considered to be implemented as a computer-readable storage medium, configured with a computer program, where the storage medium so configured causes a computer to operate in a specific and predefined manner to perform various functions described herein.

Differences in the genetic makeup of subjects can result in differences in their relative abilities to metabolize various drugs, which may modulate the symptoms or risk factors of cancer or metastatic events. Subjects that have cancer, or at risk for developing cancer or a metastatic event can vary in age, ethnicity, and other parameters. Accordingly, detection of the CLL/SF3B1 and/or other CLL driver mutations disclosed herein, both alone and together in combination with known prognostic markers for CLL, allow for a pre-determined level of predictability of the aggressiveness of the disease course and may impact on responsiveness to therapy.

### Performance and Accuracy Measures of the Invention

The performance and thus absolute and relative clinical usefulness of the invention may be assessed in multiple ways as noted above. Amongst the various assessments of performance, the invention is intended to provide accuracy in clinical diagnosis and prognosis. The accuracy of a diagnostic, predictive, or prognostic test, assay, or method concerns the ability of the test, assay, or method to distinguish between subjects responsive to chemotherapeutic treatment and those that are not, is based on whether the subjects have the one or more of the CLL/SF3B1 and/or other CLL driver mutations disclosed herein.

In the categorical diagnosis of a disease state, changing the cut point or threshold value of a test (or assay) usually changes the sensitivity and specificity, but in a qualitatively inverse relationship. Therefore, in assessing the accuracy and usefulness of a proposed medical test, assay, or method for assessing a subject's condition, one should always take both sensitivity and specificity into account and be mindful of what the cut point is at which the sensitivity and specificity are being reported because sensitivity and specificity may vary significantly over the range of cut points. Use of statistics such as AUC, encompassing all potential cut point values, is preferred for most categorical risk measures using the invention, while for continuous risk measures, statistics of goodness-of-fit and calibration to observed results or other gold standards, are preferred.

Using such statistics, an “acceptable degree of diagnostic accuracy”, is herein defined as a test or assay in which the AUC (area under the ROC curve for the test or assay) is at least 0.60, desirably at least 0.65, more desirably at least 0.70, preferably at least 0.75, more preferably at least 0.80, and most preferably at least 0.85. By a “very high degree of diagnostic accuracy”, it is meant a test or assay in which the AUC (area under the ROC curve for the test or assay) is at least 0.80, desirably at least 0.85, more desirably at least 0.875, preferably at least 0.90, more preferably at least 0.925, and most preferably at least 0.95.

The predictive value of any test depends on the sensitivity and specificity of the test, and on the prevalence of the condition in the population being tested. This notion, based on Bayes' theorem, provides that the greater the likelihood that the condition being screened for is present in an individual or in the population (pre-test probability), the greater the validity of a positive test and the greater the likelihood that the result is a true positive. Thus, the problem with using a test in any population where there is a low likelihood of the condition being present is that a positive result has limited value (i.e., more likely to be a false positive). Similarly, in populations at very high risk, a negative test result is more likely to be a false negative.

As a result, ROC and AUC can be misleading as to the clinical utility of a test in low disease prevalence tested populations (defined as those with less than 1% rate of occurrences (incidence) per annum, or less than 10% cumulative prevalence over a specified time horizon). Alternatively, absolute risk and relative risk ratios as defined elsewhere in this disclosure can be employed to determine the degree of clinical utility. Populations of subjects to be tested can also be categorized into quartiles by the test's measurement values, where the top quartile (25% of the population) comprises the group of subjects with the highest relative risk for therapeutic unresponsiveness, and the bottom quartile comprising the group of subjects having the lowest relative risk for therapeutic unresponsiveness. Generally, values derived from tests or assays having over 2.5 times the relative risk from top to bottom quartile in a low prevalence population are considered to have a “high degree of diagnostic accuracy,” and those with five to seven times the relative risk for each quartile are considered to have a “very high degree of diagnostic accuracy.” Nonetheless, values derived from tests or assays having only 1.2 to 2.5 times the relative risk for each quartile remain clinically useful are widely used as risk factors for a disease; such is the case with total cholesterol and for many inflammatory biomarkers with respect to their prediction of future events. Often such lower diagnostic accuracy tests must be combined with additional parameters in order to derive meaningful clinical thresholds for therapeutic intervention, as is done with the aforementioned global risk assessment indices.

A health economic utility function is yet another means of measuring the performance and clinical value of a given test, consisting of weighting the potential categorical test outcomes based on actual measures of clinical and economic value for each. Health economic performance is closely related to accuracy, as a health economic utility function specifically assigns an economic value for the benefits of correct classification and the costs of misclassification of tested subjects. As a performance measure, it is not unusual to require a test to achieve a level of performance which results in an increase in health economic value per test (prior to testing costs) in excess of the target price of the test.

In general, alternative methods of determining diagnostic accuracy are commonly used for continuous measures, when a disease category or risk category has not yet been clearly defined by the relevant medical societies and practice of medicine, where thresholds for therapeutic use are not yet established, or where there is no existing gold standard for diagnosis of the pre-disease. For continuous measures of risk, measures of diagnostic accuracy for a calculated index are typically based on curve fit and calibration between the predicted continuous value and the actual observed values (or a historical index calculated value) and utilize measures such as R squared, Hosmer-Lemeshow P-value statistics and confidence intervals. It is not unusual for predicted values using such algorithms to be reported including a confidence interval (usually 90% or 95% CI) based on a historical observed cohort's predictions, as in the test for risk of future breast cancer recurrence commercialized by Genomic Health, Inc. (Redwood City, Calif.).

### Detection of the CLL/SF3B1 and CLL Driver Mutations

Detection of the SF3B1 mutations and/or other CLL driver mutations can be determined at the protein or nucleic acid level using any method known in the art. Preferred SF3B1 mutations and/or CLL driver mutations of the invention are missense mutations, for example, R625L, N626H, K700E, K741N, G740E, E622D, R625G, Q659R, K666Q, K666E, G742D, or Q903R in SF3B1. Suitable sources of the nucleic acids encoding SF3B1 include, for example, the human genomic SF3B1 nucleic acid, available as GenBank Accession No: NG_{—}032903.1, the SF3B1 mRNA nucleic acid available as GenBank Accession Nos: NM_{—}001005526.1 and NM_{—}012433.2, and the human SF3B1 protein, available as GenBank Accession Nos: NP_{—}036565.2 and NP_{—}001005526.1.

Suitable sources of the nucleic acids and proteins for the following CLL drivers may be found in Table 1.2: NRAS, KRAS, BCOR, EGR2, MED12, RIPK1, SAMHD1, ITPKB, HIST1H1E, ATM, TP53, MYD88, NOTCH1, DDX3X, ZMYM3, FBXW7, XPO1, CHD2, and POT1.

TABLE 1.2

| | GenBank | GenBank | GenBank |
|---|---|---|---|
| | Accession No, | Accession No, | Accession No, |
| Gene | genomic | mRNA | protein |
| NRAS | NG_007572.1 | NM_002524.4 | NP_002515.1 |
| | | NM_004985.3; | NP_004976.2; |
| KRAS | NG_007524.1 | NM_033360.2 | NP_203524.1 |
| | | NM_001123383.1; | NP_001116855.1; |
| | | NM_001123384.1; | NP_001116856.1; |
| | | NM_001123385.1; | NP_001116857.1; |
| BCOR | NG_008880.1 | NM_017745.5 | NP_060215.4 |
| | | NM_000399.3; | NP_000390.2; |
| | | NM_001136177.1; | NP_001129649.1; |
| | | NM_001136178.1, | NP_001129650.1; |
| EGR2 | NG_008936.2 | NM_001136179.1 | NP_001129651.1 |
| MED12 | NG_012808.1 | NM_005120.2 | NP_005111.2 |
| | NC_000006.11; | | |
| | AC_000138.1; | | |
| RIPK1 | NC_018917.1 | NM_003804.3 | NP_003795.2 |
| SAMHD1 | NG_017059.1 | NM_015474.3 | NP_056289.2 |
| | NC_000001.10; | | |
| | AC_000133.1; | | |
| ITPKB | NC_018912.1 | NM_002221.3 | NP_002212.3 |
| | NC_000006.11; | | |
| | AC_000138.1; | | |
| HISTH1E | NC_018917.1 | NM_005321.2 | NP_005312.1 |
| ATM | NG_009830.1 | NM_000051.3 | NP_000042.3 |
| | | NM_000546.5; | NP_000537.3; |
| | | NM_001126112.2; | NP_001119584.1; |
| | | NM_001126113.2; | NP_001119585.1; |
| | | NM_001126114.2; | NP_001119586.1; |
| | | NM_001126115.1; | NP_001119587.1; |
| | | NM_001126116.1; | NP_001119588.1; |
| | | NM_001126117.1; | NP_001119589.1; |
| TP53 | NG_017013.2 | NM_001126118.1 | NP_001119590.1 |
| | | NM_001172566.1; | NP_001166037.1; |
| | | NM_001172567.1; | NP_001166038.1; |
| | | NM_001172568.1; | NP_001166039.1; |
| | | NM_001172569.1; | NP_001166040.1; |
| MYD88 | NG_016964.1 | NM_002468.4 | NP_002459.2 |
| NOTCH1 | NG_007458.1 | NM_017617.3 | NP_060087.3 |
| | | NM_001193416.1; | NP_001180345.1; |
| | | NM_001193417.1; | NP_001180346.1; |
| DDX3X | NG_012830.1 | NM_001356.3 | NP_001347.3 |
| | | NM_001171162.1; | NP_001164633.1; |
| | | NM_001171163.1; | NP_001164634.1; |
| | | NM_005096.3; | NP_005087.1; |
| ZMYM3 | NG_016407.1 | NM_201599.2 | NP_963893.1 |
| | | NM_001013415.1; | NP_001013433.1; |
| | | NM_001257069.1; | NP_001243998.1; |
| | | NM_018315.4; | NP_060785.2; |
| FBXW7 | NG_029466.1 | NM_033632.3 | NP_361014.1 |
| | NC_000002.11; | | |
| | AC_000134.1; | | |
| XPO1 | NC_018913.1 | NM_003400.3 | NP_003391.1 |
| | | NM_001042572.2; | NP_001036037.1; |
| CHD2 | NG_012826.1 | NM_001271.3 | NP_001262.3 |
| | | NM_001042594.1; | NP_001036059.1; |
| POT1 | NG_029232.1 | NM_015450.2 | NP_056265.2 |
| | | NM_002745.4; | NP_002736.3; |
| MAPK1 | NG_023054.1 | NM_138957.2 | NP_620407.1 |

SF3B1 mutation-specific reagents and/or CLL driver mutation-specific reagents useful in the practice of the disclosed methods include nucleic acids (polynucleotides) and amino acid based reagents such as proteins (e.g., antibodies or antibody fragments) and peptides.

SF3B1 mutation-specific reagents and/or CLL driver mutation-specific reagents useful in the practice of the disclosed methods include, among others, mutant polypeptide specific antibodies and AQUA peptides (heavy-isotope labeled peptides) corresponding to, and suitable for detection and quantification of, mutant polypeptide expression in a biological sample. A mutant polypeptide-specific reagent is any reagent, biological or chemical, capable of specifically binding to, detecting and/or quantifying the presence/level of expressed mutant polypeptide in a biological sample, while not binding to or detecting wild type. The term includes, but is not limited to, the preferred antibody and AQUA peptide reagents discussed below, and equivalent reagents are within the scope of the present invention. The mutation-specific reagents specifically recognize SF3B1 with missense mutations, for example, a SF3B1 polypeptide with mutations at R625L, N626H, K700E, K741N, G740E, E622D, R625G, Q659R, K666Q, K666E, G742D or Q903R. In some aspects, the mutation-specific reagents specifically recognize CLL driver mutations, including but not limited to mutations in HIST1H1E, NRAS, BCOR, RIPK1, SAMHD1, KRAS, MED12, ITPKB, EGR2, DDX3X, ZMYM3, FBXW7, ATM, TP53, MYD88, NOTCH1, XPO1, CHD2, POT1, del(8p), del(13q), del(11q), del(17p), and trisomy 12.

Reagents suitable for use in practice of the methods of the invention include a mutant polypeptide-specific antibody. A mutant-specific antibody of the invention is an isolated antibody or antibodies that specifically bind(s) a mutant polypeptide of the invention, but does not substantially bind either wild type or mutants with mutations at other positions.

Mutant-specific reagents provided by the invention also include nucleic acid probes and primers suitable for detection of a mutant polynucleotide. These probes are used in assays such as fluorescence in-situ hybridization (FISH) or polymerase chain reaction (PCR) amplification. These mutant-specific reagents specifically recognize or detect nucleic acids encoding a mutant SF3B1 polypeptide, wherein the mutations are at R625L, N626H, K700E, K741N, G740E, E622D, R625G, Q659R, K666Q, K666E, G742D or Q903R. In some aspects, the mutation-specific reagents specifically recognize other CLL driver mutations, including but not limited to mutations in HIST1H1E, NRAS, BCOR, RIPK1, SAMHD1, KRAS, MED12, ITPKB, EGR2, DDX3X, ZMYM3, FBXW7, ATM, TP53, MYD88, NOTCH1, XPO1, CHD2, POT1, del(8p), del(13q), del(11q), del(17p), and trisomy 12.

Mutant polypeptide-specific reagents useful in practicing the methods of the invention may also be mRNA, oligonucleotide or DNA probes that can directly hybridize to, and detect, mutant or truncated polypeptide expression transcripts in a biological sample. Briefly, and by way of example, formalin-fixed, paraffin-embedded patient samples may be probed with a fluorescein-labeled RNA probe followed by washes with formamide, SSC and PBS and analysis with a fluorescent microscope.

Polynucleotides encoding the mutant polypeptide may also be used for diagnostic/prognostic purposes. The polynucleotides that may be used include oligonucleotide sequences, antisense RNA and DNA molecules. The polynucleotides may be used to detect and quantitate gene expression in biopsied tissues, for example the expression of the S3FB1 gene and/or other CLL genes. For example, the diagnostic assay may be used to distinguish between absence, presence, and increased or excess expression of nucleic acids encoding the mutant polypeptide, and to monitor regulation of mutant polypeptide levels during therapeutic intervention.

In one preferred embodiment, hybridization with PCR probes which are capable of detecting polynucleotide sequences, including genomic sequences, encoding mutant polypeptide or truncated active polypeptide, or closely related molecules, may be used to identify nucleic acid sequences which encode mutant polypeptide. The construction and use of such probes is described above. The specificity of the probe, whether it is made from a highly specific region, e.g., 10 unique nucleotides in the mutant junction, or a less specific region, e.g., the 3′ coding region, and the stringency of the hybridization or amplification (maximal, high, intermediate, or low) will determine whether the probe identifies only naturally occurring sequences encoding mutant SF3B1 and/or other CLL mutant polypeptides, alleles, or related sequences.

Probes may also be used for the detection of related sequences, and should preferably contain at least 50% of the nucleotides from any of the mutant polypeptide encoding sequences. The hybridization probes of the subject invention may be DNA or RNA and derived from the nucleotide sequence and encompassing the mutation, or from genomic sequence including promoter, enhancer elements, and introns of the naturally occurring polypeptides but comprising the mutation.

A mutant polynucleotide may be used in Southern or Northern analysis, dot blot, or other membrane-based technologies; in PCR technologies; or in dip stick, pin, ELISA or chip assays utilizing fluids or tissues from patient biopsies to detect altered polypeptide expression. Such qualitative or quantitative methods are well known in the art. Mutant polynucleotides may be labeled by standard methods, and added to a fluid or tissue sample from a patient under conditions suitable for the formation of hybridization complexes. After a suitable incubation period, the sample is washed and the signal is quantitated and compared with a standard value. If the amount of signal in the biopsied or extracted sample is significantly altered from that of a comparable control sample, the nucleotide sequences have hybridized with nucleotide sequences in the sample, and the presence of altered levels of nucleotide sequences encoding mutant polypeptide in the sample indicates the presence of the associated disease. Such assays may also be used to evaluate the efficacy of a particular therapeutic treatment regimen in animal studies, in clinical trials, or in monitoring the treatment of an individual patient.

In order to provide a basis for the diagnosis of disease characterized by expression of mutant polypeptide, a normal or standard profile for expression is established. This may be accomplished by combining body fluids or cell extracts taken from normal subjects, either animal or human, with a sequence, or a fragment thereof, which encodes mutant polypeptide, under conditions suitable for hybridization or amplification. Standard hybridization may be quantified by comparing the values obtained from normal subjects with those from an experiment where a known amount of a substantially purified polynucleotide is used. Standard values obtained from normal samples may be compared with values obtained from samples from patients who are symptomatic for disease. Deviation between standard and subject values is used to establish the presence of disease.

Once disease is established and a treatment protocol is initiated, hybridization assays may be repeated on a regular basis to evaluate whether the level of expression in the patient begins to approximate that which is observed in the normal patient. The results obtained from successive assays may be used to show the efficacy of treatment over a period ranging from several days to months.

Additional diagnostic uses for mutant polynucleotides of the invention may involve the use of polymerase chain reaction (PCR), a preferred assay format that is standard to those of skill in the art. See, e.g., MOLECULAR CLONING, A LABORATORY MANUAL, 2nd edition, Sambrook, J., Fritsch, E. F. and Maniatis, T., eds., Cold Spring Harbor Laboratory Press, Cold Spring Harbor, N.Y. (1989). PCR oligomers may be chemically synthesized, generated enzymatically, or produced from a recombinant source. Oligomers will preferably consist of two nucleotide sequences, one with sense orientation (5′ to 3′) and another with antisense (3′ to 5′), employed under optimized conditions for identification of a specific gene or condition. The same two oligomers, nested sets of oligomers, or even a degenerate pool of oligomers may be employed under less stringent conditions for detection and/or quantitation of closely related DNA or RNA sequences.

In certain preferred embodiments, sequencing technologies, including but not limited to whole genome sequencing (WGS), whole exome sequencing (WES), deep sequencing, and targeted gene sequencing, are used to detect, measure, or analyze a sample for the presence of a CLL mutation.

WGS (also known as full genome sequencing, complete genome sequencing, or entire genome sequencing), is a process that determines the complete DNA sequence of a subject. In some aspects, WGS, as embodied in the methods of Ng and Kirkness, Methods Mol. Biol.; 628:215-26 (2010), may be employed with the methods of the present disclosure to detect CLL mutations in a sample.

WES (also known as exome sequencing, or targeted exome capture), is an efficient strategy to selectively sequence the coding regions of the genome of a subject as a cheaper but still effective alternative to WGS. As exemplified by the methods of Gnirke et al., Nature Biotechnology 27, 182-189 (2009), WES of tumors and their patient-matched normal samples is an affordable, rapid and comprehensive technology for detecting somatic coding mutations. In some aspects, WES may be employed with the methods of the present disclosure to detect CLL mutations in a sample.

Deep sequencing methods provide for greater coverage (depth) in targeted sequencing approaches. “Deep sequencing,” “deep coverage,” or “depth” refers to having a high amount of coverage for every nucleotide being sequenced. The high coverage allows not only the detection of nucleotide changes, but also the degree of heterogeneity at every single base in a genetic sample. Moreover, deep sequencing is able to simultaneously detect small indels and large deletions, map exact breakpoints, calculate deletion heterogeneity, and monitor copy number changes. In some aspects, deep sequencing strategies, as provided by Myllykangas and Ji, Biotechnol Genet Eng Rev. 27:135-58 (2010), may be employed with the methods of the present disclosure to detect CLL mutations in a sample.

In preferred embodiments, sequencing technologies, including but not limited to whole genome sequencing (WGS), whole exome sequencing (WES), deep sequencing, and targeted gene sequencing, as described herein, are used to determine whether a CLL mutation in a sample is clonal or subclonal. In some examples, WES of tumors and their patient-matched normal samples combined with analytical tools provides for analysis of subclonal mutations because: (i) the high sequencing depth obtained by WES (typically ˜100-150×) enables reliable detection of a sufficient number of subclonal mutations required for defining subclones and tracking them over time; (ii) coding mutations likely encompass many of the important driver events that provide fitness advantage for specific clones; and finally, (iii) the relatively low cost of whole-exome sequencing permits studies of large cohorts, which is key for understanding the relative fitness and temporal order of driver mutations and for assessing the impact of clonal heterogeneity on disease outcome. WES thus allows for identification of CLL subclones and the mutations that they harbor by integrative analysis of coding mutations and somatic copy number alterations, which enable estimation of the cancer cell fraction (CCF). WES analysis further provides for the study of mutation frequencies, observation of clonal evolution, and linking of subclonal mutations to clinical outcome.

In some examples, the sequencing data generated using sequencing technologies is processed using analytical tools including but not limited to the Picard data processing pipeline (DePristo et al., Nat. Genet. 43, 491-498 (2011)), the Firehose pipeline available at The Broad Institute, Inc. website, MutSig available at The Broad Institute, Inc. website, HAPSEG (Carter et al., Available from Nature Preceedings), GISTIC2.0 algorithm (Mermel et al., Genome Biol. 12(4):R41 (2011)), and ABSOLUTE available at The Broad Institute, Inc. website. Such analytical tools allow for, in some examples, the identification of sSNVs, sCNAs, indels, and other structural chromosomal rearrangements, and provide for the determination of sample purity, ploidy, and absolute somatic copy numbers. In some examples, the use of analytical tools with sequencing data obtained from a CLL sample allows for the determination of the cancer cell fraction (CCF) harboring a mutation, thus identifying whether a mutation is clonal or subclonal.

Methods which may also be used to quantitate the expression of mutant polynucleotide include radiolabeling or biotinylating nucleotides, coamplification of a control nucleic acid, and standard curves onto which the experimental results are interpolated (Melby et al., J. Immunol. Methods, 159:235-244 (1993); Duplaa et al. Anal. Biochem. 229-236 (1993)). The speed of quantitation of multiple samples may be accelerated by running the assay in an ELISA format where the oligomer of interest is presented in various dilutions and a spectrophotometric or calorimetric response gives rapid quantitation.

Other suitable methods for nucleic acid detection, such as minor groove-binding conjugated oligonucleotide probes (see, e.g. U.S. Pat. No. 6,951,930, “Hybridization-Triggered Fluorescent Detection of Nucleic Acids”) are known to those of skill in the art. Also provided by the invention is a kit for the detection of the mutation in a biological sample, the kit comprising an isolated mutant-specific reagent of the invention and one or more secondary reagents. Suitable secondary reagents for employment in a kit are familiar to those of skill in the art, and include, by way of example, buffers, detectable secondary antibodies or probes, activating agents, and the like.

In some aspects, a kit is provided for the detection of a mutation in a biological sample, the kit comprising isolated mutant-specific reagents for the detection of a mutation in one or more CLL drivers in the group consisting of SF3B1, NRAS, KRAS, BCOR, EGR2, MED12, RIPK1, SAMHD1, ITPKB, HIST1H1E, ATM, TP53, MYD88, NOTCH1, DDX3X, ZMYM3, FBXW7, XPO1, CHD2, POT1, del(8p), del(13q), del(11q), del(17p), and trisomy 12. In some aspects, the kit further comprises reagents for evaluating the degree of somatic hypermutation in the IGHV gene; and reagents for evaluating the expression status of ZAP70.

In some aspects, a kit is provided for the detection of a mutation in a biological sample, the kit comprising mutant-specific reagents comprising mutant-specific antibodies that specifically bind a mutant polypeptide encoded by a CLL gene, but does not substantially bind either wild type or mutants with mutations at other positions. Such antibodies are used in assays such as immunohistochemistry (IHC), ELISA, and flow cytometry assays such as fluorescence activated cell sorting (FACS).

In some aspects, a kit is provided for the detection of a mutation in a biological sample, the kit comprising mutant-specific reagents comprising nucleic acid probes and primers suitable for detection of a CLL mutation. These probes are used in assays such as fluorescence in-situ hybridization (FISH) or polymerase chain reaction (PCR) amplification. These mutant-specific reagents specifically recognize or detect nucleic acids of a CLL driver in a biological sample.

In some aspects, a kit is provided for the detection of a mutation in a biological sample, the kit comprising mutant-specific reagents comprising mRNA, oligonucleotide or DNA probes that can directly hybridize to, and detect, mutant or truncated expression transcripts off a CLL driver, or directly hybridize to and detect chromosomal abnormalities in a biological sample.

In some aspects, a kit is provided for the detection of a mutation in a biological sample, the kit comprising a single nucleotide polymorphism (SNP) array that detects one or more mutations in a CLL gene.

In some aspects, a kit is provided for the detection of a mutation in a biological sample, the kit comprising mutant-specific reagents for the detection of one or more mutations in one or more CLL drivers using sequencing methods such as whole genome sequencing (WGS), whole exome sequencing, deep sequencing, targeted sequencing of cancer genes, or any combination thereof, as described herein.

In preferred embodiments, any kit described herein further comprises instructions for use.

The methods of the invention may be carried out in a variety of different assay formats known to those of skill in the art.

### Other Clinical Indicators

Other clinical indicators that are useful for diagnosing, prognosing, or evaluating a subject with CLL for determining treatment regimens or predicting survival are known in the art. These other clinical indicators are referred to herein as “CLL biomarkers” or CLL-associated markers and include, for example, but are not limited to mutations in CLL-associated genes, increased expression of CLL-associated genes, chromosomal rearrangements, and micro-RNAs. These other clinical indicators can also be used in methods of the present invention in combination with identifying a SF3B1 and/or CLL driver mutation.

Other biomarkers associated with CLL that may be used in the methods described herein include, for example, mutated IGHV, increased expression of ZAP70, increased levels of β2-microglobulin, increased levels of enzyme sTK, increased CD38 expression, and increased levels of Ang-2. Other genes that are known in the art to be indicative or prognostic of CLL initiation, progression or response to treatment can also be used in the present invention. Polynucledotides encoding these biomarkers or the polypeptides of the CLL biomarkers disclosed herein can be detected or the levels can be determined by methods known in the art and described herein. For example, the mutational status of IGHV can be assessed by various DNA sequencing methods known in the art, such as Sanger sequencing. In other embodiments, CD38 and ZAP70 expression levels can be assessed by flow cytometry.

Other CLL biomarkers can include various chromosomal abnormalities, such as 11q deletion, 17p deletion, Trisomy 12, 13q deletion, monosomy 13, and rearrangements of chromosome 14. Other chromosomal rearrangements, amplifications, deletions, or other abnormalities can also be used in the methods described herein. Particularly of interest are chromosomal abnormalities, rearrangements, or deletions that affect p53 or ATM function, wherein p53 and/or ATM function is decreased or inhibited. Methods for identifying chromosomal status are well known in the art. For example, fluorescence in-situ hybridization (FISH) can be utilized to detect chromosomal abnormalities.

Additional clinical indicators for CLL include lymphocyte doubling time, which can be calculated by determining the number of months it takes for the absolute lymphocyte count to double in number. Another clinical indicator for CLL includes atypical circulating lymphocytes in the blood, wherein the lymphocytes show abnormal nuclei (such as cleaved or lobated), irregular nuclear contours, or enlarged size.

### Therapeutic Administration

The invention includes administering to a subject compositions comprising an SF3B1 modulator such as an inhibitor.

SF3B1 modulators such as inhibitors alter splicing activity, for example, reduce, decrease, increase, activate or inhibit the biological function of SF3B1, such as splicing. SF3B1 inhibitors can be readily identified by an ordinarily skilled artisan by assaying for altered SF3B1 activity, i.e., splicing.

Altered splicing of genes can be measured by detecting a certain gene or subset of genes that are known to be spliced by SF3b spliceosome complex, or SF3B1 in particular, by methods known in the art and described herein. For example, the genes are ROIK3 or BRD2.

Other therapeutic regimens are contemplated by the invention as described above.

An effective amount of a therapeutic compound is preferably from about 0.1 mg/kg to about 150 mg/kg. Effective doses vary, as recognized by those skilled in the art, depending on route of administration, excipient usage, and coadministration with other therapeutic treatments including use of other anti-proliferative agents or therapeutic agents for treating, preventing or alleviating a symptom of a cancer. A therapeutic regimen is carried out by identifying a mammal, e.g., a human patient suffering from a cancer that has a SF3B1 mutation using standard methods.

The pharmaceutical compound is administered to such an individual using methods known in the art. Preferably, the compound is administered orally, rectally, nasally, topically or parenterally, e.g., subcutaneously, intraperitoneally, intramuscularly, and intravenously. The modulators (such as inhibitors) are optionally formulated as a component of a cocktail of therapeutic drugs to treat cancers. Examples of formulations suitable for parenteral administration include aqueous solutions of the active agent in an isotonic saline solution, a 5% glucose solution, or another standard pharmaceutically acceptable excipient. Standard solubilizing agents such as PVP or cyclodextrins are also utilized as pharmaceutical excipients for delivery of the therapeutic compounds.

The therapeutic compounds described herein are formulated into compositions for other routes of administration utilizing conventional methods. For example, the therapeutic compounds are formulated in a capsule or a tablet for oral administration. Capsules may contain any standard pharmaceutically acceptable materials such as gelatin or cellulose. Tablets may be formulated in accordance with conventional procedures by compressing mixtures of a therapeutic compound with a solid carrier and a lubricant. Examples of solid carriers include starch and sugar bentonite. The compound is administered in the form of a hard shell tablet or a capsule containing a binder, e.g., lactose or mannitol, conventional filler, and a tableting agent. Other formulations include an ointment, suppository, paste, spray, patch, cream, gel, resorbable sponge, or foam. Such formulations are produced using methods well known in the art.

Therapeutic compounds are effective upon direct contact of the compound with the affected tissue. Accordingly, the compound is administered topically. Alternatively, the therapeutic compounds are administered systemically. For example, the compounds are administered by inhalation. The compounds are delivered in the form of an aerosol spray from pressured container or dispenser which contains a suitable propellant, e.g., a gas such as carbon dioxide, or a nebulizer.

Additionally, compounds are administered by implanting (either directly into an organ or subcutaneously) a solid or resorbable matrix which slowly releases the compound into adjacent and surrounding tissues of the subject.

### EXAMPLES

### Example 1

### General Methods

### Human Samples

Heparinized blood samples and skin biopsies were obtained from normal donors and patients enrolled on clinical research protocols that were approved by the Human Subjects Protection Committee at the Dana-Farber Cancer Institute (DFCI). In some cases, 2 ml of saliva was collected from study participants as a source of normal epithelial cell DNA. Peripheral blood mononuclear cells (PBMC) from normal donors and patients were isolated by Ficoll/Hypaque density gradient centrifugation. CD19+ B cells from normal volunteers were isolated by immunomagnetic selection (Miltenyi Biotec, Auburn Calif.). Mononuclear cells were used fresh or cryopreserved with FBS 10% DMSO and stored in vapor-phase liquid nitrogen until the time of analysis. Primary skin fibroblast lines were generated from five mm diameter punch biopsies of skin that were provided to the Cell Culture Core lab of the Harvard Skin Disease Research Center, as previously described (Zhang, Clin Cancer Res 2010; 16:2729-39). Second or third passage cultures were used for genomic DNA isolation.

Prognostic Factor Analysis.

Immunoglobulin heavy-chain variable (IGHV) homology (high risk unmutated was defined as greater than or equal to 98% homology to the closest germline match) and ZAP-70 expression (high risk positive defined as >20%) were determined as previously described (Rassenti, N Engl J Med, 2004, 351:893-901). Cytogenetics were evaluated by FISH for the most common CLL abnormalities (del(13q), trisomy 12, del(11q), del(17p), rearrangements of chromosome 14; all probes from Vysis, Des Plaines, Ill.) at the Brigham and Women's Hospital Cytogenetics Laboratory, Boston Mass. (Dohner, N Engl J Med, 2000, 343:1910-6). Samples were scored positive for a chromosomal aberration based on consensus cytogenetic scoring (Cancer, Genet Cytogenet, 2010, 203:141-8). Percent tumor cells harboring common CLL cytogenetic abnormalities, detected by FISH cytogenetics, are tabulated per sample in Table 9.

Whole-Genome and -Exome DNA Sequencing.

Informed consent on DFCI IRB-approved protocols for whole genome sequencing of patients' samples was obtained prior to the initiation of sequencing studies. Genomic DNA was isolated from patient CD19⁺CD5⁺ tumor cells and autologous skin fibroblasts (Wizard kit; Promega, Madison Wis.) per manufacturer's instructions. Alternatively, germline genomic DNA was extracted from autologous epithelial cells, obtained from saliva samples (DNA Genotek, Kanata, Ontario, Canada) or from autologous blood granulocytes, isolated following Ficoll/Hypaque density gradient centrifugation.

Whole genome shotgun (WG) and whole exome (WE) capture libraries were constructed as previously described (Chapman, Nature, 2011, 471:467-72; Gnirke, Nat Biotechnol, 2009, 27:182-9; Berger, Nature, 2011, 470:214-20). For 51 (56%) of the 91 CLL samples included in the analysis, sequencing was performed on capture libraries generated from whole genome amplified (WGA) samples. For those samples, 100 ng inputs of samples were whole genome amplified with the Qiagen REPLI-g Midi Kit (Valencia, Calif.). No significant differences in mutation rate were observed between data originating from WGA and non-WGA samples (see Table 3). WGS libraries were sequenced on an average of 39 lanes of an Illumina GA-II sequencer, using 101 bp paired-end reads, with the aim of reaching 30× genomic coverage of distinct molecules per sample (Chapman, Nature, 2011, 471:467-72; Berger, Nature, 2011, 470:214-20). Exome sequencing libraries were sequenced on three lanes of the same instrument, using 76 bp paired-end reads.

Sequencing data subsequently was processed using the “Picard” pipeline, developed at the Broad Institute's Sequencing Platform (Fennell T, unpublished; Cambridge, Mass.), which includes base-quality recalibration (DePristo, Nat Genet. 2011, 43:491-8), alignment to the NCBI Human Reference Genome Build hg18 using MAQ (Li, Genome Res 2008, 18:1851-8), and aggregation of lane- and library-level data.

Identification of Somatic Tumor Mutations and Calculation of Significance.

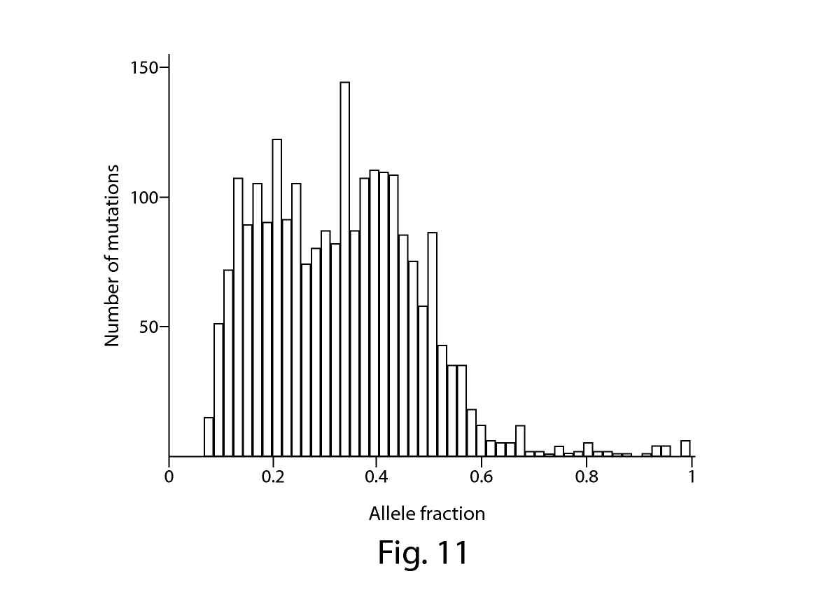

From the sequencing data, tumor-specific gene alterations were identified using a set of tools contained with the “Firehose” pipeline (Chapman, Nature, 2011, 471:467-72; Berger, Nature, 2011, 470:214-20), developed at the Broad Institute. Somatic single nucleotide variations (SSNVs) were detected using muTect, while somatic small insertions and deletions were detected using the algorithm Indelocator. The algorithm MutSig (Lawrence in preparation; (Ding, Nature 2008, 455:1069-75; Network, Nature 2008, 455:1061-8; Getz, Science 2007, 317:1500)) was applied to sequencing data from the 3 genomes and 88 exomes. Briefly, MutSig tabulates the number of mutations and the number of adequately covered bases for each gene (i.e. bases with >=14 tumor and >=8 normal reads). The counts are broken down by mutation context category (i.e. CpG transitions, other C:G transitions, any transversion, A:T transitions). For each gene, the probability of seeing the observed constellation of mutations or a more extreme one, given the background mutation rates calculated across the dataset was calculated (see Table 3 for background mutation rate). This is done by convoluting a set of binomial distributions as described previously, which results in a p and q value (Getz, Science 2007, 317:1500). The 4 samples for which normal germline DNA was derived from blood granulocytes had a significantly lower detection of somatic mutations, suggesting contamination with tumor DNA. Reanalysis excluding these 4 samples had little effect on mutation rate (increased by only 5%: 0.71 mutations/Mb to 0.75 mutations/Mb) and yielded the same results of significantly mutated genes (q<0.1). All mutations in genes that were significantly mutated or within pathways related to these significantly mutated genes were confirmed by manual inspection of the sequencing data (Robinson, Nat Biotechnol 2011; 29:24-6). Furthermore, these mutations were also validated using an independent platform (Sequenom mass spectrometry-based genotyping). There was no significant difference in non-synonymous mutation rate between IGHV-mutated and unmutated patients (despite 82% power to detect differences of 0.6 standard deviations; one-sided 0.05 level test) or between different clinical stages. The ability to detect mutations of low allele fraction depends on several factors, including the purity and ploidy of the sample, and the copy number at the locus in question. Graphical representation of the distribution of allelic fraction among the total number of 2348 mutations detected is depicted in FIG. 11. To estimate the rate of false-positive mutation calls, a subset of the putative somatic point mutations and indels were randomly chosen to be subjected to orthogonal validation by multiplexed Sequenom mass spectrometry assays. Because of the limited sensitivity of this assay at low allele fractions, the analysis was restricted to mutations that were present in the tumor at an allele fraction of at least one-third. The Sequenom assays were designed for 71 randomly selected mutations, and of these, 66 were successfully validated as somatic. The other 5 were deemed to be reference. This yields an estimated specificity of 93%.

Statistical Analysis of Mutation Rate in Association with Clinical Variables.

Clinical data were available from 91 CLL samples comprising the genome/exome sequenced discovery set, and from 101 CLL samples used for extension and validation. The association between patient characteristics and clinical variables such as time to first treatment (TTFT) and mutation rate or presence or absence of driver mutations was tested. P-values were calculated using the Wilcoxon rank sum test for quantitatively measured variables across two groups, the Fisher Exact test for categorical variables, the Kruskal-Wallis test for quantitatively measured variables across three groups and for ordered categorical data, and the log rank test for comparing Kaplan-Meier estimated censored time to event variables. Time to first therapy was defined as the elapsed time between initial diagnosis and first treatment for CLL. Patients who remained untreated for their disease at the most recent follow-up were censored at that time. All statistical tests were performed using SAS software version 9.2 and R version 2.8.0.

Univariate analysis was performed using Cox proportional hazards regression for the 19 variables potentially predictive of TTFT including (IGHV mutated vs. unmutated vs. unknown, ZAP-70 negative vs. positive vs. unknown, Rai stage at sampling 0/1 vs 2/3/4 vs unknown, age (≧55 yrs. vs. <55 yrs), sex, presence of del(17p), del(11q), trisomy(12), homozygous del(13q), heterozygous del(13q), presence of mutations in ATM, NOTCH1, SF3B1, TP53, DDX3X, ZMYM3, FBXW7, MYD88. A stepwise Cox proportional hazards regression model of TTFT was performed for the 91 discovery samples, using the 19 variables listed above. The same final model was obtained with a forward selection procedure. Step-up models using the −2 log likelihood statistic to assess goodness of fit using the appropriate degrees of freedoms were also explored. Cox modeling results are reported as hazard ratios along with the 95% confidence intervals.

Detection of Altered RNA Splicing.

Total RNA was extracted from normal B and CLL-B cells (TRIZOL; Invitrogen, Carlsbad Calif.). 2 μg total RNA from each sample was treated with DNase I (2 units/sample; New England BioLabs, Ipswich Mass.) at 37° C. for 20 minutes to remove contaminating genomic DNA, followed by heat-inactivation of DNase I at 75° C. for 15 minutes, and then used as template to synthesize cDNA by reverse transcription (SuperScript® III First-Strand kit; Invitrogen, Carlsbad Calif.). We designed in parallel quantitative Taqman assays primers to detected spliced transcripts across consecutive exons, and unspliced transcripts in which one primer was localized within the retained intron. Details of primer design the splicing assays for RIOK3, and BRD2 are noted in Table 11. All assays were run in triplicate using the 7500 Fast System (Applied Biosystems, Carlsbad Calif.), and all values were normalized to GAPDH gene expression. Relative splicing activity was measured by calculating the ratio of unspliced to spliced forms of each target gene. For some experiments, splicing was measured following treatment of 293 cells or normal B cells or CLL cells with the SF3b-complex targeting drug E7107 at 1 μM (gift of Robin Reed, HMS).

### Example 2

### CLL Carries a Low Somatic Mutation Rate

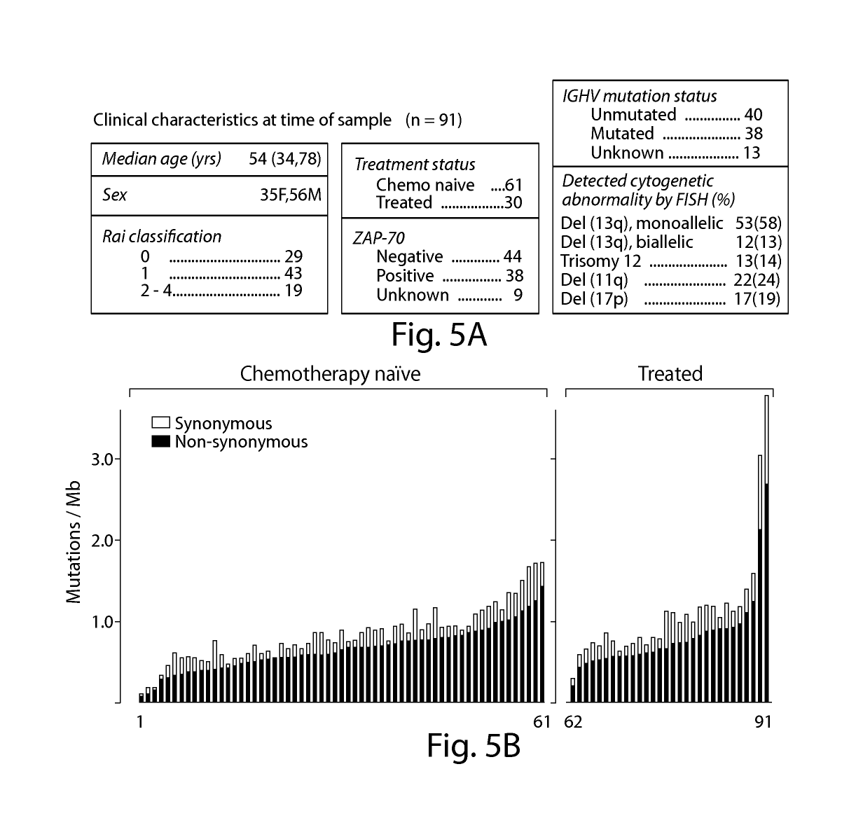

DNA derived from CD19+CD5+ leukemia cells was sequenced and matched germline DNA derived from autologous skin fibroblasts, saliva-derived epithelial cells or blood granulocytes. Samples were taken from patients displaying a broad range of clinical characteristics, including the high-risk deletions of chromosomes 11q and 17p, and both unmutated and mutated IGHV (FIG. 5A). Deep sequence coverage was obtained to enable high sensitivity in identifying mutations (Table 1). To detect point mutations and insertions or deletions (indels), sequences of each tumor were compared to its corresponding normal using well-validated algorithms (Chapman, Nature, 2011, 471:467-72; CGARN, Nature, 2011, 474:609-15; Berger, Nature, 2011, 470:214-20; Robinson, Nat Biotechnol 2011; 29:24-6)

1838 non-synonymous and 539 synonymous mutations were detected in protein-coding sequences, corresponding to an average somatic mutation rate of 0.72/Mb (SD=0.36, range 0.075-2.14), and an average of 20 non-synonymous mutations per individual (range 2-76) (Table 1; Table 2). This rate is similar to that previously reported for CLL and other hematologic malignancies (Fabbri, J Exp Med, 2011; Puente, Nature, 2011; Chapman, Nature, 2011, 471:467-72; Mardis, N Engl J Med 2009, 361:1058-66; Ley, Nature 2008; 456:66-72). There was no significant difference in non-synonymous mutation rate between IGHV-mutated and -unmutated tumors or between different clinical stages of disease (Table 3). Prior exposure to chemotherapy (30 of 91 samples) was not associated with increased non-synonymous mutation rate (p=0.14, FIG. 5B) (CGARN, Nature, 2008, 455:1061-8).

### Example 3

### Identification of Significantly Mutated Genes in CLL

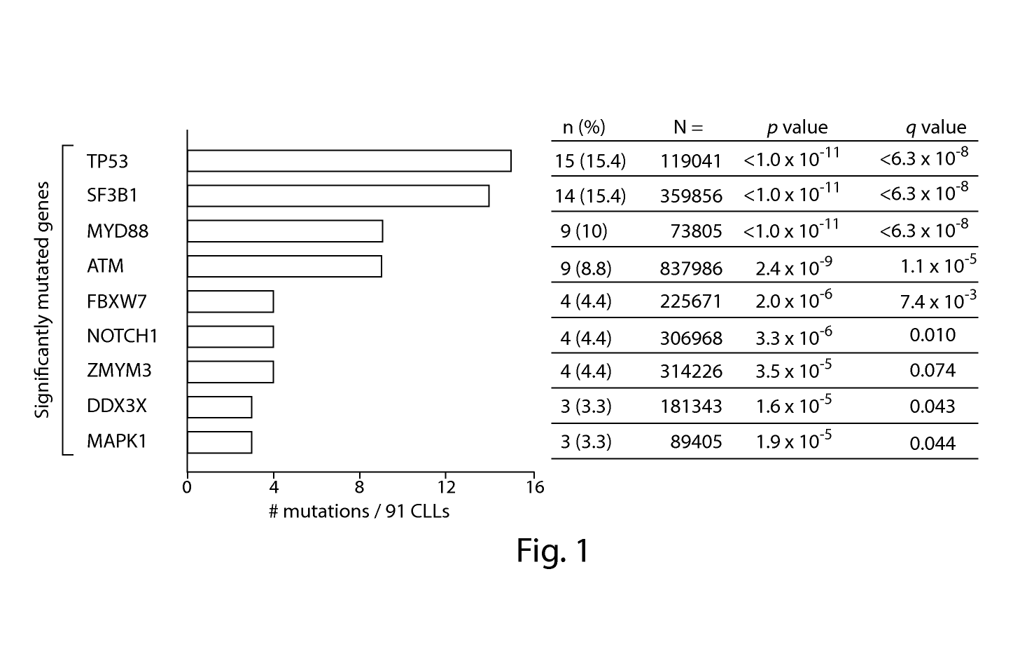

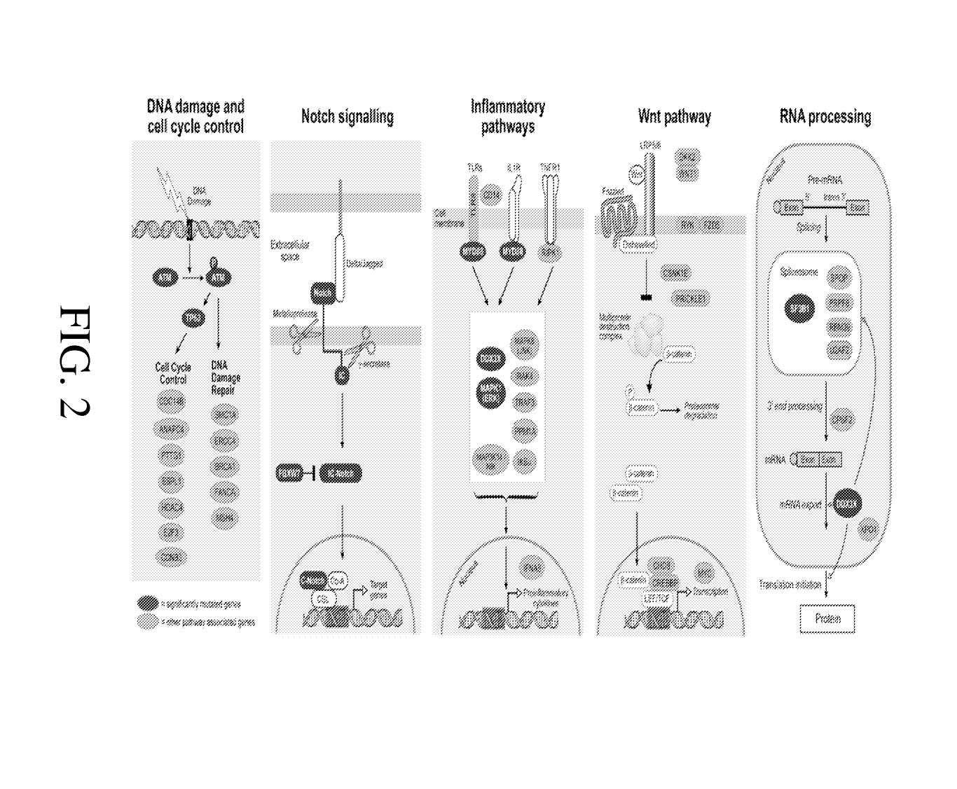

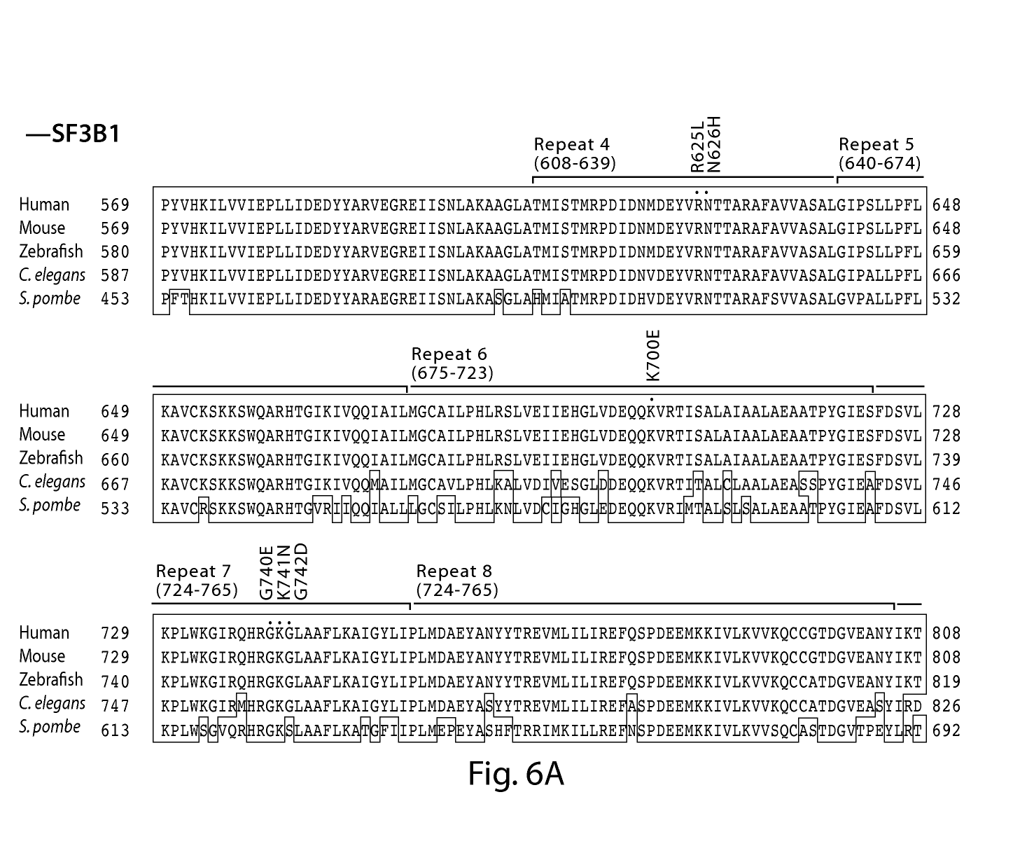

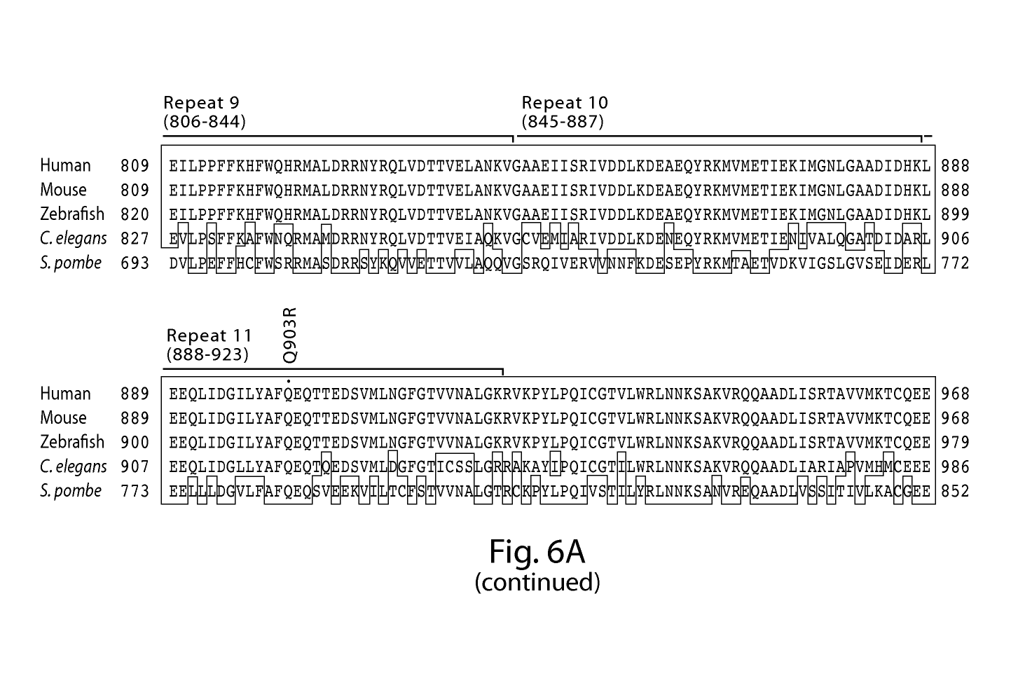

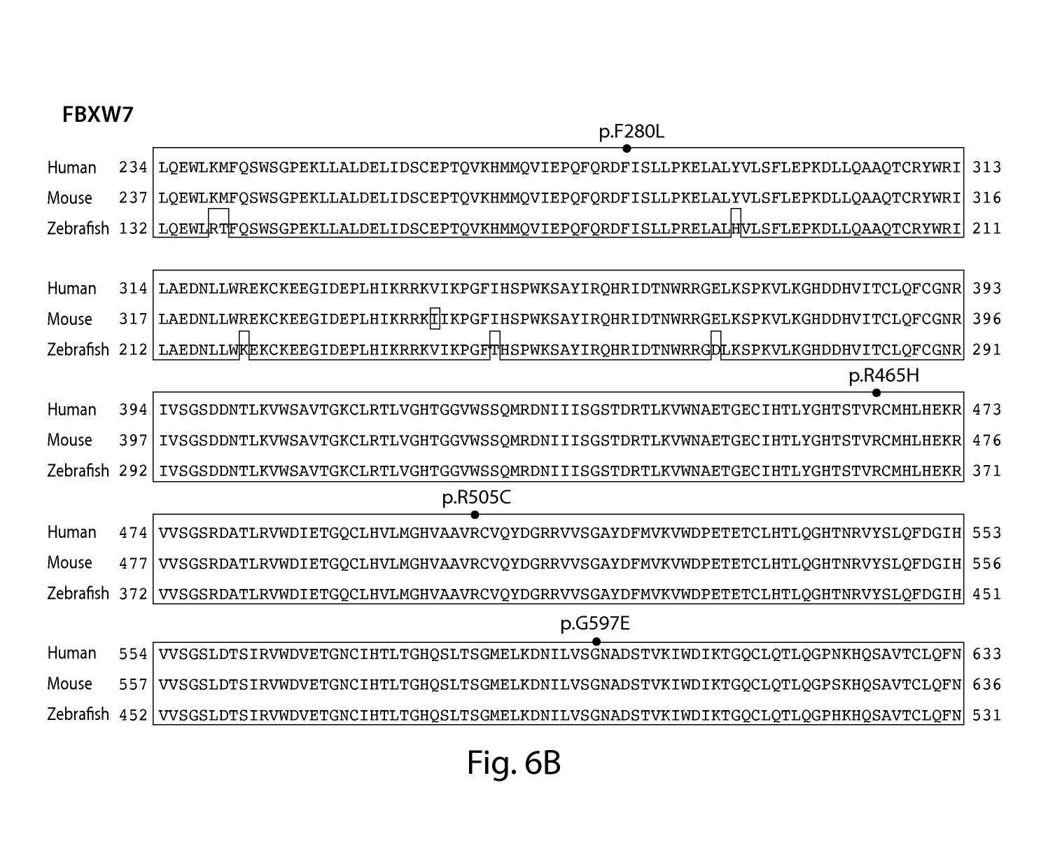

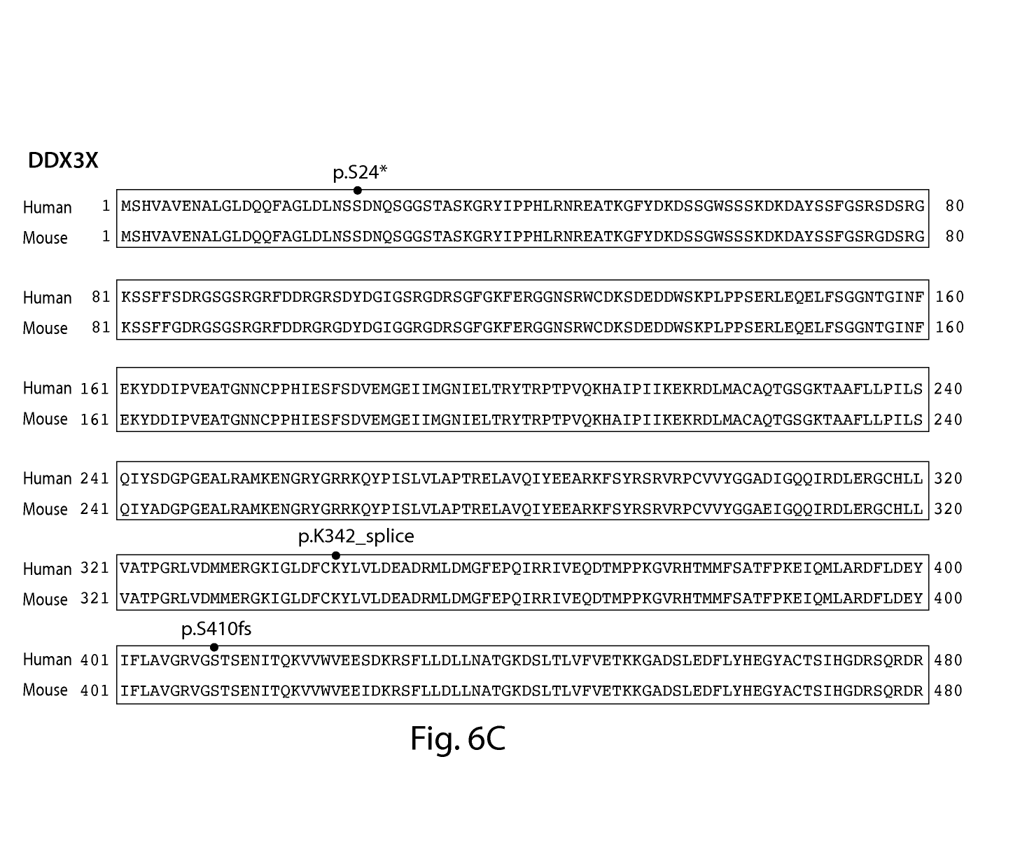

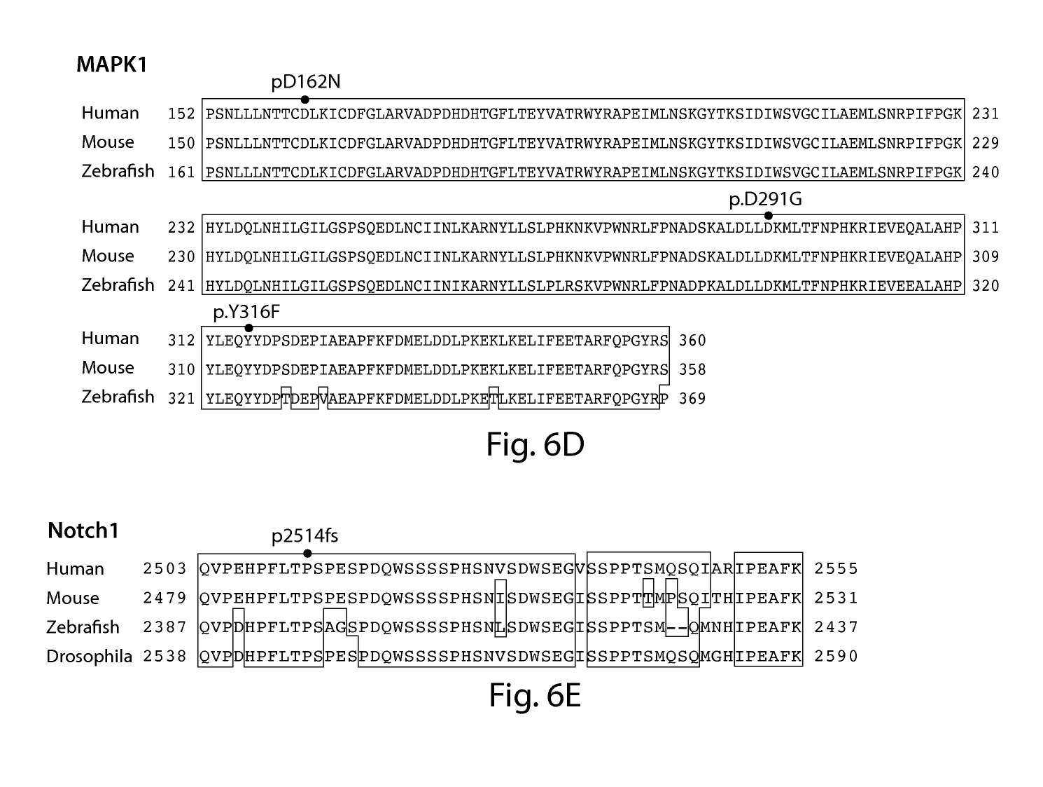

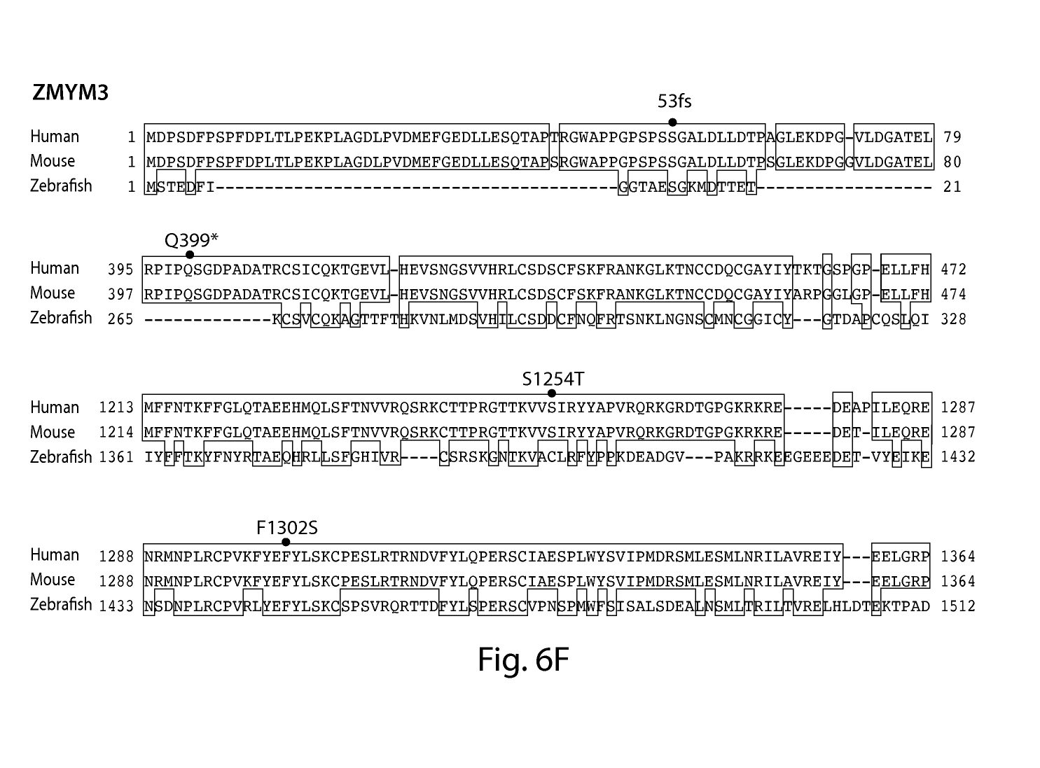

To identify genes whose mutations were associated with CLL tumorigenesis (‘driver’ mutations), all 91 leukemia/normal pairs were examined using the MutSig algorithm for genes that were mutated significantly more than the background rate given their sequence composition. Eight such genes were identified, with q<0.1 after correction for multiple hypothesis testing: TP53, SF3B1, MYD88, ATM, FBXW7, NOTCH1, ZMYM3, and DDX3X (FIG. 1). Whereas the overall ratio of non-synonymous/synonymous (NS/S) mutations was 3.1, the mutations in these 9 genes were exclusively non-synonymous (65:0, p<5×10⁻⁶, Table 2), further supporting their functional importance. Moreover, these gene mutations occurred exclusively in conserved sites across species (FIG. 6).

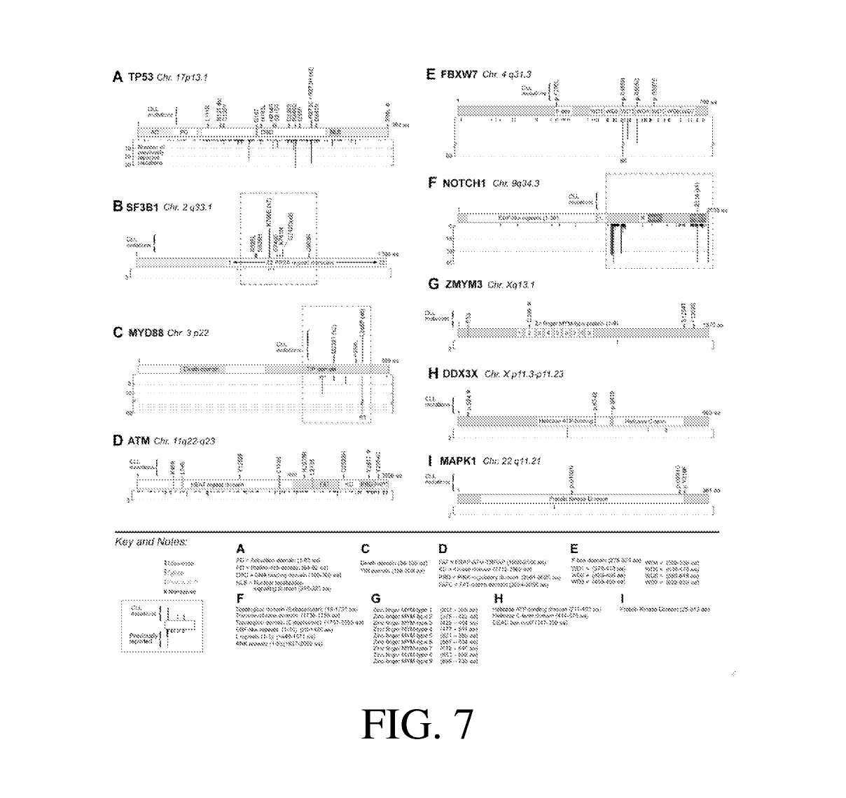

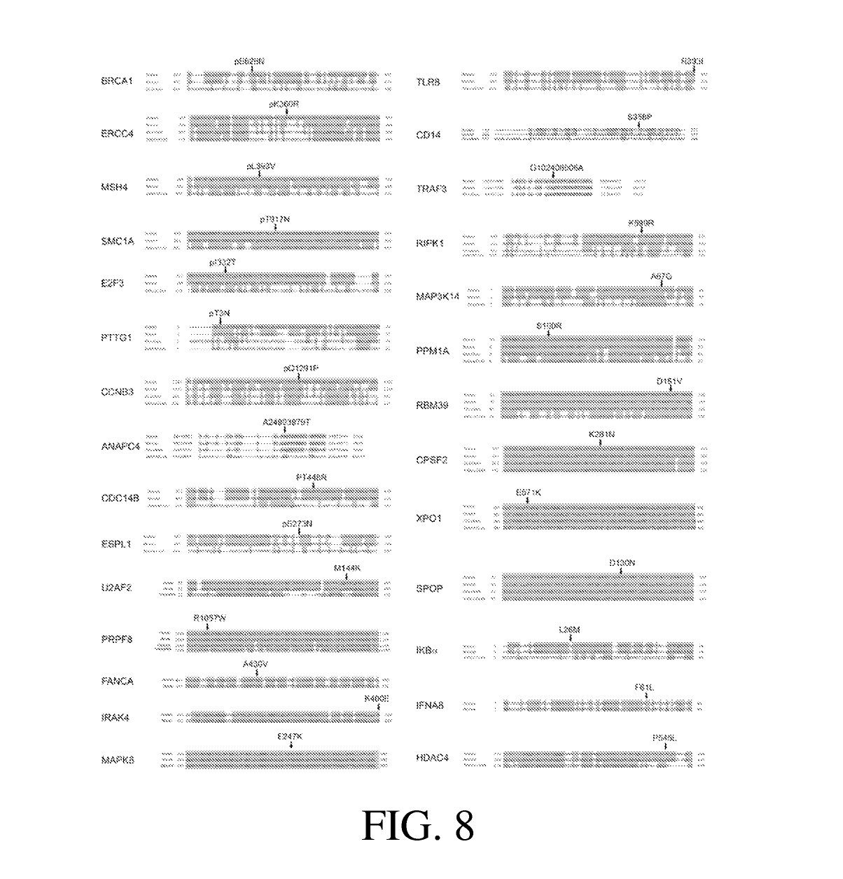

Four of the significantly mutated genes, TP53, ATM, MYD88 and NOTCH1, have been described previously in CLL (Puente, Nature, 2011; Austen, Blood, 2005, 106:3175-82; Zenz, J Clin Oncol, 2010, 28:4473-9; Trbusek, J Clin Oncol 2011; 29:2703-8). 15 TP53 mutations in 14 of 91 CLL samples (15%; q≦6.3×10⁻⁸), mostly localized to the DNA binding domain that is critical for its tumor suppressor activity (Zenz, J Clin Oncol, 2010, 28:4473-9) (FIG. 7A). In 8 samples, we detected 9 ATM mutations (9%; q<1.1×10⁻⁵) scattered across this large gene, including in regions where mutation has been associated with defective DNA repair in CLL (Austen, Blood, 2005, 106:3175-82) (FIG. 7D). MYD88, a critical adaptor molecule of the interleukin 1 receptor (IL1R)/Toll-like receptor (TLR)-mediated signaling pathway, harbored missense mutations in 9 CLL samples (10%) at 3 sites localized within 40 amino acids of the Toll/IL1R (TIR) domain. One site was novel (P258L), while the other two were identical to those recently described as activating mutations of the NF-κB/TLR pathway in diffuse large B-cell lymphoma (DLBCL) (M232T and L265P, FIG. 7C) (Ngo, Nature 2011, 470:115-9). Finally, we detected 4 CLLs (4%) with a recurrent frameshift mutation (P2514fs) in the C-terminal PEST domain of NOTCH1 identical to that recently reported in CLL (Fabbri, J Exp Med, 2011; Puente, Nature, 2011) (FIG. 7F). This mutation is associated with unmutated IGHV and poor prognosis (Fabbri, J Exp Med, 2011; Puente, Nature, 2011), and is predicted to cause impaired degradation of NOTCH1, leading to pathway activation.

Four of the significantly mutated genes (SF3B1, FBXW7, DDX3X, ZMYM3) have not been reported in CLL. Strikingly, the second most frequently mutated gene within our cohort was splicing factor 3b, subunit 1 (SF3B1), with missense mutations in 14 of 91 CLL samples (15%) (FIG. 7B). SF3B1 is a component of the SF3b complex, which associates with U2 snRNP at the catalytic center of the spliceosome (Wahl, Cell, 2009, 136:701-18). SF3B1, other U2 snRNP components, and defects in splicing have not been previously implicated in the biology of CLL. Remarkably, all 14 mutations localized within the C-terminal PP2A-repeat regions 5 to 8, which are highly conserved from human to yeast (FIGS. 6 and 7B), and 7 mutations produced an identical amino-acid change (K700E). Like MYD88 and NOTCH1, the clustering of heterozygous mutations within specific domains and at identical sites suggests that they cause specific functional changes. While the N-terminal domain of SF3B1 is known to interact directly with other spliceosome components (Wahl, Cell, 2009, 136:701-18), the precise role of its C-terminal domain remains unknown. Only 6 mutations have been reported in SF3B1, all in solid tumors and in the PP2A-repeat region (Table 5).

The four remaining significantly mutated genes are novel to CLL and appear to have functions that interact with the 5 frequently mutated genes cited above (FIG. 7). FBXW7 (4 distinct mutations) is an ubiquitin ligase and known as a tumor suppressor gene, with loss of expression in diverse cancers (Yada, EMBO J, 2004, 23:2116-25; Babaei-Jadidi, J Exp Med, 2011, 208:295-312) (FIG. 7E). Its targets include important oncoproteins such as Notch1, c-Myc, c-Jun, cyclin E1, and MCL1 (Yada, EMBO J, 2004, 23:2116-25; Babaei-Jadidi, J Exp Med, 2011, 208:295-312). Two of the 4 mutations in FBXW7 cause constitutive Notch signaling in T-cell acute lymphoblastic leukemia (O'Neil J Exp Med, 2007, 204:1813-24). DDX3X (3 distinct mutations) (FIG. 7H) is a RNA helicase that functions at multiple levels of RNA processing, including RNA splicing, transport, translation initiation, and regulation of an RNA-sensing proinflammatory pathway (Rosner, Curr Med Chem, 2007, 14:2517-25). Interestingly, DDX3X directly interacts with XPO1 (Rosner, Curr Med Chem, 2007, 14:2517-25) which was recently reported as mutated in 2.4% of CLL patients (Puente, Nature, 2011). MAPK1 (3 distinct mutations), also known as ERK, is a kinase that is involved in core cellular processes such as proliferation, differentiation, transcription regulation, development and is a key signaling component of the TLR pathway (Pepper, Blood, 2003, 101:2454-60; Muzio, Blood, 2008, 112:188-95). Two of three distinct MAPK1 mutations localize to the protein kinase domain, thus providing the first examples of somatic mutations within the protein-kinase domain of an ERK family member in a human cancer (FIG. 7I). Finally, we identified 4 distinct mutations in ZMYM3, a component of histone deacetylase-containing multiprotein complexes that function to silence genes through modifying chromatin structure (Lee, Nature, 2005, 437:432-5) (FIG. 7G).

The three most recurrent mutations, SF3B1-K700E, MYD88-L265P, and NOTCH1-P2514fs, were validated on 101 independent paired CLL-germline DNA samples, in which comparable detection frequencies was observed between the discovery and extension cohort (p=0.20, 0.58, and 0.38, respectively) (Table 6).

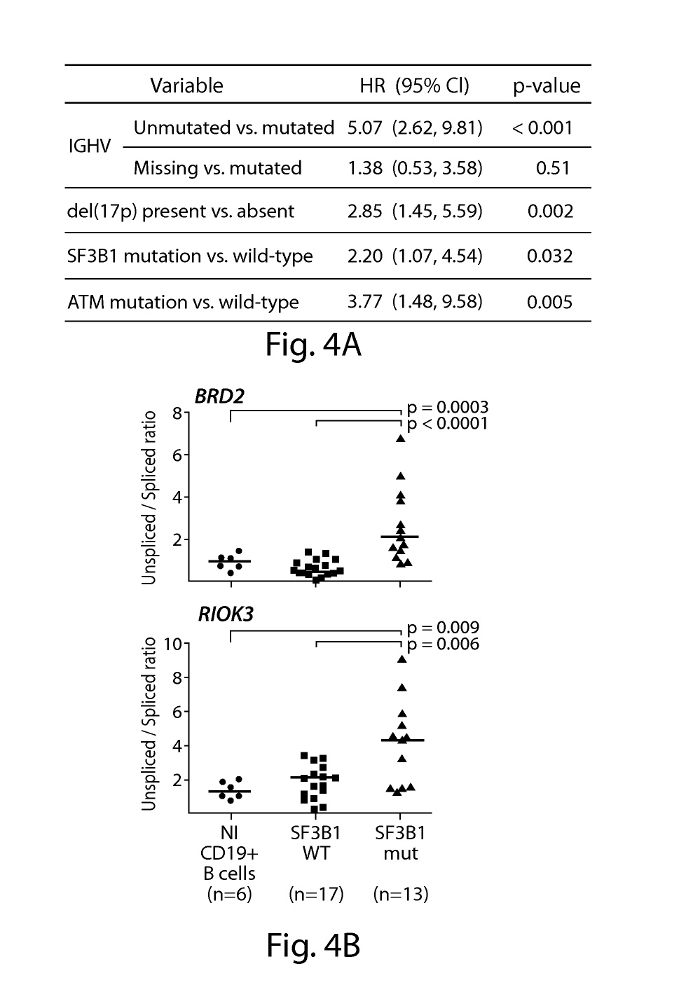

The nine significantly mutated genes fall into five core signaling pathways, in which the genes play well-established roles: DNA damage repair and cell-cycle control (TP53 and ATM), Notch signaling (FBXW7 and NOTCH1 (O'Neil J Exp Med, 2007, 204:1813-24)), inflammatory pathways (MYD88 and DDX3X) and RNA splicing/processing (SF3B1, DDX3X) (FIG. 2). We also noticed that additional genes are mutated in these pathways (as defined by the MSigDB Canonical Pathway database (Subramanian, Proc Natl Acad Sci USA, 2005, 102:15545-50) and literature) (FIG. 2; FIG. 4 and Table 7). Although these genes do not reach statistical significance alone or as a set, they might do so in a larger collection of samples. On the other hand, 19 of 59 genes classified as members of the Wnt signaling pathway, which has been implicated in CLL based on gene expression studies (Gutierrez, Blood, 2010; Klein, J Exp Med 2001, 194:1625-38), were mutated within our cohort. Although no individual gene reached significance, the Wnt pathway, as a set, showed a high frequency of mutations (p=0.048, FIG. 2).

### Example 4

### Driver Mutations are Associated with Distinct Clinical Groups

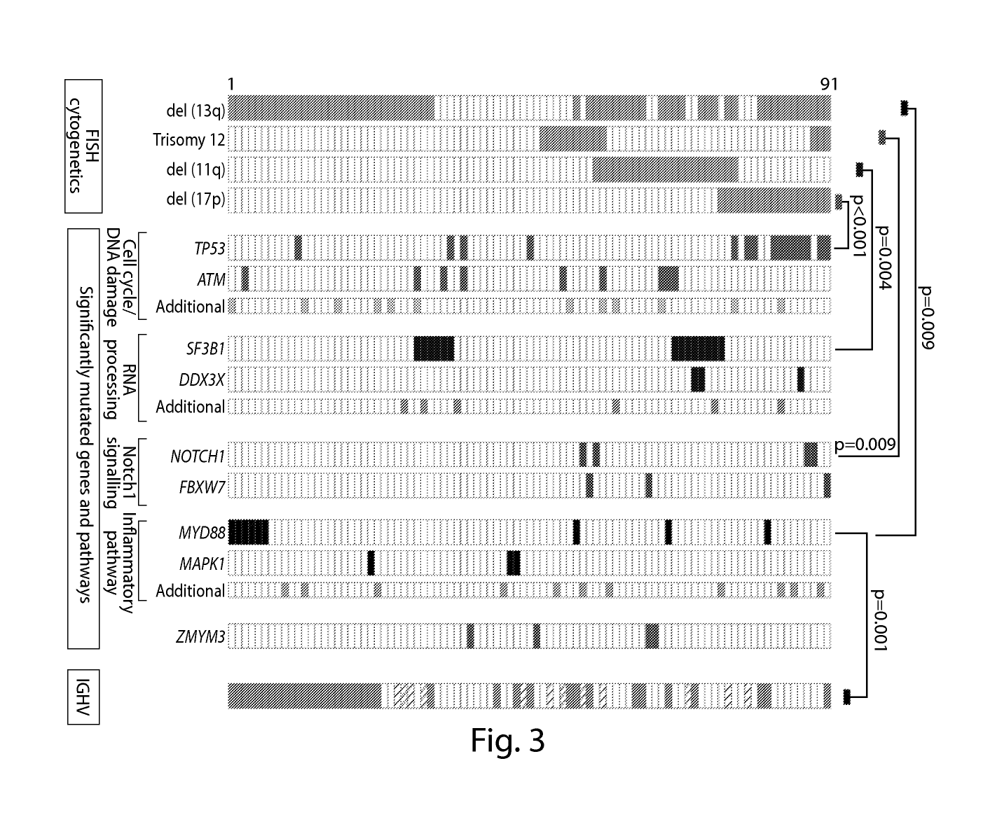

To examine the association between driver mutations and particular clinical features, CLL-associated cytogenetic aberrations and IGHV mutation status in samples harboring mutations in the 9 significantly mutated genes were assessed. Samples were ordered based on FISH cytogenetics, utilizing an established model of hierarchical risk (Dohner, N Engl J Med, 2000, 343:1910-6) (i.e. del(13q), most favorable prognosis when present alone; trisomy 12; and del(11q) and del(17p), both associated with aggressive chemotherapy-refractory disease) (FIG. 3; Tables 8-9).

The distinct prognostic implications of these cytogenetic abnormalities have suggested that they may reflect distinct pathogenesis. These data demonstrate associations of different driver mutations with different key FISH abnormalities, providing support for this hypothesis. Consistent with prior literature (Zenz, J Clin Oncol, 2010, 28:4473-9), most TP53 mutations (11 of 17) were present in samples also harboring del(17p) (p<0.001), resulting in homozygous p53 inactivation. Mutations in ATM—which lies in the minimally deleted region of chromosome 11q—were marginally associated with del(11q) (4 of 22 del(11q) samples, (p=0.09)). Strikingly, mutations in SF3B1 were associated with del(11q) (8 of 22 (36%) del(11q) samples; p=0.004). Of the six CLL samples with mutated SF3B1 and without del(11q), two also harbored a heterozygous mutation in ATM. These findings strongly suggest an interaction between del(11q) and SF3B1 mutation in the pathogenesis of this clinical subgroup of CLL.

Furthermore, the NOTCH1 and FBXW7 mutations were associated with trisomy 12 (p=0.009, and 0.05, respectively). As in previous reports (Fabbri, J Exp Med, 2011; Puente, Nature, 2011), NOTCH1 mutations consistently associated with unmutated IGHV status. The data described herein show that the NOTCH1 and FBXW7 mutations were present in independent samples, suggesting they may similarly lead to aberrant Notch signaling in this clinical subgroup.

All MYD88 mutations were present in samples harboring heterozygous del(13q) (p=0.009). As in recent reports (Fabbri, J Exp Med, 2011; Puente, Nature, 2011), the data demonstrate that MYD88 mutation was always associated with mutated IGHV status (p=0.001), which suggests a post-germinal center origin. These results indicate that, like in DLBCL, where MYD88 is frequently mutated (Ngo, Nature 2011, 470:115-9), constitutive activation of the NF-κB/TLR pathway may have larger impact in the germinal center context.

### Example 5

### Mutations in Sf3B1 are Associated with Earlier Time to First Therapy and Altered Pre-mRNA Splicing

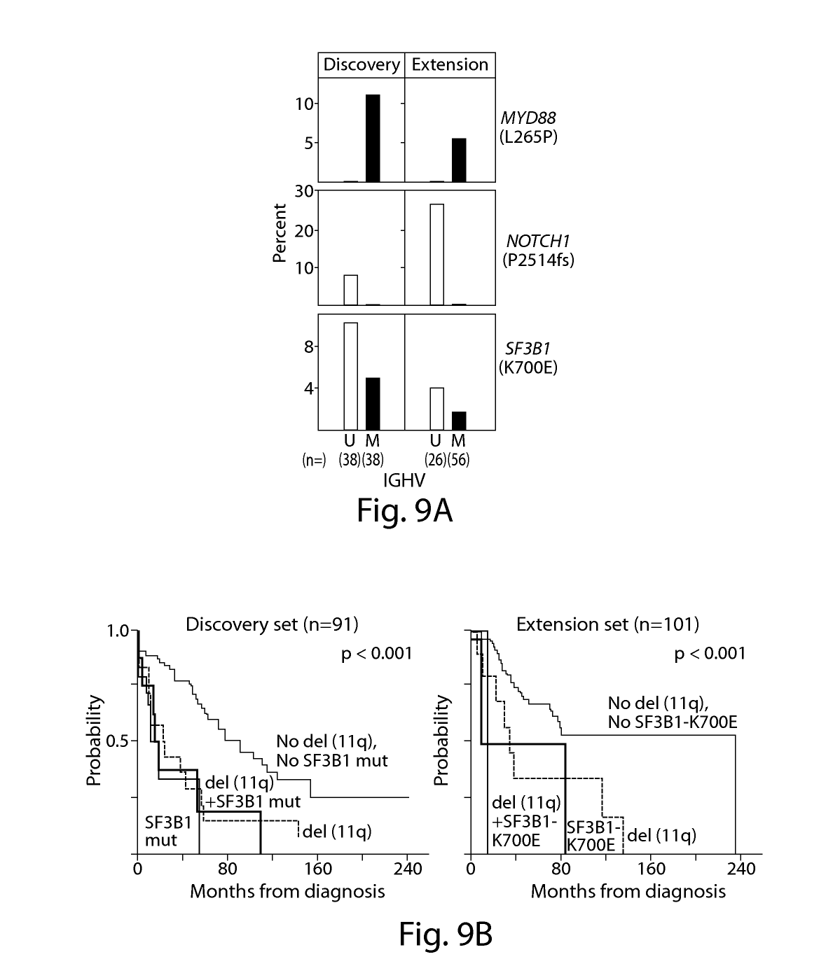

Mutations in NOTCH1 and MYD88 were respectively associated with unmutated and mutated IGHV status across the 192 CLL samples in the discovery and extension sets. Mutation SF3B1-K700E was associated with unmutated IGHV, p=0.048, but was also distributed in IGHV-mutated samples, suggesting that it is an independent risk factor (FIG. 9A). Indeed, a Cox multivariable regression model for clinical factors contributing to an earlier time to first therapy (TTFT) in the 91 CLL samples revealed that SF3B1 mutation was predictive of shorter time to requiring treatment (HR 2.20, p=0.032), independent of other established predictive markers such as IGHV mutation, presence of del(17p) or ATM mutation (FIG. 4A). Consistent with these analyses, patients harboring the SF3B1 mutation alone (without del(11q)) had TTFT similar to patients with del(11q) alone or with both del(11q) and SF3B1 mutation. All three groups demonstrated significantly shorter TTFT than patients without SF3B1 mutation or without del(11q) (FIG. 9B, p<0.001). Similar short TTFT was observed among the 3 CLL samples within the extension cohort whose tumors harbored the SF3B1-K700E mutation compared to samples without this mutation.

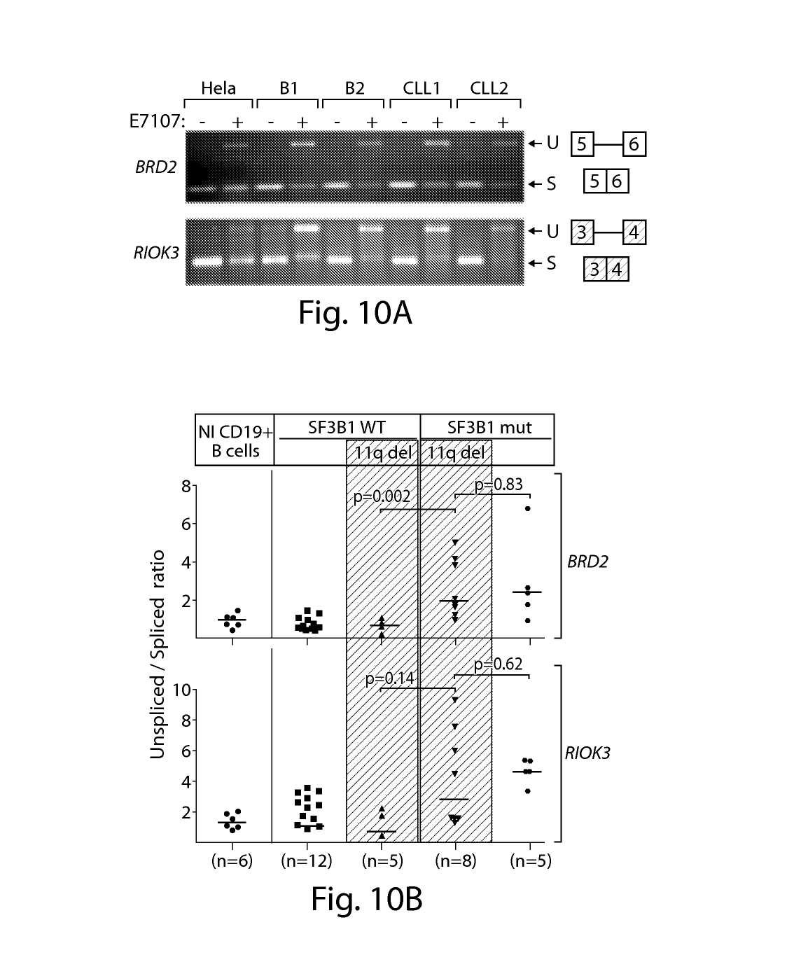

Because SF3B1 encodes a splicing factor that lies at the catalytic core of the spliceosome, functional evidence of alterations in splicing associated with SF3B1 mutation was examined. Kotake et al. previously used intron retention in the endogenous genes BRD2 and RIOK3 to assay function of the SF3b complex (Kotake, Nat Chem Biol, 2007, 3:570-5). The SF3B1 inhibitor E7107, which targets the spliceosome complex, inhibits splicing of BRD2 and RIOK3 in both normal and CLL-B cells (FIG. 10A). Using this assay, aberrant endogenous splicing activity were found in CLL samples harboring mutated SF3B1 (n=13) versus wildtype SF3B1 (n=17), in which the ratio of unspliced to spliced mRNA forms of BRD2 and RIOK3 was significantly higher in those harboring SF3B1 mutations (median ratios 2.0 vs. 0.55 [p<0.0001], and 4.6 vs. 2.1 [p=0.006], respectively) (FIG. 4B). In contrast, no splicing defects were detected in del(11q) samples with WT SF3B1 compared to del(11q) samples with mutated SF3B1 (FIG. 10B). These studies indicate that splicing function in CLL is altered as a result of mutation in SF3B1 rather than del(11q).

### Example 6

### Materials & Methods

Experimental Procedures.

149 patients with CLL provided tumor and normal DNA for sequencing and copy number assessment in this study. Tumor and normal DNA from 11 additional patients were also analyzed by DNA sequencing alone (a total of 160 CLL samples). 82 CLL samples were previously reported (Quesada et al., 2012; Wang et al., 2011), and the raw BAM files for these samples were re-processed and re-analyzed together with the new data, to ensure the consistency of the results as well as enable the detection of smaller subclones made possible with a newer version of the mutation caller [MuTect]. Written informed consent was obtained prior to sample collection according to the Declaration of Helsinki. DNA was extracted from blood- or marrow-derived lymphocytes (tumor) and autologous epithelial cells (saliva), fibroblasts or granulocytes (normal).

Libraries for whole-exome sequencing (WES) were constructed and sequenced on either an Illumina HiSeq 2000 or Illumina GA-IIX using 76 bp paired-end reads, and data were processed, as detailed elsewhere (Berger et al., 2011; Chapman et al., 2011; Fisher et al., 2011). As previously described (Chapman et al., 2011), output from Illumina software was processed by the Picard data processing pipeline to yield BAM files containing well calibrated, aligned reads (DePristo et al., 2011). BAM files were processed by the Firehose pipeline, which performs QC and identifies somatic single nucleotide variations (sSNVs), indels, and other structural chromosomal rearrangements. Recurrent sSNV and indels in 160 CLLs were identified using MutSig2.0 (Lohr et al., 2012). For 111 of 149 matched CLL-normal DNA samples, copy number profiles were obtained using the Genome-wide Human SNP Array 6.0 (Affymetrix), according to the manufacturer's protocol (Genetic Analysis Platform, Broad Institute, Cambridge Mass.), with allele-specific analysis [HAPSEG (Carter, 2011)]. Significant recurrent somatic copy number alterations (sCNAs) were identified using the GISTIC2.0 algorithm (Mermel et al., 2011). Regions with germline copy number variants were excluded from the analysis. For CLL samples with no available SNP arrays (38 of 149 CLLs), sCNAs were estimated directly from the WES data, based on the ratio of CLL sample read-depth to the average read-depth observed in normal samples for that region. We applied the algorithm ABSOLUTE (Carter et al., 2012), to estimate sample purity, ploidy, and absolute somatic copy numbers. These were used to infer the cancer cell fraction (CCF) of point mutations from the WES data. Following the framework previously described (Carter et al., 2012), we computed the posterior probability distribution over CCF c as follows. Consider a somatic mutation observed in a of N sequencing reads on a locus of absolute somatic copy-number q in a sample of purity α. The expected allele-fraction f of a mutation present in one copy in a fraction c of cancer cells is calculated by f(c)=αc/(2(1−α)+αaq, with cε[0.01,1]. Then P(c)∝Binom(a|N,f(c)), assuming a uniform prior on c. The distribution over CCF was then obtained by calculating these values over a regular grid of 100 c values and normalizing. Mutations were thereafter classified as clonal based on the posterior probability that the CCF exceeded 0.95, and subclonal otherwise. Validation of allelic fraction was performed by using deep sequencing with indexed libraries recovered on a Fluidigm chip. Resulting normalized libraries were loaded on a MiSeq instrument (Illumina) and sequenced using paired-end 150 bp sequencing reads to an average coverage depth of 4200×.

Associations between mutation rates and clinical features were assessed by the Wilcoxon rank-sum test, Fisher exact test, or the Kruskal-Wallis test, as appropriate. Time-to-event data were estimated by the method of Kaplan and Meier, and differences between groups were assessed using the log-rank test. Unadjusted and adjusted Cox modeling was performed to assess the impact of the presence of a subclonal driver on clinical outcome measures alone and in the presence of clinical features known to impact outcome, such as IGHV status, cytogenetics, and mutation identity. A chi-square test with 1 degree of freedom and the −2 Log-likelihood statistic were used to test the prognostic independence of subclonal status in Cox modeling.

Human Samples.

Heparinized blood, skin biopsies and saliva were obtained from patients enrolled on clinical research protocols at the Dana-Farber Harvard Cancer Center (DFHCC) approved by the DFHCC Human Subjects Protection Committee. The diagnosis of CLL according to WHO criteria was confirmed in all cases by flow cytometry, or by lymph node or bone marrow biopsy. Peripheral blood mononuclear cells (PBMC) from normal donors and patients were isolated by Ficoll/Hypaque density gradient centrifugation. Mononuclear cells were used fresh or cryopreserved with FBS 10% DMSO and stored in vapour-phase liquid nitrogen until the time of analysis. Primary skin fibroblast lines were generated from skin punch biopsies as previously described (Wang et al., 2011). The patients included in the cohort represent the broad clinical spectrum of CLL (data not shown).

Established CLL Prognostic Factor Analysis.

Immunoglobulin heavy-chain variable (IGHV) homology (“unmutated was defined as greater than or equal to 98% homology to the closest germline match) and ZAP-70 expression (high risk defined as >20% positive) were determined (Rassenti et al., 2008). Cytogenetics were evaluated by FISH for the most common CLL abnormalities (del(13q), trisomy 12, del(11q), del(17p), rearrangements of chromosome 14) (all probes from Vysis, Des Plaines, Ill., performed at the Brigham and Women's Hospital Cytogenetics Laboratory, Boston Mass.). Samples were scored positive for a chromosomal aberration based on consensus cytogenetic scoring (Smoley et al., 2010).

DNA Quality Control.

We used standard Broad Institute protocols as recently described (Berger et al., 2011; Chapman et al., 2011). Tumor and normal DNA concentration were measured using PicoGreen® dsDNA Quantitation Reagent (Invitrogen, Carlsbad, Calif.). A minimum DNA concentration of 60 ng/μl was required for sequencing. In select cases where concentration was <60 ng/μl, ethanol precipitation and re-suspension was performed. Gel electrophoresis confirmed that the large majority of DNA was high molecular weight. All Illumina sequencing libraries were created with the native DNA. The identities of all tumor and normal DNA samples (native and WGA product) were confirmed by mass spectrometric fingerprint genotyping of 24 common SNPs (Sequenom, San Diego, Calif.).

Whole-Exome DNA Sequencing.

Informed consent on DFCI IRB-approved protocols for whole exome sequencing of patients' samples was obtained prior to the initiation of sequencing studies. DNA was extracted from blood or marrow-derived lymphocytes (tumor) and saliva, fibroblasts or granulocytes (normal), as previously described (Wang et al., 2011). Libraries for whole exome (WE) sequencing were constructed and sequenced on either an Illumina HiSeq 2000 or Illumina GA-IIX using 76 bp paired-end reads. Details of whole exome library construction have been detailed elsewhere (Fisher et al., 2011). Standard quality control metrics, including error rates, percentage passing filter reads, and total Gb produced, were used to characterize process performance before 15 downstream analysis. Average exome coverage depth was 132×/146× for tumor/germline. The Illumina pipeline generates data files (BAM files) that contain the reads together with quality parameters. Of the 160 CLL samples reported in the current manuscript, 82 were included in a previous study (Wang et al., 2011). 340 CLL and germline samples were sequenced overall. These include 160 CLL and matched germline DNA samples as well as timepoint 2 samples for 17 of 160 CLLs, and an additional sample pair and germline for a longitudinal sample pair not included in the 160 cohort (CLL020).

Identification of Somatic Mutations.

Output from Illumina software was processed by the “Picard” data processing pipeline to yield BAM files containing aligned reads (via MAQ, to the NCBI Human Reference Genome Build hg18) with well-calibrated quality scores (Chapman et al., 2011; DePristo et al., 2011). For 51 of the 160 CLL samples included in the analysis, sequencing was performed on capture libraries generated from whole genome amplified (WGA) samples. For those samples, 100 ng inputs of samples were whole genome amplified with the Qiagen REPLI-g Midi Kit (Valencia, Calif.). From the sequencing data, somatic alterations were identified using a set of tools within the “Firehose” pipeline, developed at The Broad Institute, Inc. and available at its website. The details of our sequencing data processing have been described elsewhere (Berger et al., 2011; Chapman et al., 2011). Somatic single nucleotide variations (sSNVs) were detected using MuTect; somatic small insertions and deletions (indels) were detected using Indelocator. All mutations identified in longitudinal samples were confirmed by manual inspection of the sequencing data (Robinson et al., 2011). An estimated contamination threshold of 5% was used for all samples based on the highest contamination values seen in a formal contamination analysis done with ContEst based on matched SNP arrays (Cibulskis et al., 2011). Ig loci mutations were not included in this analysis. Somatic mutations detected in the 160 CLL samples were compiled (data not shown). WES data is deposited in dbGaP (phs000435.v1.p1).

Significance Analysis for Recurrently Mutated Genes.

The prioritization of somatic mutations in terms of conferring selective advantage was done with the statistical method MutSig2.0 (Lohr et al., 2012). In short, the algorithm takes an aggregated list of mutations and tries to detect genes that are affected more than expected by chance, as those likely reflect positive selection (i.e., driver events). There are two main components to MutSig2.0:

The first component attempts to model the background mutation rate for each gene, while taking into account various different factors. Namely, it takes into account the fact that the background mutation rate may vary depending on the base context and base change of the mutation, as well as the fact that the background rate of a gene can also vary across different patients. Given these factors and the background model, it uses convolutions of binomial distributions to calculate a P value, which represents the probability that we obtain the observed configuration of mutations, or a more significant one.

The second component of the algorithm focuses on the positional configuration of mutations and their sequence conservation (Lohr et al., 2012). For each gene, the algorithm permutes the mutations preserving their tri-nucleotide context, and for each permutation calculates two metrics: one that measures the degree of clustering into hotspots along the coding length of the gene, and one that measures the average conservation of mutations in the gene. These two null models are then combined into a joint distribution, which is used to calculate a P value that reflects the probability by chance that we can obtain by chance the observed mutational degree of clustering and conservation, or a more significant outcome.

The two P values that are produced by the two components are then combined using Fisher-Combine (Fisher, 1932) which yields a final P value which is used to sort the genes by degree of mutational significance. This is subsequently corrected for multihypothesis using the Benjamini Hochberg procedure.

Genome-Wide Copy Number Analysis.

Genome-wide copy number profiles of 111 CLL samples and their patient-matched germline DNA were obtained using the Genome-wide Human SNP Array 6.0 (Affymetrix), according to the manufacturer's protocol (Genetic Analysis Platform, The Broad Institute, Inc. Cambridge, Mass.). SNP array data were deposited in dbGaP (phs000435.v1.p1). Allele-specific analysis also allowed for the identification of copy neutral LOH events as well as quantification of the homologous copy-ratios (HSCSs) [HAPSEG (Carter, 2011)]. Significant recurrent chromosomal abnormalities were identified using the GISTIC2.0 algorithm ((Mermel et al., 2011), v87). Regions with germline copy number variants were excluded from the analysis.

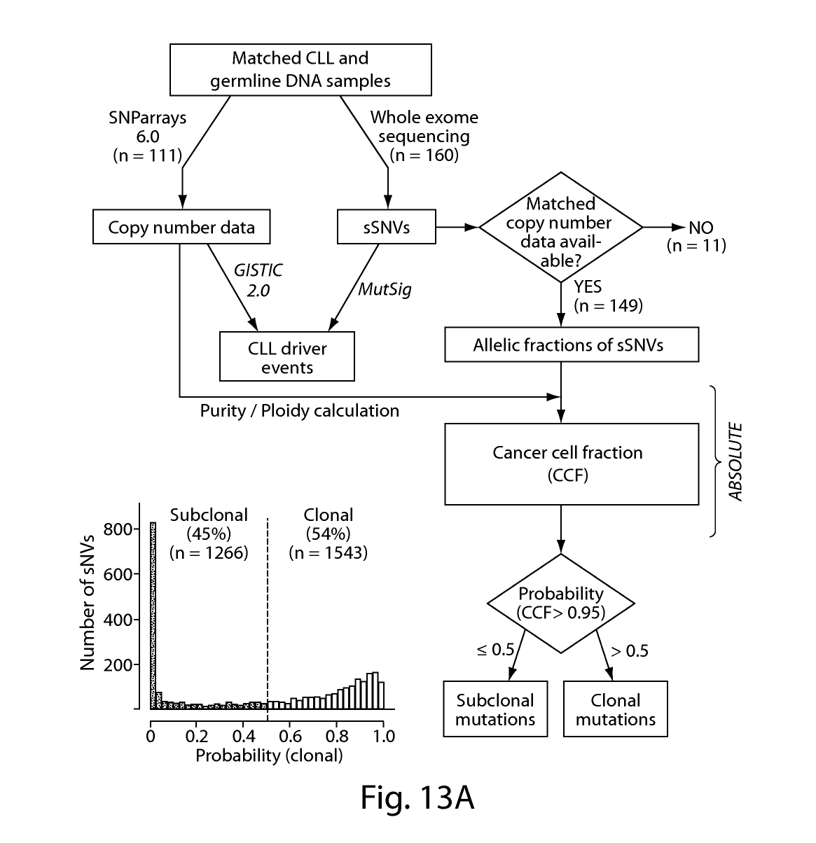

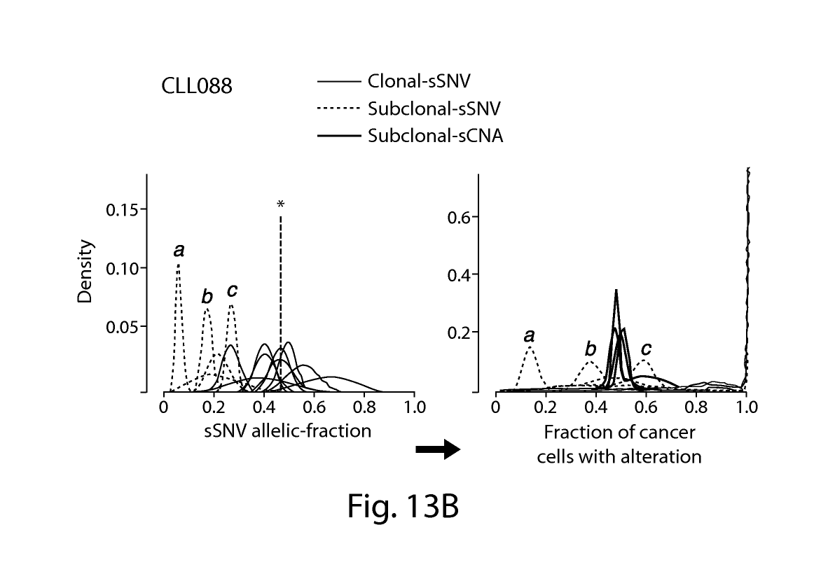

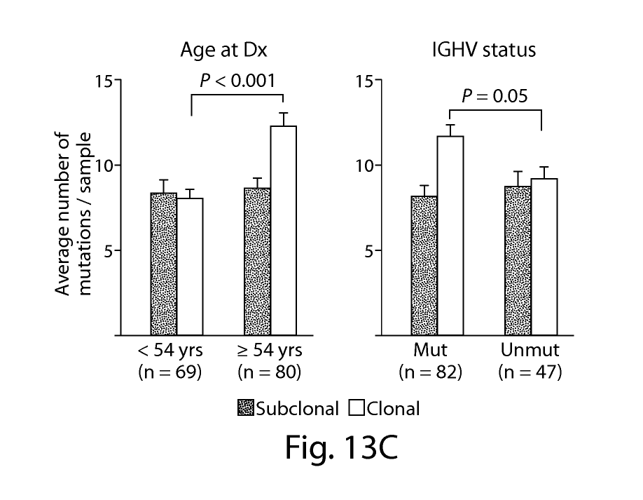

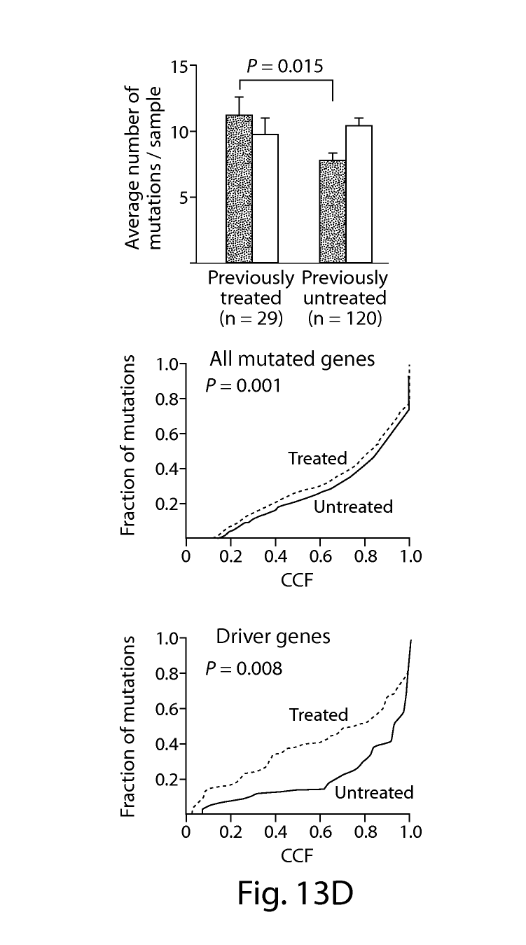

For CLL samples with no available SNP arrays (38/160), sCNAs were estimated directly from the WES data, based on the ratio of CLL sample read-depth to the average readdepth observed in normal samples for that region. 11/160 samples were excluded from this analysis due to inability to obtain copy number information from the WES data. See FIG. 13A for outline of sample processing.

Validation Deep Sequencing.

Validation targeted resequencing of 256 selected somatic mutations sSNVs was performed using microfluidic PCR. Target specific primers with Fluidigm-compatible tails were designed to flank sites of interest and produce amplicons of 200+/−20 bp. Molecular barcoded, Illumina-compatible oligonucleotides, containing sequences complementary to the primer tails were added to the Fluidigm Access Array chip (San Francisco, Calif.) in the same well as the genomic DNA samples (20-50 ng of input) such that all amplicons for a given genomic sample shared the same index, and PCR was performed according to the manufacturer's recommendations. Indexed libraries were recovered for each sample in a single collection well on the Fluidigm chip, quantified using picogreen and then normalized for uniformity across libraries. Resulting normalized libraries were loaded on a MiSeq instrument (Illumina) and sequenced using paired end 150 bp sequencing reads. 95.2% of called sSNVs were detected in the validation experiment (data not shown). For 91.8% of the mutations, the allelic fraction estimates were concordant (with the discordant events enriched in sites of lower WES coverage). RNA sequencing (dUTP Library Construction). 5 μg of total RNA was poly-A selected using oligo-dT beads to extract the desired mRNA. The purified mRNA is treated with DNAse, and cleaned up using SPRI (Solid Phase Reversible Immobilization) beads according to the manufacturers' protocol. Selected Poly-A RNA was then fragmented into ˜450 bp fragments in an acetate buffer at high heat. Fragmented RNA was cleaned with SPRI and primed with random hexamers before first strand cDNA synthesis. The first strand was reverse transcribed off the RNA template in the presence of Actinomycin D to prevent hairpinning and purified using SPRI beads. The RNA in the RNA-DNA complex was then digested using RNase H. The second strand was next synthesized with a dNTP mixture in which dTTPs had been replaced with dUTPs. After another SPRI bead purification, the resultant cDNA was processed using Illumina library construction according to manufacturers protocol (end repair, phosphorylation, adenylation, and adaptor ligation with indexed adaptors). SPRI-based size selection was performed to remove adapter dimers present in the newly constructed cDNA library. Libraries were then treated with Uracil-Specific Excision Reagent (USER) to nick the second strand at every incorporated Uracil (dUTP). Subsequently, libraries were enriched with 8 cycles of PCR using the entire volume of sample as template. After enrichment, the library is quantified using pico green, and the fragment size is measured using the Agilent Bioanalyzer according to manufactures protocol. Samples were pooled and sequenced using either 76 or 101 bp paired end reads.

RNASeq Data Analysis.

RNAseq BAMs were aligned to the hg18 genome using the TopHat suite. Each somatic base substitution detected by WES was compared to reads at the same location in RNAseq. Based on the number of alternate and reference reads, a power calculation was obtained with beta-binomial distribution (power threshold used was greater than 80%). Mutation calls were deemed validated if 2 or greater alternate allele reads were observed in RNA-Seq at the site, as long as RNAseq was powered to detect an event at the specified location.

FACS Validation of Ploidy Estimates with ABSOLUTE.

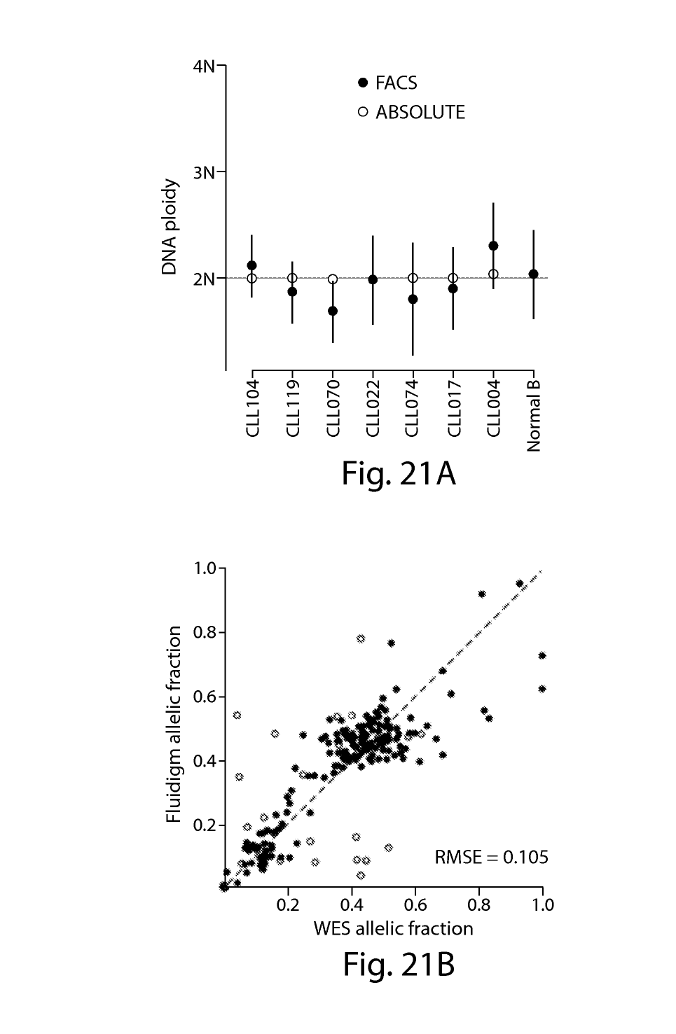

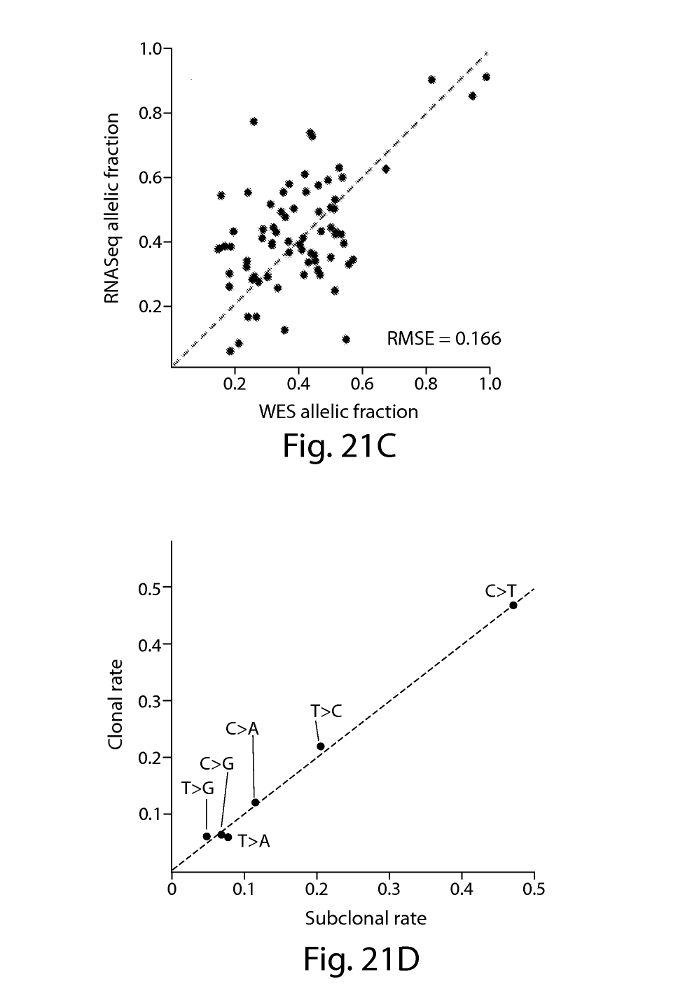

Consistent with published studies of CLL (Brown et al., 2012; Edelmann et al., 2012), ABSOLUTE measured all CLL samples to be near diploid (data not shown; median −2, range 1.95-2.1). We confirmed the measurements using a standard assay for measuring DNA content. For this analysis, peripheral blood mononuclear cells from normal volunteers and CLL patients and cell lines are first stained with anti-CD5 FITC and anti-CD19 PE antibodies in a PBS buffer containing 1% BSA for 30 minutes on ice. After extensive washes, the cells were then stained with a PBS buffer contained 1% BSA, 0.03% saponin (Sigma) and 250 ug/m17-AAD (Invitrogen) for 1 hour on ice, followed by analysis on a Beckman Coulter FC500 machine (FIG. 21A).

Estimation of Mutation Cancer Cell Fraction Using ABSOLUTE.

We used the ABSOLUTE algorithm to calculate the purity, ploidy, and absolute DNA copy-numbers of each sample (Carter et al., 2012). Modifications were made to the algorithm, which are implemented in version 1.05 of the software, available for download at The Broad Institute, Inc. website. Specifically, we added to the ability to determine sample purity from sSNVs alone, in samples where no sCNAs are present (the ploidy of such samples is 2N). In addition, estimates of sample purity and absolute copy-numbers are used to compute distributions over cancer cell fraction (CCF) values of each sSNV, as described (Experimental Procedures), and for sCNAs (described below). The current implementation of ABSOLUTE does not automatically correct for sCNA subclonality when computing CCF distributions of sSNVs (this is an area of ongoing development). Fortunately, the few sCNAs that occurred in our CLL samples were predominantly clonal. Manual corrections were made for CLL driver sSNVs occurring at site of subclonal sCNAs (5 TP53 sSNVs and 1 ATM sSNV), based on the sample purity, allelic fraction and the copy ratio of the matching sCNA.

Each sSNV was classified as clonal or subclonal based on the probability that the CCF exceeded 0.95. A probability threshold of 0.5 was used throughout the manuscript. However, as the histogram in FIG. 21 shows, the distribution of events around the threshold was observed to be fairly uniform and results were not significantly affected across a range of thresholds. For example, the results of our analyses were unchanged when we altered our definition of clonal mutations to be (Pr(CCF>0.95))>0.75, and subclonal when Pr(CCF>0.95) was <0.25, leaving uncertain mutations unclassified. Using these thresholds, CLLs with mutated IGHV and age were associated with a higher number of clonal mutations (P values of 0.05 and <0.0001, respectively). CLLs treated prior to sample collection had a higher number of subclonal mutations (P=0.01) and the subclonal set was enriched with putative drivers (P=0.0019). Importantly, the results of the clinical analysis also remained unchanged. FFS_Rx was shorter in samples in which a subclonal driver was detected (P=0.007) and regression models examining known poor prognostic indicators in CLL yielded an adjusted P value of 0.009.

One of the recurrent CLL cancer genes, NOTCH1, had 15 mutations, 14 of which were the identical canonical 2 base-pair deletions. Unlike sSNVs, the observed allelic fractions of indels events were not modeled as binomial sampling of reference and alternate sequence reads according to their true concentration in the sample (Carter et al., 2012). This was due to biases affecting the alignment of the short sequencing reads, which generally favor reference over alternate alleles. To measure the magnitude of this effect, we examined the allelic fraction (AF) of 514 germline 2 bp deletions called in 4 normal germline WES samples. We observed that the distribution (data not shown) of allelic-fractions for heterozygous events was peaked at 0.41, as opposed to the expected mode of 0.5, with nearly all AFs between 0.3 to 0.6. Therefore, the bias factor towards reference is peaked at 0.82 but may range from 0.6 to 1 (unlikely to be greater than 1). CCF distributions for the 14 somatic indels in NOTCH1 were calculated using bias factors of 1.0 (no bias), 0.82 (bias point-estimate), and 0.6 (worst case observed). Reassuringly, the classification of NOTCH1 indels as clonal or subclonal was highly robust and was essentially the same using the three values—only a single case (CLL155) was ambiguous and was classified as subclonal using 1.0 and 0.82, and clonal using 0.6. Taking a conservative approach, not classifying a mutation as sub-clonal unless there is clear evidence for it, we decided to call this event as clonal for downstream analysis.

Estimation of CCF values for subclonal sCNAs is implemented (ABSOLUTEv1.05) in a manner analogous to the procedure for sSNVs (Experimental Procedures), although the transformation is more complex, due to the need for assumptions of the subclonal structure and the error model of microarray based copy-number data. Segmental sCNAs are defined as subclonal based on the mixture model used in ABSOLUTE (Carter et al., 2012). Let the functions hx and h′x denote a variance stabilizing transformation and its derivative, respectively. For SNP microarray data, these are defined as:

$$hx = {sinh}^{- 1} ( bx ) , where b = \frac{{( {e}^{{σ}_{η}^{2}} - 1 )}^{\frac{1}{2}}}{{σ}_{ɛ}} , \\ and {h}^{′} ( x ) = \frac{b}{{( 1 + {( bx )}^{2} )}^{\frac{1}{2}}}$$

### (Huber et al., 2002).

The values σ_{ε}, and σ_{η} denote additive and multiplicative noise scales, respectively, for the microarray hybridization being analyzed; these are estimated by HAPSEG (Carter et al., 2011). The calibrated probe-level microarray data become approximately normal under this transformation, which is used by HAPSEG to estimate the segmental allelic copy-ratios rᵢ and the posterior standard deviation of their mean (under the transformation), σᵢ (Carter, 2011). An additional parameter σ_{H} is estimated by ABSOLUTE (Carter et al., 2012), which represents additional sample-level variance corresponding to regional biases not captured in the probe-level model. For a subclonal segment i, let q_{c} denote the absolute copy number in the unaffected cells, and qₛ denote the absolute copy number in the altered cells. Both of these values are unknown but we used a simplifying assumption that the difference between qc and qs is one copy with q_{c} being closer to the modal copy-number. Therefore, for subclonal deletions (copy ratios below the ratio of modal copy number), qₛ was set to the nearest copy number below the measured value, and q_{c}=qₛ+1. For subclonal gains (ratios above the modal number), qₛ was set to the nearest copy number above the measured value, and q_{c}=qₛ−1. Because the CLL genomes analyzed here were universally near diploid, this was nearly equivalent to assuming that subclonal deletions had qₛ=0 in the affected cells and gains qₛ=2, with q_{c}=1 in both cases (in allelic units). However, we note that these assumptions would not be strictly correct in genomes after doubling, or in cases of high level amplification. In these cases, calculation of posterior CCF distributions will require integration over qₛ and q_{c}, averaging over the set of plausible subclonal genomic configurations.

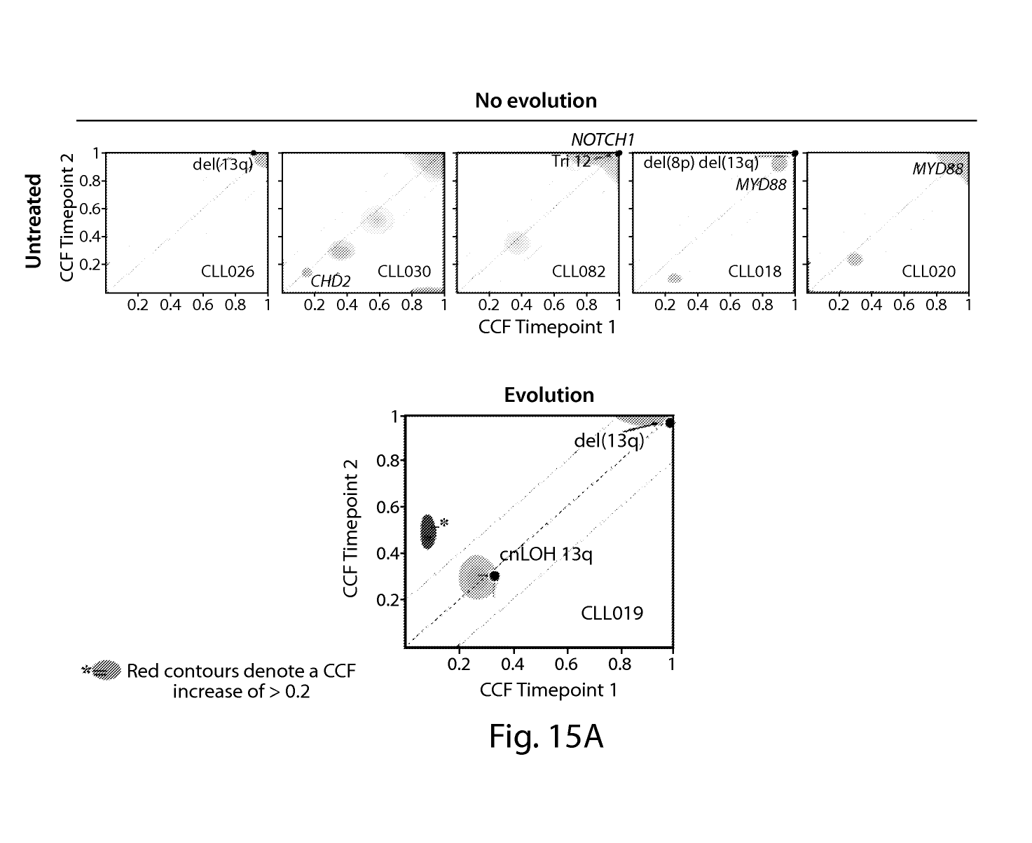

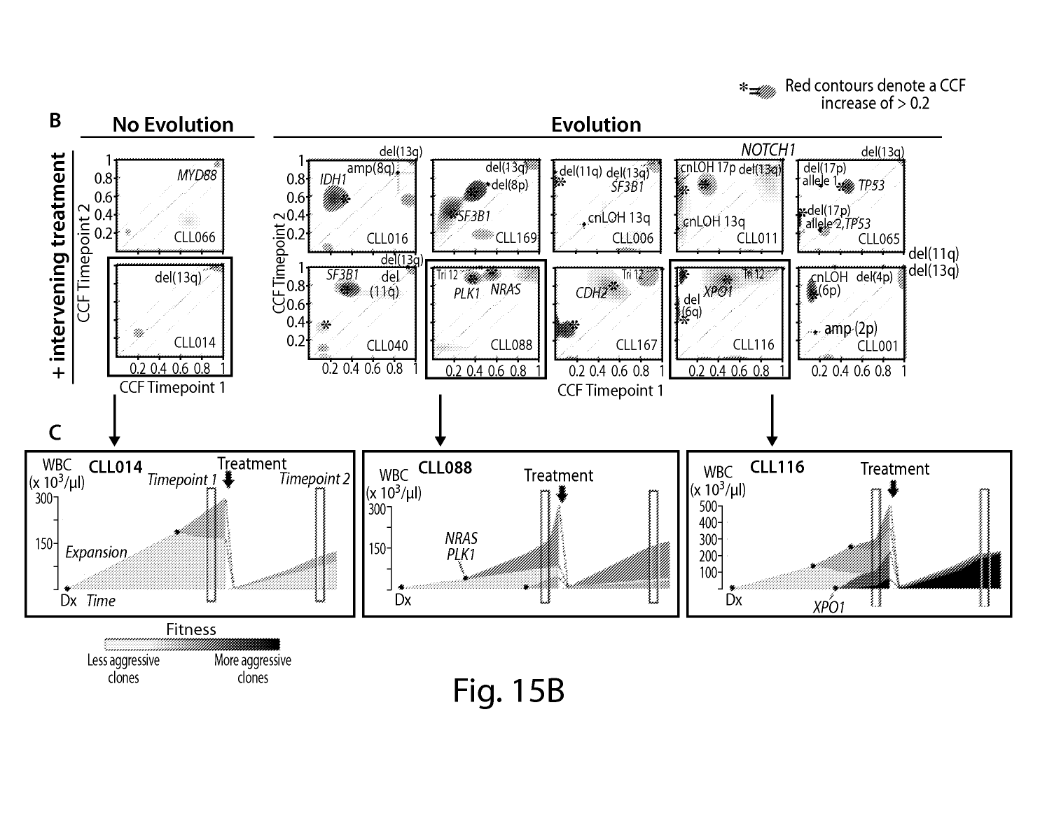

Let r_{c} and rₛ be the theoretical copy ratio values corresponding to q_{c} and qₛ (accounting for sample purity, ploidy, and the modeled attenuation rate of the microarray (Carter et al., 2011; Carter et al., 2012)). Let d=rₛ−r_{c}, then, for CCF c, let rₓ c=dc+r_{c}. Then P(c)∝ [symbol] (hrₓ(c))|h(rᵢ), (σᵢ+σ_{H})²)h′(rₓ(c)). The distribution over CCF is obtained by calculating these values over a regular grid of 100 c values and normalizing. We note that, when copy numbers are estimated directly from sequencing data, the calculation is simpler, as there is no attenuation effect and h x=x. These calculations were used to generate the 95% confidence intervals on the CCF of subclonal driver sCNAs shown in FIG. 15.

Cancer Gene Census List and Conservation Annotations.

Conservation of a specific mutated site was adapted from UCSC conservation score track. A scale of 0-100 was linearly converted from the −6 to 6 scale used in the phastCons track (Siepel et al., 2005). To confirm that driver mutations are more likely to occur in conserved sites, we quantified the conservation in the COSMIC database (Forbes et al., 2008) hotspots and compared it to non-COSMIC hotspots coding location. We matched conservation information for 5085 sites that had greater than 3 exact hits reported in mutations deposited in the COSMIC database, and compared it to conservation found for a set of non-overlapping 5085 randomly sampled coding sites. The conservation was higher in the COSMIC sites than in the non-COSMIC coding sites set (mean conservation 82.39 and 62.15, respectively, p<1e-50). We noted that the distribution of events was not uniform, and nearly one half of COSMIC hotspots had a conservation measure greater than 95 (49.65%, compared to 15.5% in the non-COSMIC set, p<1e-50). For our calculations, we used a cut off of >95 to designate conserved sites likely to contain higher proportion of cancer drivers. We complemented the analysis for putative driver event enrichment by matching the altered genes to the Cancer Gene Census (Futreal et al., 2004).

Clustering Analysis of sSNVs in 18 CLL Sample Pairs.

In order to better resolve the true cancer cell fraction (CCF) of sSNVs detected in longitudinal samples, we employed a previously described Bayesian clustering procedure (Escobar and West, 1995). This approach exploits the assumption that the observed subclonal sSNV CCF values were sampled from a smaller number of subclonal cell populations (subclones). All remaining uncertainty (including the exact number of clusters) was integrated out using a mixture of Dirichlet processes, which was fit using a Gibbs sampling approach, building on a previously described framework (Escobar and West, 1995).

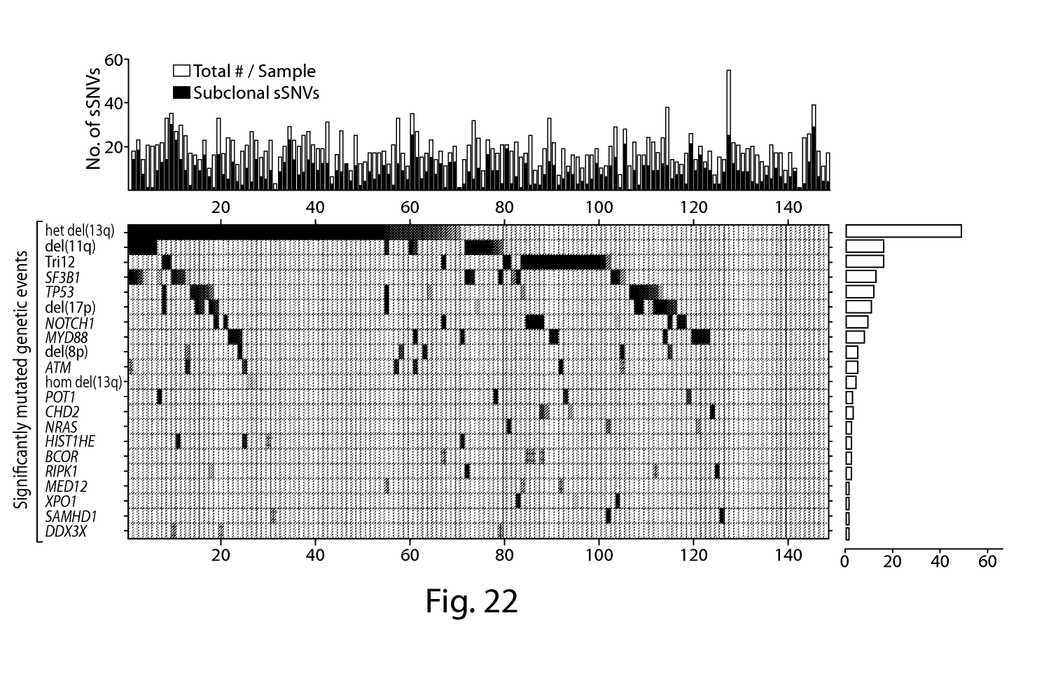

The inputs to this procedure are the posterior CCF distributions for each sSNV being considered. We note that the CCF distributions for sCNAs could be added into the model, however we did not attempt this in the present study. CCF distributions are represented as 100-bin histograms over the unit interval; the two-dimensional CCF distributions used for the 2D clustering of longitudinal samples were obtained as the outer product of the matched histogram pairs for each mutation, resulting in 10,000-bin histograms (FIG. 22). We note that the use of histograms to represent posterior distributions on CCF, although computationally less efficient than parametric forms, have the advantage that CCFs of different mutation classes may be easily combined in the model, even though their posteriors may have very different forms. We also note that the algorithm implementation is identical for the single sample and paired (longitudinal) sample cases, although only the latter was used in the present study.

At each iteration of the Gibbs sampler, each mutation is assigned to a unique cluster and the posterior CCF distribution of each cluster is computed using Bayes' rule, as opposed to drawing a sample from the posterior (a uniform prior on CCF from 0.01 to 1 is used). When considering the probability of a mutation to join an existing cluster, the likelihood calculation of the mutation arising from the cluster is integrated over the uncertainty in the cluster CCF. This allows for rapid convergence of the Gibbs sampler to its stationary distribution, which was typically obtained in fewer than 100 iterations for the analysis presented in this study. We ran the Gibbs sampler for 1,000 iterations, of which the first 500 were discarded before summarization. Because of the small number of clonal mutations in some WES samples, we make an additional modification to the standard Dirichlet process model by adding a fixed clonal cluster that persists even if no mutation is assigned to it. This reflects our prior knowledge that clonal mutations must exist, even if they are the minority of detected mutations. For the samples analyzed here, this modification had very little effect. A key aspect of implementing the Dirichlet process model on WES datasets is reparameterization of prior distributions on the number of subclones k as priors on the concentration parameter α of the Dirichlet process model. Importantly, this must take into account the number of mutations N input to the model, as the effect of α on k is strongly dependent on N (Escobar and West, 1995). We accomplish this by constructing a map from a regular grid over α to expected values of k, given N, using the fact that:

$$P ( k \vert α , N ) = {c}_{N} ( k ) N ! {α}^{k} \frac{Γ ( α )}{Γ ( α + N )}$$

(Antoniak, 1974), where the c_{N}(k) factors correspond to the unsigned Stirling numbers of the first kind. With this map in hand, we perform an optimization procedure to find parameters a and b of a prior Gamma distribution over α resulting in the minimal Kullback-Leibler divergence with the specified prior over k (the divergence was computed numerically on the histograms). Once the prior over α has been represented as a Gamma distribution, learning about α (and therefore k) from the data can be directly incorporated into the Gibbs sampling procedure, resulting in a continuous mixture of Dirichlet processes (Escobar and West, 1995). This allows consistent parameterization of prior knowledge (or lack thereof) on the number of subclonal populations in the face of vastly different numbers of input mutations, which is necessary for making consistent inferences across differing datasets (e.g. WES vs. WGS). We note that taking uncertainty about α into account is necessary for inferences on the number of subclonal populations to be strictly valid, since implementations with fixed values of α result in an implicit prior over k that depends upon N (this is especially important for smaller values of N). For the application presented in this study (FIG. 15), we specified a weak prior on k using a negative binomial distribution with r=10, μ=2 (these values favored 1-10 subclones).

Upon termination of the Gibbs sampler, we summarized the posterior probability over the CCF of each sSNV by averaging the posterior cluster distribution for all clusters to which the sSNV was assigned during sampling. This allowed shrinkage of the CCF probability distributions (as shown in FIG. 15; pre-clustering results are shown in FIG. 22A-B), without having to choose an exact number of subclonal clusters. Note that the 18 longitudinal sample pairs contain 1 CLL sample pair not initially included in the 160 CLLs (CLL020).

Gene Expression Profiling.

Total RNA was isolated from viably frozen PBMCs or B cells from CLL patients that were followed longitudinally (Midi kit; Qiagen, Valencia Calif.), and hybridized to the U133Plus 2.0 array (Affymetrix, Santa Cruz, Calif.) at the DFCI Microarray Core Facility. All expression profiles were processed using RMA, implemented by the PreprocessDataset module in GenePattern available at The Broad Institute, Inc. website (Irizarry et al., 2003; Reich et al., 2006). Probes were collapsed to unique genes by selecting the probe with the maximal average expression for each gene. Batch effects were further removed using the ComBat module in GenePattern (Johnson et al., 2007) (Reich et al., 2006). Visualizations in GENE-E, available at The Broad Institute, Inc. website, were based on logarithmic transformation (log 2) of the data and centering each gene (zero mean). These data can be accessed at NCBI website with accession number GSE37168.

RNA Pyrosequencing for Mutation Confirmation.

Quantitative targeted sequencing to detect somatic mutation within cDNA was performed, as previously described (Armistead et al., 2008). In brief, biotinylated amplicons generated from PCR of the regions of transcript surrounding the mutation of interest were generated. Immobilized biotinylated single-stranded DNA fragments were isolated per manufacturer's protocol, and sequencing undertaken using an automated pyrosequencing instrument (PSQ96; Qiagen, Valencia Calif.), followed by quantitative analysis using Pyrosequencing software (Qiagen).

Statistical Methods.

Statistical analysis was performed with MATLAB (MathWorks, Natick, Mass.), R version 2.11.1 and SAS version 9.2 (SAS Institute, Cary, N.C.). Categorical variables were compared using the Fisher Exact test, and continuous variables were compared using the Student's t-test, Wilcoxon rank sum test, or Kruskal Wallis test as appropriate; the association between two continuous variables was assessed by the Pearson correlation coefficient. The time from the date of sample to first therapy or death (failure-free survival from sample time or FFS_Sample) was calculated as the time from sample to the time of the first treatment after the sample or death and was censored at the date of last contact. FFS_Rx (failure-free survival from first treatment after sampling) was defined as the time to the 2nd treatment or death from the 1^{st} treatment following sampling, was calculated only for those patients who had a 1^{st} treatment after the sample and was censored at the date of last contact for those who had only one treatment after the sample. Time to event data were estimated by the method of Kaplan and Meier, and differences between groups were assessed using the log-rank test. Unadjusted and adjusted Cox modeling was performed to assess the impact of the presence of a subclonal driver and a driver irrespective of the CCF on FFS_Sample and FFS_Rx. A chi-square test with 1 degree of freedom and the −2 Log-likelihood statistic was used to test the prognostic independence of subclonal status in Cox modeling using a full model and one without subclonal status included. We also formally tested for nonproportionality of the hazards in FIG. 17B. First, we plotted the log (−log(survival) versus log(time) for the two categories, and demonstrated that curves do not cross, which supports the fact that they are proportional. Second, we also tested for nonproportionality by including a time varying covariate for each variable in the model. None of these were significant indicating that the hazards are proportional. Models were adjusted for known prognostic factors for CLL treatment including the presence of a 17p deletion, the presence of a 11q deletion, IGHV mutational status, and prior treatment at the time of sample. Cytogenetic abnormalities were primarily assessed by FISH and if unknown, genomic data were included. For unknown IGHV mutational status an indicator was included in adjusted modeling and was not found to be significant. All P-values are two-sided and considered significant at the 0.05 level unless otherwise noted.

### Results

Large-Scale WES Analysis of CLL Expands the Compendium of CLL Drivers and Pathways.

We performed whole-exome sequencing (WES) (Gnirke et al., 2009) of 160 matched CLL and germline DNA samples (including 82 of the 91 samples previously reported (Wang et al., 2011)). These patients represented the broad spectrum of CLL clinical heterogeneity, and included patients with both low- and high-risk features based on established prognostic risk factors (ZAP70 expression, the degree of somatic hypermutation in the variable region of the immunoglobulin heavy chain (IGHV) gene, and presence of specific cytogenetic abnormalities) (data not shown). We applied MuTect (a highly sensitive and specific mutation-calling algorithm) to the WES data to detect somatic single nucleotide variations (sSNVs) present in as few as 10% of cancer cells. Average sequencing depth of WES across samples was ˜130×. In total, we detected 2,444 nonsynonymous and 837 synonymous mutations in protein-coding sequences, corresponding to a mean (±SD) somatic mutation rate of 0.6±0.28 per megabase (range, 0.03 to 2.3), and an average of 15.3 nonsynonymous mutations per patient (range, 2 to 53) (data not shown).

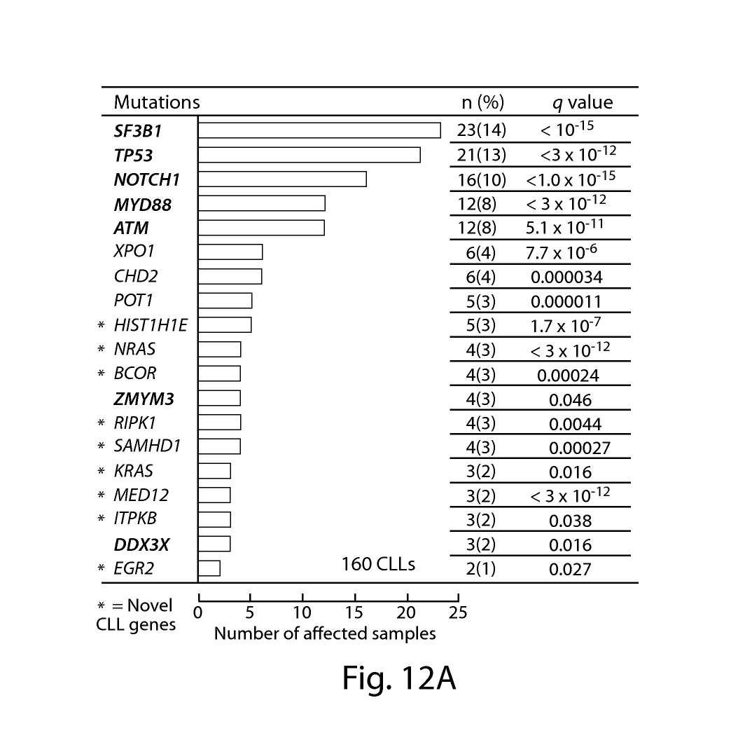

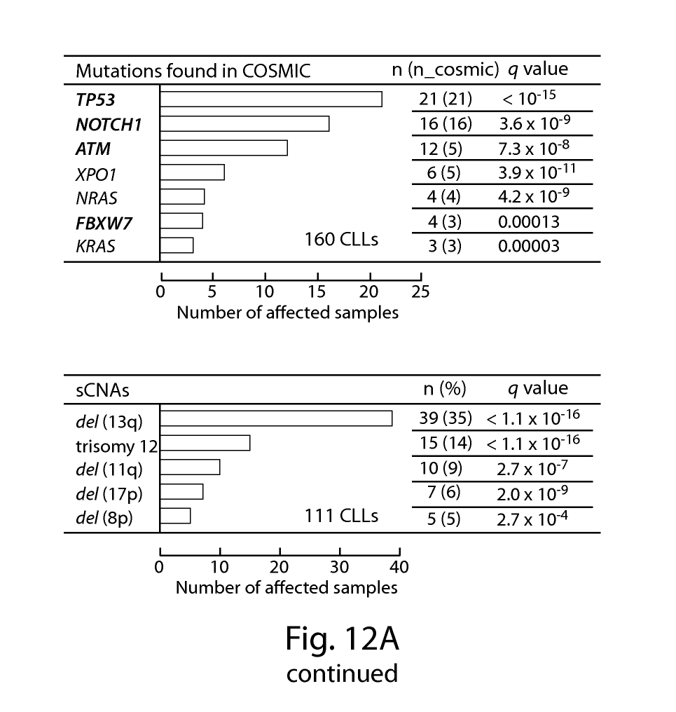

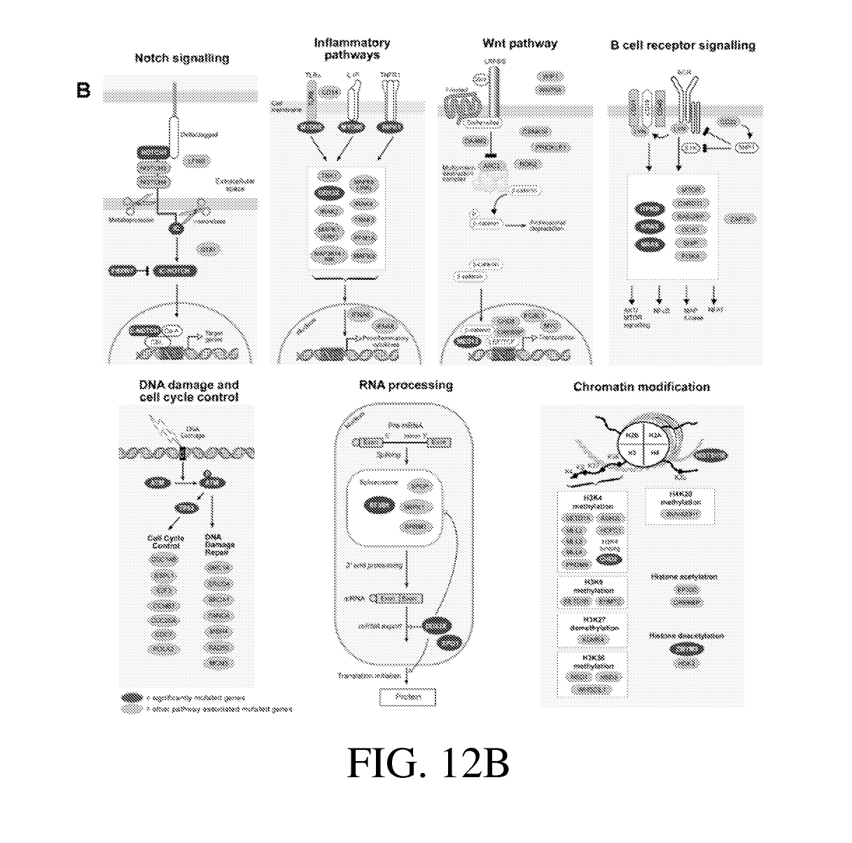

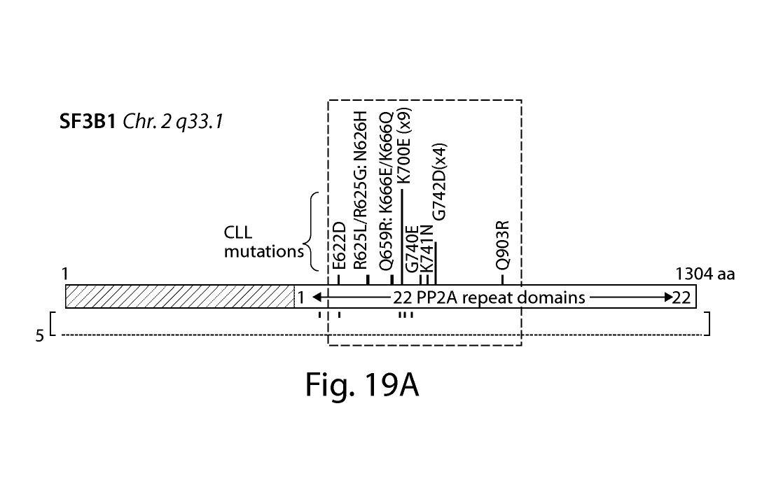

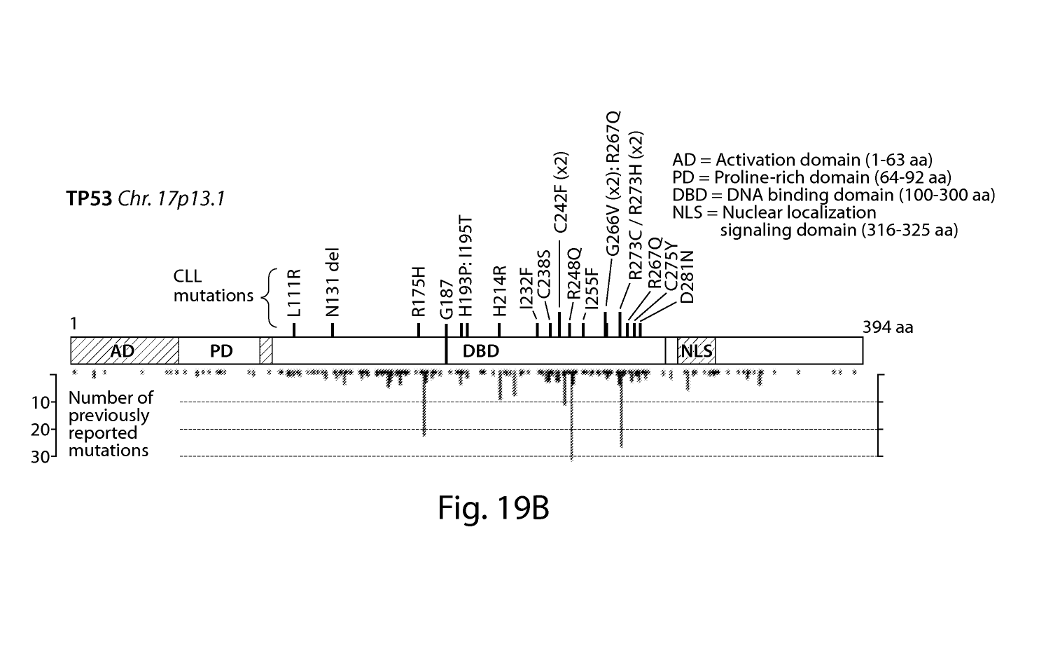

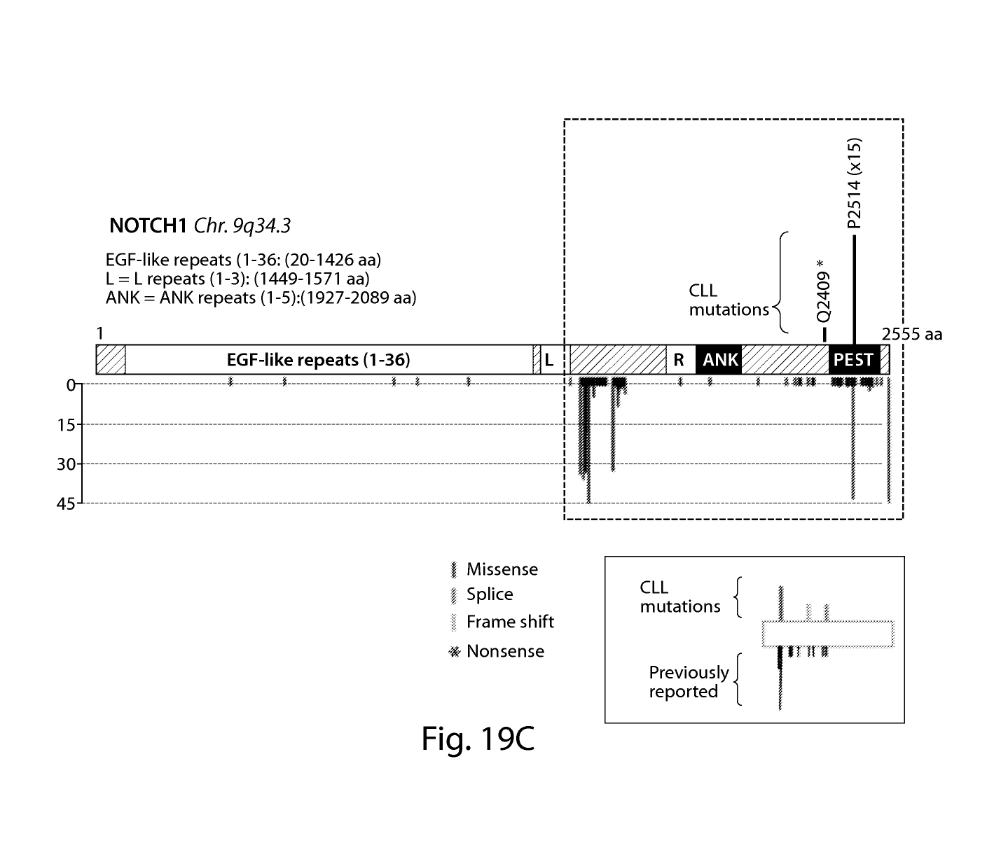

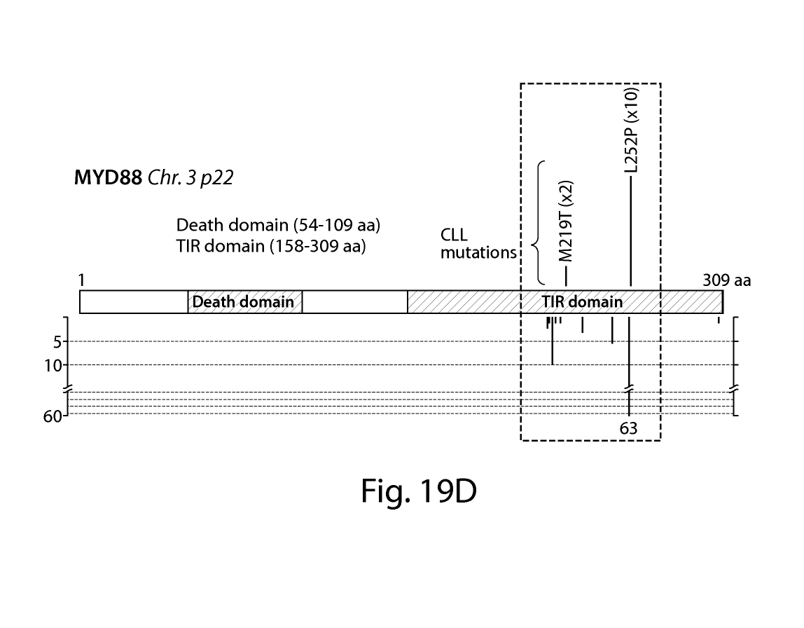

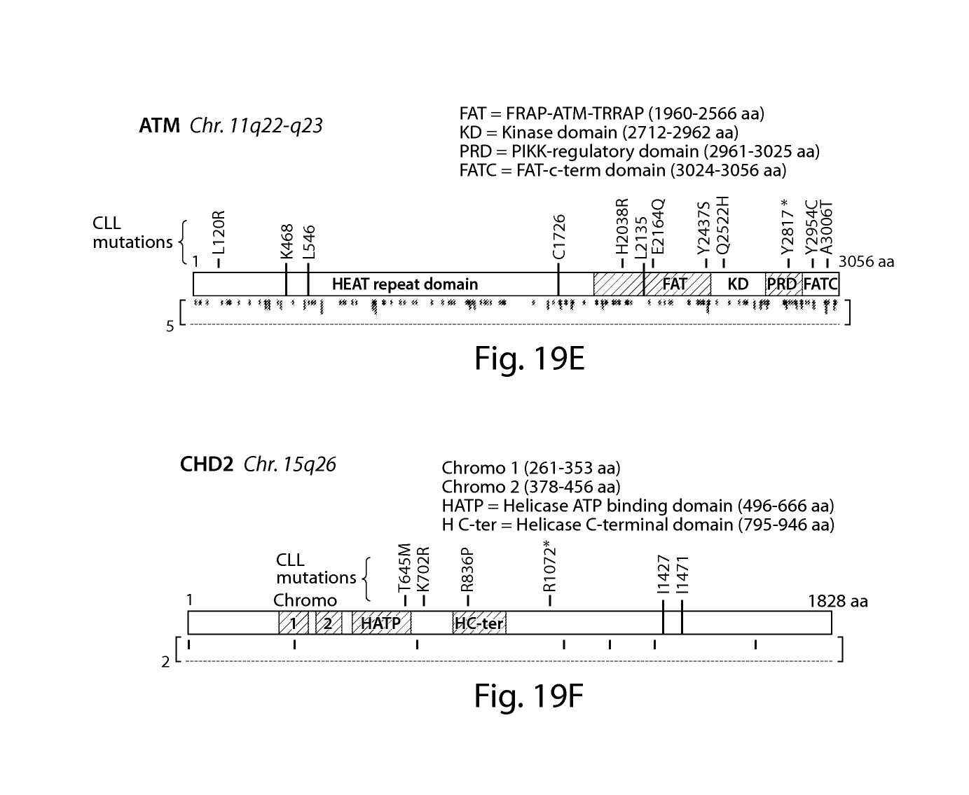

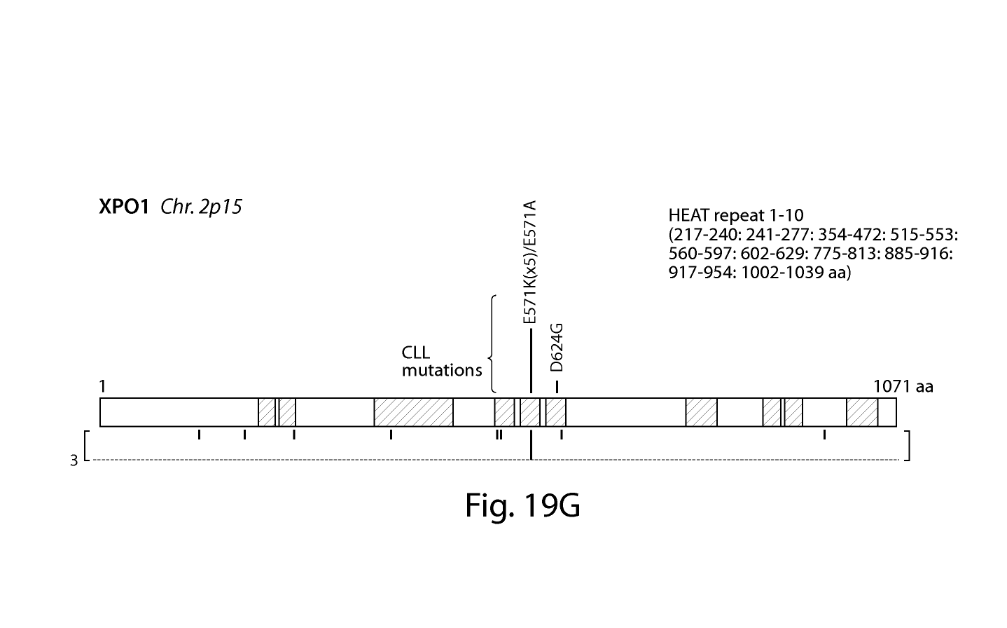

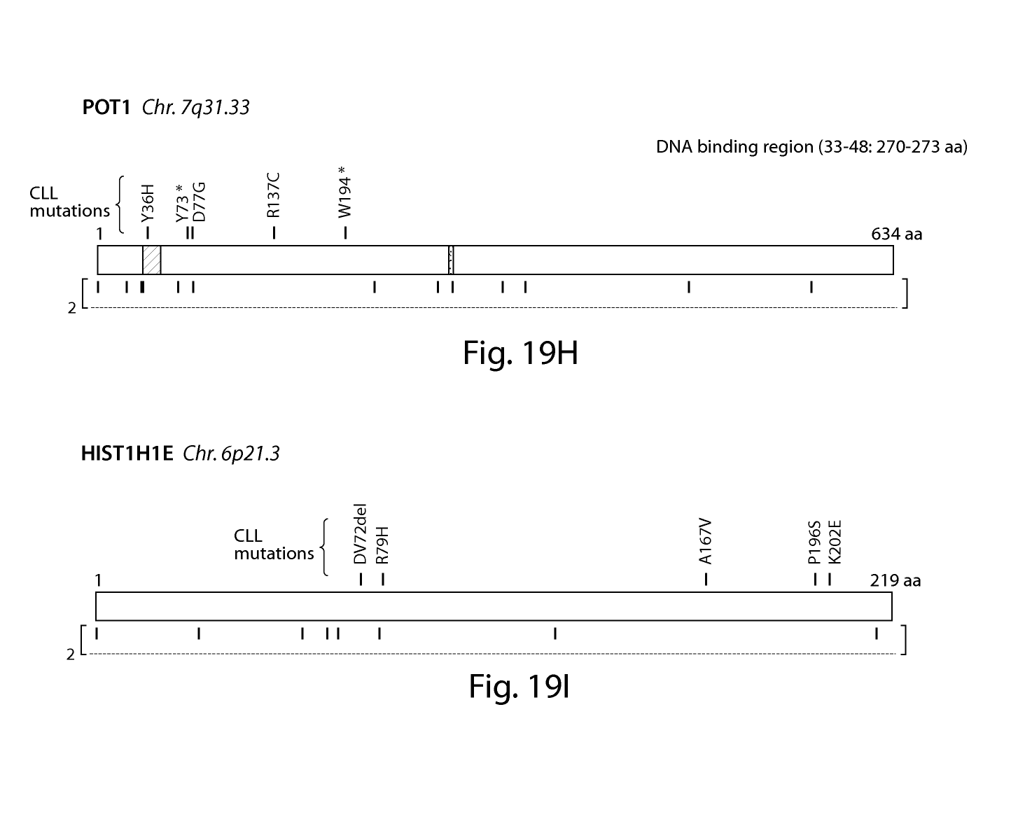

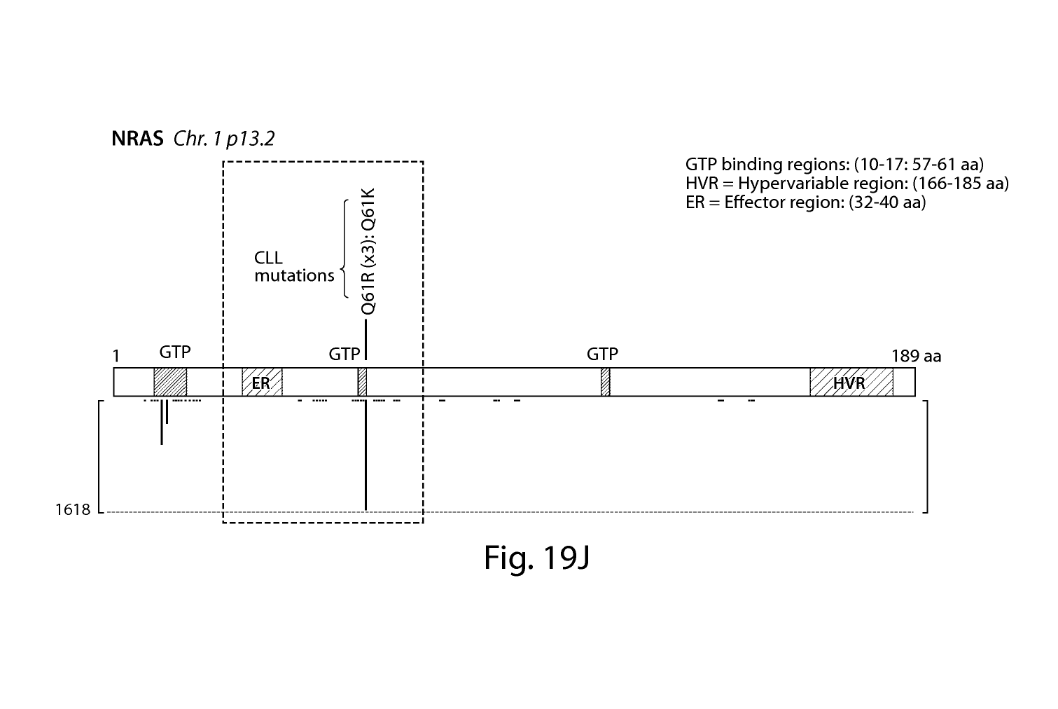

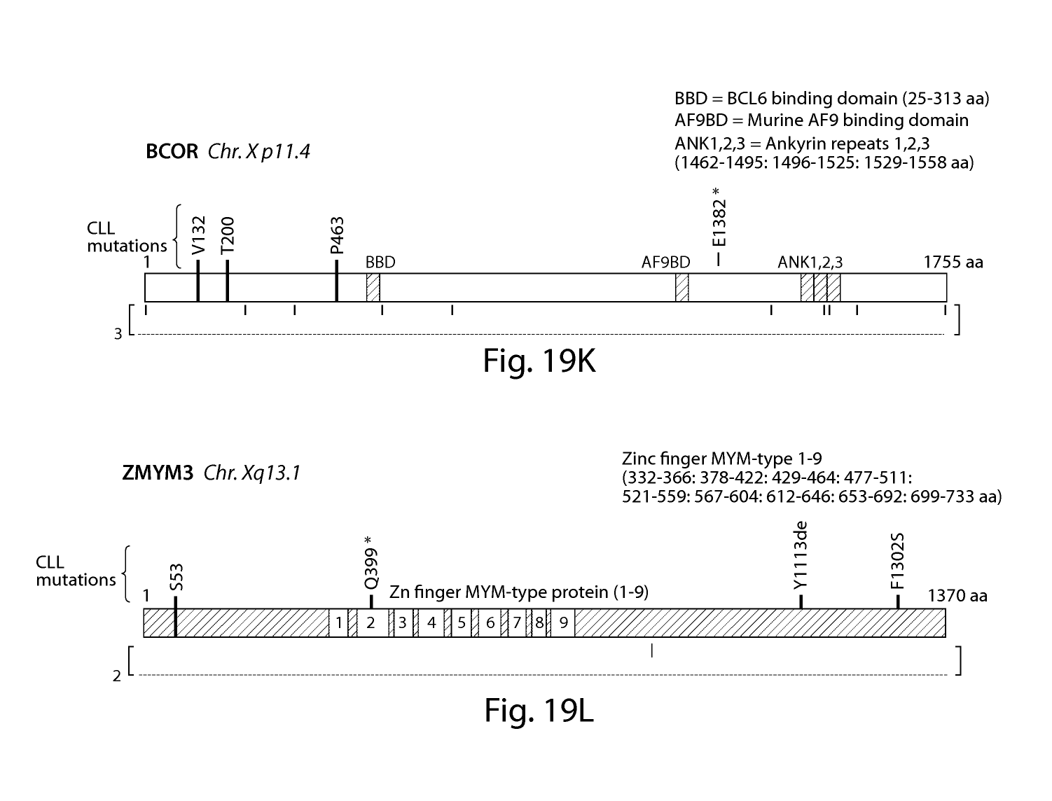

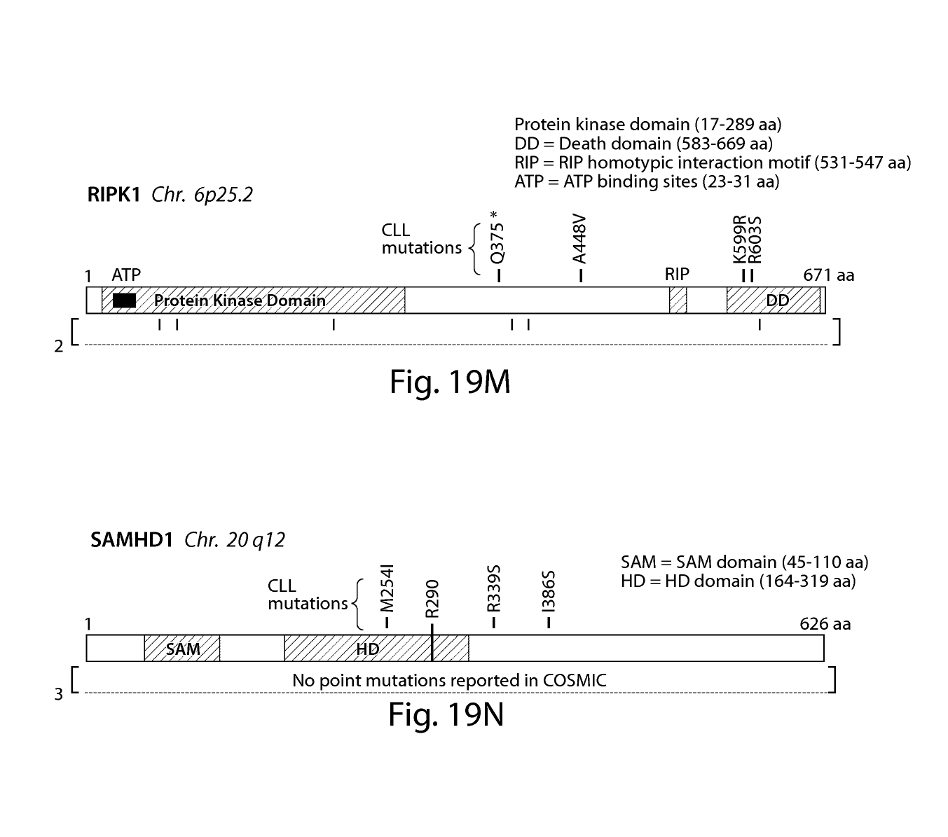

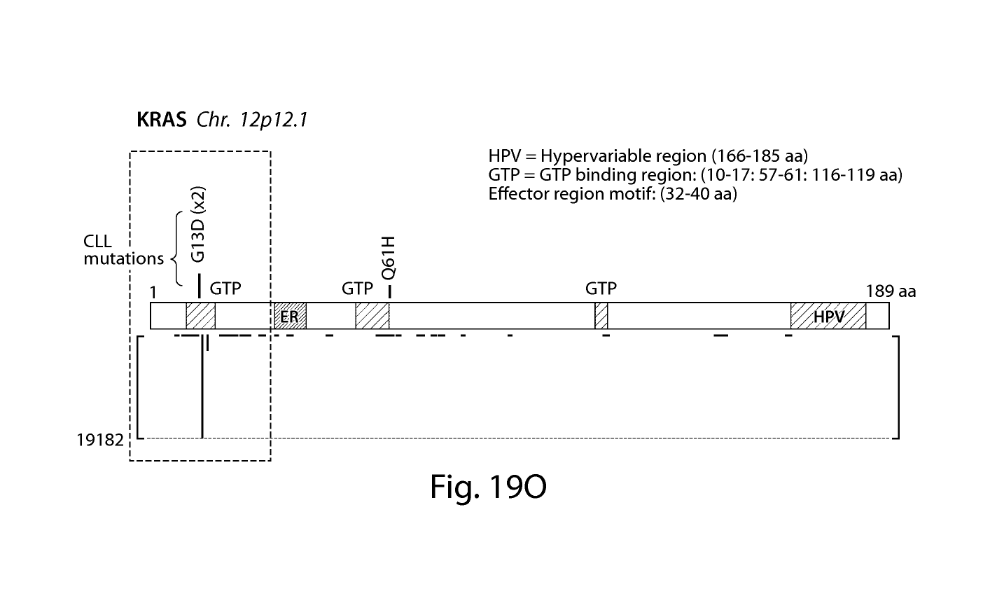

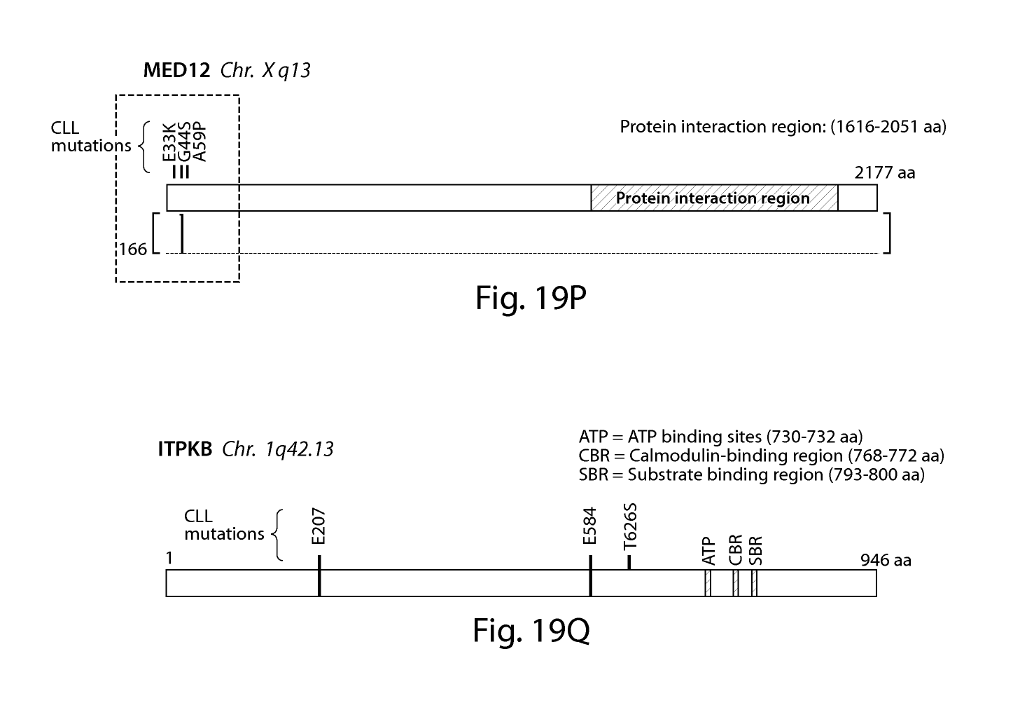

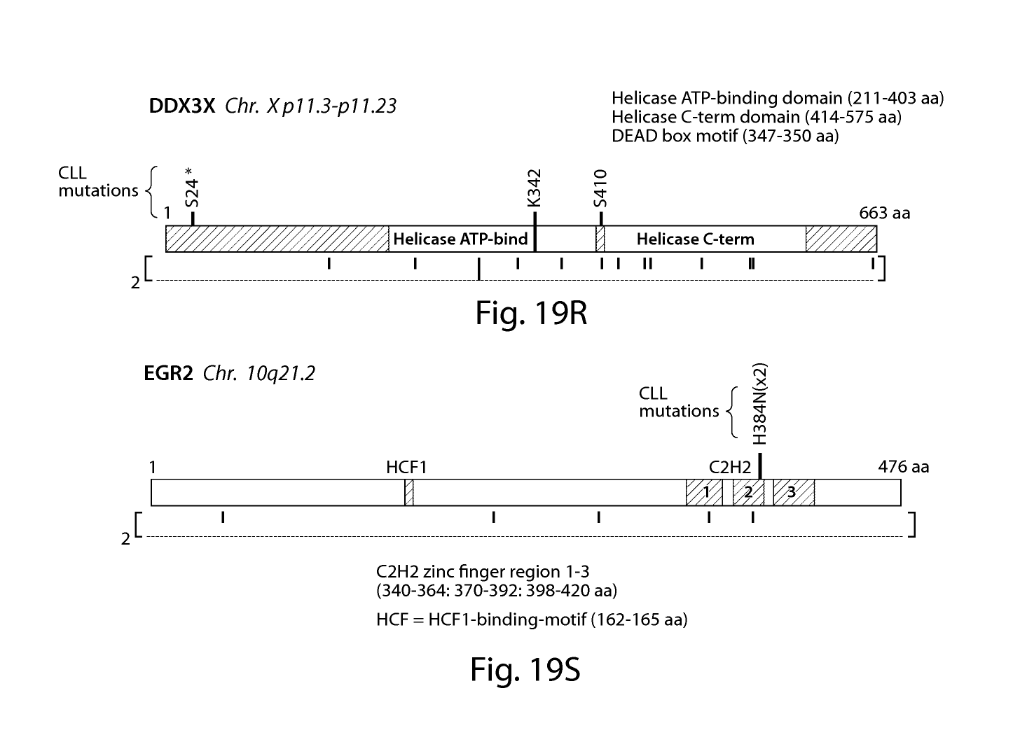

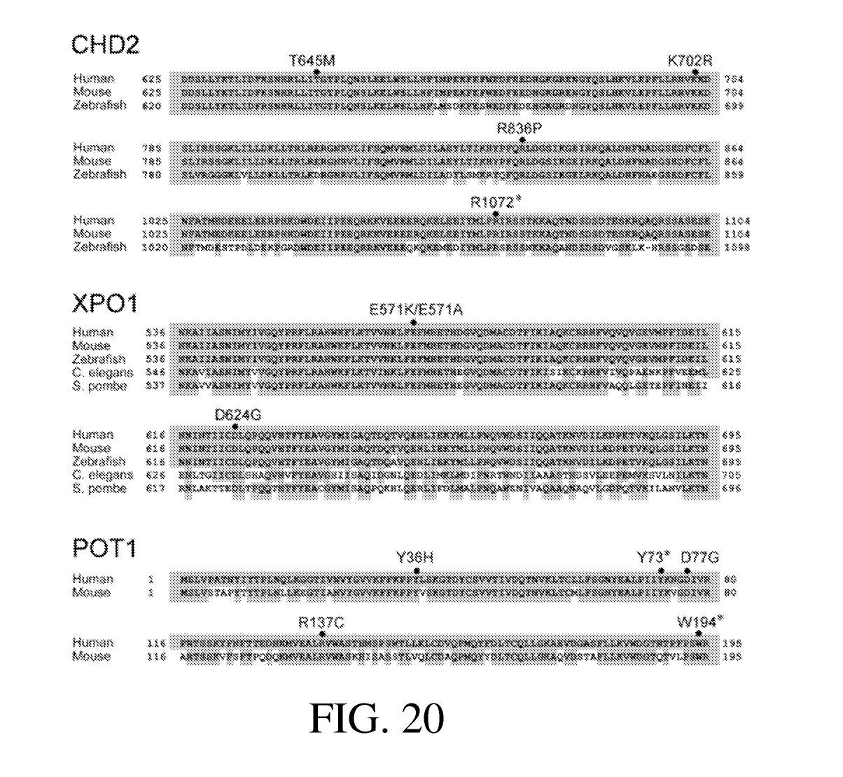

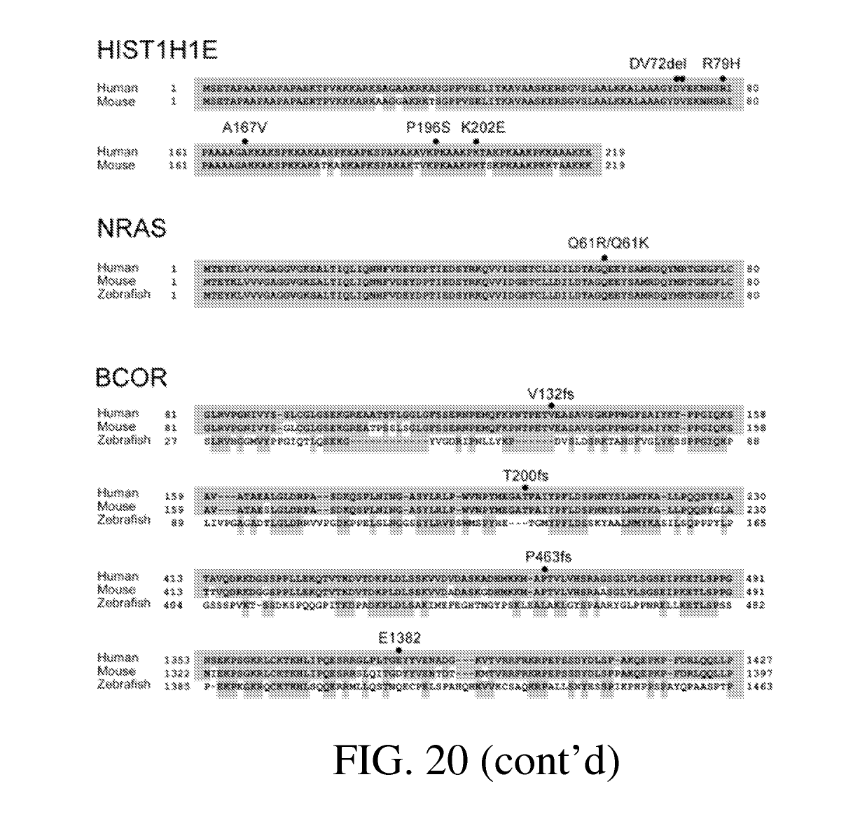

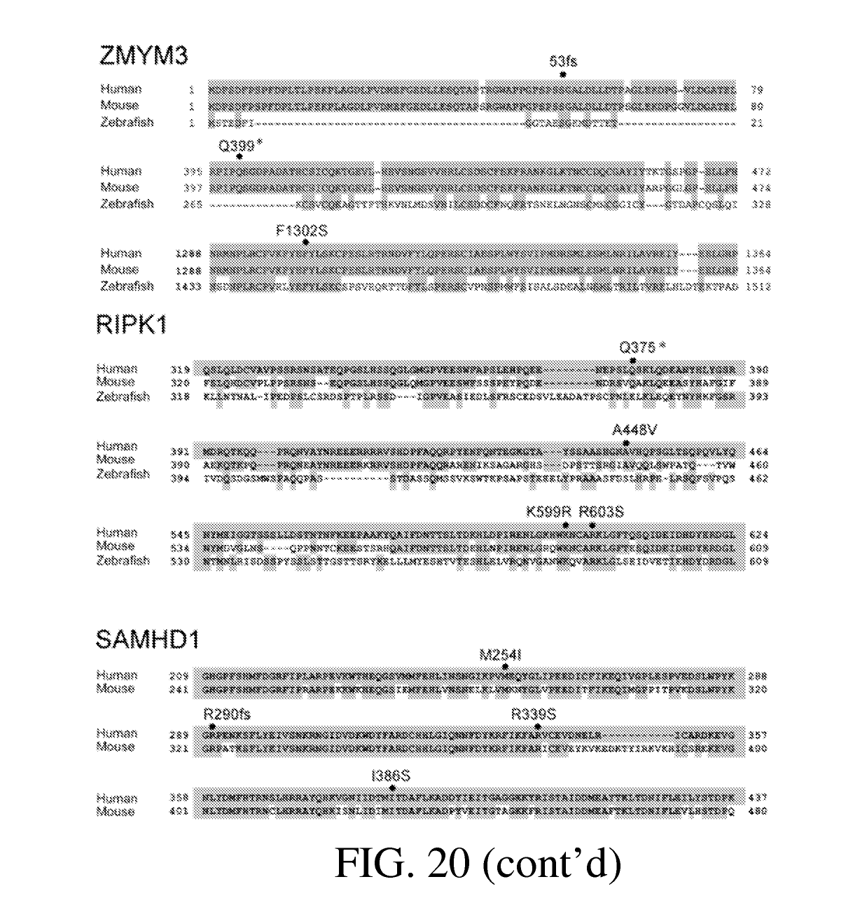

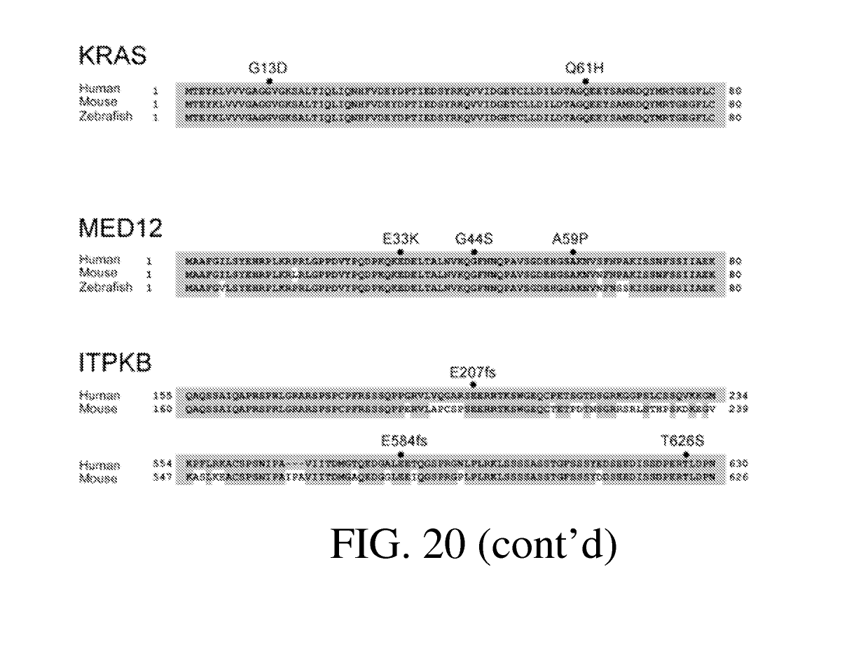

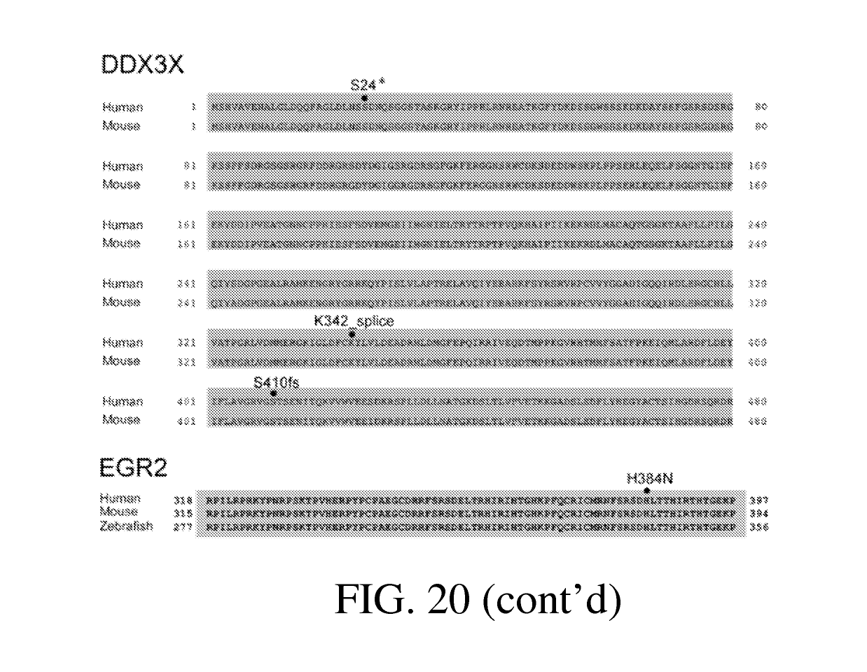

Expansion of our sample cohort provided us with the sensitivity to detect 20 putative CLL cancer genes (q<0.1), which was accomplished through recurrence analysis using the MutSig2.0 algorithm (Lohr et al., 2012) which detects genes enriched with mutations beyond the background mutation rate (FIG. 12A-top, FIG. 19) or genes with mutations that overlap with previously reported mutated sites (from COSMIC (Forbes et al., 2010); FIG. 12A-middle). These included 8 of the 9 genes identified in our initial report (TP53, ATM, MYD88, SF3B1, NOTCH1, DDX3X, ZMYM3, FBXW7) (Wang et al., 2011). The missing gene, MAPK1, did not harbor additional mutations in the increased sample set and therefore its overall mutation frequency now fell below our significance threshold. The 12 newly identified genes were mutated at lower frequencies, and hence were not detected in the subset of sequenced samples that we previously reported. Three of the 12 additional candidate driver genes were identified in recent CLL sequencing efforts (XPO1, CHD2, and POT1) (Fabbri et al., 2011; Puente et al., 2011). The 9 remaining genes represent novel candidate CLL drivers, with mutations occurring at highly conserved sites (FIG. 19). These included six genes with known roles in cancer biology (NRAS, KRAS (Bos, 1989), BCOR (Grossmann et al., 2011), EGR2 (Unoki and Nakamura, 2003), MED12 (Makinen et al., 2011) and RIPK1 (Hosgood et al., 2009)), two genes that affect immune pathways (SAMHD1 (Rice et al., 2009), ITPKB (Marechal et al., 2011)) and a histone modification gene (HIST1H1E (Alami et al., 2003)).

Together, the 20 candidate CLL driver genes appeared to fall into 7 core signaling pathways, in which the genes play roles. These include all five pathways that we previously reported to play a role in CLL (DNA repair and cell-cycle control, Notch signaling, inflammatory pathways, Wnt signaling, RNA splicing and processing). Two new pathways were implicated by our analysis: B cell receptor signaling and chromatin modification (FIG. 12B). We also noted that the CLL samples contained additional mutations in the genes that form these pathways (marked as pink ovals in FIG. 12B), some of which are known drivers in other malignancies.

Because recurrent chromosomal abnormalities have defined roles in CLL biology (Döhner et al., 2000; Klein et al., 2010), we further searched for loci that were significantly amplified or deleted by analyzing somatic copy-number alterations (sCNAs). We applied GISTIC2.0 (Mermel et al., 2011) to 111 matched tumor and normal samples which were analyzed by SNP6.0 arrays (Brown et al., 2012). Through this analysis, we identified deletions in chromosome 8p, 13q, 11q, and 17p and trisomy of chromosome 12 as significantly recurrent events (FIG. 12A-bottom). Thus, based on WES and copy number analysis, we altogether identified 20 mutated genes and 5 cytogenetic alterations as putative CLL driver events.

Inference of Genetic Evolution with Whole-Exome Sequencing Data.

In order to study clonal evolution in CLL, we performed integrative analysis of sCNAs and sSNVs using a recently reported algorithm ABSOLUTE (Carter et al., 2012), which jointly estimated the purity of the sample (fraction of cancer nuclei) and the average ploidy of the cancer cells. All samples were estimated to have near-diploid DNA content; these estimates were confirmed by FACS analysis of 7 CLL samples (FIG. 21). Our data were sufficient for resolution of these quantities in 149 of the 160 samples (data not shown), allowing for discrimination of subclonal from clonal alterations, including sCNAs, sSNVs, and selected indels. Our analysis approach is outlined in FIG. 13A. For each sSNV, we estimated its allelic fraction by calculating the ratio of alternate to total number of reads covering the mutation site in the WES data. These estimates were consistent with independent deeper genome sequencing and RNA sequencing (FIG. 21B-C, data not shown). Next, we used ABSOLUTE (Carter et al., 2012) to estimate the cancer cell fraction (CCF) harboring the mutation by correcting for sample purity and local copy-number at the sSNV sites (data not shown, FIG. 13B). We classified a mutation as clonal if the CCF harboring it was >0.95 with probability >0.5, and subclonal otherwise (FIG. 13A, inset). The results remained unchanged when more stringent cutoffs were used. For sSNVs designated as subclonal, median CCF was 0.49 with a range of 0.11 to 0.89.

Overall, we identified 1,543 clonal mutations (54% of all detected mutations, average of 10.3±5.5 mutations per sample, data not shown). These mutations were likely acquired either before or during the most recent complete selective sweep. This set therefore includes both neutral somatic mutations that preceded transformation and the driver and passenger event(s) present in each complete clonal sweep. A total of 1,266 subclonal sSNVs were detected in 146 of 149 samples called by ABSOLUTE (46%; average of 8.5±5.8 subclonal mutations per sample). These subclonal sSNVs exist in only a fraction of leukemic cells, and hence occurred after the emergence of the “most-recent common ancestor”, and by definition, also after disease initiation. The mutational spectra were similar in clonal and subclonal sSNVs (FIG. 22), consistent with a common set of mutational processes giving rise to both groups.

Age and Mutated IGHV Status are Associated with an Increased Number of Clonal Somatic Mutations.

The presence of subclones in nearly all CLL samples enabled us to analyze several aspects of leukemia progression. We first addressed how clonal and subclonal mutations relate to the salient clinical characteristics of CLL. CLL is generally a disease of the elderly with established prognostic factors, such as the IGHV mutation (Döhner, 2005) and ZAP70 expression. Patients with a high number of IGHV mutations (mutated IGHV) tend to have better prognosis than those with a low number (unmutated IGHV) (Damle et al., 1999; Lin et al., 2009). This marker may reflect the molecular differences between leukemias originating from B cells that have or have not yet, respectively, undergone the process of somatic hypermutation that occurs as part of normal B cell development. We examined the association of these factors, as well as patient age at diagnosis, with the prevalence of clonal and subclonal mutations. We found that age and mutated IGHV status were associated with greater numbers of clonal (but not subclonal) mutations (age, P<0.001; mutated vs unmutated IGHV, P=0.05; FIG. 13C) while there was no association with ZAP70 expression (data not shown). Since CLL samples with mutated IGHV derive from B-cells that have experienced a burst of mutagenesis as part of normal B cell somatic hypermutation, the increased number of clonal somatic mutations is likely related to aberrant mutagenesis that preceded clonal transformation (Deutsch et al., 2007; McCarthy et al., 2003). Furthermore, the higher number of clonal sSNVs in older individuals is consistent with the expectation that more neutral somatic mutations accumulate over the patient's lifetime prior to the onset of cancer later in life (Stephens et al., 2012; Welch et al., 2012). Subclonal mutations are increased with treatment. The effect of treatment on subclonal heterogeneity in CLL is unknown. In samples from 29 patients treated with chemotherapy prior to sample collection, we observed a significantly higher number of subclonal (but not clonal) sSNVs per sample than in the 120 patients who were chemotherapy-naïve at time of sample (FIG. 13D, top and middle panels). Using an analysis of covariance model, we observed that receipt of treatment prior to sample among the 149 patients was statistically significant (P=0.048) but time from diagnosis to sample was not (P=0.31). Because patients that do not require treatment in the long-term may have a distinct subtype of CLL, we also restricted the comparison of the 29 pre-treated CLLs to only the 42 that were eventually treated after sample collection and again confirmed this finding (P=0.02). In these 42 patients, a higher number of subclonal mutations was not correlated with a shorter time to treatment (correlation coefficient=0.03; P=0.87). Thus, therapy prior to sample was associated with a higher number of subclonal mutations, and furthermore, the number of subclonal sSNVs detected increased with the number of prior therapies (P=0.011, data not shown).

Cancer therapy has been theorized to be an evolutionary bottleneck, in which a massive reduction in malignant cell numbers results in reduced genetic variation in the cell population (Gerlinger and Swanton, 2010). The overall diversity in CLL may be diminished after therapeutic bottlenecks as well. Because most of the genetic heterogeneity within a cancer is present at very low frequencies (Gerstung et al., 2012)—below the level of detection afforded by the ˜130× sequence coverage we generated—we were unable to directly assess reduction in overall genetic variation.

However, in the range of larger subclones that were observable by our methods, (>10% of malignant cells), we witnessed increased diversity after therapy (FIG. 13D). Although, the available data cannot definitively rule out extensive diversification following therapy, this increase likely results, at least in part, from outgrowth of pre-existing minor subclones. This may result from the removal of dominant clones by cytotoxic treatment, eliminating competition for growth and allowing the expansion of one or more fit subclones to frequencies above our detection threshold. Further supporting our interpretation that fitter clones grow more effectively and become detectable after treatment, we observed an increased frequency of subclonal driver events (which are presumably fitter) in treated relative to untreated patients (FIG. 13D, bottom) (note that driver events include CLL driver mutations (FIG. 12A) and sSNVs in highly conserved sites of genes in the Cancer Gene Census (Futreal et al., 2004)).

Inferring the Order of Genetic Changes Underlying CLL.

While general aspects of temporal evolution could not be completely resolved in single timepoint WES samples, the order of driver mutation acquisition could be partially inferred from the aggregate frequencies at which they are found to be clonal or subclonal. We considered the 149 samples as a series of “snapshots” taken along a temporal axis. Clonal status in all or most mutations affecting a specific gene or chromosomal lesion would indicate that this alteration was acquired at or prior to the most recent selective sweep before sampling and hence could be defined as a stereotypically early event. Conversely, predominantly subclonal status in a specific genetic alteration implies a likely later event that is tolerated and selected for only in the presence of an additional mutation.

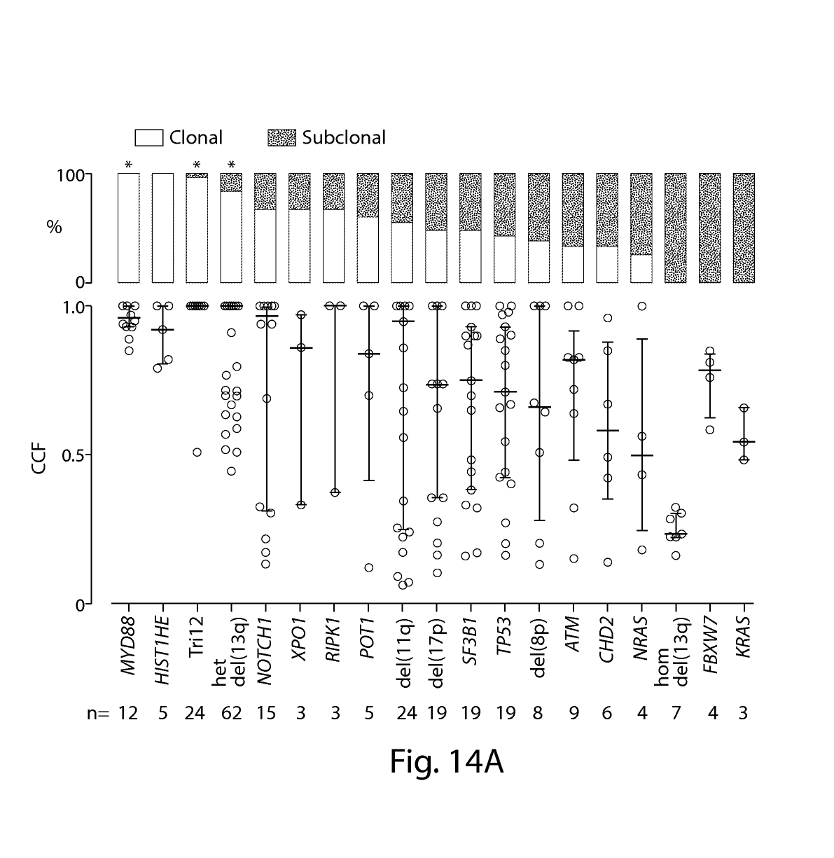

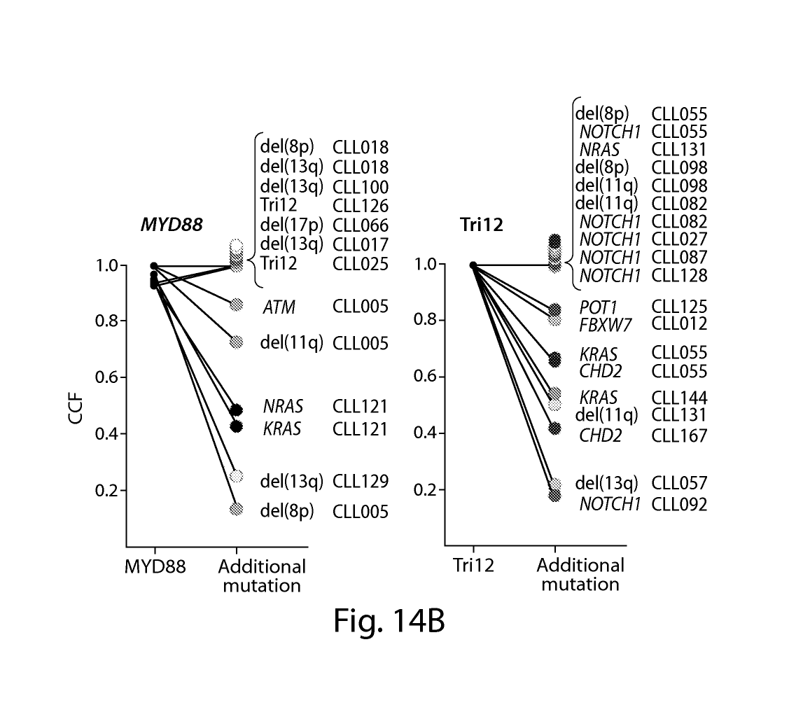

This strategy was used to infer temporal ordering of the recurrent sSNVs and sCNAs (FIG. 14A). We focused on alterations found in at least 3 samples within the cohort of 149 CLL samples. We found that three driver mutations—MYD88 (n=12), trisomy 12 (n=24), and hemizygous del(13q) (n=70)—were clonal in 80-100% of samples harboring these alterations, a significantly higher level than for other driver events (q<0.1, Fisher exact test with Benjamini-Hochberg FDR (Benjamini and Hochberg, 1995)), implying that they arise earlier in typical CLL development. Mutations in HIST1H1E, although clonal in 5 of 5 affected samples, did not reach statistical significance. Other recurrent CLL drivers—for example, ATM, TP53 and SF3B1 (9, 19 and 19 mutations in 6, 17 and 19 samples, respectively)—were more often subclonal, indicating that they tend to arise later in leukemic development and contribute to disease progression. We note that the above approach assumed that different CLL samples evolve along a common temporal progression axis. We therefore examined specifically CLL samples that harbored one ‘early’ driver mutation and any additional driver alteration(s). The ‘early’ events had either similar or a higher CCF compared to ‘later’ events (examples for trisomy 12 and MYD88 given in FIG. 14B).

Direct Observation of Clonal Evolution by Longitudinal Data Analysis of Chemotherapy-Treated CLL.

To directly assess the evolution of somatic mutations in a subset of patients, we compared CCF for each alteration across two clinical timepoints in 18 of the 149 samples (median years between timepoints was 3.5; range 3.1-4.5). Six patients (‘untreated’) did not receive treatment throughout the time of study. The remaining 12 patients (‘treated’) received chemotherapy (primarily fludarabine and/or rituxan-based) in the interval between samples (data not shown). The two patient groups were not significantly different in terms of elapsed time between first and second sample (median 3.7 years for the 6 untreated patients compared to 3.5 years for the 12 treated patients, P=0.62; exact Wilcoxon rank-sum test), nor did it differ between time of diagnosis to first sample (P=0.29).

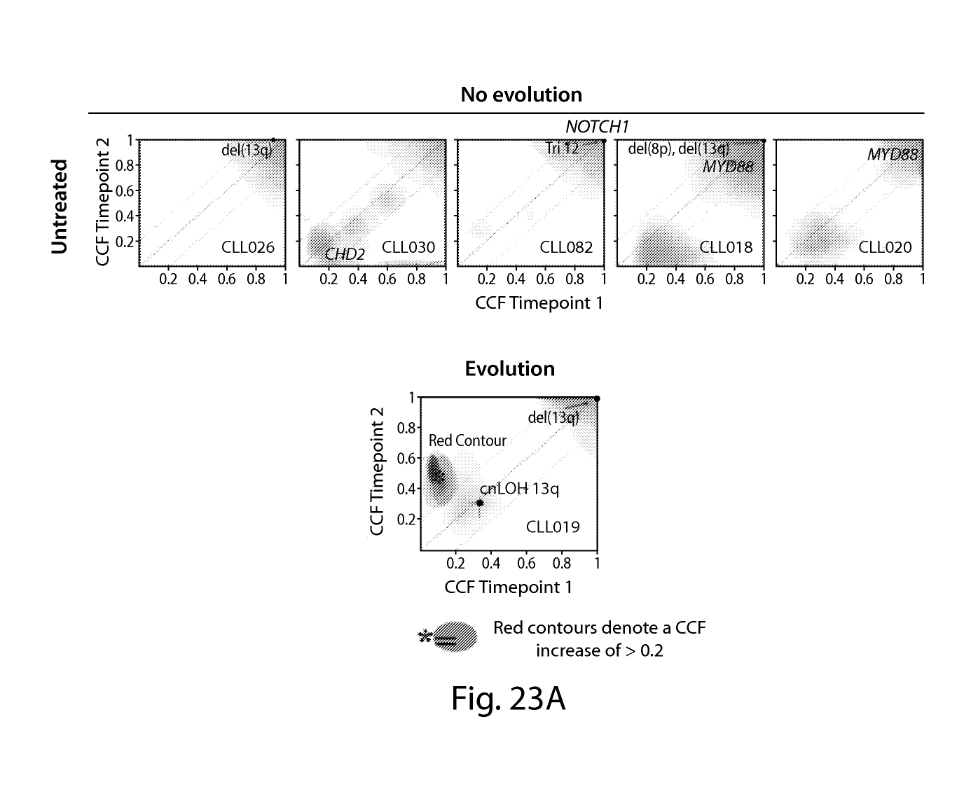

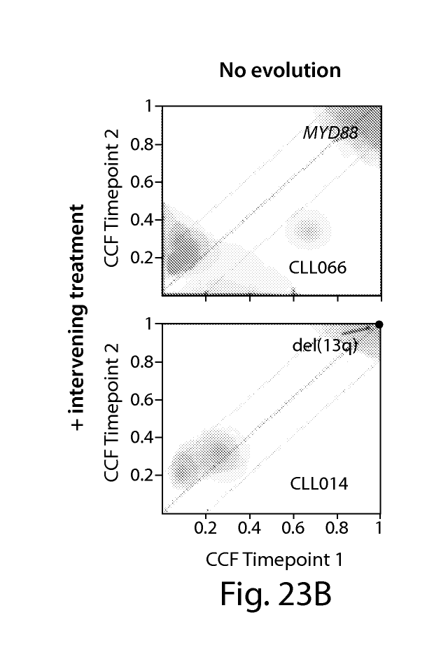

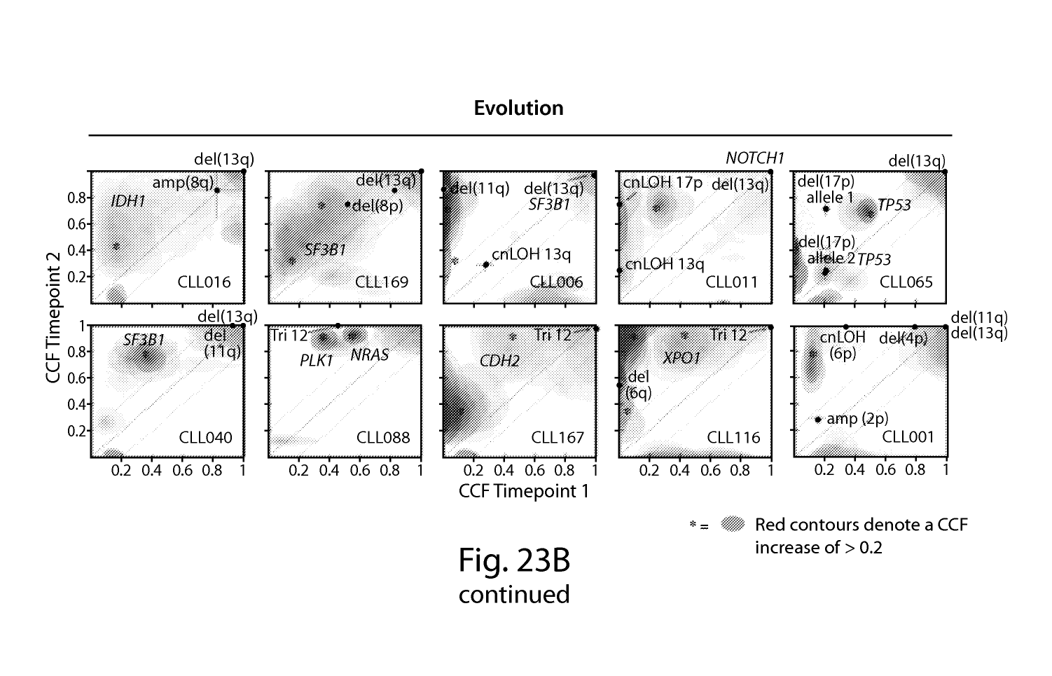

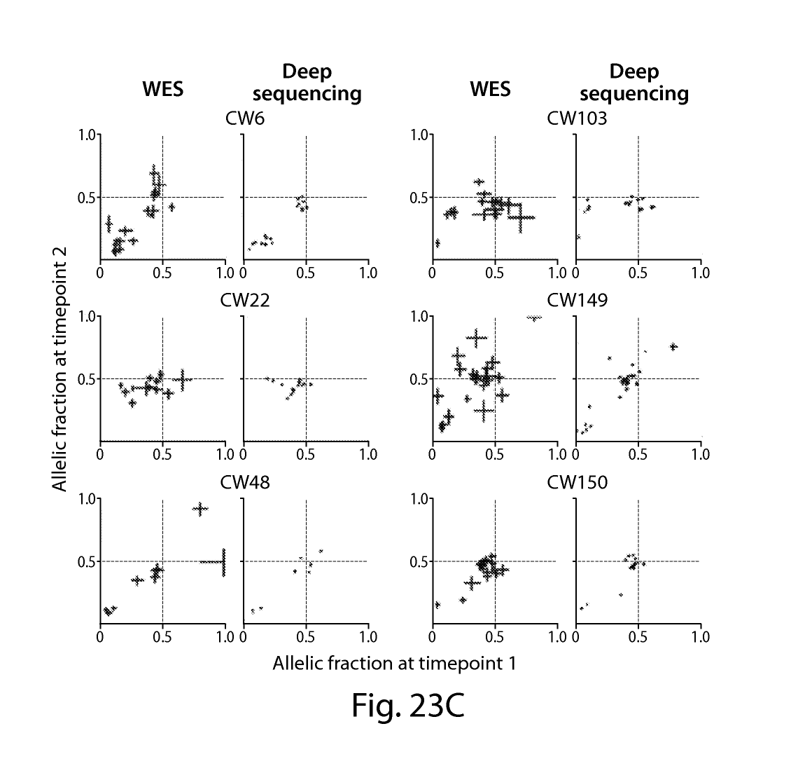

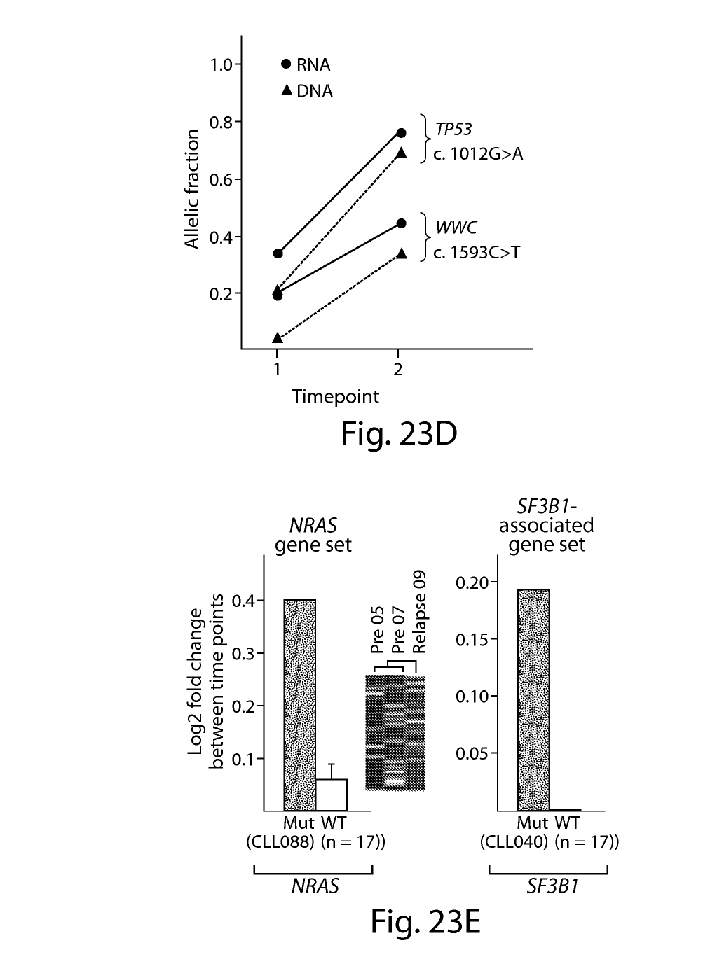

Analysis of the 18 sets of data revealed that 11% of mutations increased (34 sSNVs, 15 sCNAs), 2% decreased (6 sSNVs, 2 sCNAs) and 87% did not change their CCF over time (q<0.1 for significant change in CCF, data not shown). As shown by our single timepoint analysis, we observed a shift of subclonal driver mutations (e.g., del(11q), SF3B1 and TP53) towards clonality over time. Changes in the genetic composition of CLL cells with clonal evolution were associated with network level changes in gene expression related to emergence of specific subclonal populations (e.g. changes in signatures associated with SF3B1 or NRAS mutation, FIG. 23D, data not shown). Finally, expanding sSNVs were enriched in genes included in the Cancer Gene Census (Futreal et al., 2004) (P=0.021) and in CLL drivers (P=0.028), consistent with the expected positive selection for the subclones harboring them.

Clustering analysis of CCF distributions of individual genetic events over the two timepoints, revealed clear clonal evolution in 11 of 18 CLL sample pairs. We observed clonal evolution in 10 of 12 sample pairs which had undergone intervening treatment between timepoints 1 and 2 (FIG. 15B, FIG. 23A-C). This was contrasted with the 6 untreated CLLs, 5 of which demonstrated equilibrium between subpopulations that was maintained over several years (FIG. 15, P=0.012, Fisher exact test). Of the 11 patients with subclonal evolution across the sampling interval, 5 followed a branched evolution pattern as indicated by the disappearance of mutations with high CCF co-occurring with the expansion of other subclones (FIG. 15B). This finding demonstrates that co-existing sibling subclones are at least as common in CLL as are linear nested subclones, as demonstrated in other hematological malignancies (Ding et al., 2012; Egan et al., 2012). We conclude that chemotherapy-treated CLLs often undergo clonal evolution resulting in the expansion of previously minor subclones. Thus, these longitudinal data validate the insights obtained in the cross-sectional analysis, namely that (i) ‘later’ driver events expand over time (FIG. 14A) and (ii) treatment results in the expansion of subclones enriched with drivers (and thus presumably have higher fitness) (FIG. 13D).

Presence of Subclonal Drivers Adversely Impacts Clinical Outcome.

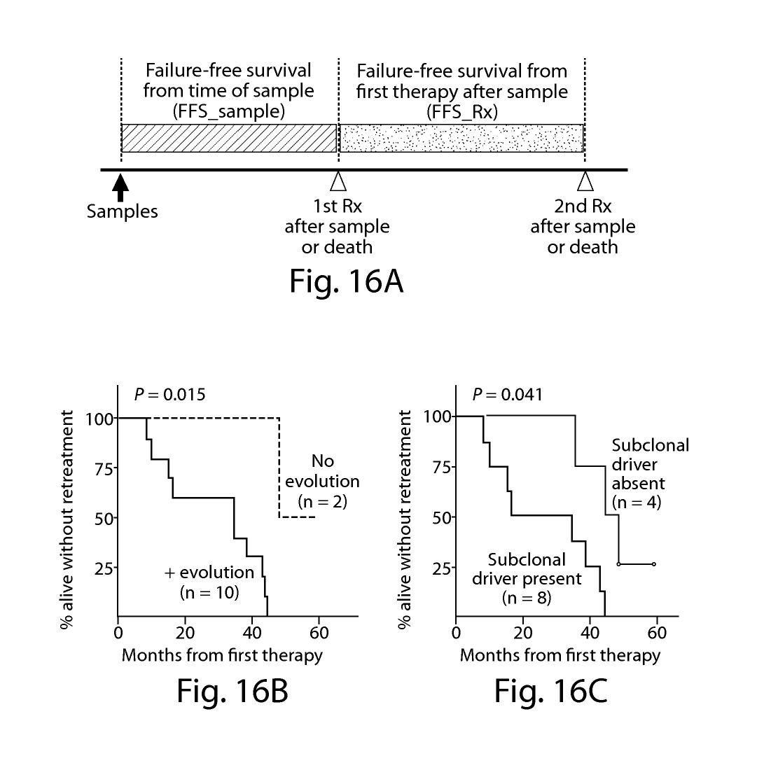

We observed treatment-associated clonal evolution to lead to the replacement of the incumbent clone by a fitter pre-existing subclone (FIG. 15B). Therefore, we would expect a shorter time to relapse in individuals with evidence of clonal evolution following treatment. As a measure of relapse, we assessed failure-free survival from time of sample (‘FFS_Sample’) and failure-free survival from time of next therapy (‘FFS_Rx’, FIG. 16A), where failure is defined as retreatment (a recognized endpoint in slow growing lymphomas (Cheson et al., 2007)) or death. For the study of clonal evolution in CLL, the use of retreatment is a preferable endpoint to other measures such as progression alone, as this is a well-defined event that is reflective of CLL disease aggressiveness. For example, disease progression alone in CLL may be asymptomatic without necessitating treatment; conversely, treatment is administered only in the setting of symptomatic disease or active disease relapse (Hallek et al., 2008).

Within the 12 of 18 longitudinally analyzed samples that received intervening treatment, we observed that the 10 samples with clonal evolution exhibited shortened FFS_Rx (log-rank test; P=0.015, FIG. 16B). Importantly, the somatic driver mutations that expanded to take over the entire population upon relapse (‘timepoint-2’), were often already detectable in the pre-treatment (‘timepoint-1’) sample (FIGS. 15B and 23B). Our results thus show that presence of detectable subclonal drivers in pre-treatment samples can anticipate clonal evolution in association with treatment. Indeed, the 8 of 12 samples with presence of subclonal drivers in pretreatment samples exhibited shorter FFS_Rx than the 4 samples with subclonal drivers absent (p=0.041; FIG. 16C). Together, the results of our longitudinally studied patient samples showed that the presence of driver events within subclones may impact prognosis and clinical outcome.

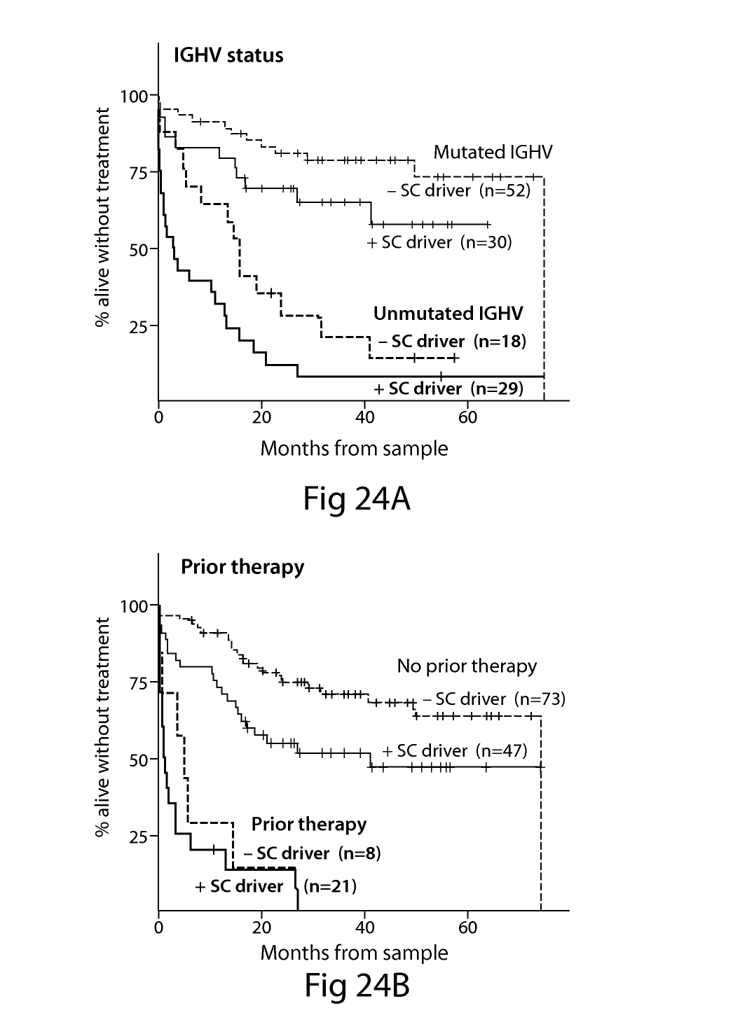

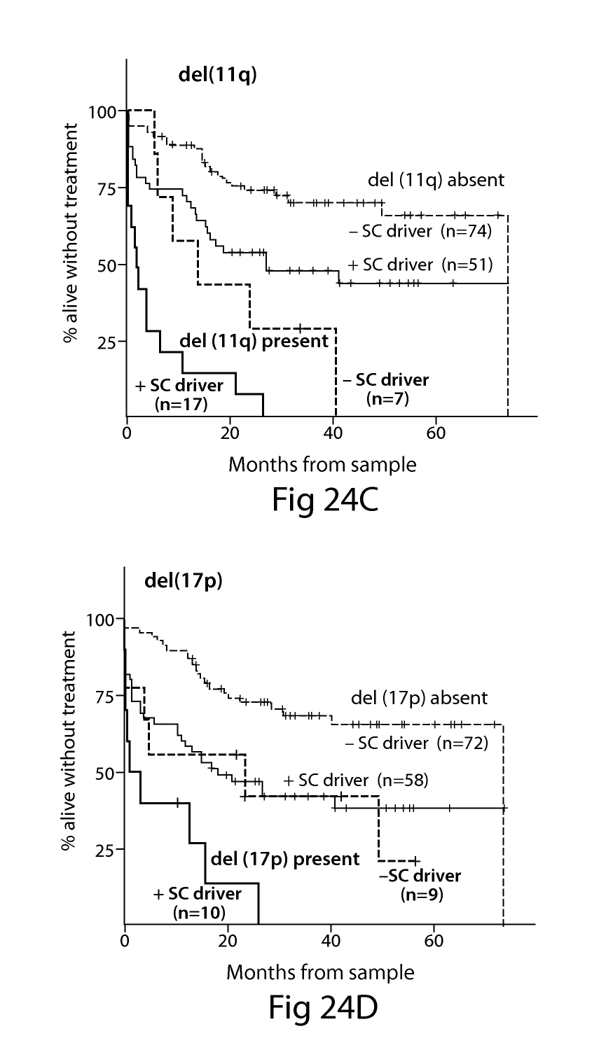

We tested this hypothesis in the set of 149 patient samples, of which subclonal driver mutations were detected in 46% (FIG. 17A; data not shown). Indeed, we found that CLL samples with subclonal driver mutations were associated with a shorter time from sample collection to treatment or death (‘FFS_Sample’, P<0.001, FIG. 17B, data not shown), that seemed to be independent of established markers of poor prognosis (i.e. unmutated IGHV, or presence of de/(11q) or del(17p), FIG. 24). Moreover, we tested specifically whether the presence of pre-treatment subclonal drivers was associated with a shorter FFS_Rx, as we observed in the longitudinal data. Therefore, we focused on the 67 patients who were treated after sample collection (median time to first therapy from time of sample was 11 months [range 1-45]). These patients could be divided into two groups based on the presence (n=39) or absence (n=29) of a subclonal driver (62% and 64%, respectively, were treated with fludarabine-based immunochemotherapy, P=0.4). The 39 of these patients in which subclonal CLL drivers were detected required earlier retreatment or died (shorter FFS_Rx; log-rank test, P=0.006; FIG. 17C, data not shown), indicative of a more rapid disease course.

Regression models adjusting for multiple CLL prognostic factors (IGHV status, prior therapy and high risk cytogenetics) supported the presence of a subclonal driver as an independent risk factor for earlier retreatment (adjusted hazard ratio (HR) of 3.61 (CI 1.42-9.18), Cox P=0.007; unadjusted HR, 3.20 (CI 1.35-7.60); FIG. 17D), comparable to the strongest known CLL risk factors. In similar modeling within a subset of 62 patients who had at least one driver (clonal or subclonal), the association of the presence of a subclonal driver with a shorter time to retreatment or death was also significant (P=0.012, data not shown) reflecting that this difference is not merely attributable to the presence of a driver. Additionally, an increased number of subclonal driver mutations per sample (but not an increased number of clonal drivers) was also associated with a stronger HR for shorter FFS_Rx (data not shown). Finally, this association retained significance (Cox P=0.033, data not shown) after adjusting for the presence of mutations previously associated with poor prognosis (ATM, TP53, SF3B1), showing that in addition to the driver's identity, its subclonal status also affects clinical outcome.

### DISCUSSION

The analysis of clonal heterogeneity in CLL provides a glimpse into the past, present and future of a patient's disease. While inter-tumoral (Quesada et al., 2012; Wang et al., 2011) and intra-tumoral (Schuh et al., 2012; Stilgenbauer et al., 2007) genetic heterogeneity had been previously demonstrated in CLL, our use of novel WES-based algorithms enabled a more comprehensive study of clonal evolution in CLL and its impact on clinical outcome. Through the cross-sectional analysis of 149 samples, we derived the number and genetic composition of clonal and subclonal mutations and thus uncovered footprints of the past history of CLL, such as the accumulation of passenger mutations related to age and aberrant somatic hypermutation preceding transformation. Furthermore, we inferred a temporal order of genetic events implicated in CLL. Finally, our combined longitudinal and cross-sectional analyses revealed that knowledge of subclonal mutations can anticipate the genetic composition of the future relapsing leukemia and the rapidity with which it will occur.

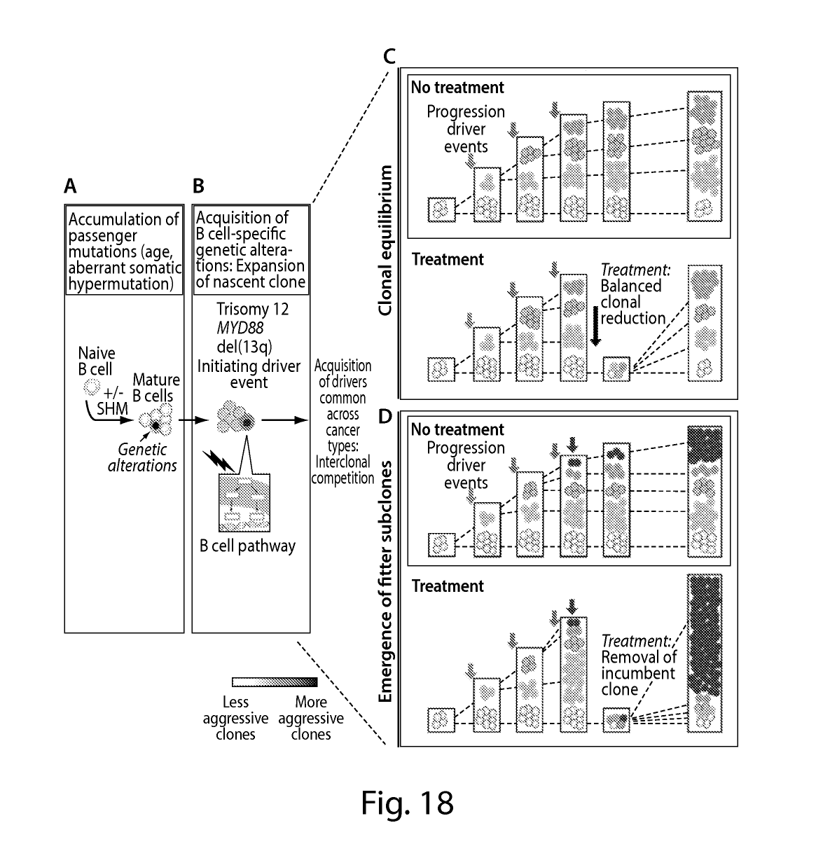

We proposed the existence of distinct periods in CLL progression, with unique selection pressures acting at each period. In the first period prior to transformation, passenger events accumulate in the cell that will eventually be the founder of the leukemia (in proportion to the age of the patient; FIG. 13C), and are thus clonal mutations (FIG. 18A). In the second period, the founding CLL mutation appears in a single cell and leads to transformation (FIG. 18B); these are also clonal mutations, but unlike passenger mutations, these are recurrent across patients. We identified driver mutations that were consistently clonal (del(13q), MYD88 and trisomy 12; FIG. 14A) and which appear to be relatively specific drivers of CLL or B cell malignancies (Beroukhim et al., 2010; Döhner et al., 2000; Ngo et al., 2010). In the third period of disease progression, subclonal mutations expand over time as a function of their fitness integrating intrinsic factors (e.g. proliferation and apoptosis) and extrinsic pressures (e.g., interclonal competition and therapy) (FIG. 18C-D). The subclonal drivers include ubiquitous cancer genes, such as ATM, TP53 or RAS mutations (FIG. 14A). These data show that mutations that selectively affect B cells may contribute more to the initiation of disease and precede selection of more generic cancer drivers that underlie disease progression—providing predictions that can be tested in human B cells or animal models of CLL.

An important question addressed here is how treatment affects clonal evolution in CLL. In the 18 patients monitored at 2 timepoints, we observed two general patterns—clonal equilibrium in which the relative sizes of each subclone were maintained and clonal evolution in which some subclones emerge as dominant (FIG. 15). Without treatment, 5 of 6 CLLs remained in stable equilibrium while 1 CLL showed clonal evolution. With treatment, only 2 of 12 patients were stable and 10 of 12 showed clonal takeover. We propose that in untreated samples, more time is needed for a new fit clone to take over the population in the presence of existing dominant clones (FIG. 18D-top). In contrast, in treated samples, cytotoxic therapy typically removes the incumbent clones (Jablonski, 2001)—acting like a ‘mass extinction’ event (Jablonski, 2001)—and shifts the evolutionary landscape (Nowak and Sigmund, 2004; Vincent and Gatenby, 2008) in favor of one or more aggressive subclones (Maley et al., 2006) (FIG. 18D-bottom). Thus, highly fit subclones likely benefit from treatment and exhibit rapid outgrowth (Greaves and Maley, 2012).

CLL is an incurable disease with a prolonged course of remissions and relapses. It has been long recognized that relapsed disease responds increasingly less well to therapy over time. We now show an association between increased clinical aggressiveness and genetic evolution, which has therapeutic implications. We found that the presence of pre-treatment subclonal driver mutations anticipated the dominant genetic composition of the relapsing tumor. Such information may eventually guide the selection of therapies to prevent the expansion of highly fit subclones. In addition, the potential hastening of the evolutionary process with treatment provides a mechanistic justification for the empirical practice of ‘watch and wait’ as the CLL treatment paradigm (CLL Trialists Collaborative Group, 1999). The detection of driver mutations in subclones (a testimony to an active evolutionary process) may thus provide a new prognostic approach in CLL, which can now be rigorously tested in larger clinical trials.

In conclusion, we demonstrate the ability to study tumor heterogeneity and clonal evolution with standard WES (coverage depth of ˜130×). These innovations will allow characterization of the subclonal mutation spectrum in large, publically available datasets (Masica and Karchin, 2011). The implementation described here may also be readily adopted for clinical applications. Even more importantly, our studies underscore the importance of evolutionary development as the engine driving cancer relapse. This new knowledge challenges us to develop novel therapeutic paradigms that not only target specific drivers (i.e., ‘targeted therapy’) but also the evolutionary landscape (Nowak and Sigmund, 2004) of these drivers.

TABLE 1
Summary metrics of whole genome and exome sequencing studies.

| | Average bases | |
|---|---|---|
| | covered per | Average exome coverage |
| | exome (34.3 Mb) | (CLL/normal) |
| Whole genomes (n = 3) | 70% | 38x/33x |
| Whole exomes (n = 88) | 81% | 132x/146x |

| | Average mutations/Mb | Average # of coding |
|---|---|---|
| | (Rate +/− SD across 91 cells | mutations (range) |
| Non-synonymous | 0.7 ± 0.36 | 20 (2-76) |
| Synonymous | 0.2 ± 0.16 | 5.8 (0-31) |

TABLE 2
A complete list of somatic non-synonymous mutations in the final analysis set of 3
CLL genomes and 88 CLL exomes.

| | | | | | | | Patient |
|---|---|---|---|---|---|---|---|
| Gene Name | Gene ID | Start_position | Variant_Classification | cDNA_Change | Protein_Change | Annotation | ID |
| APEX2 | 27301 | 55045451 | Missense | c.360C > G | p.A95G | uc004dtz.1 | P1 |
| ASXL1 | 171023 | 30488000 | Nonsense | c.4250C > G | p.S1275* | uc002wxs.1 | P1 |
| ATP13A2 | 23400 | 17196202 | Missense | c.1129T > G | p.C365W | uc001baa.1 | P1 |
| BZRAP1 | 9256 | 53745004 | Missense | c.3048C > T | p.S726F | uc002ivx.2 | P1 |
| C11orf61 | 79684 | 124175119 | Missense | c.391A > G | p.E123G | uc001qba.1 | P1 |
| C7orf51 | 222950 | 99924946 | Missense | c.1825G > A | p.A556T | uc003uvd.1 | P1 |
| CREB3L2 | 64764 | 137263565 | Missense | c.585A > G | p.M64V | uc003vtw.1 | P1 |
| DNMT3L | 29947 | 44493352 | Missense | c.1464T > C | p.I327T | uc002zeh.1 | P1 |
| GGA1 | 26088 | 36358654 | Missense | c.2275G > T | p.G637V | uc003atc.1 | P1 |
| HIPK2 | 28996 | 138908403 | Missense | c.3581A > C | p.Y1136S | uc003vvf.2 | P1 |
| INPP4B | 8821 | 143263948 | Missense | c.2559A > T | p.Q655L | uc003iix.2 | P1 |
| MAPK8 | 5599 | 49303987 | Missense | c.963G > A | p.E247K | uc009xnz.1 | P1 |
| MYO10 | 4651 | 16756173 | Missense | c.2839G > C | p.A791P | uc003jft.2 | P1 |
| R3HDM2 | 22864 | 55936537 | Missense | c.2865G > C | p.G825A | uc001snt.2 | P1 |
| SLIT2 | 9353 | 20159235 | Missense | c.2576C > T | p.T791M | uc003gpr.1 | P1 |
| TMEM51 | 55092 | 15418430 | Missense | c.914T > A | p.D122E | uc001avw.2 | P1 |
| TOLLIP | 54472 | 1273536 | Missense | c.209T > G | p.V33G | uc001lte.1 | P1 |
| TSFM | 10102 | 56476508 | Missense | c.965T > C | p.S306P | uc001sqh.2 | P1 |
| UROC1 | 131669 | 127707353 | Missense | c.726C > T | p.R232W | uc010hsi.1 | P1 |
| ZFR2 | 23217 | 3759936 | Frame_Shift_Ins | c.2490_2491insG | p.G826fs | uc002lyw.2 | P1 |
| ZNF536 | 9745 | 35731163 | Missense | c.2935G > A | p.E933K | uc002nsu.1 | P1 |
| ZNF578 | 147660 | 57705665 | Missense | c.463G > T | p.E73D | uc002pzp.2 | P1 |
| ADAMTSL3 | 57188 | 82476242 | Missense | c.4484A > C | p.E1420D | uc002bjz.2 | P2 |
| ARHGEF10L | 55160 | 17894135 | Frame_Shift_Del | c.1007_1022delTT | p.F51fs | uc001bas.1 | P2 |
| C14orf37 | 145407 | 57674770 | Missense | c.1171G > A | p.E354K | uc001xdc.1 | P2 |
| C4orf22 | 255119 | 82010250 | Missense | c.513C > T | p.T155M | uc010ijp.1 | P2 |
| CPSF2 | 53981 | 91678442 | Missense | c.1080G > T | p.K281N | uc001yah.1 | P2 |
| DMC1 | 11144 | 37265361 | Missense | c.672G > A | p.R166H | uc003avz.1 | P2 |
| EHBP1L1 | 254102 | 65114138 | Missense | c.4329G > A | p.R1355Q | uc001oeo.2 | P2 |
| GPR61 | 83873 | 109887249 | Missense | c.765G > T | p.A28S | uc001dxy.2 | P2 |
| GRIP2 | 80852 | 14556888 | Missense | c.223A > G | p.R75G | uc003byt.1 | P2 |
| KIAA1244 | 57221 | 138625638 | Missense | c.1325A > G | p.Q442R | uc003qhu.2 | P2 |
| MAK | 4117 | 10872696 | Missense | c.2076T > G | p.V616G | uc003mzl.1 | P2 |
| MORC3 | 23515 | 36654161 | Missense | c.1304G > A | p.C416Y | uc002yvi.1 | P2 |
| MYOM1 | 8736 | 3145015 | Missense | c.1907T > G | p.Y525D | uc002klp.1 | P2 |
| NAIF1 | 203245 | 129868759 | Missense | c.454C > A | p.T148K | uc004bta.1 | P2 |
| NBPF16 | 728936 | 147019954 | Frame_Shift_Del | c.1538_1544delTT | p.D449fs | uc001esf.2 | P2 |
| NET1 | 10276 | 5486369 | Frame_Shift_Del | c.1048_1066delCT | p.L304fs | uc001iia.1 | P2 |
| NSL1 | 25936 | 211024336 | Nonsense | c.470G > T | p.E146* | uc001hjn.1 | P2 |
| PCDHGB4 | 8641 | 140749175 | Missense | c.1540G > A | p.A514T | uc003lkc.1 | P2 |
| PIGX | 54965 | 197939992 | Missense | c.713A > T | p.R144S | uc010iaj.1 | P2 |
| RP1 | 6101 | 55700154 | Missense | c.1307T > C | p.F387L | uc003xsd.1 | P2 |
| RSPO4 | 343637 | 892700 | Missense | c.570G > A | p.G158D | uc002wej.1 | P2 |
| SKI | 6497 | 2150476 | Frame_Shift_Del | c.483_484delGC | p.Q137fs | uc001aja.2 | P2 |
| SLC2A14 | 144195 | 7861773 | Missense | c.2058G > C | p.R422P | uc001qtk.1 | P2 |
| TARSL2 | 123283 | 100082062 | Nonsense | c.107C > T | p.Q18* | uc002bxm.1 | P2 |
| TNNT3 | 7140 | 1916276 | Missense | c.967A > T | p.K252I | uc001luu.2 | P2 |
| TRAF7 | 84231 | 2160615 | Splice_Site_Ins | c.e5_splice_site | | uc002cow.1 | P2 |
| TRIM7 | 81786 | 180554912 | Frame_Shift_Ins | c.1462_1463insA | p.L465fs | uc003mmz.1 | P2 |
| ZNF296 | 162979 | 50267276 | Missense | c.908T > G | p.V284G | uc002pao.1 | P2 |
| ZNF462 | 58499 | 108730641 | Missense | c.4916G > A | p.V1543M | uc004bcz.1 | P2 |
| BAZ2A | 11176 | 55289786 | Splice_Site_SNP | c.e10_splice_site | | uc001slq.1 | P3 |
| CADPS2 | 93664 | 121901798 | Missense | c.2034G > A | p.R624H | uc010lkp.1 | P3 |
| CENPE | 1062 | 104251549 | Missense | c.7699G > A | p.V2537I | uc003hxb.1 | P3 |
| DCLK1 | 9201 | 35295012 | Missense | c.1620G > T | p.G470W | uc001uvf.1 | P3 |
| DDX3X | 1654 | 41081630 | Nonsense | c.926C > A | p.S24* | uc004dfe.1 | P3 |
| DNA2 | 1763 | 69901564 | Missense | c.322C > G | p.P108A | uc001jof.1 | P3 |
| EOMES | 8320 | 27734163 | Missense | c.1520G > A | p.R507H | uc003cdy.2 | P3 |
| F9 | 2158 | 138446978 | Missense | c.261T > G | p.F78V | uc004fas.1 | P3 |
| IFI16 | 3428 | 157288330 | Frame_Shift_Del | c.2025_2026delTA | p.Y579fs | uc001ftg.1 | P3 |
| MYH1 | 4619 | 10353626 | Missense | c.1582G > T | p.M496I | uc002gmo.1 | P3 |
| PLCL1 | 5334 | 198656746 | De_novo_Start_OutOfFrame | c.146G > A | | uc002uuw.2 | P3 |
| PPP1CC | 5501 | 109643278 | Nonsense | c.1112C > T | p.Q320* | uc001tru.1 | P3 |
| PRICKLE1 | 144165 | 41149628 | Missense | c.505A > T | p.E92V | uc001rnl.1 | P3 |
| PTPRT | 11122 | 40177338 | Missense | c.3255C > T | p.T1024M | uc010ggj.1 | P3 |
| RFX7 | 64864 | 54174766 | Frame_Shift_Del | c.2451_2452delGA | p.E817fs | uc010bfn.1 | P3 |
| SERPINB2 | 5055 | 59721264 | Missense | c.1065C > A | p.D331E | uc002ljo.1 | P3 |
| TP53 | 7157 | 7518263 | Missense | c.937G > A | p.R248Q | uc002gim.2 | P3 |
| ANKRD30A | 91074 | 37459205 | Missense | c.334G > A | p.V79I | uc001iza.1 | P4 |
| ATXN7L3 | 56970 | 39630295 | Splice_Site_SNP | c.e3_splice_site | | uc002ifz.1 | P4 |
| C15orf59 | 388135 | 71819930 | Missense | c.608G > A | p.G88D | uc002avy.1 | P4 |
| CPVL | 54504 | 29070353 | Missense | c.1105A > T | p.Y329F | uc003szv.1 | P4 |
| DAB1 | 1600 | 57249009 | Missense | c.2289G > A | p.E539K | uc001cys.1 | P4 |
| DES | 1674 | 219993578 | Missense | c.939G > A | p.A285T | uc002vll.1 | P4 |
| HERPUD1 | 9709 | 55533552 | Missense | c.1322G > A | p.V305I | uc002eke.1 | P4 |
| HFM1 | 164045 | 91618348 | Missense | c.1007G > A | p.A303T | uc001doa.2 | P4 |
| KCNJ2 | 3759 | 65683052 | Missense | c.678G > A | p.V93I | uc010dfg.1 | P4 |
| MAVS | 57506 | 3793248 | Missense | c.1140C > T | p.S324F | uc002wjw.2 | P4 |
| NLGN3 | 54413 | 70306007 | Missense | c.2126G > A | p.V608M | uc004dzb.1 | P4 |
| OR6A2 | 8590 | 6772980 | Missense | c.736T > C | p.I179T | uc001mes.1 | P4 |
| PPFIBP1 | 8496 | 27708589 | Missense | c.1371T > C | p.C332R | uc001ric.1 | P4 |
| RIN2 | 54453 | 19918809 | Missense | c.1958T > G | p.V641G | uc002wro.1 | P4 |
| SPAG8 | 26206 | 35800295 | Nonsense | c.1327C > A | p.Y404* | uc003zye.1 | P4 |
| ARHGEF10 | 9639 | 1812236 | Missense | c.950G > A | p.E258K | uc003wpr.1 | P5 |
| ATAD3B | 83858 | 1413149 | Missense | c.1359C > G | p.R420G | uc001afv.1 | P5 |
| ATM | 472 | 107741029 | Missense | c.9246A > G | p.Y2954C | uc001pkb.1 | P5 |
| C12orf48 | 55010 | 101113976 | Missense | c.1737A > C | p.K425T | uc001tjg.1 | P5 |
| CCDC18 | 343099 | 93492662 | Missense | c.3767G > A | p.R1200Q | uc001dpq.1 | P5 |
| FMNL3 | 91010 | 48342029 | Nonsense | c.673C > T | p.Q147* | uc001ruv.1 | P5 |
| KCNJ5 | 3762 | 128286871 | Missense | c.807A > T | p.I165F | uc001qet.1 | P5 |
| KCNJ6 | 3763 | 38008528 | Missense | c.1339G > A | p.D268N | uc002ywo.1 | P5 |
| KDR | 3791 | 55659683 | Missense | c.2614A > G | p.T771A | uc003has.1 | P5 |
| LCP1 | 3936 | 45631039 | Nonsense | c.277C > T | p.R51* | uc001vaz.2 | P5 |
| MED27 | 9442 | 133944883 | Missense | c.192A > T | p.Q57L | uc004cbe.1 | P5 |
| MTOR | 2475 | 11110752 | Missense | c.6008A > T | p.T1977S | uc001asd.1 | P5 |
| MUC6 | 4588 | 1009308 | Missense | c.4048A > C | p.T1333P | uc001lsw.2 | P5 |
| MYD88 | 4615 | 38157645 | Missense | c.794T > C | p.L265P | NM_002468 | P5 |
| PCDH17 | 27253 | 57106480 | Missense | c.2691A > T | p.N600I | uc001vhq.1 | P5 |
| PHLPP2 | 23035 | 70267992 | Missense | c.1336G > A | p.V444M | uc002fax.1 | P5 |
| PRKCQ | 5588 | 6580493 | Missense | c.596G > T | p.G171V | uc001iji.1 | P5 |
| RALYL | 138046 | 85604230 | Missense | c.292C > A | p.A53D | uc003yct.2 | P5 |
| ROS1 | 6098 | 117780921 | Missense | c.4445G > A | p.A1416T | uc003pxp.1 | P5 |
| SIM1 | 6492 | 101002763 | Missense | c.1037T > C | p.L277P | uc003pqj.2 | P5 |
| SVEP1 | 79987 | 112291768 | Missense | c.2250T > C | p.F638S | uc010mtz.1 | P5 |
| ZNHIT6 | 54680 | 85940432 | Missense | c.1149A > G | p.K339E | uc001dlh.1 | P5 |
| CCDC67 | 159989 | 92736975 | Missense | c.399T > C | p.F100S | uc001pdq.1 | P6 |
| CCDC94 | 55702 | 4218759 | Frame_Shift_Ins | c.880_881insC | p.A283fs | uc002lzv.2 | P6 |
| CFH | 3075 | 194964125 | Missense | c.2503T > C | p.S755P | uc001gtj.2 | P6 |
| COL14A1 | 7373 | 121332172 | Missense | c.3003G > T | p.G913V | uc003yox.1 | P6 |
| DDX3X | 1654 | 41089376 | Splice_Site_SNP | c.e11_splice_site | | uc004dfe.1 | P6 |
| FERMT1 | 55612 | 6048118 | De_novo_Start_OutOfFrame | c.873C > T | | uc010gbt.1 | P6 |
| MTCH1 | 23787 | 37053843 | Missense | c.580G > T | p.V194F | uc003one.2 | P6 |
| MYCBP2 | 23077 | 76540862 | Missense | c.11987G > A | p.D3966N | uc001vkf.1 | P6 |
| MYO7A | 4647 | 76573419 | Splice_Site_Del | c.e27_splice_site | | uc009yur.1 | P6 |
| OR2S2 | 56656 | 35947816 | Missense | c.336T > C | p.S84P | uc003zyt.2 | P6 |
| POU6F2 | 11281 | 39466752 | Missense | c.1526G > A | p.R495H | uc003thb.1 | P6 |
| SF3B1 | 23451 | 197975726 | Missense | c.1924A > C | p.N626H | uc002uue.1 | P6 |
| SMAD1 | 4086 | 146655259 | Missense | c.460A > G | p.K15R | uc003ikc.1 | P6 |
| SPATA6 | 54558 | 48649798 | Missense | c.495T > A | p.F110L | uc001crr.1 | P6 |
| ZNF492 | 57615 | 22639513 | Missense | c.1333C > T | p.A401V | uc002nqw.2 | P6 |
| CCNY | 219771 | 35881993 | Missense | c.800T > C | p.I207T | uc001iyw.2 | P7 |
| COL28A1 | 340267 | 7364940 | Missense | c.3344T > C | p.L1076S | uc003src.1 | P7 |
| DNAJB2 | 3300 | 219857865 | Frame_Shift_Ins | c.1124_1125insG | p.L296fs | uc002vkx.1 | P7 |
| EIF4A3 | 9775 | 75725883 | Missense | c.1058A > G | p.T294A | uc002jxs.1 | P7 |
| ELF5 | 2001 | 34458369 | Missense | c.1000C > T | p.A257V | uc001mvo.1 | P7 |
| GCNT3 | 9245 | 57698729 | Missense | c.1590G > A | p.A334T | uc002agd.1 | P7 |
| IGFBP3 | 3486 | 45922781 | Missense | c.791G > A | p.R220H | uc003tnr.1 | P7 |
| LAMA2 | 3908 | 129517441 | Missense | c.1231G > A | p.G376S | uc003qbn.1 | P7 |
| MBTPS2 | 51360 | 21810543 | Nonsense | c.1508G > A | p.W470* | uc004dac.1 | P7 |
| MYLK3 | 91807 | 45320522 | Missense | c.1803A > T | p.I563F | uc002eei.2 | P7 |
| MYOC | 4653 | 169888292 | Nonsense | c.105G > A | p.W28* | uc001ghu.1 | P7 |
| ONECUT2 | 9480 | 53254407 | Missense | c.493T > C | p.L154P | uc002lgo.1 | P7 |
| PAMR1 | 25891 | 35410637 | Missense | c.2100C > T | p.A686V | uc001mwf.1 | P7 |
| PCDHA10 | 56139 | 140217127 | Missense | c.1310C > G | p.T437R | uc003lhx.1 | P7 |
| PCDHGB3 | 56102 | 140731583 | Missense | c.1438G > A | p.D480N | uc003ljw.1 | P7 |
| POT1 | 25913 | 124290777 | Missense | c.1010C > T | p.R137C | uc003vlm.1 | P7 |
| RARS | 5917 | 167866405 | Missense | c.1378G > A | p.G446E | uc003lzx.1 | P7 |
| SPIRE1 | 56907 | 12496637 | Nonsense | c.858C > T | p.R271* | uc002kre.1 | P7 |
| TMC2 | 117532 | 2523528 | Missense | c.1006G > A | p.G331R | uc002wgf.1 | P7 |
| ZDBF2 | 57683 | 206881135 | Missense | c.3888G > A | p.R1213Q | uc002vbp.2 | P7 |
| ASH2L | 9070 | 38082335 | Missense | c.168C > T | p.A37V | uc003xkt.2 | P8 |
| ATM | 472 | 107695947 | Frame_Shift_Del | c.6789_6789delT | p.L2135fs | uc001pkb.1 | P8 |
| COL22A1 | 169044 | 139728095 | Missense | c.3907C > G | p.P1154A | uc003yvd.1 | P8 |
| DMXL2 | 23312 | 49582517 | Missense | c.2995G > A | p.A924T | uc002abf.1 | P8 |
| DYRK1A | 1859 | 37784464 | Missense | c.857T > G | p.L261R | uc002ywk.1 | P8 |
| GADL1 | 339896 | 30817419 | Missense | c.1263G > C | p.E406Q | uc003ceq.1 | P8 |
| GNB1 | 2782 | 1727802 | Missense | c.571T > C | p.I80T | uc001aif.1 | P8 |
| GRID2 | 2895 | 94909513 | Missense | c.2748C > A | p.S830R | uc003hsz.2 | P8 |
| HPS5 | 11234 | 18290111 | Missense | c.423T > G | p.L49V | uc001mod.1 | P8 |
| ITGA5 | 3678 | 53099099 | Frame_Shift_Del | c.213_219delCCA | p.P49fs | uc001sga.1 | P8 |
| LILRA4 | 23547 | 59541523 | Missense | c.368C > T | p.A104V | uc002qfj.1 | P8 |
| MAMDC2 | 256691 | 71936324 | Missense | c.1564C > T | p.P324S | uc004ahm.1 | P8 |
| SF3B1 | 23451 | 197974856 | Missense | c.2273G > A | p.G742D | uc002uue.1 | P8 |
| TMPRSS9 | 360200 | 2356419 | Missense | c.616G > T | p.G206C | uc002lvw.1 | P8 |
| ANKRD26 | 22852 | 27358293 | Splice_Site_SNP | c.e26_splice_site | | uc009xku.1 | P9 |
| BCR | 613 | 21853993 | Missense | c.1442C > G | p.I282M | uc002zww.1 | P9 |
| CBARA1 | 10367 | 73937975 | Missense | c.735G > A | p.G201E | uc001jtb.1 | P9 |
| CD14 | 929 | 139991681 | Missense | c.1426T > C | p.S358P | uc003lgi.1 | P9 |
| DIS3 | 22894 | 72245834 | Nonsense | c.1602A > T | p.R410* | uc001vix.2 | P9 |
| GBF1 | 8729 | 104129636 | Missense | c.5050A > T | p.I1604F | uc001kux.1 | P9 |
| GJB2 | 2706 | 19661627 | Missense | c.309C > T | p.R32C | uc001umy.1 | P9 |
| GNB2 | 2783 | 100113730 | Missense | c.829T > G | p.S191A | uc003uwb.1 | P9 |
| HECTD1 | 25831 | 30712649 | Missense | c.1207A > G | p.M240V | uc001wrc.1 | P9 |
| IGSF22 | 283284 | 18695022 | Missense | c.1265G > T | p.V359L | uc009yht.1 | P9 |
| IQGAP1 | 8826 | 88785740 | Splice_Site_SNP | c.e8_splice_site | | uc002bpl.1 | P9 |
| MED12 | 9968 | 70256023 | Missense | c.374G > C | p.A59P | uc004dyy.1 | P9 |
| MMP16 | 4325 | 89200142 | Missense | c.1056T > A | p.N258K | uc003yeb.2 | P9 |
| PLSCR1 | 5359 | 147722532 | Splice_Site_SNP | c.e6_splice_site | | uc003evx.2 | P9 |
| REV1 | 51455 | 99388904 | Missense | c.2923C > T | p.T904I | uc002tad.1 | P9 |
| RHO | 6010 | 130734178 | Missense | c.904G > A | p.S270N | uc003emt.1 | P9 |
| SH3BP4 | 23677 | 235627037 | Nonsense | c.3123C > G | p.Y910* | uc002wp.1 | P9 |
| SLC7A4 | 6545 | 19715741 | Missense | c.429A > G | p.N121D | uc002zud.1 | P9 |
| SNX19 | 399979 | 130255889 | Missense | c.3144A > G | p.N866D | uc001qgk.2 | P9 |
| TET1 | 80312 | 70074858 | Missense | c.2871A > T | p.N789I | uc001jok.2 | P9 |
| TP53 | 7157 | 7518243 | Missense | c.957A > T | p.I255F | uc002gim.2 | P9 |
| TTC7A | 57217 | 47127944 | Frame_Shift_Del | c.2323_2323delA | p.Q652fs | uc010fbb.1 | P9 |
| UBR5 | 51366 | 103385535 | Missense | c.2899C > G | p.L956V | uc003ykr.1 | P9 |
| ZSCAN18 | 65982 | 63292018 | Splice_Site_SNP | c.e3_splice_site | | uc002qrh.1 | P9 |
| CELSR2 | 1952 | 109594496 | Missense | c.333G > A | p.R91K | uc001dxa.2 | P10 |
| CEMP1 | 752014 | 2520913 | Missense | c.519A > G | p.K55E | uc002cqr.2 | P10 |
| FAM155B | 27112 | 68666141 | Missense | c.1084T > G | p.L346V | uc004dxk.1 | P10 |
| FAT4 | 79633 | 126592681 | Missense | c.11060A > G | p.D3687G | uc003ifj.2 | P10 |
| HSPA4L | 22824 | 128946323 | Missense | c.1422G > A | p.R390H | uc003ifm.1 | P10 |
| LRRC56 | 115399 | 541685 | Frame_Shift_Ins | c.1320_1321insT | p.D277fs | uc001lpw.1 | P10 |
| MET | 4233 | 116126605 | Missense | c.418C > A | p.D77E | uc010lkh.1 | P10 |
| MYL5 | 4636 | 664336 | Missense | c.436A > C | p.M111L | uc003gav.1 | P10 |
| NTN3 | 4917 | 2463275 | Missense | c.1476C > T | p.P425S | uc002cqj.1 | P10 |
| PRKCI | 5584 | 171496391 | Splice_Site_SNP | c.e15_splice_site | | uc003fgs.2 | P10 |
| TMPRSS6 | 164656 | 35794601 | Splice_Site_SNP | c.e17_splice_site | | uc003aqt.1 | P10 |
| UBA1 | 7317 | 46958727 | Missense | c.3047A > G | p.N966D | uc004dhj.2 | P10 |
| WDFY3 | 23001 | 85920389 | Missense | c.4669G > A | p.A1421T | uc003hpd.1 | P10 |
| ZNF423 | 23090 | 48227712 | Missense | c.3150C > T | p.T951M | uc002efs.1 | P10 |
| CDH23 | 64072 | 73170595 | Missense | c.4876C > T | p.S1500F | uc001jrx.2 | P10 |
| DIS3 | 22894 | 72235744 | Missense | c.2347A > G | p.E658G | uc001vix.2 | P10 |
| DSCAML1 | 57453 | 116897252 | Missense | c.1198C > T | p.T399M | uc001prh.1 | P11 |
| GDF15 | 9518 | 18360107 | Missense | c.321T > G | p.S97A | uc002niv.2 | P11 |
| HCFC1R1 | 54985 | 3013266 | Frame_Shift_Ins | c.382_383insC | p.P83fs | uc002csx.1 | P11 |
| HK3 | 3101 | 176248421 | Missense | c.1039T > G | p.V322G | uc003mfa.1 | P11 |
| LOXL4 | 84171 | 100010861 | Missense | c.621A > G | p.E157G | uc001kpa.1 | P11 |
| MST1 | 4485 | 49699802 | Missense | c.440A > C | p.K143Q | uc003cxg.1 | P11 |
| NIPA1 | 123606 | 20612340 | Missense | c.258T > G | p.V78G | uc001yvc.1 | P11 |
| NME6 | 10201 | 48315016 | Missense | c.65A > G | p.S7G | uc003cso.1 | P11 |
| PTGIR | 5739 | 51816468 | Missense | c.1183T > G | p.V357G | uc002pex.1 | P11 |
| RUNDC3B | 154661 | 87167736 | Missense | c.762G > T | p.C118F | uc003ujb.1 | P11 |
| SALL4 | 57167 | 49841374 | Missense | c.1156C > T | p.A352V | uc002xwh.2 | P11 |
| SPTB | 6710 | 64323005 | Missense | c.3485A > G | p.E1144G | uc001xhr.1 | P11 |
| STARD13 | 90627 | 32585045 | Missense | c.2424C > G | p.Q769E | uc001uuw.1 | P11 |
| TAS1R2 | 80834 | 19039411 | Missense | c.1790C > T | p.R597C | uc001bba.1 | P11 |
| ATRX | 546 | 76794441 | Frame_Shift_Ins | c.4607_4608insC | p.E1459fs | uc004ecp.2 | P12 |
| CXorf22 | 170063 | 35898921 | Missense | c.1989T > A | p.Y644N | uc004ddj.1 | P12 |
| DZIP1L | 199221 | 139273352 | Missense | c.1801T > C | p.S480P | uc003erq.1 | P12 |
| ELMOD2 | 255520 | 141678014 | Splice_Site_SNP | c.e5_splice_site | | uc003iik.1 | P12 |
| FAM47A | 158724 | 34059355 | Missense | c.995C > T | p.P321L | uc004ddg.1 | P12 |
| FBXW7 | 55294 | 153466739 | Missense | c.1662C > T | p.R505C | uc003ims.1 | P12 |
| GALNT13 | 114805 | 154806955 | Missense | c.582G > T | p.D160Y | uc002tyt.2 | P12 |
| ITIH2 | 3698 | 7812013 | Missense | c.1534G > A | p.D458N | uc001ijs.1 | P12 |
| KCNA2 | 3737 | 110948749 | Missense | c.675G > A | p.G60E | uc001dzu.1 | P12 |
| LTB | 4050 | 31657349 | Missense | c.208T > C | p.I67T | uc003nul.1 | P12 |
| MLL5 | 55904 | 104534235 | Missense | c.3161T > G | p.F876C | uc003vcm.1 | P12 |
| MRPS14 | 63931 | 173259164 | Missense | c.21G > A | p.A2T | uc001gkk.1 | P12 |
| NAV2 | 89797 | 20023535 | Missense | c.4075T > A | p.D1238E | uc009yhw.1 | P12 |
| NOBOX | 135935 | 143729428 | Frame_Shift_Ins | c.487_488insC | p.R163fs | uc003wen.1 | P12 |
| NUDT9 | 53343 | 88575339 | Missense | c.613T > C | p.V97A | uc003hqq.1 | P12 |
| SLITRK4 | 139065 | 142544118 | Missense | c.2849C > A | p.L825I | uc004fbx.1 | P12 |
| SUV420H1 | 51111 | 67695088 | Missense | c.1203A > G | p.N316S | uc001onm.1 | P12 |
| TRHDE | 29953 | 71343212 | Missense | c.3141C > A | p.F1015L | uc001sxa.1 | P12 |
| CCDC99 | 54908 | 168960894 | Missense | c.1636C > T | p.R453C | uc003mae.2 | P13 |
| CELSR2 | 1952 | 109615413 | Missense | c.7709C > T | p.R2550W | uc001dxa.2 | P13 |
| DNTTIP1 | 116092 | 43854757 | Missense | c.208T > G | p.V47G | uc002xpk.1 | P13 |
| EEF1D | 1936 | 144733919 | Missense | c.1989C > T | p.A587V | uc003yyq.1 | P13 |
| EGF | 1950 | 111151823 | Missense | c.993T > C | p.L251P | uc010imk.1 | P13 |
| HIGD1C | 613227 | 49650547 | Frame_Shift_Ins | c.285_286insA | p.S95fs | uc009zlu.1 | P13 |
| KIAA2022 | 340533 | 73876738 | Missense | c.4693A > G | p.E1460G | uc004eby.1 | P13 |
| KRT5 | 3852 | 51200162 | Missense | c.349C > G | p.S62R | uc001san.1 | P13 |
| MAOA | 4128 | 43456087 | Missense | c.512G > A | p.A111T | uc004dfy.1 | P13 |
| MPEG1 | 219972 | 58736287 | Missense | c.784G > A | p.D210N | uc001nnu.2 | P13 |
| NISCH | 11188 | 52499853 | Missense | c.3840A > G | p.N1236D | uc003ded.2 | P13 |
| POLA1 | 5422 | 24645622 | Missense | c.918A > G | p.S299G | uc004dbl.1 | P13 |
| PTX3 | 5806 | 158643184 | Missense | c.1011G > A | p.A290T | uc003fbl.2 | P13 |
| RFX7 | 64864 | 54175584 | Missense | c.1634C > T | p.S545L | uc010bfn.1 | P13 |
| SDCCAG3 | 10807 | 138418948 | Frame_Shift_Ins | c.1228_1229insT | p.A341fs | uc004chi.1 | P13 |
| TAF1 | 6872 | 70519409 | Missense | c.1850G > T | p.G600V | uc004dzt.2 | P13 |
| TEKT1 | 83659 | 6644089 | Missense | c.1348G > A | p.R413H | uc002gdt.1 | P13 |
| TMEM8A | 58986 | 362109 | Missense | c.2324G > A | p.S732N | uc002cgu.2 | P13 |
| USF1 | 7391 | 159279072 | De_novo_Start_OutOfFrame | c.266C > A | | uc001fxj.1 | P13 |
| ZC3H12B | 340554 | 64633878 | Splice_Site_SNP | c.e2_splice_site | | uc010nko.1 | P13 |
| ZMYM3 | 9203 | 70378786 | Missense | c.3848G > C | p.S1254T | uc004dzh.1 | P13 |
| ZNF253 | 56242 | 19863281 | Splice_Site_SNP | c.e4_splice_site | | uc002noj.1 | P13 |
| ADPRHL1 | 113622 | 113146822 | Missense | c.385G > T | p.D100Y | uc001vtq.1 | P14 |
| C3orf59 | 151963 | 194000064 | Missense | c.602G > A | p.R92Q | uc003fsz.1 | P14 |
| EML4 | 27436 | 42410840 | Missense | c.3171C > A | p.P979T | uc002rsi.1 | P14 |
| FLNA | 2316 | 153231043 | Frame_Shift_Ins | c.7885_7886insC | p.Q2546fs | uc004fkk.2 | P14 |
| KBTBD8 | 84541 | 67141034 | Splice_Site_SNP | c.e4_splice_site | | uc003dmy.1 | P14 |
| KIT | 3815 | 55290365 | Missense | c.2185G > T | p.A700S | uc010igr.1 | P14 |
| MATR3 | 9782 | 138689749 | Splice_Site_SNP | c.e15_splice_site | | uc003ldw.1 | P14 |
| MSH4 | 4438 | 76086496 | Missense | c.1218C > G | p.L393V | uc001dhd.1 | P14 |
| NCOA4 | 8031 | 51250888 | Missense | c.478A > T | p.L111F | uc009xon.1 | P14 |
| PRAMEF10 | 343071 | 12875552 | Missense | c.1280G > A | p.G403R | uc001auo.1 | P14 |
| SIGLEC1 | 6614 | 3618723 | Frame_Shift_Ins | c.4779_4780insC | p.P1593fs | uc002wja.1 | P14 |
| COL1A2 | 1278 | 93866333 | Splice_Site_SNP | c.e4_splice_site | | uc003ung.1 | P15 |
| CSMD1 | 64478 | 3598920 | Nonsense | c.1261C > T | p.R291* | uc010lrh.1 | P15 |
| KBTBD4 | 55709 | 47555943 | Missense | c.975T > G | p.V87G | uc001nfw.1 | P15 |
| PLK2 | 10769 | 57788768 | Frame_Shift_Ins | c.1131_1132insT | p.L335fs | uc003jrn.1 | P15 |
| SAFB2 | 9667 | 5541342 | Frame_Shift_Ins | c.2683_2684insG | p.G824fs | uc002mcd.1 | P15 |
| TBX4 | 9496 | 56912280 | Missense | c.1002T > A | p.I280N | uc010ddo.1 | P15 |
| TPST2 | 8459 | 25267466 | Frame_Shift_Ins | c.363_364insG | p.A44fs | uc003acx.1 | P15 |
| TRAF3 | 7187 | 102408006 | Splice_Site_SNP | c.e4_splice_site | | uc001ymc.1 | P15 |
| ZAP70 | 7535 | 97707106 | Frame_Shift_Del | c.382_382delT | p.F59fs | uc002syd.1 | P15 |
| ACADSB | 36 | 124789970 | Splice_Site_SNP | c.e4_splice_site | | uc001lhb.1 | P16 |
| CLCN3 | 1182 | 170854913 | Splice_Site_SNP | c.e9_splice_site | | uc003ish.1 | P16 |
| DLG5 | 9231 | 79251231 | Missense | c.3087G > A | p.R1006K | uc001jzk.1 | P16 |
| EIF3E | 3646 | 109316509 | Splice_Site_SNP | c.e5_splice_site | | uc003ymu.1 | P16 |
| ELF4 | 2000 | 129035759 | Nonsense | c.671G > T | p.E96* | uc004evd.2 | P16 |
| FGFRL1 | 53834 | 1008365 | Missense | c.1146C > T | p.R329C | uc003gce.1 | P16 |
| FUBP1 | 8880 | 78205334 | Frame_Shift_Ins | c.418_419insG | p.G110fs | uc001dii.1 | P16 |
| GABRG3 | 2567 | 25446672 | Splice_Site_SNP | c.e9_splice_site | | uc001zbg.1 | P16 |
| HSPA8 | 3312 | 122435409 | Missense | c.1180G > A | p.A368T | uc001pyo.1 | P16 |
| IDH1 | 3417 | 208816465 | Missense | c.875G > A | p.S210N | uc002vcs.1 | P16 |
| MMD | 23531 | 50836125 | Splice_Site_SNP | c.e5_splice_site | | uc002iui.1 | P16 |
| MTMR3 | 8897 | 28733305 | Missense | c.1202T > G | p.F292V | uc003agv.2 | P16 |
| MUC16 | 94025 | 8950417 | Missense | c.2602G > A | p.E800K | uc002mkp.1 | P16 |
| NF1 | 4763 | 26565668 | Missense | c.1799A > G | p.Y489C | uc002hgg.1 | P16 |
| NOL11 | 25926 | 63166121 | Missense | c.1873T > C | p.Y624H | uc002jgd.1 | P16 |
| NRCAM | 4897 | 107623450 | Missense | c.1925C > A | p.T485N | uc003vfb.1 | P16 |
| OSBPL3 | 26031 | 24821349 | Splice_Site_SNP | c.e19_splice_site | | uc003sxf.1 | P16 |
| PAPPA | 5069 | 118169807 | Missense | c.4939G > A | p.V1520M | uc004bjn.1 | P16 |
| POLRMT | 5442 | 581069 | Missense | c.349A > G | p.D98G | uc002lpf.1 | P16 |
| PUM1 | 9698 | 31211608 | Missense | c.2133C > A | p.P668T | uc001bsk.1 | P16 |
| ZNF251 | 90987 | 145917948 | Missense | c.2162C > G | p.Q636E | uc003zdv.2 | P16 |
| ABCB1 | 5243 | 87052934 | Splice_Site_SNP | c.e5_splice_site | | uc003uiz.1 | P17 |
| ATM | 472 | 107660172 | Missense | c.4140A > T | p.Y1252F | uc001pkb.1 | P17 |
| BTAF1 | 9044 | 93746104 | Splice_Site_SNP | c.e24_splice_site | | uc001khr.1 | P17 |
| DCBLD1 | 285761 | 117968864 | Missense | c.1465C > T | p.S447L | uc003pxs.1 | P17 |
| FAM123A | 219287 | 24642073 | Missense | c.1785C > T | p.P562L | uc001uqb.1 | P17 |
| FAT4 | 79633 | 126458429 | Missense | c.1413T > G | p.H471Q | uc003ifj.2 | P17 |
| GART | 2618 | 33805432 | Missense | c.2398G > C | p.E771Q | uc002yrx.1 | P17 |
| GPR126 | 57211 | 142756724 | Splice_Site_SNP | c.e9_splice_site | | uc010khe.1 | P17 |
| LRRC56 | 115399 | 541786 | Frame_Shift_Del | c.1421_1421delA | p.E311fs | uc001lpw.1 | P17 |
| MYD88 | 4615 | 38157645 | Missense | c.794T > C | p.L265P | NM_002468 | P17 |
| MYH9 | 4627 | 35011944 | Missense | c.5247A > G | p.E1688G | uc003apg.1 | P17 |
| PKDCC | 91461 | 42135942 | Frame_Shift_Del | c.714_714delG | p.W177fs | uc002rsg.1 | P17 |
| SLC1A1 | 6505 | 4573063 | Missense | c.1455G > A | p.G407R | uc003zij.1 | P17 |
| SLC6A16 | 28968 | 54505523 | Missense | c.706T > G | p.F158V | uc002pmz.1 | P17 |
| USP10 | 9100 | 83336655 | Missense | c.1209C > T | p.P356L | uc002fii.1 | P17 |
| ZBTB11 | 27107 | 102866877 | Missense | c.1474T > A | p.I415K | uc003dve.2 | P17 |
| ARHGAP30 | 257106 | 159287940 | Missense | c.1554G > A | p.R403H | uc001fxl.1 | P18 |
| ATAD2B | 54454 | 23896161 | Missense | c.2521T > G | p.S743A | uc002rek.2 | P18 |
| BNC1 | 646 | 81723850 | Missense | c.1245A > C | p.K386T | uc002bjt.1 | P18 |
| C1orf128 | 57095 | 23984842 | Missense | c.535A > T | p.L137F | uc001bhq.1 | P18 |
| C1orf38 | 9473 | 28079147 | Missense | c.669T > A | p.M214K | uc001bpc.2 | P18 |
| CDH9 | 1007 | 26941951 | Missense | c.854A > G | p.R229G | uc003jgs.1 | P18 |
| DNAH10 | 196385 | 122899375 | Missense | c.5766C > T | p.T1914M | uc001uft.2 | P18 |
| DNAH9 | 1770 | 11637610 | Missense | c.8195T > G | p.H2709Q | uc002gne.1 | P18 |
| DOCK4 | 9732 | 111274412 | Missense | c.2749G > A | p.R827Q | uc003vfy.1 | P18 |
| EMID2 | 136227 | 100877683 | Splice_Site_SNP | c.e3_splice_site | | uc003uyo.1 | P18 |
| ENPP1 | 5167 | 132227337 | Splice_Site_SNP | c.e10_splice_site | | uc003qcx.2 | P18 |
| FCER2 | 2208 | 7660294 | Missense | c.929A > C | p.T251P | uc002mhm.1 | P18 |
| FLJ43860 | 389690 | 142552196 | Missense | c.1886G > A | p.R602Q | uc003ywi.2 | P18 |
| GJA3 | 2700 | 19615309 | Missense | c.291C > T | p.A40V | uc001umx.1 | P18 |
| GXYLT2 | 727936 | 73089128 | Missense | c.911A > C | p.K304T | uc003dpg.1 | P18 |
| HMCN1 | 83872 | 184353212 | Splice_Site_SNP | c.e77_splice_site | | uc001grq.1 | P18 |
| IL26 | 55801 | 66905537 | Missense | c.217T > A | p.I61K | uc001stx.1 | P18 |
| ITGB1 | 3688 | 33249301 | Missense | c.1147A > T | p.I383F | uc001iwq.2 | P18 |
| ITGB1 | 3688 | 33251621 | Missense | c.991A > T | p.I331F | uc001iwq.2 | P18 |
| KALRN | 8997 | 125903617 | Missense | c.8139T > G | p.F2680C | uc003ehg.1 | P18 |
| KLKB1 | 3818 | 187410194 | Missense | c.1245G > A | p.V392I | uc003iyy.1 | P18 |
| LPA | 4018 | 160936387 | Missense | c.3578C > G | p.S1153C | uc003qtl.1 | P18 |
| MARK2 | 2011 | 63414276 | Missense | c.369G > T | p.C16F | uc009yox.1 | P18 |
| MYD88 | 4615 | 38157263 | Missense | c.695T > C | p.M232T | NM_002468 | P18 |
| OAT | 4942 | 126090558 | Missense | c.280T > C | p.L58S | uc001lhp.2 | P18 |
| OMG | 4974 | 26647400 | Missense | c.264T > C | p.C26R | uc002hgj.1 | P18 |
| PCDH17 | 27253 | 57197163 | Missense | c.4106T > G | p.L1072V | uc001vhq.1 | P18 |
| SETBP1 | 26040 | 40784471 | Missense | c.1464G > A | p.A336T | uc010dni.1 | P18 |
| SLC12A5 | 57468 | 44102661 | Splice_Site_SNP | c.e7_splice_site | | uc002xrb.1 | P18 |
| SLC8A1 | 6546 | 40196091 | Missense | c.2752T > C | p.S910P | uc002rrx.1 | P18 |
| SSR1 | 6745 | 7246564 | Missense | c.709A > G | p.N174S | uc003mxf.2 | P18 |
| SULT1C3 | 442038 | 108238538 | Missense | c.478G > C | p.D160H | uc002tdw.1 | P18 |
| TBCC | 6903 | 42821345 | Missense | c.518T > G | p.S149A | uc003osl.1 | P18 |
| TGM7 | 116179 | 41373040 | Missense | c.97A > C | p.K31T | uc001zrf.1 | P18 |
| TSPAN19 | 144448 | 83937537 | Missense | c.550C > T | p.T150I | uc009zsj.1 | P18 |
| XIRP2 | 129446 | 167809068 | Missense | c.2938G > T | p.G974C | uc002udx.1 | P18 |
| ACOT2 | 10965 | 73106164 | Missense | c.640T > G | p.V156G | uc001xon.2 | P19 |
| ADAM22 | 53616 | 87601578 | Splice_Site_SNP | c.e12_splice_site | | uc003ujp.1 | P19 |
| ANAPC4 | 29945 | 24993979 | Splice_Site_SNP | c.e4_splice_site | | uc003gro.1 | P19 |
| EPHB3 | 2049 | 185780358 | Missense | c.2551G > T | p.R705L | uc003foz.1 | P19 |
| FAT4 | 79633 | 126589966 | Missense | c.8345C > T | p.P2782L | uc003ifj.2 | P19 |
| GPRC6A | 222545 | 117234665 | Nonsense | c.918G > A | p.W299* | uc003pxj.1 | P19 |
| HYAL3 | 8372 | 50307803 | Missense | c.508G > A | p.G79S | uc003czd.1 | P19 |
| M6PR | 4074 | 8987663 | Splice_Site_SNP | c.e4_splice_site | | uc001qvf.1 | P19 |
| MAP3K14 | 9020 | 40723695 | Missense | c.309C > G | p.A67G | uc002iiw.1 | P19 |
| METTL9 | 51108 | 21531465 | Splice_Site_SNP | c.e2_splice_site | | uc002dje.1 | P19 |
| MYCBP2 | 23077 | 76559647 | Missense | c.10825T > A | p.N3578K | uc001vkf.1 | P19 |
| MYO3B | 140469 | 170966437 | Missense | c.2262G > A | p.E707K | uc002ufy.1 | P19 |
| PCLO | 27445 | 82314427 | Missense | c.14016G > A | p.S4576N | uc003uhx.2 | P19 |
| PDZD11 | 51248 | 69423689 | Missense | c.732T > G | p.Y163D | uc004dye.1 | P19 |
| PIH1D1 | 55011 | 54642141 | Nonsense | c.875G > A | p.W213* | uc002pns.1 | P19 |
| PPP1R12A | 4659 | 78693829 | Splice_Site_Del | c.e25_splice_site | | uc001syz.1 | P19 |
| RAET1E | 135250 | 150253673 | Missense | c.118C > A | p.L20I | uc003qnl.1 | P19 |
| RAI14 | 26064 | 34850443 | Splice_Site_SNP | c.e14_splice_site | | uc003jis.1 | P19 |
| SLC25A28 | 81894 | 101361042 | Missense | c.778C > A | p.Q217K | uc001kpx.2 | P19 |
| XKR8 | 55113 | 28165660 | Missense | c.627G > T | p.A184S | uc001bph.1 | P19 |
| BAZ1A | 11177 | 34334706 | Missense | c.1713G > A | p.R382H | uc001wsk.1 | P20 |
| GPR133 | 283383 | 130017020 | Missense | c.722C > T | p.H55Y | uc001uit.2 | P20 |
| IRF2 | 3660 | 185577718 | Splice_Site_SNP | c.e3_splice_site | | uc003iwf.2 | P20 |
| MUC5B | 727897 | 1222539 | Missense | c.7920C > T | p.A2621V | uc001ltb.2 | P20 |
| MYD88 | 4615 | 38157645 | Missense | c.794T > C | p.L265P | NM_002468 | P20 |
| PA2G4 | 5036 | 54789956 | Missense | c.1018C > A | p.T200N | uc001sjm.1 | P20 |
| PADI4 | 23569 | 17557919 | Splice_Site_Ins | c.e14_splice_site | | uc001baj.1 | P20 |
| PCDHAC1 | 56135 | 140287209 | Missense | c.724C > A | p.P183Q | uc003lih.1 | P20 |
| WBSCR17 | 64409 | 70523889 | Missense | c.824T > C | p.I275T | uc003tvy.1 | P20 |
| WNT1 | 7471 | 47659762 | Missense | c.547G > A | p.V117I | uc001rsu.1 | P20 |
| ABCA12 | 26154 | 215510478 | Splice_Site_SNP | c.e51_splice_site | | uc002vew.1 | P21 |
| AMBP | 259 | 115863569 | Missense | c.1072A > G | p.N270S | uc004bie.2 | P21 |
| ATP2A1 | 487 | 28821082 | Missense | c.2582G > T | p.D800Y | uc002dro.1 | P21 |
| BEST1 | 7439 | 61484025 | Missense | c.950C > T | p.P285L | uc001nsr.1 | P21 |
| BPHL | 670 | 3068948 | Missense | c.327A > G | p.T39A | uc003muy.1 | P21 |
| C4orf41 | 60684 | 184833316 | Missense | c.842A > T | p.L222F | uc003ivx.1 | P21 |
| DGAT2L6 | 347516 | 69338638 | Missense | c.743G > A | p.G216R | uc004dxx.1 | P21 |
| FRMD1 | 79981 | 168200785 | Missense | c.1556C > A | p.H497Q | uc003qwo.2 | P21 |
| GATS | 352954 | 99707409 | Missense | c.141T > C | p.F45S | uc003uua.2 | P21 |
| HSD3B2 | 3284 | 119766663 | Missense | c.1789A > C | p.Y339S | uc001ehs.1 | P21 |
| HTT | 3064 | 3116697 | Missense | c.3238G > T | p.L1031F | uc010icr.1 | P21 |
| MOCS3 | 27304 | 49008917 | Missense | c.148T > G | p.V44G | uc002xvy.1 | P21 |
| PFKFB1 | 5207 | 54992376 | Missense | c.921G > A | p.A284T | uc004dty.1 | P21 |
| PRKRIR | 5612 | 75741455 | Missense | c.387T > A | p.H129Q | uc001oxh.1 | P21 |
| PTPN14 | 5784 | 212704727 | Missense | c.314G > A | p.V15I | uc001hkk.1 | P21 |
| PTPRD | 5789 | 8490768 | Missense | c.2825G > T | p.R705L | uc003zkk.1 | P21 |
| THBS1 | 7057 | 37666983 | Missense | c.1443A > C | p.T422P | uc001zkh.1 | P21 |
| TMEM71 | 137835 | 133833342 | Missense | c.328G > A | p.R62H | uc003ytp.1 | P21 |
| ULK2 | 9706 | 19625004 | Missense | c.3145T > G | p.V882G | uc002gwm.2 | P21 |
| ALDH1L2 | 160428 | 103986645 | Frame_Shift_Ins | c.597_598insG | p.P192fs | uc001tlc.1 | P22 |
| ANKRD49 | 54851 | 93871170 | Missense | c.683G > A | p.A182T | uc001pew.1 | P22 |
| C15orf59 | 388135 | 71819444 | In_frame_Del | c.1086_1094delCC | p.247_250SRHS > R | uc002avy.1 | P22 |
| CAD | 790 | 27294389 | Splice_Site_SNP | c.e2_splice_site | | uc002rji.1 | P22 |
| CADM3 | 57863 | 157436261 | Missense | c.1330T > G | p.F384C | uc001ftk.2 | P22 |
| CASC5 | 57082 | 38731489 | Splice_Site_Del | c.e22_splice_site | | uc010bbs.1 | P22 |
| CNOT6 | 57472 | 179926774 | Frame_Shift_Del | c.1147_1147delG | p.K266fs | uc003mlx.1 | P22 |
| DGCR14 | 8220 | 17510249 | Frame_Shift_Ins | c.330_331insAC | p.P98fs | uc002zou.1 | P22 |
| DUSP7 | 1849 | 52063271 | Missense | c.584C > T | p.P175L | uc003dct.1 | P22 |
| EDEM3 | 80267 | 182929941 | In_frame_Del | c.2910_2939delAG | p.840_850LDNQLQE | uc001gqx.2 | P22 |
| ELOVL2 | 54898 | 11103308 | Missense | c.584G > T | p.Q141H | uc003mzp.2 | P22 |
| EPHB1 | 2047 | 136450026 | Missense | c.2895C > A | p.A892E | uc003eqt.1 | P22 |
| GALNT6 | 11226 | 50045526 | Missense | c.1090G > C | p.A257P | uc001ryl.1 | P22 |
| HAP1 | 9001 | 37141336 | Frame_Shift_Ins | c.1015_1016insAA | p.A335fs | uc002hxm.1 | P22 |
| HVCN1 | 84329 | 109573510 | Missense | c.703G > T | p.V180F | uc001trs.1 | P22 |
| ID2 | 3398 | 8739889 | Missense | c.326_327AG > TT | p.E48V | uc002qza.1 | P22 |
| IQSEC1 | 9922 | 12952029 | Missense | c.1538G > T | p.R510L | uc003bxt.1 | P22 |
| ITPR2 | 3709 | 26530428 | Missense | c.6104C > A | p.P1896Q | uc001rhg.1 | P22 |
| KCNK2 | 3776 | 213326342 | Missense | c.224C > T | p.P19S | uc001hkq.1 | P22 |
| KIF26B | 55083 | 243597090 | Missense | c.1237_1238GC > A | p.S266N | uc001ibf.1 | P22 |
| KRT19 | 3880 | 36933621 | Missense | c.1245A > T | p.D368V | uc002hxd.2 | P22 |
| LAT | 27040 | 28908406 | Missense | c.990C > T | p.S213F | uc002dsd.1 | P22 |
| LIMK2 | 3985 | 29993012 | Frame_Shift_Del | c.1376_1380delTT | p.L341fs | uc003akj.1 | P22 |
| MACF1 | 23499 | 39521533 | Missense | c.1001G > T | p.G266W | uc009vvo.1 | P22 |
| MAGED2 | 10916 | 54854136 | Frame_Shift_Del | c.789_807delCTC | p.T232fs | uc004dtk.1 | P22 |
| MCF2L2 | 23101 | 184408211 | Missense | c.2681G > T | p.R864L | uc003fli.1 | P22 |
| MPI | 4351 | 72969987 | Missense | c.88C > T | p.A28V | uc002azc.1 | P22 |
| MURC | 347273 | 102388017 | Missense | c.648G > T | p.R186S | uc004bba.1 | P22 |
| PCDHB8 | 56128 | 140539046 | Missense | c.1433C > T | p.A416V | uc003liu.1 | P22 |
| PITPNM2 | 57605 | 122039280 | Frame_Shift_Del | c.2963_2963delC | p.L942fs | uc001uej.1 | P22 |
| PRKCD | 5580 | 53190533 | In_frame_Del | c.763_783delCCA | p.137_144AKFPTMN | uc003dgl.1 | P22 |
| PSMC5 | 5705 | 59262618 | Missense | c.1031G > T | p.K330N | uc002jcb.1 | P22 |
| PTPRM | 5797 | 7945388 | Missense | c.1611C > A | p.L370I | uc010dkv.1 | P22 |
| SH3TC2 | 79628 | 148398234 | Missense | c.970G > T | p.C273F | uc003lpu.1 | P22 |
| SPAG9 | 9043 | 46552921 | Nonsense | c.174C > G | p.Y32* | uc002itc.1 | P22 |
| UMOD | 7369 | 20265034 | Frame_Shift_Del | c.1324_1325delTG | p.C399fs | uc002dhb.1 | P22 |
| ZNF205 | 7755 | 3109866 | Missense | c.1339A > C | p.T402P | uc002cub.1 | P22 |
| ZNF211 | 10520 | 62845282 | Missense | c.1942G > T | p.C604F | uc002qps.1 | P22 |
| ZNF461 | 92283 | 41821838 | Nonsense | c.1477G > T | p.E417* | uc002oem.1 | P22 |
| ZNF846 | 162993 | 9729483 | Frame_Shift_Ins | c.1800_1801insGA | p.E423fs | uc002mmb.1 | P22 |
| ATM | 472 | 107691965 | Missense | c.6498A > G | p.H2038R | uc001pkb.1 | P23 |
| CPE | 1363 | 166625050 | Missense | c.1094C > T | p.P273S | uc003irg.2 | P23 |
| DDX19A | 55308 | 68956002 | Missense | c.574G > A | p.V149I | uc002eys.1 | P23 |
| DENND5A | 23258 | 9148807 | Missense | c.2255C > T | p.P667L | uc001mhl.1 | P23 |
| DHX57 | 90957 | 38903839 | Missense | c.3190G > A | p.D1031N | uc002rrf.1 | P23 |
| ECT2 | 1894 | 173962997 | Missense | c.878A > G | p.K286E | uc003fil.1 | P23 |
| ELAVL3 | 1995 | 11438604 | Frame_Shift_Ins | c.427_428insG | p.G16fs | uc002mry.1 | P23 |
| LAMP1 | 3916 | 113008873 | Missense | c.415A > G | p.N45S | uc001vtm.1 | P23 |
| MED12 | 9968 | 70255426 | Missense | c.296G > A | p.E33K | uc004dyy.1 | P23 |
| MPDZ | 8777 | 13209623 | Missense | c.1072C > T | p.R341C | uc010mhy.1 | P23 |
| SLIT2 | 9353 | 20134844 | Missense | c.1588C > T | p.R462C | uc003gpr.1 | P23 |
| SMYD1 | 150572 | 88168522 | Missense | c.343T > G | p.V114G | uc002ssr.1 | P23 |
| ANTXR2 | 118429 | 81125009 | Frame_Shift_Ins | c.1599_1600insC | p.P358fs | uc003hlz.2 | P24 |
| BIRC6 | 57448 | 32554873 | Missense | c.6943A > G | p.K2270R | uc010ezu.1 | P24 |
| CAMLG | 819 | 134102256 | Missense | c.152T > G | p.V16G | uc003kzt.1 | P24 |
| CLSTN2 | 64084 | 141764403 | Missense | c.2283G > A | p.R758H | uc003etn.1 | P24 |
| COL9A1 | 1297 | 71023190 | Splice_Site_SNP | c.e21_splice_site | | uc003pfg.2 | P24 |
| DMXL2 | 23312 | 49559613 | Missense | c.6805C > A | p.Q2194K | uc002abf.1 | P24 |
| DNAH8 | 1769 | 38991084 | Missense | c.10042C > A | p.L3148I | uc003ooe.1 | P24 |
| FAT3 | 120114 | 92171426 | Missense | c.5616G > A | p.V1867I | uc001pdj.2 | P24 |
| GEMIN7 | 79760 | 50285598 | Missense | c.537T > A | p.F129Y | uc002pap.1 | P24 |
| GPC6 | 10082 | 93478090 | Missense | c.1433T > C | p.V273A | uc001vlt.1 | P24 |
| HNRNPUL1 | 11100 | 46500507 | Frame_Shift_Ins | c.2074_2075insGA | p.N595fs | uc002oqb.2 | P24 |
| HSPG2 | 3339 | 22087045 | Missense | c.716A > T | p.R226W | uc009vqd.1 | P24 |
| KCTD7 | 154881 | 65741622 | Missense | c.945C > T | p.P280S | uc003tve.1 | P24 |
| NAGLU | 4669 | 37949472 | Missense | c.2262A > C | p.N641T | uc002hzv.1 | P24 |
| NTF4 | 4909 | 54256756 | Missense | c.452T > G | p.V104G | uc002pmf.2 | P24 |
| PCLO | 27445 | 82423961 | Missense | c.4533C > G | p.T1415R | uc003uhx.2 | P24 |
| PHF19 | 26147 | 122662211 | Missense | c.1631A > C | p.T460P | uc004bks.1 | P24 |
| PLEKHG4B | 153478 | 216536 | Missense | c.2331G > T | p.V761L | uc003jak.2 | P24 |
| POLG | 5428 | 87674485 | Missense | c.968T > G | p.V229G | uc002bns.2 | P24 |
| RAPGEF2 | 9693 | 160493478 | Missense | c.3884C > T | p.R1192W | uc003iqg.2 | P24 |
| RNF150 | 57484 | 142088318 | Missense | c.1484A > G | p.N277S | uc003iio.1 | P24 |
| SH3PXD2B | 285590 | 171813903 | Missense | c.230T > G | p.V20G | uc003mbr.1 | P24 |
| SLC9A2 | 6549 | 102691271 | Frame_Shift_Del | c.2472_2472delG | p.R777fs | uc002tca.1 | P24 |
| ST6GAL2 | 84620 | 106826213 | Missense | c.828A > C | p.N218T | uc002tdr.1 | P24 |
| TMEM88 | 92162 | 7699304 | Frame_Shift_Del | c.196_199delTTC | p.F63fs | uc002giy.1 | P24 |
| TNRC18 | 84629 | 5393918 | Missense | c.2412T > G | p.V688G | uc003soi.2 | P24 |
| ZAP70 | 7535 | 97717445 | Missense | c.1127C > T | p.P307L | uc002syd.1 | P24 |
| ZNF614 | 80110 | 57210966 | Missense | c.2036G > A | p.G566D | uc002pyj.1 | P24 |
| BBS10 | 79738 | 75265672 | Missense | c.308A > G | p.H75R | uc001syd.1 | P25 |
| CCDC85A | 114800 | 56273448 | Nonsense | c.1111C > G | p.Y203* | uc002rzn.1 | P25 |
| CHCHD10 | 400916 | 22438440 | Frame_Shift_Ins | c.363_364insC | p.Q95fs | uc002zxw.1 | P25 |
| CHL1 | 10752 | 418307 | Splice_Site_SNP | c.e27_splice_site | | uc003bot.1 | P25 |
| DLX6 | 1750 | 96473321 | Splice_Site_SNP | c.e1_splice_site | | uc003uom.1 | P25 |
| EFTUD2 | 9343 | 40284618 | Missense | c.2840A > C | p.T937P | uc002ihn.1 | P25 |
| ITIH1 | 3697 | 52787996 | Splice_Site_SNP | c.e4_splice_site | | uc003dfs.2 | P25 |
| LCT | 3938 | 136277987 | Missense | c.4657A > G | p.Y1549C | uc002tuu.1 | P25 |
| LILRB4 | 11006 | 59868382 | Missense | c.1106T > A | p.F239I | uc010ers.1 | P25 |
| MGAT4C | 25834 | 84897673 | Missense | c.2212C > T | p.T321M | uc001tai.2 | P25 |
| MIB2 | 142678 | 1554448 | Frame_Shift_Ins | c.2576_2577insA | p.E817fs | uc001agg.1 | P25 |
| MYD88 | 4615 | 38157645 | Missense | c.794T > C | p.L265P | NM_002468 | P25 |
| RAB11FIP5 | 26056 | 73156170 | Missense | c.2190G > C | p.G650A | uc002siu.2 | P25 |
| SDHAF2 | 54949 | 60962050 | Splice_Site_SNP | c.e3_splice_site | | uc001nrt.1 | P25 |
| SEH1L | 81929 | 12938134 | Missense | c.152G > C | p.R5P | uc002krq.1 | P25 |
| SLIT3 | 6586 | 168120518 | Nonsense | c.1875C > T | p.R538* | uc010jjg.1 | P25 |
| ADAMTS10 | 81794 | 8556462 | Missense | c.3017G > A | p.V915I | uc002mkj.1 | P26 |
| ARID4B | 51742 | 233464430 | Frame_Shift_Del | c.1084_1084delG | p.V196fs | uc001hwq.1 | P26 |
| CD36 | 948 | 80137255 | Missense | c.1483T > G | p.F267V | uc003uhc.1 | P26 |
| CDK13 | 8621 | 40098992 | Missense | c.3601A > G | p.M1107V | uc003thh.2 | P26 |
| CECR2 | 27443 | 16383308 | Missense | c.1119T > A | p.S331R | uc010gqw.1 | P26 |
| CMYA5 | 202333 | 79122587 | Missense | c.11800G > T | p.A3910S | uc003kgc.1 | P26 |
| FAM70A | 55026 | 119329145 | Frame_Shift_Del | c.275_275delC | p.P16fs | uc004eso.2 | P26 |
| KIAA1598 | 57698 | 118633781 | Frame_Shift_Del | c.2399_2399delT | p.L634fs | uc001lcx.2 | P26 |
| MGAT4C | 25834 | 84901503 | Missense | c.1474A > T | p.D75V | uc001tai.2 | P26 |
| MYRIP | 25924 | 40060572 | Missense | c.273A > G | p.K3R | uc010hhw.1 | P26 |
| NPAS3 | 64067 | 32906141 | Splice_Site_SNP | c.e4_splice_site | | uc001wru.1 | P26 |
| PTPRN2 | 5799 | 157063530 | Missense | c.2617C > T | p.R854W | uc003wno.1 | P26 |
| RAPGEF2 | 9693 | 160494480 | Missense | c.4310G > A | p.G1334R | uc003iqg.2 | P26 |
| STT3A | 3703 | 124979323 | Missense | c.571G > A | p.R160Q | uc001qcd.1 | P26 |
| TMEM195 | 392636 | 15566366 | Missense | c.352T > C | p.L61P | uc003stb.1 | P26 |
| ZNF677 | 342926 | 58432812 | Missense | c.1165T > C | p.V327A | uc002qbf.1 | P26 |
| B3GAT3 | 26229 | 62145914 | Missense | c.111G > T | p.G28C | uc001ntw.1 | P27 |
| COL24A1 | 255631 | 85973133 | Missense | c.4927A > G | p.T1629A | uc001dlj.1 | P27 |
| DACH2 | 117154 | 85957731 | Missense | c.1723A > G | p.T575A | uc004eew.1 | P27 |
| DST | 667 | 56465026 | Missense | c.14344G > C | p.E4608D | uc003pcz.2 | P27 |
| EGR2 | 1959 | 64243254 | Missense | c.1488C > A | p.H384N | uc001jmi.1 | P27 |
| FOXO3 | 2309 | 108989631 | Missense | c.843A > G | p.K176R | uc003psk.2 | P27 |
| IGSF1 | 3547 | 130246905 | Missense | c.731G > A | p.C199Y | uc004ewd.1 | P27 |
| KIAA1632 | 57724 | 41733467 | Missense | c.4809C > T | p.P1570L | uc002lbm.1 | P27 |
| LAS1L | 81887 | 64664934 | Missense | c.959C > T | p.A296V | uc004dwa.1 | P27 |
| MICAL1 | 64780 | 109874059 | Missense | c.2808T > G | p.W852G | uc003ptj.1 | P27 |
| MYCBP2 | 23077 | 76735819 | Missense | c.1515T > C | p.L475P | uc001vkf.1 | P27 |
| NOTCH1 | 4851 | 138510470 | Frame_Shift_Del | c.7541_7542delCT | p.P2514fs | uc004chz.1 | P27 |
| PPM1A | 5494 | 59819255 | Missense | c.396C > A | p.S100R | uc001xew.2 | P27 |
| RAPGEF4 | 11069 | 173387259 | Missense | c.491G > A | p.V102M | uc002uhv.2 | P27 |
| SCN2A | 6326 | 165872675 | Missense | c.748A > T | p.D153V | uc002udc.1 | P27 |
| SLC5A7 | 60482 | 107980751 | Missense | c.750T > A | p.D158E | uc002tdv.1 | P27 |
| TGS1 | 96764 | 56861981 | Missense | c.1357A > G | p.I324V | uc003xsj.2 | P27 |
| UBP1 | 7342 | 33409058 | Splice_Site_SNP | c.e15_splice_site | | uc003cfq.2 | P27 |
| ZNF182 | 7569 | 47721598 | Missense | c.1178A > G | p.I278V | uc004dir.1 | P27 |
| ABCB1 | 5243 | 87034082 | Nonsense | c.903G > A | p.W162* | uc003uiz.1 | P28 |
| ARHGAP21 | 57584 | 24948737 | Frame_Shift_Ins | c.2526_2527insG | p.E697fs | uc001isb.1 | P28 |
| ARID4B | 51742 | 233407765 | Missense | c.3919G > A | p.V1141I | uc001hwq.1 | P28 |
| CARS | 833 | 2979017 | Missense | c.2473G > A | p.S800N | uc001lxf.1 | P28 |
| COL25A1 | 84570 | 109959922 | Missense | c.1914G > A | p.V620I | uc010imd.1 | P28 |
| FZD5 | 7855 | 208340841 | Missense | c.1278G > A | p.V290I | uc002vcj.1 | P28 |
| KYNU | 8942 | 143428880 | Missense | c.535T > A | p.N135K | uc002tvl.1 | P28 |
| PCDH1 | 5097 | 141229051 | Missense | c.287C > A | p.A57D | uc003llp.1 | P28 |
| SAMHD1 | 25939 | 34978851 | Frame_Shift_Del | c.998_998delC | p.R290fs | uc002xgh.1 | P28 |
| VWF | 7450 | 5998644 | Missense | c.4451G > A | p.V1401I | uc001qnn.1 | P28 |
| ZFP36 | 7538 | 44590543 | Missense | c.403T > A | p.S115R | uc002olh.1 | P28 |
| ANGPTL5 | 253935 | 101270859 | Missense | c.1404T > C | p.F270L | uc001pgl.1 | P29 |
| CPNE3 | 8895 | 87632388 | Splice_Site_SNP | c.e14_splice_site | | uc003ydv.1 | P29 |
| FAT4 | 79633 | 126591624 | Missense | c.10003T > G | p.Y3335D | uc003ifj.2 | P29 |
| FIBP | 9158 | 65408057 | Missense | c.1111C > G | p.P339A | uc009yqu.1 | P29 |
| HHATL | 57467 | 42709305 | Missense | c.1604G > A | p.R486H | uc003clw.1 | P29 |
| MAPK1 | 5594 | 20457181 | Missense | c.1187A > T | p.Y316F | uc002zvn.1 | P29 |
| MAPK1 | 5594 | 20457256 | Missense | c.1112A > G | p.D291G | uc002zvn.1 | P29 |
| PPP2R3C | 55012 | 34655686 | Frame_Shift_Del | c.421_421delA | p.S23fs | uc001wss.1 | P29 |
| PRKCQ | 5588 | 6593051 | Missense | c.413A > T | p.K110I | uc001iji.1 | P29 |
| RHD | 6007 | 25502530 | Missense | c.990A > C | p.Y311S | uc009vro.1 | P29 |
| SCN3A | 6328 | 165654908 | Read-through | c.6493T > A | p.*2001K | uc002ucx.1 | P29 |
| ADAMTSL4 | 54507 | 148794535 | Missense | c.1468G > A | p.G437D | uc009wlw.1 | P30 |
| AVIL | 10677 | 56487479 | Missense | c.1422C > T | p.R465W | uc001sqj.1 | P30 |
| CTSB | 1508 | 11743148 | Frame_Shift_Del | c.474_474delG | p.G60fs | uc003wul.1 | P30 |
| HERC2 | 8924 | 26151908 | Nonsense | c.4760C > T | p.R1552* | uc001zbj.1 | P30 |
| MARK2 | 2011 | 63414276 | Missense | c.369G > T | p.C16F | uc009yox.1 | P30 |
| NR4A1 | 3164 | 50734881 | Missense | c.1659G > A | p.E222K | uc001rzq.1 | P30 |
| ZNF697 | 90874 | 119970191 | Missense | c.170G > A | p.G19E | uc001ehy.1 | P30 |
| ZNF804A | 91752 | 185510424 | Missense | c.2650A > G | p.T686A | uc002uph.1 | P30 |
| ACTL7B | 10880 | 110657143 | Missense | c.889C > T | p.R297C | uc004bdi.1 | P31 |
| BTBD1 | 53339 | 81501564 | Missense | c.985T > C | p.F261S | uc002bjn.1 | P31 |
| FANCA | 2175 | 88385382 | Missense | c.1331C > T | p.A430V | uc002fou.1 | P31 |
| GPAT2 | 150763 | 96054010 | Missense | c.1784A > G | p.I521V | uc002svf.1 | P31 |
| GRIN2B | 2904 | 13608660 | Missense | c.2958C > T | p.R927W | uc001rbt.2 | P31 |
| MAP1A | 4130 | 41601424 | Missense | c.928G > A | p.R154H | uc001zrt.1 | P31 |
| MYD88 | 4615 | 38157645 | Missense | c.794T > C | p.L265P | NM_002468 | P31 |
| OR4C12 | 283093 | 49959841 | Missense | c.773G > A | p.R258H | uc001nhc.1 | P31 |
| PTRF | 284119 | 37828403 | Missense | c.398C > G | p.A80G | uc002hzo.1 | P31 |
| RAB4B | 53916 | 45984445 | Missense | c.1422G > A | p.E182K | uc002opf.1 | P31 |
| RUNX1 | 861 | 35086661 | Frame_Shift_Del | c.1333_1333delT | p.S362fs | uc010gmu.1 | P31 |
| ZBTB6 | 10773 | 124713556 | Missense | c.706C > G | p.S206C | uc004bnh.1 | P31 |
| CELF3 | 11189 | 149946321 | Nonsense | c.1640C > A | p.Y282* | uc001eys.1 | P32 |
| CETN2 | 1069 | 151747056 | Missense | c.551G > C | p.K168N | uc004fgq.1 | P32 |
| CSMD2 | 114784 | 33784266 | Missense | c.9235C > A | p.Q3020K | uc001bxm.1 | P32 |
| EIF2B2 | 8892 | 74539853 | Splice_Site_SNP | c.e2_splice_site | | uc001xrc.1 | P32 |
| FAM117A | 81558 | 45150022 | Missense | c.843C > G | p.S254R | uc002ipk.1 | P32 |
| GPR87 | 53836 | 152495273 | Missense | c.812C > T | p.R151W | uc003eyt.1 | P32 |
| IGSF3 | 3321 | 116944276 | Missense | c.2604C > A | p.F633L | uc001egq.1 | P32 |
| KIAA1109 | 84162 | 123380426 | Missense | c.4184G > T | p.R1380L | uc003ieh.1 | P32 |
| MAP3K12 | 7786 | 52167053 | Frame_Shift_Del | c.487_488delCT | p.P130fs | uc001sdn.1 | P32 |
| MUC2 | 4583 | 1082884 | Missense | c.11816C > T | p.T3930M | uc001lsx.1 | P32 |
| PHKA1 | 5255 | 71717660 | Missense | c.3890T > G | p.F1197V | uc004eax.2 | P32 |
| PNKP | 11284 | 55062237 | Missense | c.89G > C | p.E13Q | uc002pqh.1 | P32 |
| RBM19 | 9904 | 112840590 | Missense | c.2515C > G | p.R811G | uc009zwi.1 | P32 |
| SF3B1 | 23451 | 197975079 | Missense | c.2146A > G | p.K700E | uc002uue.1 | P32 |
| SGCG | 6445 | 22792811 | Missense | c.738C > T | p.A205V | uc001uom.1 | P32 |
| SLCO1A2 | 6579 | 21336396 | Missense | c.2300G > C | p.A527P | uc001res.1 | P32 |
| SPOP | 8405 | 45051434 | Missense | c.859G > A | p.D130N | uc002ipb.1 | P32 |
| TCHP | 84260 | 108830838 | Missense | c.917A > G | p.E255G | uc001tpn.1 | P32 |
| USP44 | 84101 | 94442635 | Missense | c.1829T > C | p.M562T | uc001teg.1 | P32 |
| ZNF282 | 8427 | 148552323 | Missense | c.1772A > C | p.N556T | uc003wfm.1 | P32 |
| ZNF664 | 144348 | 123063059 | Missense | c.2245G > A | p.G139R | uc001ufz.1 | P32 |
| ZNF791 | 163049 | 12600115 | Missense | c.934A > G | p.S258G | uc002mua.2 | P32 |
| ACSL6 | 23305 | 131335216 | Missense | c.1463C > T | p.R454W | uc003kvx.1 | P33 |
| ADAMTS10 | 81794 | 8574766 | De_novo_Start_OutOfFrame | c.597C > T | | uc002mkk.1 | P33 |
| ANKS6 | 203286 | 100570330 | Missense | c.2017T > C | p.S666P | uc004ayu.1 | P33 |
| ANXA10 | 11199 | 169285876 | Missense | c.230G > T | p.A29S | uc003irm.1 | P33 |
| BTNL9 | 153579 | 180412853 | Missense | c.1001A > C | p.T262P | uc003mmt.1 | P33 |
| C11orf41 | 25758 | 33561604 | Missense | c.3798A > C | p.N1225T | uc001mup.2 | P33 |
| CDH12 | 1010 | 22114407 | Missense | c.594C > T | p.R46W | uc010iuc.1 | P33 |
| CDH5 | 1003 | 64981869 | Missense | c.1000G > T | p.V282F | uc002eom.2 | P33 |
| COL11A1 | 1301 | 103119877 | Frame_Shift_Ins | c.5357_5358insC | p.P1680fs | uc001dum.1 | P33 |
| DCLK1 | 9201 | 35246793 | Missense | c.2388G > A | p.A726T | uc001uvf.1 | P33 |
| DTNA | 1837 | 30599964 | Missense | c.110A > G | p.T37A | uc010dmn.1 | P33 |
| EP300 | 2033 | 39877805 | Missense | c.3235T > C | p.I947T | uc003azl.2 | P33 |
| FOXR1 | 283150 | 118356625 | Missense | c.1052T > C | p.I276T | uc001pui.1 | P33 |
| HCFC1 | 3054 | 152878970 | Missense | c.1522A > C | p.T332P | uc004fjp.1 | P33 |
| HOOK2 | 29911 | 12744473 | Missense | c.581C > T | p.T137M | uc002muy.2 | P33 |
| KCNA10 | 3744 | 110862914 | Missense | c.407A > G | p.K7E | uc001dzt.1 | P33 |
| KRT16 | 3868 | 37022385 | Missense | c.221T > C | p.S28P | uc002hxg.2 | P33 |
| MAP1A | 4130 | 41604668 | Missense | c.4172A > C | p.E1235D | uc001zrt.1 | P33 |
| MAP3K15 | 389840 | 19308260 | Missense | c.2550G > C | p.A305P | uc004czk.1 | P33 |
| MARK1 | 4139 | 218893202 | Missense | c.2473G > A | p.V626I | uc009xdw.1 | P33 |
| NBEAL1 | 65065 | 203711189 | Missense | c.739C > T | p.P223L | uc002urt.2 | P33 |
| PDE3A | 5139 | 20657852 | Missense | c.1242G > T | p.C407F | uc001reh.1 | P33 |
| PI4K2A | 55361 | 99400858 | Missense | c.663A > T | p.K202N | uc001kog.1 | P33 |
| PLIN1 | 5346 | 88014406 | Missense | c.531C > T | p.A136V | uc002boh.1 | P33 |
| SNX7 | 51375 | 98923179 | Missense | c.406G > C | p.E47Q | uc001drz.1 | P33 |
| TERT | 7015 | 1347170 | Missense | c.889A > C | p.R277S | uc003jcb.1 | P33 |
| TNNI1 | 7135 | 199647223 | Missense | c.341G > A | p.R114H | uc009wzw.1 | P33 |
| TP53 | 7157 | 7517845 | Missense | c.1012G > A | p.R273H | uc002gim.2 | P33 |
| WNK2 | 65268 | 95094762 | Missense | c.5305C > T | p.R1769C | uc004ati.1 | P33 |
| C9orf86 | 55684 | 138854454 | In_frame_Del | c.2418_2420delAG | p.K661del | uc004cjj.1 | P34 |
| CCDC21 | 64793 | 26470129 | Nonsense | c.1818G > T | p.E563* | uc001bls.1 | P34 |
| DCAF6 | 55827 | 166301494 | Frame_Shift_Ins | c.2724_2725insC | p.G828fs | uc001gex.1 | P34 |
| DNMT3B | 1789 | 30859284 | Missense | c.2797C > T | p.R826C | uc002wyc.1 | P34 |
| DPY19L2 | 283417 | 62240621 | Missense | c.2396G > A | p.A739T | uc001srp.1 | P34 |
| E2F3 | 1871 | 20595009 | Missense | c.1322T > C | p.I332T | uc003nda.2 | P34 |
| EGR2 | 1959 | 64243338 | Missense | c.1404G > A | p.E356K | uc001jmi.1 | P34 |
| GAB3 | 139716 | 153594097 | Missense | c.718G > A | p.V224I | uc004fmk.1 | P34 |
| LGR5 | 8549 | 70264078 | Missense | c.2069C > T | p.T674M | uc001swl.1 | P34 |
| LY9 | 4063 | 159050298 | Missense | c.753C > T | p.P235S | uc001fwu.1 | P34 |
| MLXIP | 22877 | 121184519 | Frame_Shift_Ins | c.1244_1245insC | p.A339fs | uc001ubr.2 | P34 |
| MPHOSPH9 | 10198 | 122244914 | Missense | c.1864T > A | p.L586Q | uc001ue1.1 | P34 |
| NDUFA4 | 4697 | 10945050 | Splice_Site_SNP | c.e2_splice_site | | uc003srx.1 | P34 |
| PREX2 | 80243 | 69143820 | Splice_Site_SNP | c.e12_splice_site | | uc003xxv.1 | P34 |
| PSMC5 | 5705 | 59262461 | Missense | c.954C > A | p.L305M | uc002jcb.1 | P34 |
| PURB | 5814 | 44890554 | Missense | c.932G > C | p.E307Q | uc003tme.1 | P34 |
| RBM39 | 9584 | 33776456 | Missense | c.796A > T | p.D151V | uc002xeb.1 | P34 |
| RPS6KA6 | 27330 | 83259120 | Splice_Site_SNP | c.e10_splice_site | | uc004eej.1 | P34 |
| SPCS3 | 60559 | 177478252 | Missense | c.127C > A | p.L11M | uc003iur.2 | P34 |
| SSTR4 | 6754 | 22965250 | Missense | c.1194G > A | p.R377H | uc002wsr.2 | P34 |
| TET1 | 80312 | 70074514 | Nonsense | c.2527C > G | p.Y674* | uc001jok.2 | P34 |
| TGDS | 23483 | 94026580 | Missense | c.1092T > A | p.I324K | uc001vlw.1 | P34 |
| TRIM4 | 89122 | 99354609 | Missense | c.482C > A | p.H118N | uc003usd.1 | P34 |
| ACPT | 93650 | 55989580 | Missense | c.916A > C | p.T306P | uc002pta.1 | P35 |
| BRD7 | 29117 | 48920149 | Missense | c.1039T > C | p.F340S | uc002ege.1 | P35 |
| CMYA5 | 202333 | 79068135 | Missense | c.7863A > T | p.K2597N | uc003kgc.1 | P35 |
| FBXW7 | 55294 | 153464851 | Missense | c.1939G > A | p.G597E | uc003ims.1 | P35 |
| FBXW7 | 55294 | 153478425 | Missense | c.989C > A | p.F280L | uc003ims.1 | P35 |
| HOOK2 | 29911 | 12735564 | Missense | c.2027G > A | p.R619Q | uc002muy.2 | P35 |
| NCOR1 | 9611 | 15952824 | Splice_Site_SNP | c.e19_splice_site | | uc002gpo.1 | P35 |
| OPRM1 | 4988 | 154453921 | Missense | c.1022A > G | p.K324R | uc003qpq.1 | P35 |
| PAG1 | 55824 | 82068007 | Missense | c.722C > T | p.A4V | uc003ybz.1 | P35 |
| PGBD3 | 267004 | 50393887 | Missense | c.2838G > C | p.G895A | uc009xoe.1 | P35 |
| RABGGTA | 5875 | 23808717 | Missense | c.873T > G | p.F151V | uc001wof.1 | P35 |
| RLBP1 | 6017 | 87559426 | Missense | c.774T > C | p.S132P | uc002bnl.1 | P35 |
| RNF213 | 57674 | 75940090 | Missense | c.4637A > T | p.I1472L | uc002jyh.1 | P35 |
| RYK | 6259 | 135377265 | Missense | c.1552G > A | p.C485Y | uc003eqc.1 | P35 |
| SORCS3 | 22986 | 106927913 | Missense | c.2228C > G | p.H667Q | uc001kyi.1 | P35 |
| TCP11 | 6954 | 35211892 | Missense | c.426A > G | p.Y82C | uc003okd.2 | P35 |
| VPS13A | 23230 | 79171461 | Splice_Site_SNP | c.e61_splice_site | | uc004akr.1 | P35 |
| WDR72 | 256764 | 51784659 | Missense | c.1208A > G | p.Q389R | uc002acj.2 | P35 |
| WSCD2 | 9671 | 107128162 | Missense | c.1376A > C | p.N211T | uc001tms.1 | P35 |
| ZMYM3 | 9203 | 70386657 | Nonsense | c.1282C > T | p.Q399* | uc004dzh.1 | P35 |
| ZNF648 | 127665 | 180293126 | Missense | c.851A > C | p.T215P | uc001goz.1 | P35 |
| ZXDA | 7789 | 57953019 | Frame_Shift_Ins | c.773_774insC | p.P187fs | uc004dve.1 | P35 |
| CDK20 | 23552 | 89773928 | Splice_Site_SNP | c.e7_splice_site | | uc004apr.1 | P36 |
| CDT1 | 81620 | 87399944 | Frame_Shift_Ins | c.901_902insC | p.A283fs | uc002flu.1 | P36 |
| CXADR | 1525 | 17807360 | Frame_Shift_Del | c.160_160delG | p.V14fs | uc002yki.1 | P36 |
| FGD1 | 2245 | 54513876 | Missense | c.1258C > G | p.P175R | uc004dtg.1 | P36 |
| IGFBP6 | 3489 | 51777969 | Frame_Shift_Del | c.267_267delG | p.E67fs | uc001sbu.1 | P36 |
| KLF8 | 11279 | 56308797 | Missense | c.1402G > C | p.V181L | uc004dur.1 | P36 |
| NAV2 | 89797 | 19912305 | Missense | c.2369A > G | p.T670A | uc009yhw.1 | P36 |
| NBPF14 | 25832 | 146482257 | Frame_Shift_Del | c.1015_1015delA | p.N333fs | uc001eqq.1 | P36 |
| RAB11FIP4 | 84440 | 26872303 | Splice_Site_SNP | c.e5_splice_site | | uc002hgn.1 | P36 |
| SIX4 | 51804 | 60250237 | Missense | c.1987A > G | p.T663A | uc001xfc.2 | P36 |
| TRIP11 | 9321 | 91550648 | Missense | c.1638C > A | p.L284I | uc001xzy.2 | P36 |
| AMPH | 273 | 38469152 | Missense | c.905A > G | p.H279R | uc003tgu.1 | P37 |
| DACH2 | 117154 | 85954861 | Nonsense | c.1462C > T | p.R488* | uc004eew.1 | P37 |
| DDX3X | 1654 | 41089660 | Frame_Shift_Del | c.2085_2085delT | p.S410fs | uc004dfe.1 | P37 |
| GRID2 | 2895 | 94595938 | Nonsense | c.1906C > T | p.R550* | uc003hsz.2 | P37 |
| IGSF22 | 283284 | 18695110 | Missense | c.1177G > T | p.K329N | uc009yht.1 | P37 |
| MCAM | 4162 | 118690941 | Missense | c.241C > T | p.T71M | uc001pwf.1 | P37 |
| MICAL3 | 57553 | 16747051 | Frame_Shift_Ins | c.1842_1843insC | p.R472fs | uc002znj.1 | P37 |
| MYT1L | 23040 | 1822085 | Missense | c.3750G > A | p.A975T | uc002qxe.1 | P37 |
| POLL | 27343 | 103330004 | Frame_Shift_Ins | c.2119_2120insAT | p.L451fs | uc001ktg.1 | P37 |
| PTPRB | 5787 | 69251258 | Missense | c.3229G > A | p.G1062E | uc001swc.2 | P37 |
| SCN2A | 6326 | 165954314 | Missense | c.6042C > T | p.R1918C | uc002udc.1 | P37 |
| SF3B1 | 23451 | 197975079 | Missense | c.2146A > G | p.K700E | uc002uue.1 | P37 |
| SUSD4 | 55061 | 221603326 | In_frame_Del | c.697_699delGCA | p.21_22QQ > Q | uc001hnx.1 | P37 |
| ZC3H12B | 340554 | 64638489 | Missense | c.1162G > T | p.A385S | uc010nko.1 | P37 |
| NEU4 | 129807 | 242404444 | Frame_Shift_Ins | c.577_578insC | p.V42fs | uc002wcn.1 | P38 |
| ZMYM3 | 9203 | 70389672 | Frame_Shift_Del | c.246_246delC | p.S53fs | uc004dzh.1 | P38 |
| ABCB5 | 340273 | 20749137 | Missense | c.3374T > C | p.V1046A | uc010kuh.1 | P39 |
| ACSS1 | 84532 | 24942689 | Missense | c.2238G > A | p.A454T | uc002wub.1 | P39 |
| AKAP12 | 9590 | 151712279 | Missense | c.1249G > A | p.E354K | uc003qoe.1 | P39 |
| ALDH1A1 | 216 | 74733730 | Missense | c.393C > T | p.L114F | uc004ajd.1 | P39 |
| B3GALT1 | 8708 | 168434477 | Missense | c.1033C > T | p.P228S | uc002udz.1 | P39 |
| BRD7 | 29117 | 48911413 | Missense | c.1809C > T | p.L597F | uc002ege.1 | P39 |
| BSN | 8927 | 49674601 | Missense | c.10433G > C | p.S3440T | uc003cxe.2 | P39 |
| C2orf42 | 54980 | 70262594 | Missense | c.356G > C | p.V10L | uc002sgh.1 | P39 |
| CCDC9 | 26093 | 52455748 | Missense | c.420G > C | p.G92R | uc002pgh.1 | P39 |
| CDHR5 | 53841 | 609562 | Missense | c.1310G > A | p.R402Q | uc001lqj.1 | P39 |
| CHD5 | 26038 | 6108495 | Missense | c.4189T > G | p.D1363E | uc001amb.1 | P39 |
| CLCN1 | 1180 | 142758858 | Missense | c.2732C > T | p.P882L | uc003wcr.1 | P39 |
| CPNE9 | 151835 | 9721438 | Missense | c.286G > C | p.V39L | uc003bsd.1 | P39 |
| CR1 | 1378 | 205858200 | Missense | c.7191G > A | p.D2351N | uc001hfx.1 | P39 |
| CSAD | 51380 | 51852591 | Missense | c.550C > T | p.R79C | uc001sbx.1 | P39 |
| DCHS2 | 54798 | 155383276 | Missense | c.5675T > A | p.F1892Y | uc003inw.1 | P39 |
| DNMBP | 23268 | 101638648 | Missense | c.3301T > C | p.M1070T | uc001kqj.2 | P39 |
| DOLK | 22845 | 130748773 | Missense | c.1061C > T | p.R211C | uc004bwr.1 | P39 |
| DST | 667 | 56643470 | Missense | c.1029G > A | p.G170E | uc003pcz.2 | P39 |
| EXOSC8 | 11340 | 36475070 | Splice_Site_SNP | c.e4_splice_site | | uc001uwa.1 | P39 |
| F5 | 2153 | 167796473 | Missense | c.674A > G | p.N177D | uc001ggg.1 | P39 |
| GAB4 | 128954 | 15848875 | Missense | c.769G > A | p.A221T | uc002zlw.1 | P39 |
| GALNT8 | 26290 | 4740568 | Nonsense | c.1449C > T | p.Q453* | uc001qne.1 | P39 |
| GRIK5 | 2901 | 47238696 | Missense | c.1356G > A | p.E441K | uc002osj.1 | P39 |
| HDAC4 | 9759 | 239701828 | Missense | c.2426C > T | p.P545L | uc002vyk.2 | P39 |
| IFNA8 | 3445 | 21399358 | Missense | c.213C > G | p.F61L | uc003zpc.1 | P39 |
| IGSF10 | 285313 | 152648274 | Missense | c.2185C > T | p.R729C | uc003ezb.1 | P39 |
| JUB | 84962 | 22513160 | Missense | c.1803G > C | p.C476S | uc001whz.1 | P39 |
| KCNK13 | 56659 | 89720462 | Missense | c.1031G > A | p.V197I | uc001xye.1 | P39 |
| KIF7 | 374654 | 87977985 | Missense | c.1174G > A | p.R330H | uc002bof.1 | P39 |
| LIPI | 149998 | 14476028 | Missense | c.638T > G | p.F210V | uc002yjm.1 | P39 |
| LRRK1 | 79705 | 99385047 | Missense | c.2783G > A | p.V822I | uc002bwr.1 | P39 |
| LUC7L | 55692 | 196120 | Missense | c.505G > A | p.E132K | uc002cgc.1 | P39 |
| MARCKS | 4082 | 114288354 | Missense | c.1300C > A | p.A302E | uc003pvy.2 | P39 |
| MME | 4311 | 156349110 | Missense | c.1786G > T | p.K525N | uc010hvr.1 | P39 |
| PAX8 | 7849 | 113694145 | Missense | c.1437T > G | p.L424W | uc002tjk.1 | P39 |
| PELI2 | 57161 | 55714897 | Missense | c.455A > T | p.S57C | uc001xch.1 | P39 |
| PHRF1 | 57661 | 598503 | In_frame_Del | c.3175_3186delG | p.TRSG1017del | uc001lqe.1 | P39 |
| POTEB | 339010 | 19335787 | Missense | c.881C > G | p.Q172E | uc001ytu.1 | P39 |
| PRMT6 | 55170 | 107401838 | Missense | c.907C > G | p.N267K | uc001dvb.1 | P39 |
| PTPRU | 10076 | 29503025 | Missense | c.2688C > T | p.R860W | uc001bru.1 | P39 |
| RAPGEF1 | 2889 | 133491472 | Missense | c.1522C > T | p.H455Y | uc004cbb.1 | P39 |
| RCL1 | 10171 | 4831330 | Missense | c.941A > G | p.D228G | uc003zis.2 | P39 |
| RNF38 | 152006 | 36342814 | Missense | c.1294C > T | p.T368I | uc003zzh.1 | P39 |
| RPL31 | 6160 | 100988954 | Missense | c.422C > A | p.T112N | uc010fiu.1 | P39 |
| RYR3 | 6263 | 31865241 | Missense | c.9425G > A | p.E3119K | uc001zhi.1 | P39 |
| SERPINA12 | 145264 | 94034466 | Missense | c.818G > A | p.A8T | uc001ydj.1 | P39 |
| SLC10A6 | 345274 | 87965636 | Missense | c.880G > A | p.G294R | uc003hqd.1 | P39 |
| TBC1D8 | 11138 | 101037194 | Missense | c.525G > A | p.E132K | uc010fiv.1 | P39 |
| TINAG | 27283 | 54299621 | Missense | c.718G > A | p.R191H | uc003pcj.1 | P39 |
| TP53 | 7157 | 7519251 | Missense | c.598G > A | p.C135Y | uc002gim.2 | P39 |
| U2AF2 | 11338 | 60864312 | Missense | c.1486T > A | p.M144K | uc002qlu.1 | P39 |
| UPP2 | 151531 | 158682585 | Missense | c.534G > A | p.G115S | uc002tzo.1 | P39 |
| WDR73 | 84942 | 82987881 | In_frame_Del | c.960_977delATG | p.DGTRSQ315del | uc002bkw.1 | P39 |
| WNK4 | 65266 | 38201821 | Missense | c.3607A > C | p.T1196P | uc002ibj.1 | P39 |
| WNK4 | 65266 | 38201824 | Missense | c.3610T > C | p.S1197P | uc002ibj.1 | P39 |
| WWTR1 | 25937 | 150742960 | Missense | c.639A > C | p.N208T | uc003exe.1 | P39 |
| ZNF556 | 80032 | 2828320 | Missense | c.451C > T | p.R122C | uc002lwp.1 | P39 |
| ZNF777 | 27153 | 148783559 | Missense | c.651A > G | p.D163G | uc003wfv.1 | P39 |
| ZNF793 | 390927 | 42720002 | Missense | c.1044C > T | p.P201L | uc010efm.1 | P39 |
| ABLIM2 | 84448 | 8072915 | Missense | c.1327G > A | p.R395Q | uc003gko.2 | P40 |
| AMOTL2 | 51421 | 135563304 | Nonsense | c.1599C > T | p.R439* | uc003eqg.1 | P40 |
| ASB18 | 401036 | 236787746 | Frame_Shift_Ins | c.1098_1099insC | p.P366fs | uc010fyo.1 | P40 |
| BTBD3 | 22903 | 11848400 | Missense | c.811C > T | p.A151V | uc002wnz.1 | P40 |
| CSMD1 | 64478 | 3251052 | Missense | c.2563G > A | p.D725N | uc010lrh.1 | P40 |
| GRIK5 | 2901 | 47201867 | Missense | c.2146G > A | p.S704N | uc002osj.1 | P40 |
| KIAA0226 | 9711 | 198913134 | Splice_Site_SNP | c.e6_splice_site | | uc003fyc.2 | P40 |
| KIAA1199 | 57214 | 79001412 | Missense | c.2341G > A | p.G694E | uc002bfw.1 | P40 |
| KPNA5 | 3841 | 117129873 | Splice_Site_SNP | c.e6_splice_site | | uc003pxh.1 | P40 |
| OR5R1 | 219479 | 55941797 | Missense | c.488C > T | p.T163I | uc001niu.1 | P40 |
| PTPRD | 5789 | 8474233 | Missense | c.4010C > T | p.T1100M | uc003zkk.1 | P40 |
| RGS2 | 5997 | 191045917 | Splice_Site_SNP | c.e2_splice_site | | uc001gsl.1 | P40 |
| RRP1B | 23076 | 43935778 | Missense | c.2164G > T | p.V684F | uc002zdk.1 | P40 |
| SF3B1 | 23451 | 197973694 | Missense | c.2756A > G | p.Q903R | uc002uue.1 | P40 |
| TFCP2 | 7024 | 49789182 | Missense | c.1165A > G | p.K236E | uc001rxw.1 | P40 |
| VWA3B | 200403 | 98253575 | Splice_Site_SNP | c.e22_splice_site | | uc002syo.1 | P40 |
| XIRP2 | 129446 | 167823572 | Missense | c.2458G > T | p.R790I | uc010fpn.1 | P40 |
| C6 | 729 | 41185830 | Missense | c.2610C > A | p.S791Y | uc003jml.1 | P41 |
| CASP4 | 837 | 104327874 | Missense | c.404C > T | p.H111Y | uc001pid.1 | P41 |
| CMKLR1 | 1240 | 107210118 | Missense | c.1259G > A | p.R249H | uc001tmv.1 | P41 |
| DDR2 | 4921 | 160996394 | Splice_Site_SNP | c.e9_splice_site | | uc001gcf.1 | P41 |
| DRGX | 644168 | 50244225 | Missense | c.749G > A | p.G250D | uc001jhq.1 | P41 |
| FBN1 | 2200 | 46679698 | Missense | c.700G > A | p.M124I | uc001zwx.1 | P41 |
| HERC3 | 8916 | 89808138 | Missense | c.1682A > G | p.I506V | uc003hrw.1 | P41 |
| LANCL1 | 10314 | 211009349 | Missense | c.990G > C | p.E296Q | uc002ved.1 | P41 |
| MCHR2 | 84539 | 100489018 | Nonsense | c.999T > A | p.Y228* | uc003pqh.1 | P41 |
| NRXN1 | 9378 | 50700770 | Missense | c.2691A > G | p.Y405C | uc002rxe.2 | P41 |
| NRXN2 | 9379 | 64175636 | Missense | c.3024C > T | p.T862M | uc001oar.1 | P41 |
| PCDHAC2 | 56134 | 140369551 | Missense | c.3109C > T | p.R957W | uc003111.1 | P41 |
| PLEKHG3 | 26030 | 64278345 | Missense | c.2626T > A | p.L786Q | uc001xho.1 | P41 |
| PMS2 | 5395 | 5992931 | Missense | c.2078T > A | p.L664Q | uc003spl.1 | P41 |
| PTPRF | 5792 | 43836122 | Missense | c.2270G > A | p.V644M | uc001cjr.1 | P41 |
| RGS9 | 8787 | 60586832 | Missense | c.335C > A | p.N75K | uc002jfe.1 | P41 |
| RIPK1 | 8737 | 3058352 | Missense | c.2028A > G | p.K599R | uc010jni.1 | P41 |
| SON | 6651 | 33870612 | Missense | c.6331G > C | p.A1405P | uc002ysd.2 | P41 |
| SPEG | 10290 | 220056174 | Frame_Shift_Ins | c.5745_5746insG | p.S1915fs | uc010fwg.1 | P41 |
| THUMPD2 | 80745 | 39850562 | Missense | c.462T > G | p.1125R | uc002rru.1 | P41 |
| TP53 | 7157 | 7518293 | Missense | c.907G > C | p.C238S | uc002gim.2 | P41 |
| ATF7IP | 55729 | 14540430 | Splice_Site_SNP | c.e14_splice_site | | uc001rbw.1 | P42 |
| C3orf62 | 375341 | 49288927 | Missense | c.530C > A | p.A128E | uc003cwn.1 | P42 |
| CALHM1 | 255022 | 105205258 | Missense | c.929C > A | p.H264Q | uc001kxe.1 | P42 |
| CNOT1 | 23019 | 57150066 | Missense | c.2437C > A | p.A715D | uc002env.1 | P42 |
| CREBZF | 58487 | 85052735 | Missense | c.1087C > T | p.A278V | uc001pas.1 | P42 |
| CSNK1E | 1454 | 37026883 | Missense | c.823C > G | p.I119M | uc003avm.1 | P42 |
| ECT2L | 345930 | 139243830 | Missense | c.1812T > A | p.V570D | uc003qif.1 | P42 |
| EIF4ENIF1 | 56478 | 30181144 | Missense | c.1421T > A | p.N419K | uc003akz.1 | P42 |
| ELN | 2006 | 73112274 | Missense | c.1646G > A | p.V519I | uc003tzw.1 | P42 |
| FBXW7 | 55294 | 153468834 | Missense | c.1543G > A | p.R465H | uc003ims.1 | P42 |
| IFT140 | 9742 | 1513671 | Missense | c.3349C > G | p.A1101G | uc002cma.1 | P42 |
| IL17RD | 54756 | 57107155 | Missense | c.1705G > A | p.G539D | uc003dil.1 | P42 |
| MACF1 | 23499 | 39662421 | Missense | c.11735A > C | p.R3868S | uc009wr.1 | P42 |
| MPRIP | 23164 | 16922048 | Splice_Site_SNP | c.e3_splice_site | | uc002gqv.1 | P42 |
| MUC5B | 727897 | 1227452 | Missense | c.12833A > C | p.T4259P | uc001ltb.2 | P42 |
| MYH11 | 4629 | 15725594 | Missense | c.4418C > A | p.D1437E | uc002ddx.1 | P42 |
| NOVA1 | 4857 | 25987037 | Missense | c.1810G > T | p.V498F | uc001wpy.1 | P42 |
| PCDHGB7 | 56099 | 140778868 | Missense | c.1403G > A | p.V420I | uc003lkn.1 | P42 |
| PDS5B | 23047 | 32130391 | Missense | c.486A > T | p.I110L | uc010abf.1 | P42 |
| PEG3 | 5178 | 62019991 | Missense | c.1982T > C | p.F544S | uc002qnu.1 | P42 |
| PTPN21 | 11099 | 88015924 | Missense | c.1935G > A | p.G535D | uc001xwv.2 | P42 |
| SIGLEC11 | 114132 | 55153421 | Missense | c.1673C > T | p.L516F | uc002pre.1 | P42 |
| SRGAP1 | 57522 | 62807931 | Missense | c.2620C > A | p.P855H | uc001sru.1 | P42 |
| TP53 | 7157 | 7517822 | Missense | c.1035G > A | p.D281N | uc002gim.2 | P42 |
| TTN | 7273 | 179350895 | Missense | c.4485C > T | p.R1421W | uc002umr.1 | P42 |
| ANK2 | 287 | 114470861 | Missense | c.3011A > T | p.S971C | uc003ibe.2 | P43 |
| ARL6IP1 | 23204 | 18716815 | Missense | c.292A > T | p.M75L | uc002dfl.1 | P43 |
| BAZ2A | 11176 | 55279361 | Nonsense | c.5421C > T | p.Q1743* | uc001slq.1 | P43 |
| C20orf177 | 63939 | 57953517 | Missense | c.1539T > A | p.V375E | uc002yba.1 | P43 |
| C2orf3 | 6936 | 75775063 | Splice_Site_SNP | c.e6_splice_site | | uc002sno.1 | P43 |
| C4orf7 | 260436 | 71134491 | Read-through | c.341T > A | p.*86K | uc003hfd.1 | P43 |
| CCDC81 | 60494 | 85801189 | Missense | c.1759T > A | p.I444K | uc001pbx.1 | P43 |
| CHD8 | 57680 | 20938613 | Splice_Site_SNP | c.e23_splice_site | | uc001was.1 | P43 |
| ENPP7 | 339221 | 75323693 | Missense | c.676T > G | p.V219G | uc002jxa.1 | P43 |
| ESCO1 | 114799 | 17398202 | Missense | c.2715T > A | p.L594Q | uc002kth.1 | P43 |
| EVPL | 2125 | 71522748 | Missense | c.2294T > C | p.V689A | uc002jqi.2 | P43 |
| LAMC2 | 3918 | 181466811 | Missense | c.2121G > A | p.E603K | uc001gqa.2 | P43 |
| LCE1C | 353133 | 151044529 | Missense | c.101C > A | p.T17N | uc001fap.1 | P43 |
| LRP1 | 4035 | 55884745 | Missense | c.11606C > T | p.R3714C | uc001snd.1 | P43 |
| MITF | 4286 | 70097073 | Missense | c.1663C > T | p.T516M | uc003dnz.1 | P43 |
| NEUROD1 | 4760 | 182251396 | Missense | c.673C > T | p.A146V | uc002uof.1 | P43 |
| OR2K2 | 26248 | 113130506 | Missense | c.29G > A | p.S10N | uc004bfd.1 | P43 |
| PCLO | 27445 | 82346622 | Missense | c.13910G > A | p.G4541R | uc003uhx.2 | P43 |
| PDE1A | 5136 | 182759004 | Missense | c.1574C > T | p.S475L | uc002uoq.1 | P43 |
| PLEKHH2 | 130271 | 43791228 | Frame_Shift_Del | c.2421_2425delTT | p.F771fs | uc002rte.2 | P43 |
| PLG | 5340 | 161059381 | Missense | c.916G > A | p.G285R | uc003qtm.2 | P43 |
| RIPK1 | 8737 | 3050831 | Nonsense | c.1355C > T | p.Q375* | uc010jni.1 | P43 |
| SCML1 | 6322 | 17678161 | Missense | c.855G > A | p.R177H | uc004cyb.1 | P43 |
| SEMA5B | 54437 | 124123960 | Missense | c.1601C > T | p.P433S | uc003efz.1 | P43 |
| SF3B1 | 23451 | 197975079 | Missense | c.2146A > G | p.K700E | uc002uue.1 | P43 |
| SPATA19 | 219938 | 133217134 | Splice_Site_SNP | c.e6_splice_site | | uc001qgv.1 | P43 |
| TBCK | 93627 | 107385230 | Splice_Site_SNP | c.e11_splice_site | | uc010ilv.1 | P43 |
| TPR | 7175 | 184581453 | Splice_Site_SNP | c.e24_splice_site | | uc001grv.1 | P43 |
| TTC3 | 7267 | 37426856 | Missense | c.1468A > T | p.S455C | uc002yvz.1 | P43 |
| VPS13C | 54832 | 59955338 | Splice_Site_SNP | c.e76_splice_site | | uc002agz.1 | P43 |
| ZNF488 | 118738 | 47990876 | Missense | c.500T > G | p.V113G | uc001jex.1 | P43 |
| C1D | 10438 | 68127936 | Missense | c.93A > G | p.E4G | uc002sea.2 | P44 |
| CSMD3 | 114788 | 113632265 | Missense | c.4534G > T | p.A1459S | uc003ynu.1 | P44 |
| DUSP15 | 128853 | 29916435 | Missense | c.238A > T | p.D54V | uc002wwu.1 | P44 |
| FASTK | 10922 | 150405196 | Missense | c.1450T > C | p.F451S | uc003wix.1 | P44 |
| HECW1 | 23072 | 43450545 | Missense | c.1854G > A | p.E417K | uc003tid.1 | P44 |
| HSPG2 | 3339 | 22058700 | Missense | c.5282A > C | p.T1748P | uc009vqd.1 | P44 |
| KIAA0649 | 9858 | 137519296 | Frame_Shift_Del | c.3668_3668delG | p.W1040fs | uc004cfr.1 | P44 |
| LRP5L | 91355 | 24087684 | Splice_Site_Del | c.e2_splice_site | | uc003abs.1 | P44 |
| MRPL39 | 54148 | 25881941 | Missense | c.1015C > T | p.T334M | uc002yln.1 | P44 |
| NOSTRIN | 115677 | 169429609 | Missense | c.2333C > A | p.H525Q | uc002uef.1 | P44 |
| NSD1 | 64324 | 176643469 | Missense | c.6223A > G | p.T2029A | uc003mfr.2 | P44 |
| PLCB1 | 23236 | 8613596 | Frame_Shift_Del | c.883_883delG | p.G294fs | uc002wnb.1 | P44 |
| PLXNB1 | 5364 | 48426925 | Missense | c.5522T > A | p.D1821E | uc003csv.1 | P44 |
| PRKD1 | 5587 | 29466373 | In_frame_Ins | c.277_278insTCC | p.32_33insSG | uc001wqh.1 | P44 |
| SCN8A | 6334 | 50431460 | Missense | c.2364C > A | p.T729N | uc001ryw.1 | P44 |
| SEMA6C | 10500 | 149379079 | Missense | c.530C > G | p.A77G | uc001ewv.1 | P44 |
| SLCO4A1 | 28231 | 60758516 | Missense | c.470T > G | p.W89G | uc002ydb.1 | P44 |
| STOX1 | 219736 | 70322473 | Missense | c.2945C > T | p.P982L | uc001joq.1 | P44 |
| ANKRD17 | 26057 | 74229410 | Missense | c.1990C > A | p.H625N | uc003hgp.1 | P45 |
| EPHX3 | 79852 | 15199693 | Missense | c.829G > A | p.R249H | uc002naq.1 | P45 |
| KCNT2 | 343450 | 194494121 | Missense | c.3097A > G | p.K1013E | uc001gtd.1 | P45 |
| WBSCR16 | 81554 | 74127316 | Frame_Shift_Del | c.319_320delGG | p.G65fs | uc003ubr.1 | P45 |
| ZNF496 | 84838 | 245558776 | Missense | c.443G > A | p.D136N | uc009xgv.1 | P45 |
| ADAMTSL1 | 92949 | 18816273 | Missense | c.138T > C | p.F10S | uc003znf.2 | P46 |
| DKK2 | 27123 | 108064750 | Missense | c.1295G > A | p.R197H | uc003hyi.1 | P46 |
| DST | 667 | 56444018 | Missense | c.15795A > G | p.E5092G | uc003pcz.2 | P46 |
| IREB2 | 3658 | 76573361 | Missense | c.2542A > T | p.M794L | uc002bdr.2 | P46 |
| ITGA2B | 3674 | 39805263 | Missense | c.3147G > A | p.E1039K | uc002igt.1 | P46 |
| JTB | 10899 | 152216301 | Missense | c.775T > G | p.W18G | uc001fds.1 | P46 |
| MYD88 | 4615 | 38157341 | Missense | c.773C > T | p.P258L | NM_002468 | P46 |
| OR13C5 | 138799 | 106400824 | Missense | c.692C > T | p.S231L | uc004bcd.1 | P46 |
| PATE2 | 399967 | 125153035 | Missense | c.195C > T | p.S50F | uc001qcu.1 | P46 |
| PTPN3 | 5774 | 111193291 | Missense | c.2077C > T | p.T685I | uc004bed.1 | P46 |
| TLK2 | 11011 | 58033179 | Missense | c.2099A > C | p.1611L | uc010ddp.1 | P46 |
| ZNF182 | 7569 | 47720661 | Missense | c.2115G > T | p.R590I | uc004dir.1 | P46 |
| ZNF253 | 56242 | 19863538 | Missense | c.574C > T | p.T161I | uc002noj.1 | P46 |
| BICD2 | 23299 | 94521305 | Missense | c.1499_1500TC > C | p.L481P | uc004asp.1 | P47 |
| ENPEP | 2028 | 111683440 | Missense | c.2234A > T | p.Y631F | uc003iab.2 | P47 |
| JMJD5 | 79831 | 27133712 | Missense | c.853A > G | p.Q227R | uc002doh.1 | P47 |
| M6PR | 4074 | 8987663 | Splice_Site_SNP | c.e4_splice_site | | uc001qvf.1 | P47 |
| MAPK1 | 5594 | 20490147 | Missense | c.724G > A | p.D162N | uc002zvn.1 | P47 |
| SET | 6418 | 130495886 | Missense | c.921C > T | p.P227L | uc004bvt.2 | P47 |
| SLC6A5 | 9152 | 20624972 | Missense | c.2259T > G | p.C662W | uc001mqd.1 | P47 |
| ZFP37 | 7539 | 114844902 | Missense | c.1845G > A | p.C606Y | uc004bgm.1 | P47 |
| ZNF33B | 7582 | 42409608 | Nonsense | c.911C > T | p.Q266* | uc001jaf.1 | P47 |
| ANK2 | 287 | 114494351 | Frame_Shift_Del | c.5228_5228delG | p.E1710fs | uc003ibe.2 | P48 |
| ATM | 472 | 107627803 | Frame_Shift_Del | c.2022_2022delT | p.L546fs | uc001pkb.1 | P48 |
| BCL9 | 607 | 145558227 | Missense | c.2382G > A | p.G548S | uc001epq.1 | P48 |
| BRCA1 | 672 | 38499191 | Missense | c.2083G > A | p.S628N | uc002ict.1 | P48 |
| CALR | 811 | 12910993 | Missense | c.205T > A | p.F46Y | uc002mvu.1 | P48 |
| INSM2 | 84684 | 35074753 | Missense | c.1755G > T | p.G515V | uc001wth.1 | P48 |
| KATNA1 | 11104 | 149961169 | Missense | c.944A > T | p.Y300F | uc003qmr.1 | P48 |
| OR1L1 | 26737 | 124464474 | Missense | c.809G > T | p.R270I | uc004bms.1 | P48 |
| PC | 5091 | 66374384 | Frame_Shift_Del | c.2883_2883delA | p.P867fs | uc001ojo.1 | P48 |
| PDE6C | 5146 | 95408730 | Missense | c.2257G > A | p.D707N | uc001kiu.2 | P48 |
| SCN10A | 6336 | 38743465 | Missense | c.2723A > G | p.N908S | uc003ciq.1 | P48 |
| SORCS3 | 22986 | 106897468 | Missense | c.1633T > C | p.I469T | uc001kyi.1 | P48 |
| UBE3B | 89910 | 108425285 | Missense | c.1960G > A | p.D453N | uc001top.1 | P48 |
| VIPR2 | 7434 | 158522254 | Missense | c.884C > T | p.A233V | uc003woh.1 | P48 |
| WHSC1L1 | 54904 | 38306379 | Missense | c.1773A > C | p.T419P | uc003xll.1 | P48 |
| ZNF536 | 9745 | 35627300 | Missense | c.1129G > C | p.G331R | uc002nsu.1 | P48 |
| ACSF3 | 197322 | 87694810 | Missense | c.391C > A | p.R74S | uc010cig.1 | P49 |
| C3 | 718 | 6648740 | Missense | c.2568C > A | p.P836T | uc002mfm.1 | P49 |
| CACNA1C | 775 | 2484311 | Frame_Shift_Ins | c.1469_1470insT | p.V386fs | uc009zdu.1 | P49 |
| CPSF1 | 29894 | 145596346 | Missense | c.1174G > C | p.G242A | uc003zck.1 | P49 |
| ENO1 | 2023 | 8854633 | Splice_Site_SNP | c.e3_splice_site | | uc001apj.1 | P49 |
| GPS2 | 2874 | 7156874 | Frame_Shift_Del | c.1172_1172delT | p.F303fs | uc002gfv.1 | P49 |
| LRRC41 | 10489 | 46523911 | Missense | c.1249C > A | p.T402N | uc001cpn.1 | P49 |
| OPRD1 | 4985 | 29062032 | Missense | c.1011C > T | p.R257W | uc001brf.1 | P49 |
| PBRM1 | 55193 | 52638056 | Missense | c.1349A > G | p.D446G | uc003des.2 | P49 |
| PEAR1 | 375033 | 155140344 | Missense | c.118T > C | p.M1T | uc001fqj.1 | P49 |
| PPIL2 | 23759 | 20379227 | Missense | c.1450T > G | p.I445S | uc002zvh.2 | P49 |
| SOD1 | 6647 | 31960703 | Splice_Site_SNP | c.e3_splice_site | | uc002ypa.1 | P49 |
| SPP2 | 6694 | 234624182 | Missense | c.98A > G | p.M5V | uc002vvk.1 | P49 |
| SPTLC3 | 55304 | 13003045 | Missense | c.796C > A | p.N169K | uc002wod.1 | P49 |
| TP53 | 7157 | 7519260 | In_frame_Del | c.587_589delCAA | p.N131del | uc002gim.2 | P49 |
| C5orf4 | 10826 | 154180111 | Missense | c.1169G > A | p.R60H | uc003lvr.1 | P50 |
| DDX46 | 9879 | 134180077 | Missense | c.2663C > G | p.A832G | uc003kzw.1 | P50 |
| FAM83C | 128876 | 33338434 | Missense | c.1680G > A | p.G521E | uc002xca.1 | P50 |
| HMP19 | 51617 | 173467064 | Missense | c.611G > C | p.A156P | uc003mcx.1 | P50 |
| ILF3 | 3609 | 10659203 | Missense | c.1600G > C | p.R50P | uc002mpq.1 | P50 |
| ITGB8 | 3696 | 20410824 | Missense | c.2441A > G | p.E579G | uc003suu.1 | P50 |
| LRRC32 | 2615 | 76049852 | Missense | c.676C > G | p.L145V | uc001oxq.2 | P50 |
| MEI1 | 150365 | 40510635 | Missense | c.3272G > C | p.A1083P | uc003baz.1 | P50 |
| MPL | 4352 | 43591008 | Missense | c.1931T > C | p.L629P | uc001ciw.1 | P50 |
| MUC2 | 4583 | 1083364 | Missense | c.12296C > G | p.T4090S | uc001lsx.1 | P50 |
| MYBPC2 | 4606 | 55655181 | Missense | c.2915C > G | p.A955G | uc002psf.2 | P50 |
| OR10Q1 | 219960 | 57752024 | Missense | c.900G > T | p.K300N | uc001nmp.1 | P50 |
| PTPRD | 5789 | 8426680 | Missense | c.4709G > A | p.S1333N | uc003zkk.1 | P50 |
| SIN3B | 23309 | 16850080 | Missense | c.3151T > G | p.V1046G | uc002ney.1 | P50 |
| SPINK7 | 84651 | 147673154 | Splice_Site_Del | c.e2_splice_site | | uc003lpd.1 | P50 |
| STIM1 | 6786 | 3833690 | Splice_Site_Del | c.e1_splice_site | | uc001lyv.1 | P50 |
| VSIG4 | 11326 | 65159001 | Missense | c.1156C > G | p.C343W | uc004dwh.2 | P50 |
| BCL9 | 607 | 145558977 | Missense | c.3132C > T | p.R798W | uc001epq.1 | P51 |
| CCDC147 | 159686 | 106156524 | Missense | c.2373A > G | p.K747E | uc001kyh.1 | P51 |
| CDH10 | 1008 | 24629205 | Missense | c.484G > A | p.R51H | uc003jgr.1 | P51 |
| CHKB | 1120 | 49364755 | Missense | c.1263A > G | p.Q360R | uc003bms.1 | P51 |
| CLDN5 | 7122 | 17891684 | Missense | c.565T > G | p.V32G | uc002zpu.1 | P51 |
| DAO | 1610 | 107801604 | In_frame_Del | c.1074_1085delCC | p.LRGA255del | uc001tnp.1 | P51 |
| DDX11 | 1663 | 31129252 | Missense | c.814A > G | p.E188G | uc001rjt.1 | P51 |
| DIP2A | 23181 | 46743126 | Missense | c.762G > C | p.A203P | uc002zjo.1 | P51 |
| HAPLN1 | 1404 | 82973138 | Missense | c.1069G > A | p.R333H | uc003kim.1 | P51 |
| HEATR5B | 54497 | 37069379 | Missense | c.5921G > C | p.R1942T | uc002rpp.1 | P51 |
| HEPACAM | 220296 | 124300050 | Missense | c.617C > G | p.L71V | uc001qbk.1 | P51 |
| HSPG2 | 3339 | 22077280 | Missense | c.2714G > C | p.A892P | uc009vqd.1 | P51 |
| KCNK10 | 54207 | 87799346 | Missense | c.560C > T | p.R119W | uc001xwn.1 | P51 |
| KIAA0247 | 9766 | 69195053 | De_novo_Start_OutOfFrame | c.304C > T | | uc001x1k.1 | P51 |
| ME1 | 4199 | 84004180 | Missense | c.1107T > G | p.V334G | uc003pjy.1 | P51 |
| PCDH15 | 65217 | 55257313 | Missense | c.4608C > T | p.R1405C | uc001jju.1 | P51 |
| PDE3A | 5139 | 20413828 | Missense | c.365C > A | p.P115T | uc001reh.1 | P51 |
| PLXNA4 | 91584 | 131538055 | Missense | c.2705T > G | p.C826G | uc003vra.2 | P51 |
| PTCD2 | 79810 | 71651988 | Missense | c.33C > G | p.A8G | uc003kcb.1 | P51 |
| PTPRB | 5787 | 69267212 | Missense | c.2197C > T | p.S718F | uc001swc.2 | P51 |
| RBAK | 57786 | 5070610 | Missense | c.1321A > G | p.T333A | uc010kss.1 | P51 |
| RPS2 | 6187 | 1952610 | Missense | c.786A > G | p.R200G | uc002cnn.2 | P51 |
| SF3B1 | 23451 | 197974856 | Missense | c.2273G > A | p.G742D | uc002uue.1 | P51 |
| STC2 | 8614 | 172677727 | Missense | c.1948C > T | p.S213L | uc003mco.1 | P51 |
| UBASH3B | 84959 | 122165102 | Missense | c.1216A > G | p.M286V | uc001pyi.2 | P51 |
| ZC3H18 | 124245 | 87171123 | Missense | c.238G > T | p.D31Y | uc002fky.1 | P51 |
| ABT1 | 29777 | 26706674 | Missense | c.672G > A | p.R214H | uc003nii.1 | P51 |
| ANO2 | 57101 | 5542803 | Missense | c.2995G > C | p.A975P | uc001qnm.1 | P52 |
| C9orf150 | 286343 | 12811410 | Missense | c.1041G > A | p.R113K | uc003zkw.1 | P52 |
| CECR2 | 27443 | 16411744 | Missense | c.4366G > A | p.A1414T | uc010gqw.1 | P52 |
| ERCC4 | 2072 | 13933620 | Missense | c.1088A > G | p.K360R | uc002dce.2 | P52 |
| FAM160A2 | 84067 | 6189665 | Missense | c.2967C > T | p.R870W | uc001mck.2 | P52 |
| GIGYF2 | 26058 | 233420510 | Missense | c.3999G > C | p.Q1244H | uc002vtj.2 | P52 |
| GNB1 | 2782 | 1727802 | Missense | c.571T > C | p.I80T | uc001aif.1 | P52 |
| HIST1H1E | 3008 | 26264811 | In_frame_Del | c.274_279delGAC | p.DV72del | uc003ngq.1 | P52 |
| KIAA1045 | 23349 | 34962515 | Missense | c.762G > C | p.S184T | uc003zvr.1 | P52 |
| LPHN1 | 22859 | 14131965 | Missense | c.2070C > T | p.R592W | uc002myg.1 | P52 |
| LPHN2 | 23266 | 82225604 | Nonsense | c.3717T > G | p.Y1167* | uc001div.1 | P52 |
| MAGEB4 | 4115 | 30170708 | Missense | c.619G > C | p.V179L | uc004dcb.1 | P52 |
| MON1A | 84315 | 49924022 | Missense | c.783T > C | p.M185T | uc003cxz.1 | P52 |
| MTUS1 | 57509 | 17645436 | Missense | c.2678T > G | p.D748E | uc003wxv.1 | P52 |
| NLGN3 | 54413 | 70300751 | Missense | c.1005G > A | p.G234D | uc004dzb.1 | P52 |
| NLRP3 | 114548 | 245653155 | Missense | c.405G > T | p.G95V | uc001icr.1 | P52 |
| OBSL1 | 23363 | 220136503 | Missense | c.2555G > T | p.R833L | uc010fwk.1 | P52 |
| OLFML2A | 169611 | 126612507 | Missense | c.2067G > A | p.V652I | uc004bov.1 | P52 |
| RFTN1 | 23180 | 16394263 | Missense | c.1074C > A | p.N264K | uc003cay.1 | P52 |
| SI | 6476 | 166247361 | Missense | c.1911A > C | p.T617P | uc003fei.1 | P52 |
| SLC24A3 | 57419 | 19612921 | Missense | c.1200A > C | p.T335P | uc002wrl.1 | P52 |
| TADA2B | 93624 | 7106703 | Missense | c.435C > G | p.A95G | uc003gjw.2 | P52 |
| TANC1 | 85461 | 159662530 | Missense | c.471C > T | p.S66F | uc002uag.1 | P52 |
| TAS1R1 | 80835 | 6562125 | Missense | c.2420A > G | p.Y807C | uc001ant.1 | P52 |
| TLR8 | 51311 | 12848204 | Missense | c.1329G > T | p.R393I | uc004cvd.1 | P52 |
| TMEM45A | 55076 | 101758306 | Missense | c.450A > G | p.E84G | uc003dua.1 | P52 |
| VGLL1 | 51442 | 135458735 | Missense | c.706C > A | p.A179D | uc004ezy.1 | P52 |
| ZFP64 | 55734 | 50134673 | Missense | c.2117G > A | p.V590I | uc002xwk.1 | P52 |
| ZHX1 | 11244 | 124336389 | Frame_Shift_Del | c.1409_1409delC | p.Q327fs | uc003yqe.1 | P52 |
| AK1 | 203 | 129674895 | Nonsense | c.255C > A | p.Y34* | uc004bsm.2 | P53 |
| ATP6V1A | 523 | 114991359 | Missense | c.1036G > A | p.E324K | uc003eao.1 | P53 |
| CAMK1G | 57172 | 207852798 | Missense | c.1488C > G | p.S462R | uc001hhd.1 | P53 |
| CUL7 | 9820 | 43114581 | Missense | c.4720C > A | p.L1473M | uc003otq.1 | P53 |
| DCAF8 | 50717 | 158476198 | Missense | c.809C > G | p.S212R | uc001fvn.1 | P53 |
| DLG1 | 1739 | 198279777 | Missense | c.1422C > G | p.C386W | uc003fxm.2 | P53 |
| FAM71E1 | 112703 | 55662825 | Missense | c.971A > C | p.T205P | uc002psh.1 | P53 |
| GAK | 2580 | 874327 | Nonsense | c.1273C > A | p.Y358* | uc003gbm.2 | P53 |
| GTF2H1 | 2965 | 18336153 | Missense | c.1499C > A | p.Q447K | uc001moh.1 | P53 |
| NEK10 | 152110 | 27328660 | Missense | c.776G > T | p.V168L | uc003cdt.1 | P53 |
| SHB | 6461 | 37964819 | Missense | c.1422A > G | p.E285G | uc004aax.1 | P53 |
| SNX1 | 6642 | 62213964 | Missense | c.1306C > A | p.Q424K | uc002amv.1 | P53 |
| TLN2 | 83660 | 60898861 | Missense | c.6865G > A | p.E2289K | uc002alb.2 | P53 |
| TMCO4 | 255104 | 19979805 | Missense | c.276C > G | p.P12A | uc001bcn.1 | P53 |
| TTF1 | 7270 | 134257325 | Missense | c.1998G > T | p.S649I | uc004cbl.1 | P53 |
| UBR4 | 23352 | 19288057 | Missense | c.14217T > G | p.V4738G | uc001bbi.1 | P53 |
| ULK4 | 54986 | 41263441 | Missense | c.4012C > A | p.Q1271K | uc003ckv.2 | P53 |
| WHSC1L1 | 54904 | 38308135 | Missense | c.1554C > A | p.Q346K | uc003xli.1 | P53 |
| ZNF628 | 89887 | 60686239 | Missense | c.2420A > C | p.T619P | uc002qld.2 | P53 |
| ALG1 | 56052 | 5073760 | Splice_Site_SNP | c.e12_splice_site | | uc002cyn.1 | P54 |
| ANK3 | 288 | 61505733 | Missense | c.5104T > C | p.S1638P | uc001jky.1 | P54 |
| ANKRD30A | 91074 | 37461178 | Missense | c.446G > A | p.S116N | uc001iza.1 | P54 |
| ANO6 | 196527 | 44068315 | Missense | c.1472G > T | p.A424S | uc001roo.1 | P54 |
| ASPM | 259266 | 195382133 | Missense | c.155C > G | p.P20A | uc001gtu.1 | P54 |
| ATF2 | 1386 | 175690980 | Missense | c.693C > T | p.T144I | uc002ujl.1 | P54 |
| BEND2 | 139105 | 18131898 | Missense | c.705C > G | p.P184R | uc004cyj.2 | P54 |
| C4orf39 | 152756 | 166097930 | Missense | c.381C > G | p.S102R | uc003iqx.1 | P54 |
| C9orf152 | 401546 | 112009610 | Missense | c.625A > G | p.E3G | uc004beo.2 | P54 |
| CD163L1 | 283316 | 7440251 | Missense | c.1783T > G | p.V586G | uc001qsy.1 | P54 |
| CDCA2 | 157313 | 25381826 | Missense | c.1194A > G | p.T239A | uc003xep.1 | P54 |
| COL1A2 | 1278 | 93892413 | Missense | c.3193C > T | p.P908S | uc003ung.1 | P54 |
| CYP4V2 | 285440 | 187359341 | Missense | c.1142G > A | p.E280K | uc003iyw.2 | P54 |
| DBN1 | 1627 | 176817699 | Missense | c.2030C > G | p.T583S | uc003mgx.2 | P54 |
| FAM129B | 64855 | 129327247 | Missense | c.534T > G | p.V111G | uc004brh.1 | P54 |
| FAM83B | 222584 | 54913393 | Missense | c.1781A > C | p.E555D | uc003pck.1 | P54 |
| GDAP2 | 54834 | 118264329 | Missense | c.377T > C | p.W59R | uc001ehf.1 | P54 |
| GPATCH8 | 23131 | 39832053 | Missense | c.2982G > A | p.R973K | uc002igw.1 | P54 |
| GPR135 | 64582 | 59000331 | Missense | c.1482A > G | p.D456G | uc010apj.1 | P54 |
| HPSE2 | 60495 | 100364751 | Missense | c.1280T > C | p.L407S | uc001kpn.1 | P54 |
| IQSEC2 | 23096 | 53296786 | Missense | c.1898G > T | p.G566V | uc004dsd.1 | P54 |
| IRS4 | 8471 | 107864076 | Missense | c.2233C > A | p.P719T | uc004eoc.1 | P54 |
| JPH4 | 84502 | 23109985 | Missense | c.2572G > C | p.A599P | uc001wkr.1 | P54 |
| KIAA1467 | 57613 | 13100124 | Missense | c.433G > C | p.S137T | uc001rbi.1 | P54 |
| MIA3 | 375056 | 220867582 | Missense | c.406C > T | p.H133Y | uc001hnl.1 | P54 |
| NRG1 | 3084 | 32740945 | Missense | c.1913G > T | p.S474I | uc003xiu.1 | P54 |
| ORMDL2 | 29095 | 54499049 | Splice_Site_SNP | c.e2_splice_site | | uc001shw.1 | P54 |
| OTOF | 9381 | 26555972 | Missense | c.2093C > T | p.R656W | uc002rhk.1 | P54 |
| PLOD1 | 5351 | 11937535 | Nonsense | c.732T > A | p.L214* | uc001atm.1 | P54 |
| WDR78 | 79819 | 67071951 | Missense | c.1974G > C | p.A640P | uc001dcx.1 | P54 |
| ZNF155 | 7711 | 49187582 | Nonsense | c.263G > T | p.E20* | uc002oxy.1 | P54 |
| 8-Sep | 23176 | 132122134 | Missense | c.1538T > G | p.S434A | uc003kxu.2 | P55 |
| ACBD3 | 64746 | 224413665 | Missense | c.793C > G | p.A249G | uc001hpy.1 | P55 |
| ADCY5 | 111 | 124504619 | Missense | c.2697C > G | p.S899R | uc003egh.1 | P55 |
| AOC3 | 8639 | 38260200 | Missense | c.1970C > T | p.R604C | uc002ibv.1 | P55 |
| ARHGEF1 | 9138 | 47091286 | Missense | c.937G > A | p.R283Q | uc002osb.1 | P55 |
| ARHGEF2 | 9181 | 154194296 | Missense | c.1934A > G | p.D560G | uc001fmu.1 | P55 |
| BCOR | 54880 | 39806972 | Nonsense | c.4436G > T | p.E1382* | uc004den.2 | P55 |
| C17orf64 | 124773 | 55861506 | Missense | c.512T > G | p.V34G | uc002iyq.1 | P55 |
| C6orf27 | 80737 | 31844806 | Missense | c.1709G > C | p.A491P | uc003nxb.2 | P55 |
| CD6 | 923 | 60533671 | Missense | c.997T > G | p.V278G | uc001nqq.1 | P55 |
| CHD2 | 1106 | 91300817 | Missense | c.2509C > T | p.T645M | uc002bsp.1 | P55 |
| DUOX2 | 50506 | 43191311 | Missense | c.663A > G | p.R154G | uc010bea.1 | P55 |
| EGFL8 | 80864 | 32243180 | Missense | c.782A > G | p.E226G | uc003oac.1 | P55 |
| EGFR | 1956 | 55191052 | Missense | c.1171C > G | p.R309G | uc003tqk.1 | P55 |
| EPHB6 | 2051 | 142274181 | Missense | c.2234A > C | p.T468P | uc003wbq.1 | P55 |
| FAM120A | 23196 | 95329296 | Missense | c.1482G > A | p.G486E | uc004atw.1 | P55 |
| FCGBP | 8857 | 45057933 | Missense | c.14149C > A | p.T4714N | uc002omp.2 | P55 |
| FRMD7 | 90167 | 131055783 | Missense | c.528G > A | p.C117Y | uc004ewn.1 | P55 |
| FRYL | 285527 | 48206847 | Missense | c.8985G > A | p.E2794K | uc003gyh.1 | P55 |
| GJB1 | 2705 | 70360607 | Nonsense | c.420G > T | p.E109* | uc004dzf.2 | P55 |
| GLB1 | 2720 | 33074745 | Missense | c.690C > G | p.S191R | uc003cfi.1 | P55 |
| GRIN2C | 2905 | 70354510 | Missense | c.2302A > C | p.T716P | uc002jlt.1 | P55 |
| GUCY1A3 | 2982 | 156870986 | Splice_Site_Del | c.e11_splice_site | | uc003iov.1 | P55 |
| HAS3 | 3038 | 67705822 | Missense | c.1038G > C | p.A272P | uc010cfh.1 | P55 |
| HCN3 | 57657 | 153521711 | Missense | c.1229C > G | p.S407R | uc001fjz.1 | P55 |
| HOXA11 | 3207 | 27190885 | Missense | c.476T > C | p.V135A | uc003syx.1 | P55 |
| KRAS | 3845 | 25289548 | Missense | c.219G > A | p.G13D | uc001rgp.1 | P55 |
| LRBA | 987 | 151946990 | Nonsense | c.5875G > T | p.E1801* | uc010ipj.1 | P55 |
| PODNL1 | 79883 | 13904594 | Missense | c.1737T > G | p.V488G | uc002mxr.1 | P55 |
| REPIN1 | 29803 | 149700156 | Missense | c.1257G > C | p.G355A | uc010lpr.1 | P55 |
| SFT2D1 | 113402 | 166663046 | Missense | c.202C > T | p.P58S | uc003qux.1 | P55 |
| SLC24A6 | 80024 | 112228736 | Missense | c.1649G > C | p.R480P | uc001tvc.1 | P55 |
| STOML2 | 30968 | 35092804 | Missense | c.125C > T | p.S21F | uc003zwi.1 | P55 |
| UNC5D | 137970 | 35660758 | Missense | c.1050G > A | p.R241K | uc003xjr.1 | P55 |
| C16orf93 | 90835 | 30676404 | Missense | c.1416T > G | p.V362G | uc002dzn.1 | P56 |
| EPHA7 | 2045 | 94013300 | Missense | c.2870T > G | p.I886R | uc003poe.1 | P56 |
| EXOC4 | 60412 | 133230962 | Missense | c.1840A > G | p.D602G | uc003vrk.1 | P56 |
| PKD1L1 | 168507 | 47863826 | Missense | c.4492C > T | p.H1498Y | uc003tny.1 | P56 |
| RBM28 | 55131 | 127767030 | Missense | c.285A > T | p.D57V | uc003vmp.2 | P56 |
| SPEF2 | 79925 | 35828295 | Missense | c.4655C > T | p.T1515I | uc003jjo.1 | P56 |
| SYCP1 | 6847 | 115254564 | Nonsense | c.1553T > G | p.Y448* | uc001efr.1 | P56 |
| SYNE1 | 23345 | 152597570 | Missense | c.21057G > A | p.E6819K | uc010kiw.1 | P56 |
| TMEM67 | 91147 | 94869261 | Missense | c.1476C > T | p.P466S | uc003ygd.2 | P56 |
| TRAK2 | 66008 | 201957085 | Missense | c.2509C > T | p.T688I | uc002uyb.2 | P56 |
| ACTB | 60 | 5535517 | Missense | c.200G > C | p.G55A | uc003sos.2 | P57 |
| C5 | 727 | 122784822 | Missense | c.3352G > A | p.V1108I | uc004bkv.1 | P57 |
| C9orf98 | 158067 | 134688446 | Missense | c.1412G > A | p.A286T | uc004cbu.1 | P57 |
| DTX2 | 113878 | 75950336 | Missense | c.1400T > C | p.S282P | uc003uff.2 | P57 |
| FAM47A | 158724 | 34059857 | Missense | c.493G > T | p.D154Y | uc004ddg.1 | P57 |
| GTPBP8 | 29083 | 114192654 | Missense | c.165C > G | p.P40A | uc003dzn.1 | P57 |
| MTERFD3 | 80298 | 105895678 | Missense | c.2764G > T | p.Q315H | uc001tme.1 | P57 |
| NAA40 | 79829 | 63478517 | Missense | c.831G > C | p.C235S | uc009yoz.1 | P57 |
| ODF2L | 57489 | 86625253 | Missense | c.393T > G | p.C16G | uc001dln.1 | P57 |
| PKD1 | 5310 | 2100723 | Missense | c.4655G > C | p.Q1482H | uc002cos.1 | P57 |
| PLEKHG3 | 26030 | 64268907 | Missense | c.1491G > T | p.A408S | uc001xho.1 | P57 |
| PRKG2 | 5593 | 82293862 | Missense | c.964G > T | p.G317V | uc003hmh.1 | P57 |
| PTAFR | 5724 | 28349788 | Missense | c.459T > G | p.I111S | uc001bpl.1 | P57 |
| RPGR | 6103 | 38030527 | In_frame_Del | c.2835_2837delG | p.889_890EE > E | uc004ded.1 | P57 |
| SMC1A | 8243 | 53439984 | Missense | c.2819C > A | p.T917N | uc004dsg.1 | P57 |
| SON | 6651 | 33849541 | Missense | c.6183G > C | p.R2045T | uc002yse.1 | P57 |
| TFR2 | 7036 | 100066571 | Missense | c.1188A > G | p.S383G | uc003uvv.1 | P57 |
| TP63 | 8626 | 191069813 | Missense | c.1225G > A | p.R379H | uc003fry.2 | P57 |
| TTC7B | 145567 | 90225656 | Missense | c.1053C > T | p.R311C | uc001xyp.1 | P57 |
| XIRP2 | 129446 | 167823940 | Missense | c.2826G > A | p.V913I | uc010fpn.1 | P57 |
| XKR5 | 389610 | 6666955 | Missense | c.675T > G | p.V218G | uc003wqp.1 | P57 |
| ATP8A2 | 51761 | 25015445 | Missense | c.938C > T | p.P266S | uc001uqk.1 | P58 |
| CDC14B | 8555 | 98324609 | Missense | c.1795C > G | p.T448R | uc004awj.1 | P58 |
| CELF4 | 56853 | 33109144 | Missense | c.905G > A | p.R170H | uc002lae.2 | P58 |
| CYB5R4 | 51167 | 84687597 | Missense | c.774A > T | p.L214F | uc003pkf.1 | P58 |
| DAB2 | 1601 | 39411864 | Missense | c.2770C > A | p.Q747K | uc003jlx.2 | P58 |
| DNER | 92737 | 229980218 | Missense | c.1844C > T | p.T566M | uc002vpv.1 | P58 |
| GATA5 | 140628 | 60473859 | Missense | c.1032G > C | p.A324P | uc002ycx.1 | P58 |
| GCNT4 | 51301 | 74361401 | Missense | c.1079G > C | p.C73S | uc003kdn.1 | P58 |
| IMPG1 | 3617 | 76771906 | Missense | c.1083C > T | p.P318L | uc003pik.1 | P58 |
| MLL5 | 55904 | 104539509 | Missense | c.4604C > T | p.P1357L | uc003vcm.1 | P58 |
| MNS1 | 55329 | 54510964 | Missense | c.1459T > G | p.L432V | uc002adr.1 | P58 |
| MYC | 4609 | 128819862 | Missense | c.741A > G | p.T73A | uc003ysi.1 | P58 |
| MYO9A | 4649 | 69957617 | Missense | c.6222G > A | p.G1917R | uc002atl.2 | P58 |
| PREPL | 9581 | 44413214 | Missense | c.1276C > T | p.P414L | uc002ruf.1 | P58 |
| SF3B1 | 23451 | 197974958 | Missense | c.2267G > A | p.G740E | uc002uue.1 | P58 |
| SREBF1 | 6720 | 17663713 | Missense | c.859C > T | p.S222F | uc002grt.1 | P58 |
| SRRM3 | 222183 | 75732067 | Missense | c.932G > C | p.K241N | uc003uer.2 | P58 |
| 8-Sep | 23176 | 132122134 | Missense | c.1538T > G | p.S434A | uc003kxu.2 | P59 |
| ABCC9 | 10060 | 21980741 | Missense | c.155G > T | p.L45F | uc001rfh.1 | P59 |
| ACACB | 32 | 108174243 | Missense | c.5919C > G | p.R1934G | uc001tob.1 | P59 |
| ADH1C | 126 | 100479799 | Missense | c.1146A > G | p.E354G | uc003huu.1 | P59 |
| ALS2 | 57679 | 202278388 | Missense | c.4821A > C | p.K1541T | uc002uyo.1 | P59 |
| AMBN | 258 | 71497342 | Missense | c.197G > A | p.S41N | uc003hfl.1 | P59 |
| ARAP3 | 64411 | 141021488 | Missense | c.3144C > G | p.C1022W | uc003llm.1 | P59 |
| ASPM | 259266 | 195364414 | Missense | c.2862G > A | p.S922N | uc001gtu.1 | P59 |
| ATXN7L3 | 56970 | 39630128 | Missense | c.441A > C | p.N117T | uc002ifz.1 | P59 |
| BAT2L1 | 84726 | 133342991 | Missense | c.4501C > G | p.C1482W | uc004can.2 | P59 |
| C10orf2 | 56652 | 102738034 | Missense | c.733G > C | p.G26A | uc001ksf.1 | P59 |
| C16orf7 | 9605 | 88303277 | Missense | c.1581A > C | p.T486P | uc002fom.1 | P59 |
| C16orf79 | 283870 | 2199695 | Missense | c.629G > T | p.W151L | uc010bsh.1 | P59 |
| CADM2 | 253559 | 86093417 | Missense | c.879T > A | p.N293K | uc003dql.1 | P59 |
| CADM2 | 253559 | 86197508 | Missense | c.1133T > G | p.V378G | uc003dql.1 | P59 |
| CCDC27 | 148870 | 3670243 | Missense | c.1519C > A | p.Q479K | uc001akv.1 | P59 |
| CDHR5 | 53841 | 608063 | Missense | c.2114C > G | p.A670G | uc001lqj.1 | P59 |
| CDK17 | 5128 | 95241998 | Missense | c.631C > G | p.P48A | uc001tep.1 | P59 |
| COBL | 23242 | 51255118 | Missense | c.244G > C | p.R20P | uc003tpr.2 | P59 |
| COL5A1 | 1289 | 136806558 | Nonsense | c.2746C > A | p.Y788* | uc004cfe.1 | P59 |
| CSRP2BP | 57325 | 18071545 | Missense | c.863C > A | p.Q81K | uc002wqj.1 | P59 |
| DAZAP1 | 26528 | 1385835 | Missense | c.1337G > C | p.G383A | uc002lsn.1 | P59 |
| DSCAM | 1826 | 40387535 | Missense | c.4285T > G | p.V1278G | uc002yyq.1 | P59 |
| ERBB2IP | 55914 | 65410018 | Nonsense | c.4209C > A | p.Y1384* | uc010iwx.1 | P59 |
| FAM84B | 157638 | 127638104 | Missense | c.997G > C | p.R238P | uc003yrz.1 | P59 |
| FGF3 | 2248 | 69334469 | Missense | c.996A > C | p.T169P | uc001oph.1 | P59 |
| FZD5 | 7855 | 208341571 | Nonsense | c.548C > A | p.Y46* | uc002vcj.1 | P59 |
| GRPEL1 | 80273 | 7113630 | Missense | c.555C > T | p.P172S | uc003gjy.1 | P59 |
| HEATR7B2 | 133558 | 41054271 | Missense | c.3182G > T | p.V898F | uc003jmj.2 | P59 |
| HIST1H1T | 3010 | 26216200 | Missense | c.144G > C | p.S34T | uc003ngj.1 | P59 |
| HMG20A | 10363 | 75557872 | Missense | c.1073T > G | p.V291G | uc002bcr.1 | P59 |
| INSL3 | 3640 | 17788847 | Missense | c.312A > G | p.R103G | uc010ebf.1 | P59 |
| ITGA10 | 8515 | 144239962 | Missense | c.478C > G | p.S134R | uc001eoa.1 | P59 |
| ITGAX | 3687 | 31298601 | Missense | c.2958A > C | p.D964A | uc002ebt.2 | P59 |
| KCNK15 | 60598 | 42808189 | Missense | c.288G > C | p.G75A | uc002xmr.1 | P59 |
| KIAA1267 | 284058 | 41472921 | Missense | c.2282A > C | p.T733P | uc002lkb.1 | P59 |
| LANCL3 | 347404 | 37403650 | Missense | c.1016G > T | p.L238F | uc004ddp.1 | P59 |
| LMTK2 | 22853 | 97661455 | Missense | c.4035T > C | p.S1248P | uc003upd.1 | P59 |
| MAPK7 | 5598 | 19224729 | Missense | c.968G > C | p.R205P | uc002gvn.1 | P59 |
| MLPH | 79083 | 238125802 | Missense | c.1986G > C | p.A587P | uc002vwt.1 | P59 |
| MYH4 | 4622 | 10308535 | Missense | c.738A > C | p.E209D | uc002gmn.1 | P59 |
| MYOM1 | 8736 | 3119302 | Missense | c.3056A > C | p.T908P | uc002klp.1 | P59 |
| NANOS3 | 342977 | 13849199 | Missense | c.250T > G | p.L46R | uc002mxj.2 | P59 |
| NUP160 | 23279 | 47813840 | Missense | c.1125C > T | p.A347V | uc001ngm.1 | P59 |
| OBSCN | 84033 | 226529063 | Missense | c.5895G > C | p.A1951P | uc009xez.1 | P59 |
| PCDHGB7 | 56099 | 140777657 | Missense | c.192T > G | p.V16G | uc003lkn.1 | P59 |
| PITPNM3 | 83394 | 6316797 | Missense | c.1484G > C | p.E445Q | uc002gdd.2 | P59 |
| PPP1R12C | 54776 | 60315715 | Missense | c.519A > C | p.D168A | uc002qix.1 | P59 |
| PPP1R9A | 55607 | 94741195 | Missense | c.3455T > G | p.V1058G | uc010lfj.1 | P59 |
| PPT2 | 9374 | 32230457 | Nonsense | c.229C > A | p.Y42* | uc003nzw.1 | P59 |
| PSD | 5662 | 104162257 | Missense | c.2146C > G | p.A540G | uc001kvg.1 | P59 |
| PTCH2 | 8643 | 45065524 | Missense | c.2428A > C | p.T806P | uc001cms.1 | P59 |
| PTPRB | 5787 | 69220938 | Missense | c.5605T > G | p.V1854G | uc001swc.2 | P59 |
| RALGPS2 | 55103 | 177120882 | Missense | c.1294C > G | p.A318G | uc001glz.1 | P59 |
| RBM4B | 83759 | 66193265 | Missense | c.1155C > G | p.C162W | uc001oja.1 | P59 |
| RELT | 84957 | 72783321 | Missense | c.1105G > C | p.A314P | uc001otv.1 | P59 |
| RFX2 | 5990 | 5967270 | Missense | c.769T > G | p.L204V | uc002meb.1 | P59 |
| RNF152 | 220441 | 57634248 | Missense | c.841A > T | p.Q143H | uc002llh.1 | P59 |
| SCML4 | 256380 | 108174684 | Missense | c.640T > G | p.V130G | uc010kdf.1 | P59 |
| SERINC2 | 347735 | 31678427 | Missense | c.1190T > G | p.V347G | uc001bst.1 | P59 |
| SETD5 | 55209 | 9445684 | Missense | c.497C > G | p.A21G | uc003brt.1 | P59 |
| SETD8 | 387893 | 122458184 | Missense | c.1082A > C | p.H347P | uc001uew.1 | P59 |
| SF3B1 | 23451 | 197975079 | Missense | c.2146A > G | p.K700E | uc002uue.1 | P59 |
| SLC35B1 | 10237 | 45140157 | Missense | c.125G > C | p.R13P | uc002iph.1 | P59 |
| SPATS2 | 65244 | 48204929 | Missense | c.2298T > C | p.Y437H | uc001rud.2 | P59 |
| SSPO | 23145 | 149146122 | Splice_Site_Del | c.e81_splice_site | | uc010lpk.1 | P59 |
| SYNE2 | 23224 | 63520215 | Missense | c.2239A > C | p.N670T | uc001xgl.1 | P59 |
| TAF6L | 10629 | 62306382 | Missense | c.929G > T | p.W276C | uc009yof.1 | P59 |
| THSD7B | 80731 | 137879814 | Missense | c.2569G > A | p.E857K | uc002tva.1 | P59 |
| TIMD4 | 91937 | 156279126 | Missense | c.1114G > A | p.D353N | uc003lwh.1 | P59 |
| TM4SF19 | 116211 | 197538250 | Missense | c.377C > G | p.C84W | uc010iad.1 | P59 |
| TMPRSS12 | 283471 | 49523108 | Missense | c.141G > C | p.G32R | uc001rwx.2 | P59 |
| UCN3 | 114131 | 5406125 | Missense | c.666G > C | p.A148P | uc001ihx.1 | P59 |
| USP39 | 10713 | 85699890 | Missense | c.341G > C | p.S102T | uc002sqe.2 | P59 |
| WNT10A | 80326 | 219455261 | Missense | c.711C > G | p.A83G | uc002vjd.1 | P59 |
| WWC2 | 80014 | 184419542 | Missense | c.1954G > T | p.S591I | uc010irx.1 | P59 |
| ZC3H18 | 124245 | 87171086 | Missense | c.201G > C | p.E18D | uc002fky.1 | P59 |
| ZNF264 | 9422 | 62408622 | Missense | c.619G > A | p.G69E | uc002qob.1 | P59 |
| CAPRIN1 | 4076 | 34030556 | Missense | c.202A > C | p.T5P | uc001mvh.1 | P60 |
| CHST11 | 50515 | 103675235 | Missense | c.878G > C | p.A195P | uc001tkx.1 | P60 |
| CLCN3 | 1182 | 170793687 | Nonsense | c.542C > A | p.Y11* | uc003ish.1 | P60 |
| CNN1 | 1264 | 11521223 | Missense | c.751A > C | p.D196A | uc002msc.1 | P60 |
| COL5A3 | 50509 | 9938022 | Missense | c.4836A > C | p.T1584P | uc002mmq.1 | P60 |
| CUL1 | 8454 | 148094596 | Missense | c.1326G > C | p.R267P | uc010lpg.1 | P60 |
| DGKH | 160851 | 41632171 | Nonsense | c.775G > T | p.E252* | uc001uyl.1 | P60 |
| FLI1 | 2313 | 128133281 | Missense | c.252C > G | p.A27G | uc001qem.1 | P60 |
| KDM5D | 8284 | 20360855 | Missense | c.891C > A | p.Q202K | uc004fug.1 | P60 |
| KIF2C | 11004 | 45005103 | Nonsense | c.2105G > T | p.E664* | uc001cmg.2 | P60 |
| KRTAP19-5 | 337972 | 30796183 | Missense | c.97C > T | p.R33C | uc002yoi.1 | P60 |
| LANCL1 | 10314 | 211028170 | Missense | c.417A > G | p.T105A | uc002ved.1 | P60 |
| LGALS8 | 3964 | 234768842 | Missense | c.375C > G | p.R59G | uc001hxw.1 | P60 |
| LOXL2 | 4017 | 23273567 | Missense | c.851C > T | p.S171L | uc003xdh.1 | P60 |
| MAPK14 | 1432 | 36103947 | Missense | c.397A > G | p.E12G | uc003olp.1 | P60 |
| MPDZ | 8777 | 13098980 | Missense | c.5985C > G | p.S1978R | uc010mhy.1 | P60 |
| MUC2 | 4583 | 1083069 | Missense | c.12001A > G | p.T3992A | uc001lsx.1 | P60 |
| NLGN2 | 57555 | 7260977 | Missense | c.1716A > C | p.N548T | uc002ggt.1 | P60 |
| NUP98 | 4928 | 3722354 | Missense | c.1660A > C | p.T457P | uc001lyh.1 | P60 |
| ODZ2 | 57451 | 167554855 | Nonsense | c.2850C > A | p.Y950* | uc010jjd.1 | P60 |
| PIGT | 51604 | 43487690 | Missense | c.1620A > C | p.N516T | uc002xoh.1 | P60 |
| PPP2R2C | 5522 | 6431145 | Missense | c.248G > C | p.S75T | uc003gja.1 | P60 |
| ROR2 | 4920 | 93526125 | Nonsense | c.2671C > A | p.Y824* | uc004arj.1 | P60 |
| SCYL2 | 55681 | 99209422 | Missense | c.238T > G | p.V63G | uc001thn.1 | P60 |
| SF3B1 | 23451 | 197975728 | Missense | c.1922G > T | p.R625L | uc002uue.1 | P60 |
| TBC1D25 | 4943 | 48288244 | Missense | c.388G > A | p.G93R | uc004dka.1 | P60 |
| VWC2 | 375567 | 49812926 | Missense | c.1326C > T | p.T257M | uc003tot.1 | P60 |
| ZNF330 | 27309 | 142373133 | Missense | c.795G > T | p.C192F | uc003iiq.2 | P60 |
| 10-Sep | 151011 | 109659184 | Missense | c.1818T > C | p.I480T | uc002tey.1 | P61 |
| ATM | 472 | 107626804 | Frame_Shift_Del | c.1787_1788delAA | p.K468fs | uc001pkb.1 | P61 |
| BPIL1 | 80341 | 31069818 | Splice_Site_Del | c.e8_splice_site | | uc002wyj.1 | P61 |
| C18orf8 | 29919 | 19364517 | Missense | c.1958T > C | p.F613L | uc010dlt.1 | P61 |
| CDK5R2 | 8941 | 219533742 | Missense | c.1101C > A | p.T319K | uc002vjf.1 | P61 |
| CES1 | 1066 | 54410992 | Nonsense | c.970C > T | p.R288* | uc002eil.1 | P61 |
| GPR162 | 27239 | 6803460 | Missense | c.670C > A | p.H45Q | uc001qqw.1 | P61 |
| HAPLN4 | 404037 | 19229935 | Frame_Shift_Del | c.918_919delTG | p.V300fs | uc002nmb.1 | P61 |
| IMP3 | 55272 | 73719132 | Missense | c.1377G > C | p.D145H | uc002bat.2 | P61 |
| MICALCL | 84953 | 12328035 | Nonsense | c.2095C > T | p.R602* | uc001mkg.1 | P61 |
| MKRN3 | 7681 | 21362042 | Missense | c.496C > T | p.P7L | uc001ywh.2 | P61 |
| SF3B1 | 23451 | 197975079 | Missense | c.2146A > G | p.K700E | uc002uue.1 | P61 |
| SLC6A5 | 9152 | 20632917 | Missense | c.2594T > A | p.I774N | uc001mqd.1 | P61 |
| SPOCK1 | 6695 | 136342286 | Missense | c.1467G > C | p.D426H | uc003lbo.1 | P61 |
| SPP2 | 6694 | 234632271 | Missense | c.348G > A | p.R88Q | uc002vvk.1 | P61 |
| ZNF527 | 84503 | 42571176 | Missense | c.496G > A | p.A129T | uc010efk.1 | P61 |
| ALMS1 | 7840 | 73466540 | In_frame_Del | c.147_152delGGA | p.EE27del | uc002sje.1 | P62 |
| DUOX2 | 50506 | 43190176 | Missense | c.1110C > G | p.P303A | uc010bea.1 | P62 |
| RFT1 | 91869 | 53113134 | Missense | c.1024C > G | p.A326G | uc003dgj.1 | P62 |
| TP53 | 7157 | 7517846 | Missense | c.1011C > T | p.R273C | uc002gim.2 | P62 |
| ABRA | 137735 | 107851012 | Missense | c.637G > A | p.G195S | uc003ymm.2 | P63 |
| APAF1 | 317 | 97595380 | Missense | c.2417C > G | p.H614D | uc001tfz.1 | P63 |
| C9orf86 | 55684 | 138853337 | Missense | c.1896C > G | p.A480G | uc004cjj.1 | P63 |
| COL4A2 | 1284 | 109956830 | Nonsense | c.4759C > A | p.Y1490* | uc001vqx.1 | P63 |
| CSMD3 | 114788 | 113632184 | Missense | c.4615A > T | p.S1486C | uc003ynu.1 | P63 |
| DSG4 | 147409 | 27226246 | Missense | c.1085G > T | p.W317L | uc002kwr.1 | P63 |
| GAS2L1 | 10634 | 28034328 | Missense | c.432C > G | p.A78G | uc003afa.1 | P63 |
| GPR113 | 165082 | 26390905 | Missense | c.1015G > C | p.R338P | uc002rhe.2 | P63 |
| GPR135 | 64582 | 59001142 | Missense | c.671G > C | p.A186P | uc010apj.1 | P63 |
| GPR172A | 79581 | 145554721 | Missense | c.918C > T | p.P254L | uc003zcc.1 | P63 |
| GRM3 | 2913 | 86253850 | Missense | c.1905C > T | p.A269V | uc003uid.1 | P63 |
| KRT26 | 353288 | 36181001 | Missense | c.501C > T | p.T152I | uc002hvf.1 | P63 |
| LRP1 | 4035 | 55876248 | Missense | c.9279G > C | p.G2938A | uc001snd.1 | P63 |
| MRM1 | 79922 | 32032797 | Missense | c.660G > C | p.V149L | uc002hne.1 | P63 |
| PRPF8 | 10594 | 1524616 | Missense | c.3283C > T | p.R1057W | uc002fte.1 | P63 |
| RBBP6 | 5930 | 24480693 | Frame_Shift_Del | c.2039_2039delA | p.R333fs | uc002dmh.1 | P63 |
| RLTPR | 146206 | 66238135 | Frame_Shift_Del | c.604_604delT | p.Y162fs | uc002etn.1 | P63 |
| SEMA6D | 80031 | 45848127 | Missense | c.2183C > A | p.P608H | uc010bek.1 | P63 |
| THBD | 7056 | 22977192 | Missense | c.1110C > G | p.A317G | uc002wss.1 | P63 |
| ZNF449 | 203523 | 134308854 | Frame_Shift_Del | c.285_285delA | p.N49fs | uc004eys.1 | P63 |
| ANKRD13B | 124930 | 24959220 | Missense | c.454C > G | p.A114G | uc002hei.1 | P64 |
| ANP32D | 23519 | 47152794 | Missense | c.80G > A | p.S27N | uc001rrq.1 | P64 |
| DLAT | 1737 | 111419435 | Missense | c.1824A > G | p.I389V | uc001pmo.2 | P64 |
| DUSP27 | 92235 | 165353302 | Missense | c.319A > C | p.T107P | uc001geb.1 | P64 |
| EIF5 | 1983 | 102871991 | Missense | c.563A > T | p.Y14F | uc001ymq.1 | P64 |
| ELOVL6 | 79071 | 111190493 | Splice_Site_Del | c.e4_splice_site | | uc003iaa.1 | P64 |
| ERBB2IP | 55914 | 65386515 | Missense | c.3658G > A | p.E1201K | uc010iwx.1 | P64 |
| FSCB | 84075 | 44044152 | Missense | c.2057G > A | p.A597T | uc001wvn.1 | P64 |
| GRM7 | 2917 | 7595194 | Missense | c.1750A > C | p.K534T | uc003bql.1 | P64 |
| HIPK3 | 10114 | 33317497 | Missense | c.1724G > C | p.G485A | uc001mul.1 | P64 |
| KCNJ16 | 3773 | 65616093 | De_novo_Start_OutOfFrame | c.272_273insT | | uc002jin.1 | P64 |
| MDFI | 4188 | 41721911 | Missense | c.475C > A | p.P49Q | uc003oqp.2 | P64 |
| NRP2 | 8828 | 206300937 | Nonsense | c.1859C > A | p.Y356* | uc002vaw.1 | P64 |
| PER2 | 8864 | 238834304 | Nonsense | c.1683C > A | p.Y482* | uc002vyc.1 | P64 |
| POP7 | 10248 | 100142684 | Missense | c.557G > C | p.A99P | uc003uwh.2 | P64 |
| SETDB1 | 9869 | 149190120 | Missense | c.2260A > G | p.K715E | uc001evu.1 | P64 |
| SLC7A4 | 6545 | 19715788 | Missense | c.382T > A | p.F105Y | uc002zud.1 | P64 |
| SPTBN2 | 6712 | 66232330 | Missense | c.1280A > G | p.E403G | uc001ojd.1 | P64 |
| SRGAP2 | 23380 | 204633613 | Missense | c.863T > C | p.V177A | uc001hdy.1 | P64 |
| TM7SF2 | 7108 | 64638857 | Missense | c.1360G > A | p.V255M | uc001ocv.1 | P64 |
| TNK2 | 10188 | 197093554 | Missense | c.986C > A | p.R281S | uc003fvt.1 | P64 |
| USP34 | 9736 | 61369506 | Missense | c.4581A > T | p.D1520V | uc002sbe.1 | P64 |
| VIPR2 | 7434 | 158595268 | Missense | c.441A > C | p.K85N | uc003woh.1 | P64 |
| ACAN | 176 | 87196231 | Missense | c.2603A > C | p.E743D | uc002bmy.1 | P65 |
| BID | 637 | 16602132 | Missense | c.808A > C | p.T162P | uc002znc.1 | P65 |
| C9orf93 | 203238 | 15961794 | Missense | c.4256T > C | p.M1314T | uc003zmd.1 | P65 |
| FLG2 | 388698 | 150594244 | Missense | c.2715A > C | p.Y881S | uc001ezw.2 | P65 |
| GAN | 8139 | 79953652 | Missense | c.1165C > G | p.L341V | uc002fgo.1 | P65 |
| GRK7 | 131890 | 143009376 | Missense | c.1334A > G | p.D417G | uc003euf.1 | P65 |
| HCN1 | 348980 | 45432433 | Missense | c.1173C > T | p.A383V | uc003jok.1 | P65 |
| MGA | 23269 | 39815981 | Frame_Shift_Del | c.4408_4408delT | p.A1409fs | uc001zoh.1 | P65 |
| NOTCH1 | 4851 | 138510470 | Frame_Shift_Del | c.7541_7542delCT | p.P2514fs | uc004chz.1 | P65 |
| PLXNA2 | 5362 | 206282243 | Missense | c.4867G > A | p.R1370H | uc001hgz.1 | P65 |
| PTPRH | 5794 | 60400300 | Missense | c.2028A > G | p.T663A | uc002qjq.1 | P65 |
| RBM6 | 10180 | 50070866 | Missense | c.2130C > T | p.S666F | uc003cyc.1 | P65 |
| RIMKLB | 57494 | 8817563 | Frame_Shift_Ins | c.1328_1329insC | p.E359fs | uc001quu.2 | P65 |
| RPLPO | 6175 | 119121066 | Missense | c.676A > T | p.I147F | uc001txp.1 | P65 |
| SLITRK3 | 22865 | 166388975 | Missense | c.2782C > A | p.P780T | uc003fej.2 | P65 |
| SPEN | 23013 | 16128464 | Nonsense | c.3346G > T | p.E1048* | uc001axk.1 | P65 |
| SPERT | 220082 | 45185415 | Missense | c.334C > T | p.A85V | uc001van.1 | P65 |
| TP53 | 7157 | 7517845 | Missense | c.1012G > A | p.R273H | uc002gim.2 | P65 |
| ZC3H12B | 340554 | 64639529 | Nonsense | c.2202C > A | p.Y731* | uc010nko.1 | P65 |
| ZFHX3 | 463 | 71403304 | Splice_Site_SNP | c.e6_splice_site | | uc002fck.1 | P65 |
| ARID1B | 57492 | 157264338 | Missense | c.1852A > C | p.Y567S | uc003qqn.1 | P66 |
| ASTE1 | 28990 | 132215847 | Missense | c.2066G > T | p.S620I | uc010htm.1 | P66 |
| C14orf43 | 91748 | 73263919 | De_novo_Start_OutOfFrame | c.1714C > G | | uc001xos.1 | P66 |
| CD2BP2 | 10421 | 30272477 | Missense | c.774C > G | p.A174G | uc002dxr.1 | P66 |
| CHRNB4 | 1143 | 76714907 | Missense | c.245C > T | p.R45C | uc002bed.1 | P66 |
| CNOT3 | 4849 | 59339217 | Missense | c.489G > A | p.D60N | uc002qdj.1 | P66 |
| COL4A3 | 1285 | 227867981 | Missense | c.3638G > A | p.R1159H | uc002vom.1 | P66 |
| CPS1 | 1373 | 211175209 | Nonsense | c.1843C > A | p.Y588* | uc010fur.1 | P66 |
| DST | 667 | 56579872 | Missense | c.6010T > A | p.Y1968N | uc003pdb.2 | P66 |
| FLNC | 2318 | 128257948 | Nonsense | c.230C > A | p.Y7* | uc003vnz.2 | P66 |
| FTH1 | 2495 | 61489465 | Missense | c.448G > A | p.M71I | uc001nsu.1 | P66 |
| GFI1B | 8328 | 134853570 | Missense | c.555T > C | p.V135A | uc004ccg.1 | P66 |
| GJC2 | 57165 | 226413328 | Missense | c.1421G > A | p.G416R | uc001hsk.1 | P66 |
| KLF9 | 687 | 72218065 | Missense | c.1329C > G | p.A12G | uc004aht.1 | P66 |
| MAEL | 84944 | 165225305 | Missense | c.163G > C | p.R31P | uc001gdy.1 | P66 |
| MANBA | 4126 | 103811592 | Missense | c.1224T > C | p.L375P | uc003hwg.1 | P66 |
| MYD88 | 4615 | 38157645 | Missense | c.794T > C | p.L265P | NM_002468 | P66 |
| PHLDB1 | 23187 | 118020040 | Missense | c.3412G > T | p.R1020L | uc001ptr.1 | P66 |
| PLEKHH1 | 57475 | 67118588 | Missense | c.3476C > G | p.L1112V | uc001xjl.1 | P66 |
| PLEKHN1 | 84069 | 898186 | In_frame_Del | c.1276_1278delGC | p.414_415RT > P | uc001ace.1 | P66 |
| SCN8A | 6334 | 50401812 | Nonsense | c.2029C > A | p.Y617* | uc001ryw.1 | P66 |
| SF3A2 | 8175 | 2199165 | In_frame_Del | c.1137_1157delCC | p.PAPGVHP360del | uc002lvg.1 | P66 |
| SIRPA | 140885 | 1851282 | Missense | c.1087A > C | p.T360P | uc002wft.1 | P66 |
| SMC3 | 9126 | 112351888 | Missense | c.3193C > T | p.L1023F | uc001kze.1 | P66 |
| SMYD1 | 150572 | 88168501 | Missense | c.322C > G | p.A107G | uc002ssr.1 | P66 |
| TNRC6A | 27327 | 24649089 | Missense | c.134A > G | p.K7R | uc002dmm.1 | P66 |
| UPK1A | 11045 | 40856258 | Missense | c.439A > C | p.T147P | uc010eeh.1 | P66 |
| ZNF711 | 7552 | 84409954 | Splice_Site_SNP | c.e8_splice_site | | uc004eeq.1 | P66 |
| AATK | 9625 | 76708382 | Frame_Shift_Ins | c.3911_3912insC | p.P1277fs | uc010dia.1 | P67 |
| ACTL8 | 81569 | 18022392 | Missense | c.482C > T | p.T101M | uc001bat.1 | P67 |
| AHDC1 | 27245 | 27746594 | Missense | c.5589C > G | p.C1540W | uc009vsy.1 | P67 |
| CD22 | 933 | 40518809 | Missense | c.520C > T | p.P148L | uc010edt.1 | P67 |
| CDH15 | 1013 | 87786248 | Missense | c.1827A > C | p.K584Q | uc002fmt.1 | P67 |
| CDH9 | 1007 | 26926414 | Missense | c.1439C > T | p.H424Y | uc003jgs.1 | P67 |
| CNBD1 | 168975 | 88318317 | Missense | c.680C > A | p.T211K | uc003ydy.2 | P67 |
| CREBBP | 1387 | 3718097 | Missense | c.7156C > G | p.Q2318E | uc002cvv.1 | P67 |
| CSMD3 | 114788 | 113416867 | Missense | c.7191C > A | p.D2344E | uc003ynu.1 | P67 |
| DUSP2 | 1844 | 96173628 | Missense | c.808G > A | p.G241D | uc002svk.2 | P67 |
| ERAL1 | 26284 | 24206186 | Missense | c.18C > G | p.A3G | uc002hcy.1 | P67 |
| JAG2 | 3714 | 104685720 | Missense | c.2526A > C | p.T708P | uc001yqg.1 | P67 |
| MUT | 4594 | 49516005 | Missense | c.2084G > A | p.R610H | uc003ozg.2 | P67 |
| MYBL2 | 4605 | 41743895 | Missense | c.387G > C | p.A58P | uc002xlb.1 | P67 |
| MYD88 | 4615 | 38157263 | Missense | c.695T > C | p.M232T | NM_002468 | P67 |
| NBEA | 26960 | 34415190 | Missense | c.439T > C | p.I78T | uc001uvb.1 | P67 |
| PBX2 | 5089 | 32265621 | Frame_Shift_Del | c.321_321delG | p.G17fs | uc003oav.1 | P67 |
| PVRL2 | 5819 | 50073438 | In_frame_Del | c.1551_1553delGA | p.R391del | uc002ozv.1 | P67 |
| SI | 6476 | 166265856 | Missense | c.756C > T | p.R232C | uc003fei.1 | P67 |
| SLC44A3 | 126969 | 95129325 | Missense | c.1641A > G | p.K512E | uc001dqv.2 | P67 |
| SMCHD1 | 23347 | 2695692 | Missense | c.2032G > A | p.V615I | uc002klm.2 | P67 |
| SYT7 | 9066 | 61047911 | Missense | c.1102C > T | p.R366W | uc009ynr.1 | P67 |
| TRIM11 | 81559 | 226649486 | Missense | c.1205C > G | p.A317G | uc001hss.1 | P67 |
| ZNF697 | 90874 | 119966957 | Missense | c.1646A > C | p.H511P | uc001ehy.1 | P67 |
| ABI3BP | 25890 | 101954474 | Missense | c.2901G > A | p.D946N | uc003dun.1 | P68 |
| C11orf41 | 25758 | 33587964 | Missense | c.4406G > A | p.A1428T | uc001mup.2 | P68 |
| CLASP1 | 23332 | 121861233 | Missense | c.3699C > A | p.H1103Q | uc002tnc.1 | P68 |
| CTTNBP2NL | 55917 | 112800515 | Missense | c.1046C > T | p.P293L | uc001ebx.1 | P68 |
| DMXL1 | 1657 | 118512389 | Missense | c.3149A > G | p.T990A | uc010jcl.1 | P68 |
| DOCK8 | 81704 | 410429 | Missense | c.3981C > T | p.A1290V | uc003zgf.1 | P68 |
| GOLGA3 | 2802 | 131900009 | Missense | c.1035A > G | p.E159G | uc001ukz.1 | P68 |
| KRT83 | 3889 | 51001206 | Missense | c.244G > A | p.A61T | uc001saf.2 | P68 |
| LRRC4C | 57689 | 40093863 | Missense | c.2520T > C | p.S186P | uc001mxa.1 | P68 |
| MUC2 | 4583 | 1083430 | Missense | c.12362C > A | p.T4112N | uc001lsx.1 | P68 |
| OR13C8 | 138802 | 106371360 | Missense | c.91A > G | p.I31V | uc004bcc.1 | P68 |
| RIMS4 | 140730 | 42818349 | Missense | c.653G > C | p.R218P | uc010ggu.1 | P68 |
| RPUSD2 | 27079 | 38651347 | Missense | c.859G > A | p.A287T | uc001zmd.1 | P68 |
| RXFP1 | 59350 | 159774068 | Missense | c.1043C > A | p.L321M | uc003ipz.1 | P68 |
| SDC1 | 6382 | 20267419 | Missense | c.562C > A | p.A88D | uc002rdo.1 | P68 |
| SKA3 | 221150 | 20633928 | Splice_Site_SNP | c.e5_splice_site | | uc001unt.1 | P68 |
| TAS2R41 | 259287 | 142885224 | Missense | c.137T > C | p.M46T | uc003wdc.1 | P68 |
| TERF2IP | 54386 | 74239345 | Missense | c.161G > T | p.V22L | uc002fet.1 | P68 |
| TRYX3 | 136541 | 141601831 | Splice_Site_SNP | c.e2_splice_site | | uc003vxb.1 | P68 |
| ALS2CR8 | 79800 | 203527027 | Missense | c.762C > A | p.T161N | uc002uzo.2 | P69 |
| ARRDC1 | 92714 | 139628914 | Missense | c.952C > T | p.P293L | uc004cnp.1 | P69 |
| CALHM1 | 255022 | 105208073 | Missense | c.563C > G | p.S142R | uc001kxe.1 | P69 |
| CCNB3 | 85417 | 50107426 | Missense | c.4170A > C | p.Q1291P | uc004dox.2 | P69 |
| CPXM1 | 56265 | 2726933 | Missense | c.519G > A | p.G152D | uc002wgu.1 | P69 |
| DICER1 | 23405 | 94630229 | Missense | c.5295G > A | p.E1705K | uc001ydw.2 | P69 |
| DLGAP5 | 9787 | 54695137 | Missense | c.1946G > C | p.A577P | uc001xbs.1 | P69 |
| DOCK7 | 85440 | 62892051 | Missense | c.356G > A | p.E108K | uc001daq.1 | P69 |
| FAM135B | 51059 | 139224514 | Missense | c.3732T > A | p.F1187L | uc003yuy.1 | P69 |
| GRB14 | 2888 | 165112505 | Missense | c.933G > C | p.R131P | uc002ucl.1 | P69 |
| ITGA9 | 3680 | 37801572 | Missense | c.2941C > T | p.T963M | uc003chd.1 | P69 |
| MED1 | 5469 | 34817880 | Missense | c.4332T > C | p.S1374P | uc002hrv.2 | P69 |
| MIIP | 60672 | 12011694 | Missense | c.745T > C | p.S189P | uc001ato.1 | P69 |
| NHEDC1 | 150159 | 104047234 | Missense | c.1225T > G | p.I368S | uc003hww.1 | P69 |
| PAK7 | 57144 | 9572897 | Missense | c.625C > T | p.P27L | uc002wnl.2 | P69 |
| PXN | 5829 | 119138181 | Missense | c.940G > A | p.E20K | uc001txu.2 | P69 |
| ABCC3 | 8714 | 46088335 | Nonsense | c.259C > A | p.Y63* | uc002isl.1 | P70 |
| ACLY | 47 | 37297388 | Missense | c.2032G > C | p.G676A | uc002hyi.1 | P70 |
| AGTR1 | 185 | 149942251 | Missense | c.1185C > A | p.L247I | uc003ewg.1 | P70 |
| ALMS1 | 7840 | 73532015 | Missense | c.4967G > T | p.S1619I | uc002sje.1 | P70 |
| APOB | 338 | 21083778 | Frame_Shift_Ins | c.9594_9595insA | p.T3156fs | uc002red.1 | P70 |
| ATP2B2 | 491 | 10362785 | Missense | c.2760T > G | p.V814G | uc003bvt.1 | P70 |
| CACNA1G | 8913 | 46031978 | Missense | c.3821G > A | p.R1150Q | uc002irk.1 | P70 |
| CERCAM | 51148 | 130236577 | Missense | c.1797G > A | p.V467M | uc004buz.2 | P70 |
| DOK3 | 79930 | 176862778 | In_frame_Del | c.1026_1028delCT | p.L289del | uc003mhi.2 | P70 |
| FBXL21 | 26223 | 135304105 | Missense | c.539T > C | p.V173A | uc010jec.1 | P70 |
| HIST1H4F | 8361 | 26348931 | Missense | c.299G > A | p.G100D | uc003nhe.1 | P70 |
| KIAA1244 | 57221 | 138697762 | Missense | c.6086A > G | p.Y2029C | uc003qhu.2 | P70 |
| KIF26A | 26153 | 103688444 | Missense | c.628G > A | p.A210T | uc001yos.2 | P70 |
| KIF26B | 55083 | 243916439 | Missense | c.3971G > C | p.Q1177H | uc001ibf.1 | P70 |
| NR2F2 | 7026 | 94678458 | Missense | c.1173A > G | p.S198G | uc002btq.1 | P70 |
| RAG2 | 5897 | 36572292 | Missense | c.191G > A | p.M1I | uc001mwv.2 | P70 |
| RIF1 | 55183 | 151981368 | Missense | c.458A > G | p.N110D | uc002txm.1 | P70 |
| ROBO2 | 6092 | 77696868 | Missense | c.2399C > T | p.R586W | uc003dpy.2 | P70 |
| SELO | 83642 | 48991188 | Missense | c.1130T > G | p.Y358D | uc003bjx.1 | P70 |
| TAF4B | 6875 | 22149232 | Missense | c.2378T > G | p.V630G | uc002kvt.2 | P70 |
| TAF7L | 54457 | 100434548 | Missense | c.154G > A | p.D48N | uc004ehb.1 | P70 |
| TMEM79 | 84283 | 154528792 | Splice_Site_Del | c.e4_splice_site | | uc001foe.1 | P70 |
| ZBTB10 | 65986 | 81562423 | Missense | c.1421T > G | p.C275G | uc003ybx.2 | P70 |
| ABI3BP | 25890 | 102066342 | Frame_Shift_Del | c.1174_1174delT | p.F363fs | uc003dup.2 | P71 |
| FASN | 2194 | 77639393 | Nonsense | c.2790C > A | p.Y891* | uc002kdu.1 | P71 |
| FOXJ3 | 22887 | 42549329 | Missense | c.335G > T | p.C8F | uc001che.1 | P71 |
| SUSD3 | 203328 | 94877910 | Missense | c.148C > A | p.P38T | uc004atb.1 | P71 |
| BPIL3 | 128859 | 31093481 | Missense | c.1187A > G | p.N396S | uc002wyk.1 | P72 |
| C12orf5 | 57103 | 4331955 | Missense | c.729T > C | p.L217S | uc001qmp.1 | P72 |
| CELSR1 | 9620 | 45308699 | Missense | c.3033C > G | p.N1011K | uc003bhw.1 | P72 |
| CFC1B | 653275 | 131072730 | Missense | c.593T > G | p.W68G | uc002tro.1 | P72 |
| CSMD1 | 64478 | 2953627 | Missense | c.7052C > A | p.T2221K | uc010lrh.1 | P72 |
| DTNA | 1837 | 30711720 | Missense | c.1863C > G | p.A621G | uc010dmn.1 | P72 |
| DYNC1LI2 | 1783 | 65319629 | Missense | c.1150A > C | p.Q373H | uc002eqb.1 | P72 |
| DYRK1B | 9149 | 45008557 | Missense | c.1808T > C | p.S510P | uc002omj.1 | P72 |
| ELF1 | 1997 | 40416032 | Missense | c.787T > C | p.S187P | uc001uxr.1 | P72 |
| FAM179A | 165186 | 29103252 | Missense | c.2234C > T | p.A628V | uc010ezl.1 | P72 |
| FOXJ2 | 55810 | 8091870 | Missense | c.2028T > C | p.S315P | uc001qtu.1 | P72 |
| GFM1 | 85476 | 159866794 | Missense | c.1633A > G | p.E509G | uc003fce.1 | P72 |
| IFT122 | 55764 | 130715978 | Missense | c.3403C > G | p.A1066G | uc003eml.1 | P72 |
| IGFN1 | 91156 | 199452330 | Missense | c.1673C > T | p.R301W | uc001gwc.1 | P72 |
| KCNS2 | 3788 | 99510478 | Missense | c.1445G > C | p.W365C | uc003yin.1 | P72 |
| LYPD5 | 284348 | 48994512 | Missense | c.533C > G | p.S151C | uc002oxm.2 | P72 |
| MAGEA8 | 4107 | 148774495 | Missense | c.1006C > T | p.A264V | uc004fdw.1 | P72 |
| MESP2 | 145873 | 88121151 | In_frame_Del | c.559_570delGGG | p.GQGQ199del | uc002bon.1 | P72 |
| METTL13 | 51603 | 170019650 | Missense | c.648T > G | p.L101V | uc001ghz.1 | P72 |
| PLCD3 | 113026 | 40550913 | Nonsense | c.1348C > T | p.Q412* | uc002iib.1 | P72 |
| PRKCI | 5584 | 171463881 | Missense | c.572C > T | p.R112C | uc003fgs.2 | P72 |
| PTTG1 | 9232 | 159781905 | Missense | c.53C > A | p.T3N | uc003lyj.1 | P72 |
| RAB21 | 23011 | 70450651 | Missense | c.484C > G | p.Q78E | uc001swt.1 | P72 |
| RPS15 | 6209 | 1391458 | Missense | c.576G > C | p.K152N | uc002lsq.1 | P72 |
| TBC1D25 | 4943 | 48304293 | Missense | c.2164A > C | p.T685P | uc004dka.1 | P72 |
| TNK2 | 10188 | 197079875 | Missense | c.2025C > G | p.A627G | uc003fvt.1 | P72 |
| TOPBP1 | 11073 | 134821752 | Missense | c.3393C > T | p.S1016F | uc003eps.1 | P72 |
| TP53 | 7157 | 7519095 | Splice_Site_SNP | c.e5_splice_site | | uc002gim.2 | P72 |
| 12-Sep | 124404 | 4767885 | Missense | c.1080G > A | p.A331T | uc002cxq.1 | P73 |
| ADAMTS7 | 11173 | 76838856 | Missense | c.5234G > T | p.A1675S | uc002bej.2 | P73 |
| ASB12 | 142689 | 63361600 | Missense | c.690G > C | p.R219P | uc004dvq.1 | P73 |
| ATM | 472 | 107707431 | Missense | c.7951A > T | p.Q2522H | uc001pkb.1 | P73 |
| ATM | 472 | 107721712 | Nonsense | c.8836T > G | p.Y2817* | uc001pkb.1 | P73 |
| ATP1A1 | 476 | 116742907 | Missense | c.2564G > A | p.A756T | uc001ege.1 | P73 |
| ATP8B3 | 148229 | 1747166 | Missense | c.2086G > C | p.V618L | uc002ltw.1 | P73 |
| BRD8 | 10902 | 137504292 | Splice_Site_SNP | c.e26_splice_site | | uc003lcf.1 | P73 |
| C14orf43 | 91748 | 73263933 | Missense | c.2926A > T | p.T715S | uc001xot.1 | P73 |
| CHD5 | 26038 | 6129398 | Missense | c.1604C > T | p.P502S | uc001amb.1 | P73 |
| CNTN5 | 53942 | 99675173 | Missense | c.2794C > T | p.R819C | uc001pga.1 | P73 |
| DAPK1 | 1612 | 89501868 | Missense | c.2678A > T | p.E847V | uc004apc.1 | P73 |
| DOK6 | 220164 | 65659552 | Missense | c.1139C > T | p.R317W | uc0021kl.1 | P73 |
| ERBB2IP | 55914 | 65385399 | Missense | c.2542C > T | p.P829S | uc010iwx.1 | P73 |
| ESPL1 | 9700 | 51949811 | Missense | c.909G > A | p.S273N | uc001sck.2 | P73 |
| FAM92A1 | 137392 | 94809636 | Missense | c.908C > G | p.Q269E | uc010maq.1 | P73 |
| FAT4 | 79633 | 126462093 | Missense | c.5077G > A | p.A1693T | uc003ifj.2 | P73 |
| FCER1A | 2205 | 157542410 | Missense | c.439C > G | p.L114V | uc001ftq.1 | P73 |
| GABRA5 | 2558 | 24765119 | Missense | c.961G > A | p.G208S | uc001zbd.1 | P73 |
| GJC3 | 349149 | 99364644 | Missense | c.536C > T | p.T179I | uc003usg.1 | P73 |
| IGSF11 | 152404 | 120127650 | Missense | c.815A > G | p.T190A | uc003ebw.1 | P73 |
| ITK | 3702 | 156570945 | Missense | c.395G > T | p.A105S | uc003lwo.1 | P73 |
| KCNK18 | 338567 | 118959115 | Missense | c.470C > T | p.T157I | uc001ldc.1 | P73 |
| LMLN | 89782 | 199171558 | Missense | c.91C > T | p.P12S | uc003fyt.1 | P73 |
| LRIT2 | 340745 | 85975249 | Missense | c.16C > T | p.S3L | uc001kcy.1 | P73 |
| NVL | 4931 | 222554957 | Missense | c.1035C > G | p.A331G | uc001hok.1 | P73 |
| OBSCN | 84033 | 226573385 | Missense | c.14353G > C | p.R4770P | uc009xez.1 | P73 |
| PHF3 | 23469 | 64471502 | Frame_Shift_Del | c.3375_3381delAA | p.N1117fs | uc003pep.1 | P73 |
| RHBDD3 | 25807 | 27991524 | Missense | c.464T > G | p.V31G | uc003aeq.1 | P73 |
| RSAD2 | 91543 | 6944657 | Missense | c.785G > A | p.A217T | uc002qyp.1 | P73 |
| SLC4A11 | 83959 | 3157652 | Missense | c.2120T > G | p.V691G | uc002wig.1 | P73 |
| TNFAIP2 | 7127 | 102662697 | In_frame_Del | c.281_283delGAA | p.K54del | uc001ymm.1 | P73 |
| TSHZ2 | 128553 | 51303553 | Missense | c.1105C > T | p.T50M | uc002xwo.2 | P73 |
| TSPAN33 | 340348 | 128588805 | Missense | c.381A > T | p.K51M | uc003vop.1 | P73 |
| UGT1A4 | 54657 | 234293054 | Missense | c.878C > G | p.N283K | uc002vux.1 | P73 |
| USH2A | 7399 | 214078036 | Missense | c.9678A > T | p.K3097N | uc001hku.1 | P73 |
| USP19 | 10869 | 49124422 | Nonsense | c.2990C > A | p.Y943* | uc003cvz.2 | P73 |
| A2M | 2 | 9145502 | Nonsense | c.1415C > A | p.Y434* | uc001qvk.1 | P74 |
| ABCC6 | 368 | 16167016 | Missense | c.3308A > C | p.I1091L | uc002den.2 | P74 |
| ADAM15 | 8751 | 153297363 | Missense | c.1840A > C | p.Q580P | uc001fgr.1 | P74 |
| C11orf88 | 399949 | 110892001 | Missense | c.295C > A | p.L99I | uc009yyd.1 | P74 |
| C15orf2 | 23742 | 22472528 | Missense | c.895A > C | p.I141L | uc001ywo.1 | P74 |
| COL11A2 | 1302 | 33261505 | Missense | c.1055A > T | p.E276V | uc003ocx.1 | P74 |
| CYP27C1 | 339761 | 127669546 | Missense | c.685C > A | p.T185K | uc002tod.2 | P74 |
| DERL2 | 51009 | 5330180 | Missense | c.42A > G | p.E9G | uc002gcc.1 | P74 |
| FAM103A1 | 83640 | 81449669 | Missense | c.388A > G | p.D68G | uc002bjl.1 | P74 |
| FAM151B | 167555 | 79873378 | Missense | c.945C > A | p.Q268K | uc003kgv.1 | P74 |
| FUT7 | 2529 | 139045459 | Missense | c.1402C > G | p.L185V | uc004ckq.2 | P74 |
| HECTD1 | 25831 | 30683882 | Missense | c.3002G > C | p.R838P | uc001wrc.1 | P74 |
| KLHL31 | 401265 | 53624918 | Missense | c.1483C > G | p.P448A | uc003pcb.2 | P74 |
| MLL3 | 58508 | 151495360 | Missense | c.9773A > C | p.Q3185P | uc003wla.1 | P74 |
| OLFML3 | 56944 | 114325104 | Nonsense | c.520C > A | p.Y137* | uc001eer.1 | P74 |
| PTCH1 | 5727 | 97260245 | Nonsense | c.3227C > A | p.Y1013* | uc004avk.2 | P74 |
| RBKS | 64080 | 27919537 | Missense | c.426G > C | p.A139P | uc002rlo.1 | P74 |
| RELT | 84957 | 72783932 | Missense | c.1364G > A | p.G400E | uc001otv.1 | P74 |
| RNF10 | 9921 | 119457061 | Missense | c.547A > C | p.N22H | uc001typ.2 | P74 |
| SEMA3A | 10371 | 83448699 | Missense | c.1841T > G | p.V509G | uc003uhz.1 | P74 |
| SLC25A33 | 84275 | 9562832 | Missense | c.939C > G | p.A239G | uc001apw.1 | P74 |
| TP53 | 7157 | 7520080 | Missense | c.526T > G | p.L111R | uc002gim.2 | P74 |
| CA10 | 56934 | 47065998 | Missense | c.1556C > T | p.R274C | uc002itv.2 | P75 |
| CRAMP1L | 57585 | 1643068 | Missense | c.687G > C | p.R112P | uc002cme.1 | P75 |
| DUS3L | 56931 | 5740592 | Missense | c.574A > C | p.T176P | uc002mdc.1 | P75 |
| DYNC2H1 | 79659 | 102844538 | Missense | c.12825G > T | p.L4227F | uc001phn.1 | P75 |
| ELN | 2006 | 73104225 | Missense | c.1016G > C | p.A309P | uc003tzw.1 | P75 |
| EPM2A | 7957 | 145990417 | Missense | c.1181C > T | p.A275V | uc003qkw.1 | P75 |
| GAP43 | 2596 | 116878018 | Missense | c.980C > A | p.Q203K | uc003ebr.1 | P75 |
| ITPKB | 3707 | 224990032 | Frame_Shift_Ins | c.1786_1787insG | p.E584fs | uc001hqg.1 | P75 |
| KIAA0182 | 23199 | 84248567 | Missense | c.1570C > G | p.A499G | uc002fix.1 | P75 |
| NFASC | 23114 | 203214793 | Frame_Shift_Del | c.2150_2150delG | p.G651fs | uc001hbj.1 | P75 |
| PRR21 | 643905 | 240630159 | Frame_Shift_Del | c.901_914delGCC | p.A301fs | uc002vys.1 | P75 |
| SLAMF1 | 6504 | 158873631 | Missense | c.735G > A | p.R130H | uc001fwl.2 | P75 |
| TFEB | 7942 | 41761846 | Missense | c.1047G > A | p.R318H | uc003oqu.1 | P75 |
| ADAMTS19 | 171019 | 129047739 | Missense | c.2674G > A | p.G892S | uc003kvb.1 | P76 |
| BID | 637 | 16602132 | Missense | c.808A > C | p.T162P | uc002znc.1 | P76 |
| C17orf71 | 55181 | 54642204 | Missense | c.52C > G | p.P4A | uc002ixi.1 | P76 |
| CASKIN1 | 57524 | 2170623 | Missense | c.2779G > C | p.R916P | uc010bsg.1 | P76 |
| CHMP7 | 91782 | 23169973 | Missense | c.1361A > G | p.D238G | uc003xdc.2 | P76 |
| COPG | 22820 | 130478921 | Missense | c.2689G > C | p.E863D | uc003els.1 | P76 |
| DLG5 | 9231 | 79265503 | Missense | c.1691C > T | p.R541W | uc001jzk.1 | P76 |
| GALNT3 | 2591 | 166319480 | Missense | c.1916A > G | p.K510R | uc010fph.1 | P76 |
| KLHL11 | 55175 | 37274800 | Missense | c.356C > G | p.A117G | uc002hyf.1 | P76 |
| LRRIQ1 | 84125 | 84024438 | Missense | c.3402A > T | p.K1097N | uc001tac.1 | P76 |
| MRC2 | 9902 | 58097890 | Missense | c.1302G > T | p.L300F | uc002jad.1 | P76 |
| NEU4 | 129807 | 242406849 | Nonsense | c.1744C > A | p.Y431* | uc002wcn.1 | P76 |
| NINJ2 | 4815 | 544793 | Nonsense | c.527C > T | p.R146* | uc001qil.1 | P76 |
| PCDHA8 | 56140 | 140202654 | Missense | c.1564C > T | p.P522S | uc003lhs.1 | P76 |
| RGS9 | 8787 | 60594848 | Missense | c.679T > G | p.V190G | uc002jfe.1 | P76 |
| SSPO | 23145 | 149124440 | Missense | c.6583G > A | p.G2195S | uc010lpk.1 | P76 |
| STAB1 | 23166 | 52529348 | Splice_Site_SNP | c.e52_splice_site | | uc003dej.1 | P76 |
| STOX1 | 219736 | 70314588 | Missense | c.1030G > A | p.V344I | uc001joq.1 | P76 |
| TAOK1 | 57551 | 24849472 | Missense | c.1204A > G | p.H337R | uc002hdz.1 | P76 |
| TBC1D23 | 55773 | 101517661 | Missense | c.1634A > G | p.K543E | uc003dtt.1 | P76 |
| TBC1D28 | 254272 | 18483233 | Missense | c.590G > A | p.V60I | uc002gud.2 | P76 |
| TP53 | 7157 | 7518996 | Missense | c.772A > T | p.H193L | uc002gim.2 | P76 |
| TP53AIP1 | 63970 | 128312725 | Missense | c.409C > T | p.L67F | uc001qex.1 | P76 |
| VPS41 | 27072 | 38764580 | Missense | c.1475G > T | p.W483C | uc003tgy.1 | P76 |
| BMPER | 168667 | 33943462 | Missense | c.629G > T | p.V86L | uc003tdw.1 | P77 |
| CSMD1 | 64478 | 3876895 | Missense | c.940A > G | p.T184A | uc010lrh.1 | P77 |
| DENND1A | 57706 | 125184134 | Missense | c.2661C > T | p.P810S | uc004bnz.1 | P77 |
| DHX37 | 57647 | 124031223 | In_frame_Del | c.601_603delGAG | p.E168del | uc001ugy.1 | P77 |
| DOCK6 | 57572 | 11222606 | Missense | c.705C > G | p.L222V | uc002mqs.2 | P77 |
| DSP | 1832 | 7500957 | Missense | c.457G > A | p.G60S | uc003mxp.1 | P77 |
| FAT1 | 2195 | 187865514 | Missense | c.2650A > T | p.E821V | uc003izf.1 | P77 |
| IL12RB2 | 3595 | 67589273 | Missense | c.1811G > A | p.G391R | uc001ddu.1 | P77 |
| IRAK4 | 51135 | 42466478 | Missense | c.1322A > G | p.K400E | uc001rnu.2 | P77 |
| MAN1C1 | 57134 | 25952568 | Nonsense | c.1171C > T | p.R281* | uc001bkm.2 | P77 |
| NBPF1 | 55672 | 16781708 | Frame_Shift_Del | c.2112_2112delC | p.D408fs | uc009vos.1 | P77 |
| NPL | 80896 | 181030157 | Missense | c.168G > A | p.G10S | uc009wyb.1 | P77 |
| PRKAR1B | 5575 | 717535 | Missense | c.240G > A | p.R45H | uc003siu.1 | P77 |
| PRR21 | 643905 | 240630790 | Frame_Shift_Del | c.256_283delAGT | p.S86fs | uc002vys.1 | P77 |
| PSD3 | 23362 | 18774161 | Missense | c.596T > C | p.S165P | uc003wza.1 | P77 |
| PTK2B | 2185 | 27352517 | Missense | c.2504G > A | p.G566R | uc003xfn.1 | P77 |
| RAMP3 | 10268 | 45164003 | Frame_Shift_Del | c.112_112delG | p.L17fs | uc003tnb.1 | P77 |
| SCN7A | 6332 | 167037099 | Nonsense | c.673G > A | p.W182* | uc002udu.1 | P77 |
| TAF6 | 6878 | 99543163 | Missense | c.1984C > T | p.S616L | uc003uth.1 | P77 |
| UCK2 | 7371 | 164141791 | Missense | c.788T > A | p.Y203N | uc001gdp.1 | P77 |
| WARS | 7453 | 99889892 | Missense | c.694A > C | p.K204Q | uc001yhf.1 | P77 |
| C6orf1 | 221491 | 34322597 | Missense | c.744C > A | p.T51N | uc003ojf.1 | P78 |
| CPNE7 | 27132 | 88189378 | Missense | c.1760A > G | p.I544V | uc002fnp.1 | P78 |
| DAPK1 | 1612 | 89511703 | Missense | c.4035C > A | p.D1299E | uc004apc.1 | P78 |
| DLG5 | 9231 | 79271650 | Missense | c.1502C > T | p.R478W | uc001jzk.1 | P78 |
| FAT3 | 120114 | 92173109 | Missense | c.7299C > T | p.R2428W | uc001pdj.2 | P78 |
| GABRA2 | 2555 | 46007001 | Missense | c.1178C > T | p.H169Y | uc003gxc.2 | P78 |
| GRIK4 | 2900 | 120338435 | Missense | c.2388G > A | p.V701M | uc001pxn.2 | P78 |
| HDGFRP2 | 84717 | 4448957 | Missense | c.1424C > T | p.P444L | uc002mao.1 | P78 |
| IL28B | 282617 | 44426941 | Missense | c.218C > T | p.R72C | uc002oks.1 | P78 |
| MAOB | 4129 | 43587945 | Missense | c.232C > G | p.A19G | uc004dfz.2 | P78 |
| MED12 | 9968 | 70255978 | Missense | c.329G > A | p.G44S | uc004dyy.1 | P78 |
| SYTL2 | 54843 | 85096119 | Splice_Site_SNP | c.e8_splice_site | | uc001pbb.1 | P78 |
| WDR7 | 23335 | 52597686 | Missense | c.3185T > C | p.C992R | uc002lgk.1 | P78 |
| WDR72 | 256764 | 51812596 | Frame_Shift_Del | c.85_85delG | p.A15fs | uc002acj.2 | P78 |
| AKAP8L | 26993 | 15390730 | Missense | c.104G > A | p.S2N | uc002naw.1 | P79 |
| ALDH5A1 | 7915 | 24623476 | Missense | c.896G > A | p.V290M | uc003nef.1 | P79 |
| C1QL1 | 10882 | 40400864 | Missense | c.307A > C | p.T27P | uc002ihv.1 | P79 |
| DOCK5 | 80005 | 25205517 | Frame_Shift_Ins | c.519_520insGG | p.R128fs | uc003xeg.1 | P79 |
| EPPK1 | 83481 | 145015572 | Missense | c.3851C > G | p.L1255V | uc003zaa.1 | P79 |
| FAM120A | 23196 | 95254365 | Missense | c.372G > C | p.R116P | uc004atw.1 | P79 |
| KCNU1 | 157855 | 36761243 | Frame_Shift_Del | c.244_244delA | p.K53fs | uc010lvw.1 | P79 |
| KIAA1524 | 57650 | 109784542 | Missense | c.598G > C | p.S110T | uc003dxb.2 | P79 |
| MED12L | 116931 | 152391387 | Missense | c.1985G > T | p.K649N | uc003eyp.1 | P79 |
| PFN1 | 5216 | 4790826 | Missense | c.303G > A | p.R56Q | uc002gaa.1 | P79 |
| PLXNA1 | 5361 | 128219828 | Missense | c.3597T > G | p.V1198G | uc003ejg.1 | P79 |
| PODXL | 5420 | 130891570 | In_frame_Del | c.342_347delGTC | p.28_30PSP > P | uc003vqw.2 | P79 |
| PPFIA2 | 8499 | 80179892 | Read-through | c.3935A > C | p.*1258C | uc001szo.1 | P79 |
| RFTN2 | 130132 | 198206795 | Missense | c.1012G > A | p.G204R | uc002uuo.2 | P79 |
| SPG20 | 23111 | 35807294 | Missense | c.768C > A | p.P225Q | uc001uvm.1 | P79 |
| STAB2 | 55576 | 102624878 | Missense | c.4361G > C | p.G1392A | uc001tjw.1 | P79 |
| TNS3 | 64759 | 47375185 | Missense | c.1950A > G | p.N528S | uc003tnv.1 | P79 |
| ZMAT5 | 55954 | 28464404 | Missense | c.549C > A | p.L100I | uc003agm.1 | P79 |
| BAI3 | 577 | 69405721 | Missense | c.881G > A | p.G145R | uc003pev.2 | P80 |
| CCDC62 | 84660 | 121852039 | Missense | c.1538A > T | p.S465C | uc001udc.1 | P80 |
| COL5A2 | 1290 | 189607103 | Missense | c.4713G > A | p.V1480M | uc002uqk.1 | P80 |
| DPP9 | 91039 | 4653620 | Missense | c.1156G > T | p.W293L | uc002mba.1 | P80 |
| KAL1 | 3730 | 8461076 | Missense | c.2153G > A | p.R668H | uc004csf.1 | P80 |
| KNTC1 | 9735 | 121639218 | Frame_Shift_Del | c.4064_4064delG | p.G1301fs | uc001ucv.1 | P80 |
| LAD1 | 3898 | 199622274 | Missense | c.1073G > A | p.A280T | uc001gwm.1 | P80 |
| LRRK1 | 79705 | 99410672 | Missense | c.4031C > G | p.L1238V | uc002bwr.1 | P80 |
| LRRN4CL | 221091 | 62212012 | Missense | c.852C > G | p.P182R | uc001nun.1 | P80 |
| MYH6 | 4624 | 22935397 | Missense | c.2432C > T | p.R789C | uc001wjv.2 | P80 |
| NINJ1 | 4814 | 94936257 | Missense | c.135C > A | p.P22T | uc004atg.2 | P80 |
| NPR3 | 4883 | 32748081 | Missense | c.660T > C | p.S148P | uc003jhv.1 | P80 |
| PLB1 | 151056 | 28705507 | Missense | c.3769G > T | p.A1257S | uc002rmb.1 | P80 |
| PTPRZ1 | 5803 | 121403502 | Missense | c.891T > A | p.F166I | uc003vjy.1 | P80 |
| RAPGEF5 | 9771 | 22297387 | Splice_Site_Ins | c.e6_splice_site | | uc003svg.1 | P80 |
| SENP6 | 26054 | 76463931 | Missense | c.2885A > G | p.I756V | uc003pid.2 | P80 |
| SHANK1 | 50944 | 55867161 | Missense | c.2619G > A | p.R867H | uc002psx.1 | P80 |
| SIM2 | 6493 | 37035984 | Missense | c.1003A > C | p.N316T | uc002yvr.1 | P80 |
| SLC22A17 | 51310 | 22887326 | Missense | c.778G > A | p.R241Q | uc001wjl.1 | P80 |
| TLE2 | 7089 | 2964816 | Missense | c.847G > A | p.D243N | uc010dth.1 | P80 |
| TNPO2 | 30000 | 12678017 | Missense | c.2399A > C | p.N646T | uc002mup.1 | P80 |
| UCK1 | 83549 | 133394153 | Missense | c.696C > T | p.P201L | uc004cay.1 | P80 |
| ZMYND15 | 84225 | 4593457 | Missense | c.1285C > A | p.H419N | uc002fyu.1 | P80 |
| ADCY1 | 107 | 45628857 | Missense | c.1028A > G | p.E337G | uc003tne.2 | P81 |
| APOC2 | 344 | 50144278 | Missense | c.341G > C | p.A80P | uc002pah.1 | P81 |
| ARID4B | 51742 | 233411660 | Missense | c.3695A > G | p.D1066G | uc001hwq.1 | P81 |
| ATP2B3 | 492 | 152483672 | Missense | c.3385G > A | p.E1087K | uc004fht.1 | P81 |
| C14orf183 | 196913 | 49620246 | Missense | c.848T > C | p.L283S | uc001wxm.1 | P81 |
| C17orf82 | 388407 | 56844386 | Missense | c.493G > T | p.G90W | uc002izh.1 | P81 |
| C22orf42 | 150297 | 30877000 | Missense | c.513T > C | p.S158P | uc003amd.1 | P81 |
| CAMKK2 | 10645 | 120182506 | Missense | c.919G > T | p.M265I | uc001tzu.1 | P81 |
| CD74 | 972 | 149772471 | Missense | c.55A > G | p.D12G | uc00lsf.1 | P81 |
| CLDN1 | 9076 | 191513372 | Missense | c.591C > T | p.A124V | uc003fsh.1 | P81 |
| EXOC3L | 283849 | 65776586 | Missense | c.1944G > T | p.G568V | uc002erx.1 | P81 |
| FAM116B | 414918 | 49097454 | Frame_Shift_Del | c.548_548delT | p.L102fs | uc003bkx.1 | P81 |
| HSD17B6 | 8630 | 55462229 | Missense | c.628T > C | p.V173A | uc001smg.1 | P81 |
| IDH1 | 3417 | 208812085 | Missense | c.1355G > A | p.G370D | uc002vcs.1 | P81 |
| KNDC1 | 85442 | 134877599 | Missense | c.4661A > C | p.T1554P | uc001llz.1 | P81 |
| MCM6 | 4175 | 136325461 | Missense | c.1974G > C | p.R633P | uc002tuw.1 | P81 |
| NALCN | 259232 | 100827062 | Missense | c.823A > G | p.T212A | uc001vox.1 | P81 |
| PLA2G4A | 5321 | 185129859 | Missense | c.476T > A | p.L91I | uc001gsc.1 | P81 |
| PLA2G4A | 5321 | 185182666 | Missense | c.1419T > G | p.L405W | uc001gsc.1 | P81 |
| RYR2 | 6262 | 236038954 | Missense | c.14549T > C | p.L4810P | uc001hyl.1 | P81 |
| SCN7A | 6332 | 167030735 | Missense | c.800T > A | p.S225T | uc002udu.1 | P81 |
| SIRPA | 140885 | 1843955 | Missense | c.299C > A | p.T97K | uc002wft.1 | P81 |
| SLC22A7 | 10864 | 43374283 | Missense | c.308C > T | p.P70L | uc003out.1 | P81 |
| SLIT2 | 9353 | 20139695 | Missense | c.1692A > C | p.L496F | uc003gpr.1 | P81 |
| SULT1A2 | 6799 | 28514733 | Missense | c.371T > C | p.I7T | uc002dqg.1 | P81 |
| TECTA | 7007 | 120528887 | Missense | c.4193G > C | p.C1398S | uc001pxr.1 | P81 |
| TEX15 | 56154 | 30823876 | Missense | c.2200G > A | p.E734K | uc003xil.1 | P81 |
| TPRX1 | 284355 | 52997355 | In_frame_Del | c.773_796delGAA | p.234_242PNPGPIP | uc002php.1 | P81 |
| ALKBH1 | 8846 | 77244084 | Missense | c.26C > G | p.A6G | uc001xuc.1 | P82 |
| ATP1A4 | 480 | 158395908 | Missense | c.1225A > C | p.N249T | uc001fve.2 | P82 |
| BCOR | 54880 | 39818154 | Frame_Shift_Del | c.1680_1681delCC | p.P463fs | uc004den.2 | P82 |
| BRSK2 | 9024 | 1389377 | Missense | c.420T > C | p.L56P | uc001ltm.2 | P82 |
| CAND1 | 55832 | 65961968 | Missense | c.517C > A | p.T27K | uc001stn.2 | P82 |
| DNAH10 | 196385 | 122965416 | Missense | c.10310G > C | p.A3429P | uc001uft.2 | P82 |
| DNAH9 | 1770 | 11588941 | Missense | c.6282C > T | p.R2072C | uc002gne.1 | P82 |
| ENPEP | 2028 | 111688855 | Frame_Shift_Del | c.2417_2417delG | p.W692fs | uc003iab.2 | P82 |
| GRM5 | 2915 | 87940496 | Missense | c.2203G > A | p.R668H | uc001pcq.1 | P82 |
| KIAA0430 | 9665 | 15610904 | Missense | c.4124G > A | p.E1311K | uc002ddr.1 | P82 |
| KIAA0802 | 23255 | 8708540 | Missense | c.234G > A | p.R31Q | uc002knr.2 | P82 |
| KIRREL3 | 84623 | 125800111 | Nonsense | c.1997C > A | p.Y637* | uc001qea.1 | P82 |
| MAP1B | 4131 | 71526245 | Missense | c.1548A > G | p.Y436C | uc003kbw.2 | P82 |
| MEMO1 | 51072 | 31948527 | Splice_Site_SNP | c.e8_splice_site | | uc002rnx.1 | P82 |
| MMP12 | 4321 | 102244005 | Frame_Shift_Ins | c.674_675insA | p.T210fs | uc001phk.1 | P82 |
| NOTCH1 | 4851 | 138510470 | Frame_Shift_Del | c.7541_7542delCT | p.P2514fs | uc004chz.1 | P82 |
| PPM1F | 9647 | 20615657 | Missense | c.868C > G | p.Q252E | uc002zvp.1 | P82 |
| PTH2 | 113091 | 54618366 | Missense | c.145C > G | p.L15V | uc002pnn.1 | P82 |
| SCAPER | 49855 | 74808099 | Missense | c.2091C > G | p.A685G | uc002bby.1 | P82 |
| SEC16B | 89866 | 176196666 | Missense | c.1688G > A | p.M274I | uc001glj.1 | P82 |
| SETBP1 | 26040 | 40785584 | Missense | c.2577G > A | p.V707M | uc010dni.1 | P82 |
| SMCR7 | 125170 | 18108688 | Missense | c.1440T > C | p.L417P | uc002gst.1 | P82 |
| TSNAXIP1 | 55815 | 66412286 | Missense | c.423C > A | p.T10K | uc002euj.1 | P82 |
| TTC7A | 57217 | 47132436 | Missense | c.2505A > G | p.M713V | uc010fbb.1 | P82 |
| VARS | 7407 | 31854805 | Missense | c.4067C > G | p.A1215G | uc003nxe.1 | P82 |
| WWC1 | 23286 | 167804132 | Missense | c.2555A > G | p.E830G | uc003izu.1 | P82 |
| ZP4 | 57829 | 236115706 | Missense | c.943C > T | p.L315F | uc001hym.1 | P82 |
| ATG9B | 285973 | 150352416 | Frame_Shift_Ins | c.103_104insG | p.G9fs | uc010lpv.1 | P83 |
| C11orf35 | 256329 | 545379 | Missense | c.1762A > C | p.T567P | uc001lpx.1 | P83 |
| CNTROB | 116840 | 7780825 | Nonsense | c.1712C > T | p.Q265* | uc002gjp.1 | P83 |
| EVC | 2121 | 5784069 | Missense | c.585C > T | p.S134F | uc003gil.1 | P83 |
| FLNB | 2317 | 58063032 | Missense | c.1573C > T | p.R470W | uc010hne.1 | P83 |
| HYDIN | 54768 | 69499816 | Missense | c.8361G > T | p.V2745L | uc002ezr.1 | P83 |
| IFLTD1 | 160492 | 25564170 | Missense | c.992G > A | p.R281H | uc001rgs.1 | P83 |
| IRF2 | 3660 | 185548886 | Frame_Shift_Del | c.902_906delAAC | p.E234fs | uc003iwf.2 | P83 |
| MADCAM1 | 8174 | 452762 | Missense | c.771A > C | p.Q254P | uc002los.1 | P83 |
| PANK4 | 55229 | 2436988 | Missense | c.1256A > G | p.E416G | uc001ajm.1 | P83 |
| PDE2A | 5138 | 71970570 | Missense | c.2082C > T | p.R641W | uc001osm.1 | P83 |
| PRKG1 | 5592 | 53711936 | Frame_Shift_Del | c.1680_1680delA | p.A521fs | uc001jjo.2 | P83 |
| PXDNL | 137902 | 52547417 | Missense | c.796A > G | p.Q232R | uc003xqu.2 | P83 |
| SAMD5 | 389432 | 147871912 | Missense | c.157G > C | p.R52P | uc003qmc.1 | P83 |
| TMEM59 | 9528 | 54270424 | Missense | c.1212A > G | p.H321R | uc001cwq.1 | P83 |
| TRPV4 | 59341 | 108705926 | Missense | c.2594C > A | p.N833K | uc001tpj.1 | P83 |
| TTN | 7273 | 179171917 | Missense | c.49285A > G | p.E16354G | uc002umr.1 | P83 |
| TXLNB | 167838 | 139633330 | Nonsense | c.756G > T | p.E215* | uc010kha.1 | P83 |
| ANGPT2 | 285 | 6353944 | Missense | c.1576T > C | p.I416T | uc003wqj.2 | P84 |
| EVC2 | 132884 | 5675411 | Missense | c.2309G > A | p.R752Q | uc003gij.1 | P84 |
| MICALCL | 84953 | 12272963 | In_frame_Del | c.1700_1702delCT | p.T471del | uc001mkg.1 | P84 |
| OR5AS1 | 219447 | 55555423 | Missense | c.953G > A | p.R318H | uc001nif.1 | P84 |
| OR8J3 | 81168 | 55661275 | Missense | c.496G > A | p.V166M | uc001nij.1 | P84 |
| PGM5 | 5239 | 70189275 | Missense | c.795T > A | p.F189Y | uc004agr.1 | P84 |
| PHLPP1 | 23239 | 58648424 | Missense | c.395C > A | p.L73M | uc002lis.1 | P84 |
| PIWIL4 | 143689 | 93940400 | Missense | c.219G > C | p.R23P | uc001pfa.1 | P84 |
| RECQL5 | 9400 | 71138513 | Splice_Site_Ins | c.e12_splice_site | | uc010dgl.1 | P84 |
| SF3B1 | 23451 | 197974954 | Missense | c.2271G > T | p.K741N | uc002uue.1 | P84 |
| SLC22A13 | 9390 | 38292790 | Missense | c.1295G > A | p.V416M | uc003chz.2 | P84 |
| XPO1 | 7514 | 61572976 | Missense | c.1840G > A | p.E571K | uc002sbi.1 | P84 |
| AGPAT9 | 84803 | 84744924 | Missense | c.1503G > A | p.G429S | uc003how.1 | P85 |
| ATM | 472 | 107677584 | Splice_Site_SNP | c.e35_splice_site | | uc001pkb.1 | P85 |
| CDHR3 | 222256 | 105440555 | Missense | c.1144G > A | p.E356K | uc003vdl.2 | P85 |
| CHD9 | 80205 | 51899194 | Missense | c.7045C > T | p.S2294F | uc002ehb.1 | P85 |
| CIDEB | 27141 | 23845524 | Missense | c.356G > C | p.E78Q | uc001won.1 | P85 |
| CXorf26 | 51260 | 75311728 | Missense | c.378C > T | p.P59S | uc004ecl.1 | P85 |
| F8 | 2157 | 153744594 | Missense | c.6703C > T | p.R2178C | uc004fmt.1 | P85 |
| MAN1C1 | 57134 | 25816933 | Missense | c.388C > G | p.P20A | uc001bkm.2 | P85 |
| MDC1 | 9656 | 30781379 | Missense | c.4000C > G | p.T1187S | uc003nrg.2 | P85 |
| MNT | 4335 | 2237491 | Missense | c.1455G > C | p.Q401H | uc002fur.1 | P85 |
| NEK10 | 152110 | 27301134 | Missense | c.2251C > A | p.N659K | uc003cdt.1 | P85 |
| NLGN2 | 57555 | 7259157 | Missense | c.1076C > T | p.R335W | uc002ggt.1 | P85 |
| PKHD1L1 | 93035 | 110477524 | Missense | c.1008G > A | p.V302I | uc003yne.1 | P85 |
| SF3B1 | 23451 | 197975079 | Missense | c.2146A > G | p.K700E | uc002uue.1 | P85 |
| SLC25A42 | 284439 | 19079734 | Missense | c.680A > T | p.I177F | uc002nlf.1 | P85 |
| TBC1D26 | 353149 | 15582353 | Missense | c.564C > T | p.A105V | uc010cov.1 | P85 |
| ZIC2 | 7546 | 99432896 | Nonsense | c.577C > T | p.Q193* | uc001von.1 | P85 |
| ZNF711 | 7552 | 84412580 | Missense | c.2400G > A | p.S505N | uc004eeq.1 | P85 |
| ACTRT1 | 139741 | 127013502 | Missense | c.557T > C | p.L122P | uc004eum.1 | P86 |
| ACVR2A | 92 | 148401270 | Missense | c.1669T > C | p.M500T | uc002twg.1 | P86 |
| G1orf113 | 79729 | 36558374 | Missense | c.1052C > T | p.S154L | uc001cah.1 | P86 |
| C8orf76 | 84933 | 124322660 | Missense | c.139C > G | p.C36W | uc003yqc.1 | P86 |
| CAPN6 | 827 | 110381154 | Missense | c.1078C > G | p.Q304E | uc004epc.1 | P86 |
| DCN | 1634 | 90082554 | Nonsense | c.377G > T | p.E95* | uc001tbs.1 | P86 |
| DDX11 | 1663 | 31133692 | Splice_Site_SNP | c.e8_splice_site | | uc001rjt.1 | P86 |
| DLGAP1 | 9229 | 3869890 | Missense | c.246C > T | p.P60L | uc002kmf.1 | P86 |
| ERC2 | 26059 | 56158107 | Missense | c.1499C > A | p.R415S | uc003dhr.1 | P86 |
| FAM132A | 388581 | 1168345 | Missense | c.723T > C | p.C231R | uc001adl.1 | P86 |
| FAM53B | 9679 | 126301801 | Read-through | c.1792A > G | p.*423W | uc001lhv.1 | P86 |
| GUCY1A2 | 2977 | 106393739 | Missense | c.643G > A | p.V85I | uc009yxn.1 | P86 |
| KIF4A | 24137 | 69489226 | Missense | c.1610G > A | p.E495K | uc004dyg.1 | P86 |
| LGALS3 | 3958 | 54674798 | Missense | c.452T > C | p.Y101H | uc001xbr.1 | P86 |
| MFSD7 | 84179 | 666077 | Missense | c.1206C > G | p.P373R | uc003gbb.1 | P86 |
| NBEAL2 | 23218 | 47015888 | Missense | c.3802A > G | p.Q1208R | uc003cqp.2 | P86 |
| NOS1 | 4842 | 116252978 | Missense | c.965G > C | p.V94L | uc001twm.1 | P86 |
| PRIC285 | 85441 | 61663879 | Missense | c.7411G > T | p.Q2173H | uc002yfm.2 | P86 |
| ProSAPiP1 | 9762 | 3093302 | Missense | c.3218A > G | p.E607G | uc002wia.1 | P86 |
| RPS28 | 6234 | 8292862 | Missense | c.144C > G | p.T38R | uc002mjn.1 | P86 |
| SAMHD1 | 25939 | 34981271 | Missense | c.892G > A | p.M254I | uc002xgh.1 | P86 |
| SEMA4C | 54910 | 96890742 | Missense | c.2240C > G | p.A670G | uc002sxg.2 | P86 |
| SLCO2A1 | 6578 | 135148892 | Missense | c.1466_14670C > T | p.P398F | uc003eqa.2 | P86 |
| USP6NL | 9712 | 11545726 | Missense | c.1301A > G | p.R420G | uc001iks.1 | P86 |
| YIPF3 | 25844 | 43591402 | Missense | c.479A > G | p.K108E | uc010jyr.1 | P86 |
| ZMYM3 | 9203 | 70377817 | Missense | c.3992T > C | p.F1302S | uc004dzh.1 | P86 |
| BCOR | 54880 | 39819146 | Frame_Shift_Del | c.688_689delGG | p.V132fs | uc004den.2 | P87 |
| C11orf16 | 56673 | 8905208 | Missense | c.538A > T | p.L138F | uc001mhb.2 | P87 |
| C19orf35 | 374872 | 2226747 | Missense | c.1448T > G | p.C452G | uc002lvn.1 | P87 |
| CEP350 | 9857 | 178297999 | Missense | c.5667G > A | p.E1762K | uc001gnt.1 | P87 |
| GPR128 | 84873 | 101856634 | Missense | c.1901G > C | p.A549P | uc003duc.1 | P87 |
| GRIN3A | 116443 | 103379932 | Missense | c.3548A > C | p.I983L | uc004bbp.1 | P87 |
| IGSF10 | 285313 | 152647512 | Missense | c.2947C > T | p.P983S | uc003ezb.1 | P87 |
| INPP5D | 3635 | 233633454 | Missense | c.175G > A | p.G8S | uc002vtv.1 | P87 |
| KCNC2 | 3747 | 73730870 | Missense | c.1726G > T | p.L394F | uc001sxg.1 | P87 |
| NCKAP5 | 344148 | 133257568 | Missense | c.3660G > A | p.A1096T | uc002ttp.1 | P87 |
| NOTCH1 | 4851 | 138510470 | Frame_Shift_Del | c.7541_7542delCT | p.P2514fs | uc004chz.1 | P87 |
| NR4A1 | 3164 | 50738769 | Missense | c.2728T > G | p.V578G | uc001rzq.1 | P87 |
| OR2G6 | 391211 | 246752085 | Missense | c.515G > A | p.R172H | uc001ien.1 | P87 |
| PBX2 | 5089 | 32262573 | Missense | c.1379T > G | p.S370A | uc003oav.1 | P87 |
| PLEKHA5 | 54477 | 19327644 | Missense | c.1465G > A | p.G487R | uc001rea.1 | P87 |
| TDRD5 | 163589 | 177897973 | Missense | c.2629G > C | p.A812P | uc001gng.1 | P87 |
| CAMK4 | 814 | 110740514 | Missense | c.461G > A | p.V121I | uc003kpf.1 | P88 |
| GPR39 | 2863 | 132891393 | Missense | c.777G > A | p.S103N | uc002ttl.1 | P88 |
| INPP4A | 3631 | 98528924 | Missense | c.1113A > G | p.N337S | uc002syy.1 | P88 |
| MYO15A | 51168 | 17993350 | Missense | c.7390G > C | p.S2351T | uc010cpt.1 | P88 |
| NRAS | 4893 | 115058052 | Missense | c.436A > G | p.Q61R | uc009wgu.1 | P88 |
| PIK3C2A | 5286 | 17114687 | Missense | c.1832A > G | p.D589G | uc001mmq.2 | P88 |
| PLK1 | 5347 | 23599822 | Missense | c.717G > T | p.V222L | uc002dlz.1 | P88 |
| SAMHD1 | 25939 | 34973148 | Missense | c.1287T > G | p.I386S | uc002xgh.1 | P88 |
| SLC27A5 | 10998 | 63714890 | Frame_Shift_Del | c.268_268delC | p.P82fs | uc002qtc.1 | P88 |
| SOX8 | 30812 | 973775 | Missense | c.584C > T | p.R157C | uc002ckn.1 | P88 |
| STX16 | 8675 | 56684652 | Nonsense | c.1612G > T | p.E293* | uc002xzi.1 | P88 |
| TSC2 | 7249 | 2061594 | Missense | c.2028G > A | p.S641N | uc002con.1 | P88 |
| ZNF146 | 7705 | 41419850 | Missense | c.2191A > G | p.Q223R | uc002odq.2 | P88 |
| ZNF668 | 79759 | 30980681 | Missense | c.1426G > T | p.V357L | uc010caf.1 | P88 |
| GALK2 | 2585 | 47249819 | Missense | c.106C > A | p.T3K | uc001zxj.1 | P89 |
| MYH7B | 57644 | 33046900 | Missense | c.3019A > G | p.E976G | uc002xbi.1 | P89 |
| NFKBIA | 4792 | 34943526 | Missense | c.186C > A | p.L26M | uc001wtf.2 | P89 |
| PASD1 | 139135 | 150583294 | Missense | c.1221T > C | p.Y297H | uc004fev.2 | P89 |
| PHKA2 | 5256 | 18825279 | Missense | c.3635C > G | p.R1069G | uc004cyv.2 | P89 |
| SEMA4G | 57715 | 102733150 | Missense | c.2188C > A | p.L602I | uc001krw.1 | P89 |
| TCF3 | 6929 | 1583078 | Missense | c.287G > C | p.S86T | uc002ltp.1 | P89 |
| TJP2 | 9414 | 71039274 | Missense | c.1971C > G | p.R591G | uc004ahe.1 | P89 |
| VASH1 | 22846 | 76306148 | Splice_Site_SNP | c.e2_splice_site | | uc001xst.2 | P89 |
| DNAH1 | 25981 | 52379823 | Missense | c.6543A > G | p.E2156G | uc003dds.1 | P90 |
| DNHD1 | 144132 | 6545248 | Missense | c.6207G > C | p.R2032P | uc001mdw.2 | P90 |
| HACE1 | 57531 | 105305028 | Nonsense | c.2501C > T | p.Q742* | uc003pqu.1 | P90 |
| HIST1H1D | 3007 | 26342680 | Missense | c.516A > G | p.K154R | uc003nhd.1 | P90 |
| ICA1L | 130026 | 203361882 | Missense | c.1323G > T | p.G387W | uc002uzh.1 | P90 |
| LGSN | 51557 | 64053489 | Missense | c.326G > A | p.V98M | uc003peh.1 | P90 |
| NOC2L | 26155 | 881356 | Nonsense | c.648C > T | p.Q197* | uc009vjq.1 | P90 |
| OGFR | 11054 | 60915226 | Missense | c.1849G > T | p.R605L | uc002ydj.1 | P90 |
| PGBD5 | 79605 | 228564713 | Missense | c.350C > T | p.T117M | uc001htv.1 | P90 |
| ROBO1 | 6091 | 79070687 | Missense | c.253G > A | p.A85T | uc003dqe.1 | P90 |
| SEMA3E | 9723 | 82835175 | Missense | c.2457C > T | p.T664M | uc003uhy.1 | P90 |
| TP53 | 7157 | 7518933 | Missense | c.835A > G | p.H214R | uc002gim.2 | P90 |
| XRCC5 | 7520 | 216700595 | Missense | c.923T > C | p.L297S | uc002vfy.1 | P90 |
| ZNF142 | 7701 | 219217088 | Nonsense | c.2831G > T | p.E799* | uc002vin.1 | P90 |
| ZNF579 | 163033 | 60781946 | Missense | c.925T > G | p.V291G | uc002qlh.1 | P90 |
| ACSM2A | 123876 | 20390445 | Missense | c.1066C > A | p.P276H | uc010bwe.1 | P91 |
| AFTPH | 54812 | 64633697 | Missense | c.1617A > G | p.K529E | uc002sdc.1 | P91 |
| C16orf57 | 79650 | 56611602 | Missense | c.833A > C | p.Q250H | uc002emz.1 | P91 |
| C8orf47 | 203111 | 99170605 | Missense | c.332T > A | p.L62I | uc003yih.1 | P91 |
| CELF3 | 11189 | 149946729 | Missense | c.1444C > A | p.A217D | uc001eys.1 | P91 |
| DNHD1 | 144132 | 6536735 | Missense | c.3513C > T | p.A1134V | uc001mdw.2 | P91 |
| F2R | 2149 | 76064393 | Missense | c.852T > C | p.I196T | uc003ken.2 | P91 |
| FAM50A | 9130 | 153331803 | Missense | c.1028T > A | p.I318N | uc004flk.1 | P91 |
| FNDC3B | 64778 | 173495877 | Missense | c.937T > A | p.L294M | uc010hwt.1 | P91 |
| GDF2 | 2658 | 48033667 | Missense | c.1370G > A | p.V403I | uc001jfa.1 | P91 |
| GOLGA4 | 2803 | 37344179 | Missense | c.6168C > G | p.A1955G | uc003cgw.1 | P91 |
| HCK | 3055 | 30131240 | Nonsense | c.602G > A | p.W144* | uc002wxh.1 | P91 |
| KIAA0467 | 23334 | 43671006 | Missense | c.3316C > T | p.R952W | uc001cjk.1 | P91 |
| KIAA0947 | 23379 | 5516221 | Frame_Shift_Del | c.3996_3999delTC | p.T1258fs | uc003jdm.2 | P91 |
| KRT17 | 3872 | 37033980 | Missense | c.356G > A | p.R103H | uc002hxh.1 | P91 |
| MAGEC1 | 9947 | 140821627 | Missense | c.1057G > C | p.Q257H | uc004fbt.1 | P91 |
| MLL | 4297 | 117880825 | Missense | c.9031A > T | p.D3003V | uc001ptb.1 | P91 |
| NIN | 51199 | 50302815 | Missense | c.1786G > C | p.R532T | uc001wyi.1 | P91 |
| NPC1 | 4864 | 19390537 | Missense | c.1157G > C | p.E332Q | uc002kum.2 | P91 |
| OLR1 | 4973 | 10204214 | Missense | c.793T > G | p.L227V | uc001qxo.1 | P91 |
| PDE1C | 5137 | 31759666 | Missense | c.2761A > C | p.K723Q | uc003tco.1 | P91 |
| POLRMT | 5442 | 575894 | Missense | c.1021A > T | p.Q322L | uc002lpf.1 | P91 |
| RBMX | 27316 | 135785210 | Splice_Site_SNP | c.e7_splice_site | | uc004fae.1 | P91 |
| RNF150 | 57484 | 142008975 | Missense | c.1861G > A | p.E403K | uc003iio.1 | P91 |
| SF3B1 | 23451 | 197975079 | Missense | c.2146A > G | p.K700E | uc002uue.1 | P91 |
| SLC46A1 | 113235 | 23755946 | Missense | c.992G > C | p.W299S | uc002hbf.1 | P91 |
| SYT15 | 83849 | 46382034 | Missense | c.1361G > T | p.S403I | uc001jea.1 | P91 |
| TP53 | 7157 | 7518931 | Missense Mutation | c.643A > C | p.S215R | NM_000546 | P91 |
| TP53 | 7157 | 7513653 | Read-through | c.1375G > T | p.*394L | uc002gim.2 | P91 |
| TRO | 7216 | 54972506 | Missense | c.2731C > T | p.T875M | uc004dtq.1 | P91 |
| VDAC2 | 7417 | 76650736 | Splice_Site_SNP | c.e8_splice_site | | uc001jxa.1 | P91 |

TABLE 3
Analysis of mutation rate in CLL in relation to clinical characteristics.

| | | | Silent mutation rate | | | Non-silent mutation rate | | Total mutation rate | |
|---|---|---|---|---|---|---|---|---|---|
| | | N | Median, range | | p-value* | Median, range | p-value* | Median, range | p-value* |
| Clinical | Rai at sample | | | | 0.41 | | 0.27 | | 0.28 |
| Characteristics | 0-1 | 72 | 0.19 | (0.0, 1.09) | | 0.69 (0.08, 2.70) | | 0.88 (0.11, 3.79) | |
| | 2-4 | 19 | 0.16 | (0.04, 0.38) | | 0.57 (0.21, 1.25) | | 0.75 (0.29, 1.60) | |
| | Treatment status at | | | | 0.006 | | 0.14 | | 0.033 |
| | sample | | | | | | | | |
| | Chemotherapy na•ve | 61 | 0.17 | (0.0, 0.49) | | 0.66 (0.08, 1.44) | | 0.77 (0.11, 1.73) | |
| | Prior treatment | 30 | 0.21 | (0.07, 1.09) | | 0.70 (0.21, 2.70) | | 0.99 (0.29, 3.79) | |
| | Prior exposure to | | | | 0.005 | | 0.088 | | 0.019 |
| | nucleoside analogue | | | | | | | | |
| | No | 64 | 0.17 | (0, 0.49) | | 0.64 (0.08, 1.44) | | 0.77 (0.11, 1.73) | |
| | Yes | 27 | 0.22 | (0.07, 1.09) | | 0.73 (0.21, 2.70) | | 1.00 (0.29, 3.79) | |
| | IGHV mutation status | | | | 0.28 | | 0.5 | | 0.32 |
| | Unmutated | 40 | 0.19 | (0.04, 0.92) | | 0.69 (0.08, 2.14) | | 0.92 (0.11, 3.06) | |
| | mutated | 38 | 0.17 | (0, 1.09) | | 0.68 (0.11, 2.70) | | 0.82 (0.18, 3.79) | |
| | ZAP-70 | | | | 0.64 | | 0.99 | | 0.86 |
| | Negative | 44 | 0.18 | (0.04, 1.09) | | 0.69 (0.11, 2.70) | | 0.87 (0.18, 3.79) | |
| | Positive | 38 | 0.16 | (0, 0.92) | | 0.68 (0.08, 2.14) | | 0.88 (0.11, 3.06) | |
| FISH | 13q heterozygous | | | | 0.70 | | 0.66 | | 0.59 |
| Cytogenetics | deletion | | | | | | | | |
| | No | 38 | 0.18 | (0, 1.09) | | 0.63 (0.08, 2.70) | | 0.84 (0.11, 3.79) | |
| | Yes | 53 | 0.17 | (0.0, 0.92) | | 0.69 (0.11, 2.14) | | 0.87 (0.18, 3.06) | |
| | 13q homozygous | | | | 0.48 | | 0.24 | | 0.23 |
| | deletion | | | | | | | | |
| | No | 79 | 0.18 | (0, 1.09) | | 0.67 (0.08, 2.70) | | 0.81 (0.11, 3.79) | |
| | Yes | 12 | 0.20 | (0.10, 0.38) | | 0.77 (0.52, 1.07) | | 0.90 (0.71, 1.36) | |
| | Trisomy 12 | | | | 0.98 | | 0.66 | | 0.84 |
| | No | 78 | 0.19 | (0, 1.09) | | 0.67 (0.08, 2.70) | | 0.86 (0.11, 3.79) | |
| | Yes | 13 | 0.17 | (0.07, 0.49) | | 0.69 (0.35, 1.25) | | 0.77 (0.54, 1.68) | |
| | 11q deletion | | | | 0.85 | | 0.85 | | 0.96 |
| | No | 69 | 0.18 | (0.04, 1.09) | | 0.66 (0.14, 2.70) | | 0.86 (0.18, 3.79) | |
| | Yes | 22 | 0.19 | (0, 0.46) | | 0.69 (0.08, 1.25) | | 0.93 (0.11, 1.60) | |
| | 17p deletion | | | | 0.035 | | 0.12 | | 0.07 |
| | No | 74 | 0.17 | (0, 1.09) | | 0.67 (0.08, 2.70) | | 0.84 (0.11, 3.79) | |
| | Yes | 17 | 0.21 | (0.08, 0.92) | | 0.77 (0.49, 2.14) | | 1.11 (0.61, 3.06) | |
| Frequent | p53 | | | | 0.41 | | 0.14 | | 0.17 |
| Mutations | Unmutated | 77 | 0.17 | (0, 1.09) | | 0.66 (0.08, 2.70) | | 0.81 (0.11, 3.79) | |
| | Mutated | 14 | 0.20 | (0.04, 0.92) | | 0.78 (0.14, 2.14) | | 1.09 (0.18, 3.06) | |
| | SF3B1 | | | | 0.69 | | 0.57 | | 0.61 |
| | Unmutated | 77 | 0.18 | (0.04, 0.92) | | 0.68 (0.08, 2.14) | | 0.86 (0.11, 3.06) | |
| | Mutated | 14 | 0.20 | (0, 1.09) | | 0.63 (0.40, 2.70) | | 0.83 (0.50, 3.79) | |
| | ATM | | | | 0.80 | | 0.53 | | 0.78 |
| | Unmutated | 83 | 0.18 | (0, 1.09) | | 0.69 (0.08, 2.70) | | 0.86 (0.11, 3.79) | |
| | Mutated | 8 | 0.19 | (0.07, 0.46) | | 0.58 (0.42, 1.25) | | 0.76 (0.59, 1.60) | |
| | MYD88 | | | | 0.61 | | 0.84 | | 0.70 |
| | Unmutated | 82 | 0.18 | (0, 1.09) | | 0.68 (0.08, 2.70) | | 0.86 (0.11, 3.79) | |
| | Mutated | 9 | 0.19 | (0.04, 0.47) | | 0.59 (0.38, 1.26) | | 0.74 (0.47, 1.73) | |
| | NOTCH1 | | | | 0.41 | | 0.94 | | 0.81 |
| | Unmutated | 87 | 0.19 | (0, 1.09) | | 0.67 (0.08, 2.70) | | 0.86 (0.11, 3.79) | |
| | Mutated | 4 | 0.14 | (0.07, 0.27) | | 0.65 (0.53, 0.92) | | 0.74 (0.70, 1.19) | |
| | DDX3X | | | | 0.17 | | 0.30 | | 0.18 |
| | Unmutated | 88 | 0.19 | (0.04, 1.09) | | 0.69 (0.08, 2.70) | | 0.87 (0.11, 3.79) | |
| | Mutated | 3 | 0.12 | (0, 0.19) | | 0.57 (0.55, 0.58) | | 0.70 (0.55, 0.76) | |
| | MAPK1** | | | | | | | | |
| | Unmutated | 89 | 0.18 | (0, 1.09) | NA | 0.67 (0.08, 2.70) | NA | 0.86 (0.11, 3.79) | NA |
| | Mutated | 2 | (0.27, 0.36) | | | (0.34, 0.82) | | (0.61, 1.18) | |
| | FBXW3** | | | | 0.74 | | 0.37 | | 0.36 |
| | Unmutated | 88 | 0.18 | (0, 1.09) | | 0.67 (0.08, 2.70) | | 0.86 (0.11, 3.79) | |
| | Mutated | 3 | 0.25 | (0.08, 0.29) | | 0.74 (0.69, 0.90) | | 0.99 (0.77, 1.19) | |
| | ZMYM3 | | | | 0.12 | | 0.83 | | 0.94 |
| | Unmutated | 87 | 0.19 | (0, 1.09) | | 0.67 (0.11, 2.70) | | 0.86 (0.18, 3.79) | |
| | Mutated | 4 | 0.09 | (0.04, 0.25) | | 0.79 (0.08, 0.87) | | 0.94 (0.11, 0.99) | |
| Sequencing | Whole genome amplified | | | | 0.33 | | 0.31 | | 0.28 |
| Source Material | DNA (for exomes) | | | | | | | | |
| | No | 40 | 0.20 | (0.04, 1.09) | | 0.70 (0.14, 2.70) | | 0.90 (0.18, 3.79) | |
| | Yes | 51 | 0.16 | (0, 0.92) | | 0.67 (0.08, 2.14) | | 0.77 (0.11, 3.06) | |
| | Source of germline DNA | | | | 0.01 | | 0.006 | | 0.006 |
| | Buccal epithelia | 80 | 0.18 | (0, 1.09) | | 0.69 (0.29, 2.70) | | 0.87 (0.33, 3.79) | |
| | Skin fibroblasts | 7 | 0.29 | (0.08, 0.46) | | 0.67 (0.21, 1.12) | | 1.13 (0.29, 1.41) | |
| | Granulocytes | 4 | 0.05 | (0.04, 0.17) | | 0.13 (0.08, 0.42) | | 0.18 (0.11, 0.59) | |

*Testing excludes unknown category.
**One patient had two mutations of the same gene.

TABLE 4
Calculation of background rate of
non-synonymous mutation in CLL.

| Category | Rate |
|---|---|
| CpG transition | 1.91E−06 |
| Other C:G transition | 2.24E−07 |
| A:T transition | 2.05E−07 |
| Any transversion | 2.90E−07 |
| Indel + null | 1.33E−07 |
| Total | 7.25E−07 |

TABLE 5
Summary of mutations that have been previously identified in the COSMIC database (v76) in the significantly mutated genes.

| | Total | | | | | | | | | | |
|---|---|---|---|---|---|---|---|---|---|---|---|
| | num- | | | | | | | | | | |
| | ber | Total | # | | | | | | | | |
| | sam- | num- | cases | | | | | | | | |
| | ples | ber | per | | | | | | | | |
| | exam- | muta- | muta- | | | Endo- | | Pan- | GI/ | Mela- | |
| Gene | ined | tions | tion | AA change | Breast | metrial | Ovary | creas | colon | noma | Lung |
| SF3B1 | 93 | 6 | 1 | p.Q534P | 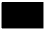 | | | | | | |
| | | | 1 | P.L1211L | | | 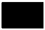 | | | | |
| | | | 1 | p.R568H | | | | 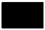 | | | |
| | | | 1 | p.Q699H | | | | 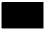 | | | |
| | | | 1 | p.K700E | | | | 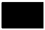 | | | |
| | | | 1 | p.P718L | | | | | | 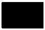 | |
| MYD88 | 445 | 12 | 2 | p.V217F | | | | | | | |
| | | | 1 | p.W218R | | | | | | | |
| | | | 2 | p.I220T | | | | | | | |
| | | | 11 | p.S219C | | | | | | | |
| | | | 2 | p.S222R | | | | | | | |
| | | | 3 | p.M232T | | | | | | | |
| | | | 5 | p.S243N | | | | | | | |
| | | | 64 | p.L265P | | | | | | | |
| | | | 1 | p.V52M | | | | | | | |
| | | | 1 | p.S149G | | | | | | | |
| | | | 1 | p.S149I | | | | | | | |
| | | | 1 | p.T294P | | | | | | | |
| FBXW7 | 5385 | 84 | 1 | p.A315T | | | | | 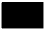 | | |
| | | | 1 | p.C386W | | | | | 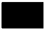 | | |
| | | | 1 | p.D130fs*41 | | | | | 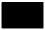 | | |
| | | | 1 | p.D440N | | | | | 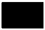 | | |
| | | | 1 | p.D480Y | | | | | | | 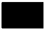 |
| | | | 1 | p.D520N | | | | | 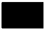 | | |
| | | | 1 | p.D527G | | | | | | | |
| | | | 1 | p.E110* | | | | | | | |
| | | | 1 | p.E117del | | | | | | | |
| | | | 1 | p.E117del | | | | | | | |
| | | | 1 | p.E121Y | | 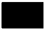 | | | | | |
| | | | 1 | p.E693K | | | | | 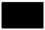 | | |
| | | | 1 | p.F549fs*6 | 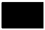 | | | | | | |
| | | | 1 | p.G397D | | | | | 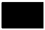 | | |
| | | | 1 | p.G423V | | | | | 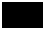 | | |
| | | | 2 | p.G423V | | | | | | | |
| | | | 1 | p.G423V | | | | | | | 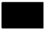 |
| | | | 1 | p.G579_Q581>E | | | | | 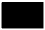 | | |
| | | | 1 | p.H379R | | | | | | | 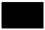 |
| | | | 1 | p.H420Y | | | | | | | |
| | | | 1 | p.H460R | | | | 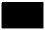 | | | |
| | | | 1 | p.H470P | | | | | | | |
| | | | 1 | p.H540Y | | | | | | | |
| | | | 1 | p.I435fs*9 | | | | | | | |
| | | | 1 | p.I563T | | | | 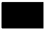 | | | |
| | | | 1 | p.K11R | | | | | | | 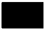 |
| | | | 1 | p.K164* | | 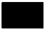 | | | | | |
| | | | 1 | p.K371fs*7 | | 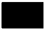 | | | | | |
| | | | 1 | p.K444fs*32 | | | | | | | |
| | | | 1 | p.L288fs*45 | | | | | | | |
| | | | 1 | p.L403fs*34 | | | | | 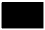 | | |
| | | | 1 | p.L594F | | | 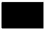 | | | | |
| | | | 1 | p.L651* | | | 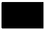 | | | | |
| | | | 1 | p.M467fs*5 | | | 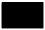 | | | | |
| | | | 1 | p.P298R | | | | | 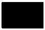 | | |
| | | | 2 | p.P298S | | | | | 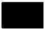 | | |
| | | | 1 | p.Q156E | | | 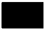 | | | | |
| | | | 1 | p.Q220* | | | | | 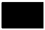 | | |
| | | | 1 | p.Q264R | 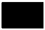 | | | | | | |
| | | | 1 | p.Q303* | | | | | 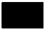 | | |
| | | | 1 | p.Q98* | | | | | 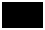 | | |
| | | | 1 | p.R13* | | | | | | | |
| | | | 3 | p.R224* | | | | | 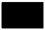 | | |
| | | | 6 | p.R278* | | | | | 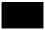 | | |
| | | | 1 | p.R312S | | | | | 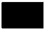 | | |
| | | | 1 | p.R367* | | 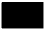 | | | | | |
| | | | 1 | p.R367* | | | | | 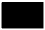 | | |
| | | | 1 | p.R367* | | | | | | | |
| | | | 4 | p.R393* | | | | | 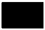 | | |
| | | | 1 | p.R393* | | | | | 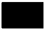 | | |
| | | | 1 | p.R441W | | 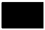 | | | | | |
| | | | 28 | p.R465C | | | | | | | |
| | | | 10 | p.R465C | | | | | 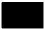 | | |
| | | | 6 | p.R465C | | | | | 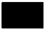 | | |
| | | | 4 | p.R465C | | | | 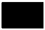 | | | |
| | | | 1 | p.R465C | | | | 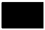 | | | |
| | | | 1 | p.R465C | | 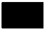 | | | | | |
| | | | 22 | p.R465H | | | | | | | |
| | | | 1 | p.R465H | | 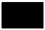 | | | | | |
| | | | 6 | p.R465H | | | | | 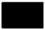 | | |
| | | | 1 | p.R465H | | | | 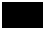 | | | |
| | | | 1 | p.R465H | | | | | | | |
| | | | 2 | p.R465L | | | | | | | |
| | | | 2 | p.R473fs*25 | | | | | | | |
| | | | 2 | p.R473fs*25 | | | 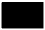 | | | | |
| | | | 1 | p.R473fs*4 | | 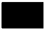 | | | | | |
| | | | 2 | p.R473fs*4 | | | | | 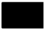 | | |
| | | | 1 | p.R479G | | 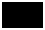 | | | | | |
| | | | 2 | p.R479G | | | | | | | |
| | | | 1 | p.R479L | | | | | 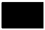 | | |
| | | | 4 | p.R479L | | | | | | | |
| | | | 1 | p.R479Q | | | | | | | |
| | | | 16 | p.R479Q | | | | | | | |
| | | | 7 | p.R479Q | | | | | 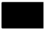 | | |
| | | | 1 | p.R479Q | | 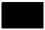 | | | | | |
| | | | 1 | p.R479Q | | 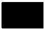 | | | | | |
| | | | 1 | p.R479Q | | | | | | | |
| | | | 1 | p.R479Q | | | | | | | |
| | | | 1 | p.R479Q | | | | | 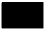 | | |
| | | | 1 | p.R484M | | | | | | | |
| | | | 1 | p.R484T | | | | | 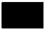 | | |
| | | | 5 | p.R505C | | | | | 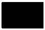 | | |
| | | | 1 | p.R505C | | | | 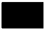 | | | |
| | | | 18 | p.R505C | | | | | | | |
| | | | 1 | p.R505C | | | 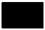 | | | | |
| | | | 1 | p.R505C | | | | | | | |
| | | | 2 | p.R505H | | | | | 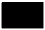 | | |
| | | | 1 | p.R505L | | | | | | | |
| | | | 1 | p.R505L | | | | | 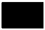 | | |
| | | | 1 | p.R505L | | | 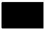 | | | | |
| | | | 1 | p.R505P | | | | | 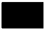 | | |
| | | | 1 | p.R505S | | | | | | | |
| | | | 1 | p.R543K | | | | | 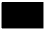 | | |
| | | | 1 | p.R658* | | 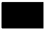 | | | | | |
| | | | 1 | p.R674Q | | | | | 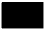 | | |
| | | | 1 | p.R689W | | | | | | | |
| | | | 1 | p.R689W | | | | | 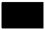 | | |
| | | | 1 | p.S182fs*57 | | 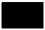 | | | | | |
| | | | 1 | p.S282* | | | | | 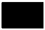 | | |
| | | | 1 | p.S294* | | | | 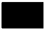 | | | |
| | | | 1 | p.S438F | | | | | 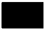 | | |
| | | | 6 | p.S582P | | | | | 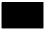 | | |
| | | | 1 | p.S596F | | | | | 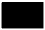 | | |
| | | | 1 | p.S668fs*26 | | | | | | | 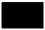 |
| | | | 1 | p.S668fs*39 | | | | | 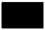 | | |
| | | | 1 | p.S668fs*39 | | | | | 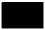 | | |
| | | | 1 | p.T15_G16insP | | 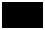 | | | | | |
| | | | 1 | p.T532N | | | | | 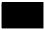 | | |
| | | | 1 | p.T653fs*8 | | | | | | | |
| | | | 1 | p.V504I | | | | | 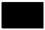 | | |
| | | | 1 | p.V504I | | | | | 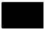 | | |
| | | | 1 | p.V627A | | | | | | | |
| | | | 1 | p.V672M | | 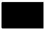 | | | | | |
| | | | 2 | p.W446* | | | | | 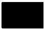 | | |
| | | | 2 | p.W526R | | | | | 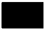 | | |
| | | | 1 | p.W649* | | | | | 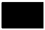 | | |
| | | | 1 | p.Y519C | | | | 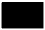 | | | |
| | | | 1 | p.Y545C | | | | | 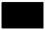 | | |
| MAPK1 | 902 | 1 | 1 | p.A143A | | | 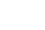 | | | | |
| DDX3X | 659 | 4 | 1 | p.R294T | 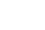 | | | | | | |
| | | | 1 | p.A502T | | | | | | 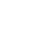 | |
| | | | 1 | p.R548T | | | 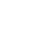 | | | | |
| | | | 1 | p.N551H | | | 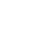 | | | | |
| ATM | 2852 | 179 | 2 | p.A1309T | | | | | | | |
| | | | 2 | p.A1742P | | | | | | | |
| | | | 1 | p.A1945T | | | | | 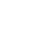 | | |
| | | | 1 | p.A2274T | | | | | | | |
| | | | 1 | p.A2420P | | | | | | | |
| | | | 1 | p.A2622V | | | | | | | |
| | | | 1 | p.A2631fs*2 | | | | | | | |
| | | | 1 | p.A2893fs*3 | | | | | | | |
| | | | 2 | p.A3006P | | | | | | | |
| | | | 1 | p.A350T | | | | | | | |
| | | | 1 | p.C2349W | | | | | | | |
| | | | 1 | p.C353fs*5 | | | | | | | |
| | | | 1 | p.C540Y | | | | | 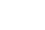 | | |
| | | | 1 | p.C693_Q700>E | | | | | | | |
| | | | 1 | p.D1208H | | | 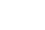 | | | | |
| | | | 1 | p.D126E | | | | | | | |
| | | | 1 | p.D1682H | | | | | | | |
| | | | 2 | p.D1682Y | | | | | | | |
| | | | 9 | p.D1853N | 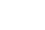 | | | | | | |
| | | | 1 | p.D1853V | | | | | | | |
| | | | 1 | p.D2708N | | | | | | | 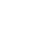 |
| | | | 1 | p.D2725G | | | | | | | |
| | | | 2 | p.D2725V | | | | | | | |
| | | | 1 | p.E1612_Q1620>* | | | | | | | |
| | | | 1 | p.E1991D | | | | | | | |
| | | | 1 | p.E2052* | | | | | | | |
| | | | 1 | p.E2164K | | | | | | | |
| | | | 1 | p.E2423G | | | | | | | |
| | | | 1 | p.E2423K | | | | | | | |
| | | | 1 | p.E26fs*7 | | | | | | | |
| | | | 2 | p.E522fs*43 | | | | | | | |
| | | | 1 | p.E770* | | | | | | | |
| | | | 2 | p.E848Q | | | | | | | 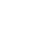 |
| | | | 1 | p.F1209fs*19 | | | | | | | |
| | | | 1 | p.F1463L | | | | | | | |
| | | | 1 | p.F1463S | | | | | | | |
| | | | 1 | p.F168_V170>L | | | | | | | |
| | | | 1 | p.F1683fs*7 | | | | | | | |
| | | | 1 | p.F2732L | | | | | | | |
| | | | 1 | p.F2799fs*4 | | | | | | | 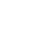 |
| | | | 1 | p.F570S | | | | | | | |
| | | | 3 | p.F858L | | | | | | | |
| | | | 1 | p.G138R | | | | | | | |
| | | | 1 | p.G2023R | | | | | | | |
| | | | 1 | p.G2063E | | | | | | | |
| | | | 2 | p.G2695A | | | | | | | |
| | | | 2 | p.G2867E | | | | | | | |
| | | | 1 | p.G2925D | | | | | | | |
| | | | 1 | p.G2925V | | | | | | | |
| | | | 1 | p.G3051V | | | | | | | |
| | | | 1 | p.G558* | | | | | | | 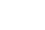 |
| | | | 1 | p.H1380Y | | | | | | | |
| | | | 1 | p.H2872Q | | | | | | | |
| | | | 1 | p.H996Q | | | | | | | 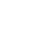 |
| | | | 1 | p.I1237fs*2 | | | | | | | |
| | | | 2 | p.I1332fs*27 | | | | | | | |
| | | | 1 | p.I1407S | | | | | | | |
| | | | 1 | p.I1407T | | | | | | | |
| | | | 1 | p.I1469M | | | | | | | |
| | | | 2 | p.I1681V | | | | | | | |
| | | | 1 | p.I2055fs*33 | | | | | | | |
| | | | 1 | p.I2076S | | | | | | | |
| | | | 1 | p.I2356F | | | | | | | |
| | | | 1 | p.I2888T | | | | | | | |
| | | | 1 | p.I352T | | | | | | | |
| | | | 1 | p.K1454N | | | | | | | |
| | | | 1 | p.K1994E | | | | | | | |
| | | | 1 | p.K2213fs*22 | | | | | | | |
| | | | 1 | p.K2237fs*11 | | | | | | | |
| | | | 1 | p.K2418_R2419insK | | | | | | | |
| | | | 1 | p.K2717M | | | | | | | |
| | | | 1 | p.K2810del | | | | | | | |
| | | | 1 | p.K3018N | | | | | | | |
| | | | 1 | p.K902fs*18 | | | | | | | |
| | | | 1 | p.L1322I | | | | | | | |
| | | | 1 | p.L1322P | | | | | | | 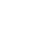 |
| | | | 1 | p.L1472F | | | 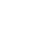 | | | | |
| | | | 1 | p.L1708fs*6 | | | | | | | |
| | | | 1 | p.L1764fs*12 | | | | | | | |
| | | | 2 | p.L1794L | | | | | | | 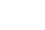 |
| | | | 1 | p.L1910H | | | | | | | |
| | | | 1 | p.L1939V | | | | | | | |
| | | | 1 | p.L2004R | | | | | | | |
| | | | 1 | p.L2417P | | | | | | | 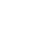 |
| | | | 1 | p.L2427R | | | | | | | |
| | | | 1 | p.L2445P | | | | | | | |
| | | | 1 | p.L2450fs*11 | | | | | | | |
| | | | 1 | p.L2722R | | | | | | | |
| | | | 2 | p.L2890V | | | | | | | |
| | | | 1 | p.L2945fs*7 | | | | | | | |
| | | | 1 | p.L3017P | | | | | | | |
| | | | 1 | p.L895fs*4 | | | | | | | |
| | | | 1 | p.M1040V | | | | | | | |
| | | | 1 | p.M1916I | 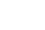 | | | | | | |
| | | | 1 | p.M1L | | | | | | | |
| | | | 1 | p.M2616I | | | | | | | |
| | | | 1 | p.M2805fs*1 | | | | | | | |
| | | | 1 | p.M855fs*24 | 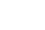 | | | | | | |
| | | | 1 | p.N1739T | | | | | 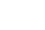 | | |
| | | | 1 | p.N1801Y | | | | | | | |
| | | | 1 | p.N750K | | | | | | | |
| | | | 1 | p.P1054R | | | | | | | |
| | | | 1 | p.P1829fs*5 | | | | | | | 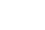 |
| | | | 1 | p.P2699R | | | | | | | |
| | | | 1 | p.P2842R | | | | | | | 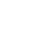 |
| | | | 4 | p.P604S | | | | | | | |
| | | | 1 | p.Q1128R | | | | | | | |
| | | | 1 | p.Q1361* | | | | | | | |
| | | | 1 | p.Q162* | | | | | | | |
| | | | 1 | p.Q163* | | | | | | | |
| | | | 1 | p.Q2414* | | | 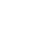 | | | | |
| | | | 1 | p.Q2442P | | | | | | | |
| | | | 2 | p.Q2442P | | | | | | | 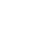 |
| | | | 1 | p.Q2593* | | | | | | | |
| | | | 1 | p.Q466* | | | | | | | |
| | | | 1 | p.Q747H | | | | | | | 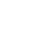 |
| | | | 1 | p.R1086L | | | | | | | 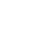 |
| | | | 1 | p.R1304fs*43 | | | | | | | |
| | | | 1 | p.R2263S | | | | | | | |
| | | | 1 | p.R2273fs*37 | | | | | | | |
| | | | 1 | p.R23Q | | | | | 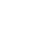 | | |
| | | | 1 | p.R2400fs*6 | | | | | | | |
| | | | 1 | p.R2443* | | | | | | | |
| | | | 1 | p.R2443Q | | | | | | | |
| | | | 2 | p.R2443Q | | | | | 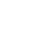 | | |
| | | | 1 | p.R2453P | | | | | | | 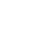 |
| | | | 1 | p.R2486G | | | | | | | |
| | | | 1 | p.R2713K | | | | | | | |
| | | | 1 | p.R2832C | | | | | | | |
| | | | 1 | p.R2871_H2872>S | | | | | | | |
| | | | 1 | p.R2912K | | | | | | | |
| | | | 4 | p.R3008C | | | | | | | |
| | | | 4 | p.R3008H | | | | | | | |
| | | | 3 | p.R3047* | | | | | | | |
| | | | 2 | p.R337C | | | | | 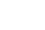 | | |
| | | | 1 | p.R337H | | | | | 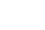 | | |
| | | | 2 | p.R337S | | | | | | | |
| | | | 1 | p.R717W | | | | | | | |
| | | | 1 | p.S1179F | | | | | | | |
| | | | 1 | p.S151fs*2 | | | | | | | |
| | | | 1 | p.S1770* | | | | | | | |
| | | | 1 | p.S1905L | | | | | | | 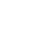 |
| | | | 1 | p.S207C | | | | | | | |
| | | | 1 | p.S2375I | | | | | | | 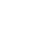 |
| | | | 1 | p.S2394L | | | | | | | |
| | | | 1 | p.S2408L | | | | | 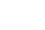 | | |
| | | | 1 | p.S2546_I2548del | | | | | | | |
| | | | 1 | p.S2859F | | | | | | | |
| | | | 1 | p.S707fs*29 | | | | | | | |
| | | | 2 | p.S707P | | | | | | | |
| | | | 1 | p.S853* | | | | | | | 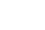 |
| | | | 1 | p.S978P | | | | | | | |
| | | | 1 | p.T1735fs*11 | | | | | | | |
| | | | 1 | p.T1743I | | | | | | | |
| | | | 1 | p.T1953R | | | | | | | |
| | | | 1 | p.T2396S | | | | | | | |
| | | | 1 | p.T2438K | | | | | | | |
| | | | 1 | p.T261fs*10 | | | | | | | |
| | | | 2 | p.T2666A | | | | | | | 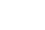 |
| | | | 1 | p.T2911del | | | | | | | |
| | | | 2 | p.T2947S | | | | | | | |
| | | | 1 | p.T935T | 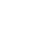 | | | | | | |
| | | | 1 | p.V1292_Q1331del | | | | | | | |
| | | | 2 | p.V1941L | | | | | | | |
| | | | 1 | p.V2424G | | | | | | | |
| | | | 1 | p.V245A | | | | | | | |
| | | | 2 | p.V410A | | | | | | | |
| | | | 1 | p.W1221* | | | | | | | |
| | | | 1 | p.W2845* | | | | | | | |
| | | | 1 | p.W308* | | | | | | | |
| | | | 1 | p.W393* | | | | | | | |
| | | | 1 | p.W57* | | | | | | | |
| | | | 1 | p.Y1392fs*7 | | | | | | | |
| | | | 1 | p.Y1475C | | | | | | | |
| | | | 1 | p.Y1961C | | | | | | | |
| | | | 1 | p.Y2019S | | | | | | | |
| | | | 1 | p.Y2627fs*29 | | | | | | | |
| | | | 1 | p.Y2817* | | | | | | | |
| | | | 1 | p.Y2954C | | | 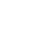 | | | | |
| | | | 1 | p.Y332C | | | | | | | |
| NOTCH1 | 5090 | 645 | 1 | p.1719_1720>QKGPLAAFLGA LASLGSLTIPYLI | | | | | | | |
| | | | 1 | p.1741_1742>MKLVEPPPPAQ LHFMYVA | | | | | | | |
| | | | 1 | p.A1611_A1636>A | | | | | | | |
| | | | 1 | p.A1611T | | | | | | | |
| | | | 1 | p.A1635S | | | | | | | |
| | | | 1 | p.A1651T | | | | | | | |
| | | | 2 | p.A1697D | | | | | | | |
| | | | 1 | p.A1701P | | | | | | | |
| | | | 1 | p.A1701V | | | | | | | |
| | | | 6 | p.A1702P | | | | | | | |
| | | | 1 | p.A1721_V1722>YG | | | | | | | |
| | | | 1 | p.A1741_A1742ins11 | | | | | | | |
| | | | 1 | p.A1741_A1742ins17 | | | | | | | |
| | | | 1 | p.A1741_A1742ins36 | | | | | | | |
| | | | 1 | p.A1742_A1743ins GALHFMYVA | | | | | | | |
| | | | 3 | p.A2280V | | | | | | | |
| | | | 1 | p.A2332T | | | | | | | |
| | | | 1 | p.A2340fs*15 | | | | | | | |
| | | | 1 | p.A2357_S2358>TN | | | | | | | |
| | | | 1 | p.A2425V | | | | | | | |
| | | | 1 | p.A2426fs | | | | | | | |
| | | | 1 | p.A2426fs*15 | | | | | | | |
| | | | 1 | p.A2442V | | | | | | | |
| | | | 1 | p.A2444fs*39 | | | | | | | |
| | | | 1 | p.A2453T | | | | | | | |
| | | | 5 | p.A2464fs*14 | | | | | | | |
| | | | 1 | p.A2554D | | | | | | | |
| | | | 1 | p.C1117C | 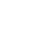 | | | | | | |
| | | | 1 | p.C1686F | | | | | | | |
| | | | 3 | p.C1693R | | | | | | | |
| | | | 1 | p.D1547G | | | | | | | |
| | | | 1 | p.D1610_A1611insPQP | | | | | | | |
| | | | 1 | p.D1610_R1634> | | | | | | | |
| | | | 1 | p.D1610_R1634del | | | | | | | |
| | | | 2 | p.D1610V | | | | | | | |
| | | | 1 | p.D1643H | | | | | | | 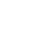 |
| | | | 1 | p.D1682G | | | | | | | |
| | | | 11 | p.D1699D | | | | | | | |
| | | | 1 | p.D2443fs | | | | | | | |
| | | | 1 | p.D2443fs*2 | | | | | | | |
| | | | 1 | p.D2443fs*35 | | | | | | | |
| | | | 3 | p.D2443fs*39 | | | | | | | |
| | | | 1 | p.D620Y | 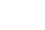 | | | | | | |
| | | | 1 | p.E1584_Q1585insPVELMPPE | | | | | | | |
| | | | 1 | p.E1584>AQ | | | | | | | |
| | | | 1 | p.E1584>GTHPKE | | | | | | | |
| | | | 1 | p.E1584G | | | | | | | |
| | | | 1 | p.E2268fs*31 | | | | | | | |
| | | | 1 | p.E2268fs*89 | | | | | | | |
| | | | 1 | p.E2507* | | | | | | | |
| | | | 1 | p.E2507fs*6 | | | | | | | |
| | | | 1 | p.E2516fs*1 | | | | | | | |
| | | | 1 | p.E2516fs*3 | | | | | | | |
| | | | 1 | p.E2516fs*71 | | | | | | | |
| | | | 1 | p.F1541L | | | | | | | |
| | | | 1 | p.F1591_E1596>LLGG | | | | | | | |
| | | | 1 | p.F1591>SI | | | | | | | |
| | | | 1 | p.F1593_F1594>TA | | | | | | | |
| | | | 2 | p.F1593_L1594ins12 | | | | | | | |
| | | | 1 | p.F1593_L1594insC | | | | | | | |
| | | | 1 | p.F1593_L1594insDLS | | | | | | | |
| | | | 1 | p.F1593_L1594insGVN | | | | | | | |
| | | | 1 | p.F1593_L1594insSP | | | | | | | |
| | | | 1 | p.F1593_R1595>HFDG | | | | | | | |
| | | | 1 | p.F1593>KED | | | | | | | |
| | | | 3 | p.F1593>LA | | | | | | | |
| | | | 1 | p.F1593>LG | | | | | | | |
| | | | 2 | p.F1593>LGA | | | | | | | |
| | | | 1 | p.F1593>LGP | | | | | | | |
| | | | 3 | p.F1593>LS | | | | | | | |
| | | | 1 | p.F1593>LSP | | | | | | | |
| | | | 1 | p.F1593>PEH | | | | | | | |
| | | | 3 | p.F1593C | | | | | | | |
| | | | 1 | p.F1593L | | | | | | | |
| | | | 12 | p.F1593S | | | | | | | |
| | | | 1 | p.F1607_A1611>LVPSK | | | | | | | |
| | | | 1 | p.F1607_F>LPL | | | | | | | |
| | | | 1 | p.F1607_K1608>LCPEM | | | | | | | |
| | | | 1 | p.F1607_K1608>LGLWRQ | | | | | | | |
| | | | 1 | p.F1607_K1608>RSE | | | | | | | |
| | | | 1 | p.F1607_K1608ins15 | | | | | | | |
| | | | 1 | p.F1607_K1608insGS | | | | | | | |
| | | | 1 | p.F1607_K1608insLVGCGQ | | | | | | | |
| | | | 1 | p.F1607_K1608insNPNVVLFK | | | | | | | |
| | | | 1 | p.F1607_K1608insVRVTHTK | | | | | | | |
| | | | 1 | p.F1607_R1609>LG | | | | | | | |
| | | | 1 | p.F1607>FVA | | | | | | | |
| | | | 2 | p.F1607>LD | | | | | | | |
| | | | 1 | p.F1607>LDP | | | | | | | |
| | | | 1 | p.F1607>LGM | | | | | | | |
| | | | 1 | p.F1607>LGT | | | | | | | |
| | | | 1 | p.F1607>LNPLS | | | | | | | |
| | | | 1 | p.F1607>LPPHP | | | | | | | |
| | | | 2 | p.F1607>LPRNED | | | | | | | |
| | | | 1 | p.F1607>LRFL | | | | | | | |
| | | | 1 | p.F1607>LSMPP | | | | | | | |
| | | | 1 | p.F1607>WNS | | | | | | | |
| | | | 1 | p.F1618_P1619del | | | | | | | |
| | | | 1 | p.F1618_P1619insEPP | | | | | | | |
| | | | 1 | p.F1618_Y1620>WSP | | | | | | | |
| | | | 1 | p.F1618>FKN | | | | | | | |
| | | | 3 | p.F1618del | | | | | | | |
| | | | 1 | p.F1694S | | | | | | | |
| | | | 1 | p.F1737_M1738ins13 | | | | | | | |
| | | | 1 | p.F2267fs*87 | | | | | | | |
| | | | 1 | p.F2482fs*5 | | | | | | | |
| | | | 1 | p.F2510fs*1 | | | | | | | |
| | | | 1 | p.G1137V | 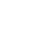 | | | | | | |
| | | | 1 | p.G1216D | 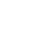 | | | | | | |
| | | | 1 | p.G135W | | | | | | | 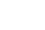 |
| | | | 1 | p.G1559V | 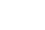 | | | | | | |
| | | | I | p.G1647S | | | | | | | |
| | | | 1 | p.G1657S | | | | | | | |
| | | | 1 | p.G1660D | | | | | | | |
| | | | 1 | p.G1705* | | | | | | | 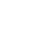 |
| | | | 2 | p.G2153R | | | | | | | |
| | | | 1 | p.G2153S | 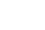 | | | | | | |
| | | | 1 | p.G2246R | | | | | | | |
| | | | 1 | p.G2263fs*6 | | | | | | | |
| | | | 1 | p.G2334fs*21 | | | | | | | |
| | | | 1 | p.G2421fs*3 | | | | | | | |
| | | | 3 | p.H1592_F1593>QT | | | | | | | |
| | | | 1 | p.H1592_F1593insY | | | | | | | |
| | | | 1 | p.H1592Y | | | | | | | |
| | | | 1 | p.H1602_T1603insQ | | | | | | | |
| | | | 1 | p.H1602P | | | | | | | |
| | | | 1 | p.H1612_G1613>QIVVFKRDA HG | | | | | | | |
| | | | 1 | p.H1612_P1619>RGT | | | | | | | |
| | | | 1 | p.H2276fs*79 | | | | | | | 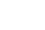 |
| | | | 1 | p.H2419fs*16 | | | | | | | |
| | | | 1 | p.H2429fs*8 | | | | | | | |
| | | | 1 | p.H2508Y | | | | | | | |
| | | | 1 | p.I1617_R1623>G | | | | | | | |
| | | | 7 | p.I1617N | | | | | | | |
| | | | 1 | p.I1632V | | | | | | | |
| | | | 1 | p.I1633I | | | | | | | |
| | | | 1 | p.I1676_V1677>I | | | | | | | |
| | | | 1 | p.I1676_V1677>MF | | | | | | | |
| | | | 1 | p.I1676>TAFL | | | | | | | |
| | | | 4 | p.I1681N | | | | | | | |
| | | | 2 | p.I1681S | | | | | | | |
| | | | 2 | p.I1719T | | | | | | | |
| | | | 1 | p.I2457fs.21 | | | | | | | |
| | | | 1 | p.K1608_R1609insPAK | | | | | | | |
| | | | 1 | p.K1608>GPPLQ | | | | | | | |
| | | | 1 | p.K1608N | | | | | | | |
| | | | 2 | p.K1783_R1784ins31 | | | | | | | |
| | | | 1 | p.L122fs.3 | | | | | 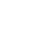 | | |
| | | | 31 | p.L1575P | | | | | | | |
| | | | 2 | p.L1575Q | | | | | | | |
| | | | 1 | p.L1586>PPEAV | | | | | | | |
| | | | 35 | p.L1586P | | | | | | | |
| | | | 1 | p.L1594_R1595ins12 | | | | | | | |
| | | | 1 | p.L1594_R1595insA | | | | | | | |
| | | | 1 | p.L1594>NPM | | | | | | | |
| | | | 34 | p.L1594P | | | | | | | |
| | | | 1 | p.L1597_S1598insG | | | | | | | |
| | | | 3 | p.L1597H | | | | | | | |
| | | | 1 | p.L1601_H1602insA | | | | | | | |
| | | | 1 | p.L1601_H1602insL | | | | | | | |
| | | | 40 | p.L1601P | | | | | | | |
| | | | 5 | p.L1601Q | | | | | | | |
| | | | 29 | p.L1679P | | | | | | | |
| | | | 5 | p.L1679Q | | | | | | | |
| | | | 1 | p.L1707_A1708ins14 | | | | | | | |
| | | | 4 | p.L1710P | | | | | | | |
| | | | 1 | p.L2327fs*5 | | | | | | | |
| | | | 1 | p.L2336fs*19 | | | | | | | |
| | | | 1 | p.L2336fs*20 | | | | | | | |
| | | | 1 | p.L2343fs*12 | | | | | | | |
| | | | 1 | p.L2391_Q2392>PFPF* | | | | | | | |
| | | | 1 | p.L2430_G2431>CLVSR | | | | | | | |
| | | | 1 | p.L2435fs*1 | | | | | | | |
| | | | 1 | p.L2435fs*2 | | | | | | | |
| | | | 1 | p.L2447fs*33 | | | | | | | |
| | | | 2 | p.L2458V | | | | | | | |
| | | | 1 | p.L2465L | | | | | | | |
| | | | 2 | p.L2469fs*10 | | | | | | | |
| | | | 1 | p.L2469fs*11 | | | | | | | |
| | | | 1 | p.L2473fs*1 | | | | | | | |
| | | | 1 | p.L2473fs*7 | | | | | | | |
| | | | 1 | p.L2511L | | | | | | | |
| | | | 1 | p.M1581_P1582del | | | | | | | |
| | | | 1 | p.M1581_P1582insLMHLAF | | | | | | | |
| | | | 1 | p.M1581_P1582insPRYEL | | | | | | | |
| | | | 2 | p.M1581del | | | | | | | |
| | | | 1 | p.M1616_F1618>L | | | | | | | |
| | | | 1 | p.M1738_Y1739ins35 | | | | | | | |
| | | | 1 | p.M2057fs*211 | | | | | | | |
| | | | 1 | p.M2347fs*16 | | | | | | | |
| | | | 1 | p.M2347fs*9 | | | | | | | |
| | | | 1 | p.N1900I | | | | | | | |
| | | | 1 | p.N2296_F2297insWV | | | | | | | |
| | | | 1 | p.N2390fs*33 | | | | | | | |
| | | | 1 | p.N2402_I2403>GPSLNN | | | | | | | |
| | | | 1 | p.N2402fs*21 | | | | | | | |
| | | | 1 | p.P1582_E1584>Q | | | | | | | |
| | | | 1 | p.P1583_E1584insP | | | | | | | |
| | | | 1 | p.P1583_L1586>IEA | | | | | | | |
| | | | 4 | p.P1583del | | | | | | | |
| | | | 1 | p.P2272S | | | | | | | |
| | | | 1 | p.P2333fs*22 | | | | | | | |
| | | | 1 | p.P2411fs*12 | | | | | | | |
| | | | 1 | p.P2412del | | | | | | | |
| | | | 1 | p.P2412P | | | | | | | |
| | | | 1 | p.P2413S | | | | | | | |
| | | | 3 | p.P2413T | | | | | | | |
| | | | 1 | p.P2414L | | | | | | | |
| | | | 2 | p.P2416del | | | | | | | |
| | | | 2 | p.P2418L | | | | | | | |
| | | | 1 | p.P2439fs*40 | | | | | | | |
| | | | 1 | p.P2439fs*41 | | | | | | | |
| | | | 1 | p.P2439L | | | | | | | |
| | | | 1 | p.P2459fs*21 | | | | | | | |
| | | | 1 | p.P2459fs*61 | | | | | | | |
| | | | 2 | p.P2463fs*15 | | | | | | | |
| | | | 1 | p.P2475fs*1 | | | | | | | |
| | | | 1 | p.P2475fs*3 | | | | | | | |
| | | | 2 | p.P2475fs*34 | | | | | | | |
| | | | 2 | p.P2475fs*5 | | | | | | | |
| | | | 1 | p.P2476fs | | | | | | | |
| | | | 1 | p.P2476fs*2 | | | | | | | |
| | | | 1 | p.P2494fs*13 | | | | | | | |
| | | | 3 | p.P2494fs*3 | | | | | | | |
| | | | 1 | p.P2506fs*6 | | | | | | | |
| | | | 3 | p.P2506P | | | | | | | |
| | | | 1 | p.P2509fs*8 | | | | | | | |
| | | | 2 | p.P2513fs*3 | | | | | | | |
| | | | 5 | p.P2513L | | | | | | | |
| | | | 1 | p.P2515fs | | | | | | | |
| | | | 29 | p.P2515fs*4 | | | | | | | |
| | | | 2 | p.P2518* | | | | | | | |
| | | | 1 | p.P2518fs*6 | | | | | | | |
| | | | 1 | p.Q1050L | | | | | | | |
| | | | 2 | p.Q1585>PVELMPPE | | | | | | | |
| | | | 1 | p.Q1585del | | | | | | | |
| | | | 1 | p.Q1615_F1618>LCR | | | | | | | |
| | | | 1 | p.Q1615_M1616>L | | | | | | | |
| | | | 1 | p.Q1615K | | | | | | | |
| | | | 1 | p.Q1685_C1686insLEGQR | | | | | | | |
| | | | 1 | p.Q2316* | | | | | | | |
| | | | 1 | p.Q2344fs*11 | | | | | | | |
| | | | 2 | p.Q2392* | | | | | | | |
| | | | 2 | p.Q2394* | | | | | | | |
| | | | 1 | p.Q2395* | | | | | | | |
| | | | 1 | p.Q2396* | | | | | | | |
| | | | 1 | p.Q2399* | | | | | | | |
| | | | 1 | p.Q2404* | | | | | | | |
| | | | 1 | p.Q2406* | | | | | | | |
| | | | 1 | p.02407* | | | | | | | |
| | | | 1 | p.Q2410* | | | | | | | |
| | | | 2 | p.Q2417* | | | | | | | |
| | | | 4 | p.Q2441* | | | | | | | |
| | | | 1 | p.Q2445* | | | | | | | |
| | | | 1 | p.Q2445fs*65 | | | | | | | |
| | | | 9 | p.Q2460* | | | | | | | |
| | | | 2 | p.Q2460fs*18 | | | | | | | |
| | | | 2 | p.Q2502* | | | | | | | |
| | | | 3 | p.Q2504* | | | | | | | |
| | | | 1 | p.Q2504fs | | | | | | | |
| | | | 1 | p.Q2504fs*5 | | | | | | | |
| | | | 2 | p.Q2520* | | | | | | | |
| | | | 3 | p.R1587P | | | | | | | |
| | | | 3 | p.R1595_E1596ins12 | | | | | | | |
| | | | 1 | p.R1595_L1597>L | | | | | | | |
| | | | 2 | p.R1595>PRLPHNSSFHFLR | | | | | | | |
| | | | 1 | p.R1595>PRLPHNSSSHFL | | | | | | | |
| | | | 1 | p.R1599>QS | | | | | | | |
| | | | 20 | p.R1599P | | | | | | | 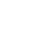 |
| | | | 1 | p.R1609_A1611>T | | | | | | | |
| | | | 1 | p.R1609_D1610ins12 | | | | | | | |
| | | | 1 | p.R1609S | | | | | | | |
| | | | 1 | p.R1628H | | | | | | | |
| | | | 1 | p.R1628Q | | | | | | | |
| | | | 1 | p.R1634L | | | | | | | |
| | | | 1 | p.R1663L | | | | | | | |
| | | | 1 | p.R2160H | 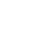 | | | | | | |
| | | | 1 | p.R2273fs*78 | | | | | | | |
| | | | 1 | p.R2328W | | | | | | | 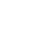 |
| | | | 1 | p.S1598I | | | | | | | |
| | | | 3 | p.S1675_I1676insG | | | | | | | |
| | | | 1 | p.S1675P | | | | | | | |
| | | | 1 | p.S1709S | | | | | | | |
| | | | 1 | p.S2290R | | | | | | | |
| | | | 1 | p.S2291S | | | | | | | |
| | | | 2 | p.S2330fs*25 | | | | | | | |
| | | | 1 | p.S2330fs*7 | | | | | | | |
| | | | 1 | p.S2337fs*18 | | | | | | | |
| | | | 1 | p.S2342fs*1 | | | | | | | |
| | | | 1 | p.S2342fs*13 | | | | | | | |
| | | | 1 | p.S2342fs*7 | | | | | 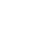 | | |
| | | | 2 | p.S2408N | | | | | | | |
| | | | 1 | p.S2423fs*1 | | | | | | | |
| | | | 1 | p.S2424* | | | | | | | |
| | | | 2 | p.S2427fs*4 | | | | | | | |
| | | | 1 | p.S2433fs*5 | | | | | | | |
| | | | 1 | p.S2436fs*2 | | | | | | | |
| | | | 1 | p.S2440fs*1 | | | | | | | |
| | | | 4 | p.S2440fs*4 | | | | | | | |
| | | | 1 | p.S2440G | | | | | | | |
| | | | 1 | p.S2450fs*28 | | | | | | | |
| | | | 1 | p.S2468* | | | | | | | |
| | | | 1 | p.S2468fs*1 | | | | | | | |
| | | | 1 | p.S2468fs*10 | | | | | | | |
| | | | 2 | p.S2468fs*11 | | | | | | | |
| | | | 1 | p.S2468fs*15 | | | | | | | |
| | | | 2 | p.S2487* | | | | | | | |
| | | | 1 | p.S2487fs*7 | | | | | | | |
| | | | 1 | p.S2492fs*67 | | | | | | | |
| | | | 3 | p.S2493* | | | | | | | |
| | | | 1 | p.S2493>S* | | | | | | | |
| | | | 1 | p.S2493>SP* | | | | | | | |
| | | | 1 | p.S2493fs*100 | | | | | | | |
| | | | 1 | p.S2493fs*3 | | | | | | | |
| | | | 1 | p.S2514F | | | | | | | |
| | | | 1 | p.S2514fs*4 | | | | | | | |
| | | | 4 | p.S2524* | | | | | | | |
| | | | 1 | p.S2528fs*80 | | | | | | | |
| | | | 1 | p.S356del | | | | | | | |
| | | | 1 | p.T1574_V1576del | | | | | | | |
| | | | 1 | p.T1603_N1604ins17 | | | | | | | |
| | | | 1 | p.T1997M | | | | | | | 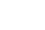 |
| | | | 1 | p.T2467fs*11 | | | | | | | |
| | | | 1 | p.T2467fs*12 | | | | | | | |
| | | | 1 | p.T2467M | | | | | | | |
| | | | 1 | p.T2484A | | | | | | | |
| | | | 2 | p.T2484M | | | | | | | |
| | | | 1 | p.T2512fs*1 | | | | | | | |
| | | | 1 | p.T445T | | | | | | | 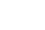 |
| | | | 1 | p.T971I | 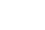 | | | | | | |
| | | | 1 | p.V1576_V1578del | | | | | | | |
| | | | 1 | p.V1577_V1578>FRP | | | | | | | |
| | | | 1 | p.V1577A | | | | | | | |
| | | | 3 | p.V1577E | | | | | | | |
| | | | 1 | p.V1578_V1579insA | | | | | | | |
| | | | 1 | p.V1578_V1579insGV | | | | | | | |
| | | | 1 | p.V1578A | | | | | | | |
| | | | 1 | p.V1579A | | | | | | | |
| | | | 20 | p.V1579del | | | | | | | |
| | | | 2 | p.V1579E | | | | | | | |
| | | | 1 | p.V1579G | | | | | | | |
| | | | 1 | p.V1605_R1609>LKGCD | | | | | | | |
| | | | 1 | p.V1605_V1606del | | | | | | | |
| | | | 1 | p.V1605_V1606insN | | | | | | | |
| | | | 1 | p.V1605E | | | | | | | |
| | | | 1 | p.V1605G | | | | | | | |
| | | | 1 | p.V1606_F1607insLGR | | | | | | | |
| | | | 1 | p.V1606_F1607insLVY | | | | | | | |
| | | | 1 | p.V1606del | | | | | | | |
| | | | 5 | p.V1672I | | | | | | | 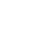 |
| | | | 1 | p.V1677_Y1678insA | | | | | | | |
| | | | 1 | p.V1677 > GIV | | | | | | | |
| | | | 3 | p.V1677D | | | | | | | |
| | | | 1 | p.V1677H | | | | | | | |
| | | | 2 | p.V1722_V1722ins? | | | | | | | |
| | | | 1 | p.V1722>ARWGSLNIPYLIEA | | | | | | | |
| | | | 1 | p.V1722>PPGSL | | | | | | | |
| | | | 1 | p.V1722E | | | | | | | |
| | | | 1 | p.V1722G | | | | | | | |
| | | | 4 | p.V1722M | | | | | | | |
| | | | 1 | p.V1740_A1741ins14 | | | | | | | |
| | | | 1 | p.V1740_A1741ins15 | | | | | | | |
| | | | 3 | p.V2286I | | | | | | | |
| | | | 1 | p.V2331fs*23 | | | | | | | |
| | | | 1 | p.V2422fs*2 | | | | | | | |
| | | | 1 | p.V2422M | | | | | | | |
| | | | 1 | p.V2444A | | | | | | | |
| | | | 1 | p.V2444fs*27 | | | | | | | |
| | | | 2 | p.V2444fs*3 | | | | | | | |
| | | | 1 | p.V2444fs*34 | | | | | | | |
| | | | 11 | p.V2444fs*35 | | | | | | | 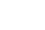 |
| | | | 2 | p.V2444fs*36 | | | | | | | |
| | | | 6 | p.V2444fs*37 | | | | | | | |
| | | | 1 | p.V2444fs*39 | | | | | | | |
| | | | 1 | p.V2444fs*69 | | | | | | | |
| | | | 1 | p.V2444fs*73 | | | | | | | |
| | | | 1 | p.V2454fs*25 | | | | | | | |
| | | | 1 | p.V2474fs*4 | | | | | | | |
| | | | 1 | p.V2474fs*5 | | | | | | | |
| | | | 1 | p.V2537I | | | | | | | |
| | | | 1 | p.W2521* | | | | | | | |
| | | | 1 | p.Y1620_Y1621>PGG | | | | | | | |
| | | | 1 | p.Y1620N | | | | | | | |
| | | | 1 | p.Y1678>RAS | | | | | | | |
| | | | 1 | p.Y1717F | | | | | | | |
| | | | 1 | p.Y1739_V1740ins11 | | | | | | | |
| | | | 1 | p.Y2491* | | | | | | | |
| | | | 1 | p.Y2491fs*1 | | | | | | | |
| ZMYM3 | 122 | 1 | 1 | p.C883* | | | | 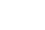 | | | |

| | Total | | | | | | | | | |
|---|---|---|---|---|---|---|---|---|---|---|
| | num- | | | | | | | | | |
| | ber | Total | # | | | | | | | |
| | sam- | num- | cases | | Lym- | | Bur- | | | |
| | ples | ber | per | | phoid | | kitts | MALT | | |
| | exam- | muta- | muta- | | neo- | | lym- | lym- | | |
| Gene | ined | tions | tion | AA change | plasms | DLBCL | phoma | phoma | ALL | other* |
| SF3B1 | 93 | 6 | 1 | p.Q534P | | | | | | |
| | | | 1 | P.L1211L | | | | | | |
| | | | 1 | p.R568H | | | | | | |
| | | | 1 | p.Q699H | | | | | | |
| | | | 1 | p.K700E | | | | | | |
| | | | 1 | p.P718L | | | | | | |
| MYD88 | 445 | 12 | 2 | p.V217F | | 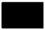 | | | | |
| | | | 1 | p.W218R | | 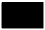 | | | | |
| | | | 2 | p.I220T | | 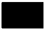 | | | | |
| | | | 11 | p.S219C | | 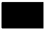 | 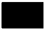 | | | |
| | | | 2 | p.S222R | | 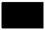 | | | | |
| | | | 3 | p.M232T | | 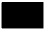 | | | | |
| | | | 5 | p.S243N | | 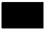 | | | | |
| | | | 64 | p.L265P | | 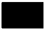 | 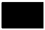 | 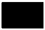 | | |
| | | | 1 | p.V52M | | 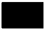 | | | | |
| | | | 1 | p.S149G | | 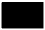 | | | | |
| | | | 1 | p.S149I | | 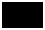 | | | | |
| | | | 1 | p.T294P | | 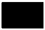 | | | | |
| FBXW7 | 5385 | 84 | 1 | p.A315T | | | | | | |
| | | | 1 | p.C386W | | | | | | |
| | | | 1 | p.D130fs*41 | | | | | | |
| | | | 1 | p.D440N | | | | | | |
| | | | 1 | p.D480Y | | | | | | |
| | | | 1 | p.D520N | | | | | | |
| | | | 1 | p.D527G | | | | | 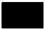 | |
| | | | 1 | p.E110* | | | | | | 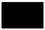 |
| | | | 1 | p.E117del | | | | | | 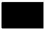 |
| | | | 1 | p.E117del | | | | | | 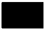 |
| | | | 1 | p.E121Y | | | | | | |
| | | | 1 | p.E693K | | | | | | |
| | | | 1 | p.F549fs*6 | | | | | | |
| | | | 1 | p.G397D | | | | | | |
| | | | 1 | p.G423V | | | | | | |
| | | | 2 | p.G423V | | | | | 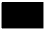 | |
| | | | 1 | p.G423V | | | | | | |
| | | | 1 | p.G579_Q581>E | | | | | | |
| | | | 1 | p.H379R | | | | | | |
| | | | 1 | p.H420Y | | | | | 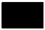 | |
| | | | 1 | p.H460R | | | | | | |
| | | | 1 | p.H470P | | | | | 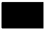 | |
| | | | 1 | p.H540Y | | | | | | 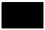 |
| | | | 1 | p.I435fs*9 | | | | | 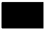 | |
| | | | 1 | p.I563T | | | | | | |
| | | | 1 | p.K11R | | | | | | |
| | | | 1 | p.K164* | | | | | | |
| | | | 1 | p.K371fs*7 | | | | | | |
| | | | 1 | p.K444fs*32 | | | | | 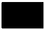 | |
| | | | 1 | p.L288fs*45 | | | | | 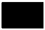 | |
| | | | 1 | p.L403fs*34 | | | | | | |
| | | | 1 | p.L594F | | | | | | |
| | | | 1 | p.L651* | | | | | | |
| | | | 1 | p.M467fs*5 | | | | | | |
| | | | 1 | p.P298R | | | | | | |
| | | | 2 | p.P298S | | | | | | |
| | | | 1 | p.Q156E | | | | | | |
| | | | 1 | p.Q220* | | | | | | |
| | | | 1 | p.Q264R | | | | | | |
| | | | 1 | p.Q303* | | | | | | |
| | | | 1 | p.Q98* | | | | | | |
| | | | 1 | p.R13* | | | | | | 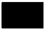 |
| | | | 3 | p.R224* | | | | | | |
| | | | 6 | p.R278* | | | | | | |
| | | | 1 | p.R312S | | | | | | |
| | | | 1 | p.R367* | | | | | | |
| | | | 1 | p.R367* | | | | | | |
| | | | 1 | p.R367* | | | | | | 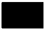 |
| | | | 4 | p.R393* | | | | | | |
| | | | 1 | p.R393* | | | | | | |
| | | | 1 | p.R441W | | | | | | |
| | | | 28 | p.R465C | | | | | 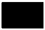 | |
| | | | 10 | p.R465C | | | | | | |
| | | | 6 | p.R465C | | | | | | |
| | | | 4 | p.R465C | | | | | | |
| | | | 1 | p.R465C | | | | | | |
| | | | 1 | p.R465C | | | | | | |
| | | | 22 | p.R465H | | | | | 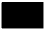 | |
| | | | 1 | p.R465H | | | | | | |
| | | | 6 | p.R465H | | | | | | |
| | | | 1 | p.R465H | | | | | | |
| | | | 1 | p.R465H | | | | | | 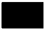 |
| | | | 2 | p.R465L | | | | | 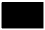 | |
| | | | 2 | p.R473fs*25 | | | | | | 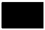 |
| | | | 2 | p.R473fs*25 | | | | | | |
| | | | 1 | p.R473fs*4 | | | | | | |
| | | | 2 | p.R473fs*4 | | | | | | |
| | | | 1 | p.R479G | | | | | | |
| | | | 2 | p.R479G | | | | | 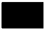 | |
| | | | 1 | p.R479L | | | | | | |
| | | | 4 | p.R479L | | | | | 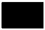 | |
| | | | 1 | p.R479Q | | | | | 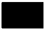 | |
| | | | 16 | p.R479Q | | | | | 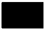 | |
| | | | 7 | p.R479Q | | | | | | |
| | | | 1 | p.R479Q | | | | | | |
| | | | 1 | p.R479Q | | | | | | |
| | | | 1 | p.R479Q | | | | | 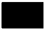 | |
| | | | 1 | p.R479Q | | | | | | 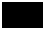 |
| | | | 1 | p.R479Q | | | | | | |
| | | | 1 | p.R484M | | | | | 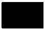 | |
| | | | 1 | p.R484T | | | | | | |
| | | | 5 | p.R505C | | | | | | |
| | | | 1 | p.R505C | | | | | | |
| | | | 18 | p.R505C | | | | | 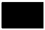 | |
| | | | 1 | p.R505C | | | | | | |
| | | | 1 | p.R505C | | | | | | 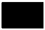 |
| | | | 2 | p.R505H | | | | | | |
| | | | 1 | p.R505L | | | | | 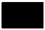 | |
| | | | 1 | p.R505L | | | | | 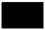 | |
| | | | 1 | p.R505L | | | | | | |
| | | | 1 | p.R505P | | | | | 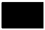 | |
| | | | 1 | p.R505S | | | | | 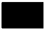 | |
| | | | 1 | p.R543K | | | | | | |
| | | | 1 | p.R658* | | | | | | |
| | | | 1 | p.R674Q | | | | | | |
| | | | 1 | p.R689W | | | | | 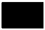 | |
| | | | 1 | p.R689W | | | | | | |
| | | | 1 | p.S182fs*57 | | | | | | |
| | | | 1 | p.S282* | | | | | | |
| | | | 1 | p.S294* | | | | | | |
| | | | 1 | p.S438F | | | | | | |
| | | | 6 | p.S582P | | | | | | |
| | | | 1 | p.S596F | | | | | | |
| | | | 1 | p.S668fs*26 | | | | | | |
| | | | 1 | p.S668fs*39 | | | | | | |
| | | | 1 | p.S668fs*39 | | | | | | |
| | | | 1 | p.T15_G16insP | | | | | | |
| | | | 1 | p.T532N | | | | | | |
| | | | 1 | p.T653fs*8 | | | | | 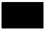 | |
| | | | 1 | p.V504I | | | | | | |
| | | | 1 | p.V504I | | | | | | |
| | | | 1 | p.V627A | | | | | 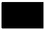 | |
| | | | 1 | p.V672M | | | | | | |
| | | | 2 | p.W446* | | | | | | |
| | | | 2 | p.W526R | | | | | | |
| | | | 1 | p.W649* | | | | | | |
| | | | 1 | p.Y519C | | | | | | |
| | | | 1 | p.Y545C | | | | | | |
| MAPK1 | 902 | 1 | 1 | p.A143A | | | | | | |
| DDX3X | 659 | 4 | 1 | p.R294T | | | | | | |
| | | | 1 | p.A502T | | | | | | |
| | | | 1 | p.R548T | | | | | | |
| | | | 1 | p.N551H | | | | | | |
| ATM | 2852 | 179 | 2 | p.A1309T | 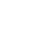 | | | | | |
| | | | 2 | p.A1742P | 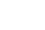 | | | | | |
| | | | 1 | p.A1945T | | | | | | |
| | | | 1 | p.A2274T | 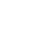 | | | | | |
| | | | 1 | p.A2420P | 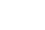 | | | | | |
| | | | 1 | p.A2622V | 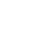 | | | | | |
| | | | 1 | p.A2631fs*2 | 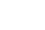 | | | | | |
| | | | 1 | p.A2893fs*3 | 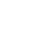 | | | | | |
| | | | 2 | p.A3006P | 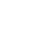 | | | | | |
| | | | 1 | p.A350T | 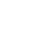 | | | | | |
| | | | 1 | p.C2349W | 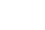 | | | | | |
| | | | 1 | p.C353fs*5 | 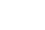 | | | | | |
| | | | 1 | p.C540Y | | | | | | |
| | | | 1 | p.C693_Q700>E | 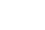 | | | | | |
| | | | 1 | p.D1208H | | | | | | |
| | | | 1 | p.D126E | 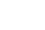 | | | | | |
| | | | 1 | p.D1682H | 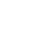 | | | | | |
| | | | 2 | p.D1682Y | 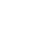 | | | | | |
| | | | 9 | p.D1853N | | | | | | |
| | | | 1 | p.D1853V | 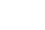 | | | | | |
| | | | 1 | p.D2708N | | | | | | |
| | | | 1 | p.D2725G | 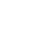 | | | | | |
| | | | 2 | p.D2725V | 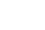 | | | | | |
| | | | 1 | p.E1612_Q1620>* | 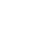 | | | | | |
| | | | 1 | p.E1991D | | | | | | 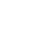 |
| | | | 1 | p.E2052* | | | | | | |
| | | | 1 | p.E2164K | 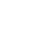 | | | | | |
| | | | 1 | p.E2423G | 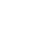 | | | | | |
| | | | 1 | p.E2423K | 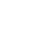 | | | | | |
| | | | 1 | p.E26fs*7 | 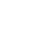 | | | | | |
| | | | 2 | p.E522fs*43 | 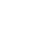 | | | | | |
| | | | 1 | p.E770* | 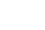 | | | | | |
| | | | 2 | p.E848Q | | | | | | |
| | | | 1 | p.F1209fs*19 | 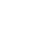 | | | | | |
| | | | 1 | p.F1463L | 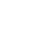 | | | | | |
| | | | 1 | p.F1463S | 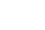 | | | | | |
| | | | 1 | p.F168_V170>L | 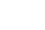 | | | | | |
| | | | 1 | p.F1683fs*7 | 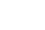 | | | | | |
| | | | 1 | p.F2732L | 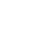 | | | | | |
| | | | 1 | p.F2799fs*4 | | | | | | |
| | | | 1 | p.F570S | 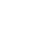 | | | | | |
| | | | 3 | p.F858L | 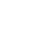 | | | | | |
| | | | 1 | p.G138R | | | | | | 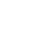 |
| | | | 1 | p.G2023R | 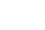 | | | | | |
| | | | 1 | p.G2063E | 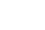 | | | | | |
| | | | 2 | p.G2695A | 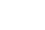 | | | | | |
| | | | 2 | p.G2867E | 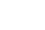 | | | | | |
| | | | 1 | p.G2925D | 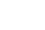 | | | | | |
| | | | 1 | p.G2925V | 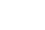 | | | | | |
| | | | 1 | p.G3051V | 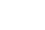 | | | | | |
| | | | 1 | p.G558* | | | | | | |
| | | | 1 | p.H1380Y | 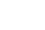 | | | | | |
| | | | 1 | p.H2872Q | | | | | | |
| | | | 1 | p.H996Q | 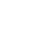 | | | | | |
| | | | 1 | p.I1237fs*2 | 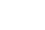 | | | | | |
| | | | 2 | p.I1332fs*27 | 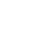 | | | | | |
| | | | 1 | p.I1407S | 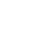 | | | | | |
| | | | 1 | p.I1407T | 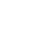 | | | | | |
| | | | 1 | p.I1469M | | | | | | 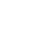 |
| | | | 2 | p.I1681V | 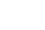 | | | | | |
| | | | 1 | p.I2055fs*33 | 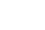 | | | | | |
| | | | 1 | p.I2076S | 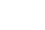 | | | | | |
| | | | 1 | p.I2356F | | | | | | 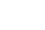 |
| | | | 1 | p.I2888T | 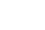 | | | | | |
| | | | 1 | p.I352T | 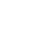 | | | | | |
| | | | 1 | p.K1454N | 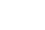 | | | | | |
| | | | 1 | p.K1994E | 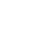 | | | | | |
| | | | 1 | p.K2213fs*22 | 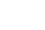 | | | | | |
| | | | 1 | p.K2237fs*11 | 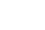 | | | | | |
| | | | 1 | p.K2418_R2419insK | 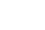 | | | | | |
| | | | 1 | p.K2717M | 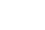 | | | | | |
| | | | 1 | p.K2810del | 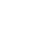 | | | | | |
| | | | 1 | p.K3018N | 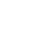 | | | | | |
| | | | 1 | p.K902fs*18 | 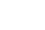 | | | | | |
| | | | 1 | p.L1322I | 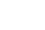 | | | | | |
| | | | 1 | p.L1322P | | | | | | |
| | | | 1 | p.L1472F | | | | | | |
| | | | 1 | p.L1708fs*6 | 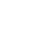 | | | | | |
| | | | 1 | p.L1764fs*12 | 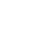 | | | | | |
| | | | 2 | p.L1794L | | | | | | |
| | | | 1 | p.L1910H | 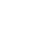 | | | | | |
| | | | 1 | p.L1939V | | | | | | 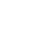 |
| | | | 1 | p.L2004R | 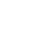 | | | | | |
| | | | 1 | p.L2417P | | | | | | |
| | | | 1 | p.L2427R | 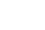 | | | | | |
| | | | 1 | p.L2445P | 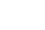 | | | | | |
| | | | 1 | p.L2450fs*11 | 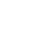 | | | | | |
| | | | 1 | p.L2722R | 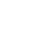 | | | | | |
| | | | 2 | p.L2890V | 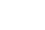 | | | | | |
| | | | 1 | p.L2945fs*7 | 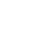 | | | | | |
| | | | 1 | p.L3017P | 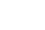 | | | | | |
| | | | 1 | p.L895fs*4 | 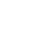 | | | | | |
| | | | 1 | p.M1040V | 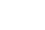 | | | | | |
| | | | 1 | p.M1916I | | | | | | |
| | | | 1 | p.M1L | 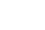 | | | | | |
| | | | 1 | p.M2616I | 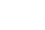 | | | | | |
| | | | 1 | p.M2805fs*1 | 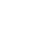 | | | | | |
| | | | 1 | p.M855fs*24 | | | | | | |
| | | | 1 | p.N1739T | | | | | | |
| | | | 1 | p.N1801Y | 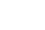 | | | | | |
| | | | 1 | p.N750K | 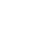 | | | | | |
| | | | 1 | p.P1054R | 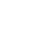 | | | | | |
| | | | 1 | p.P1829fs*5 | | | | | | |
| | | | 1 | p.P2699R | 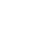 | | | | | |
| | | | 1 | p.P2842R | | | | | | |
| | | | 4 | p.P604S | 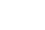 | | | | | |
| | | | 1 | p.Q1128R | 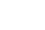 | | | | | |
| | | | 1 | p.Q1361* | 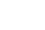 | | | | | |
| | | | 1 | p.Q162* | 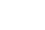 | | | | | |
| | | | 1 | p.Q163* | 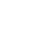 | | | | | |
| | | | 1 | p.Q2414* | | | | | | |
| | | | 1 | p.Q2442P | 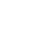 | | | | | |
| | | | 2 | p.Q2442P | | | | | | |
| | | | 1 | p.Q2593* | 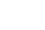 | | | | | |
| | | | 1 | p.Q466* | 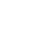 | | | | | |
| | | | 1 | p.Q747H | | | | | | |
| | | | 1 | p.R1086L | | | | | | |
| | | | 1 | p.R1304fs*43 | 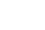 | | | | | |
| | | | 1 | p.R2263S | 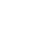 | | | | | |
| | | | 1 | p.R2273fs*37 | 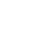 | | | | | |
| | | | 1 | p.R23Q | | | | | | |
| | | | 1 | p.R2400fs*6 | 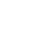 | | | | | |
| | | | 1 | p.R2443* | 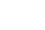 | | | | | |
| | | | 1 | p.R2443Q | 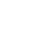 | | | | | |
| | | | 2 | p.R2443Q | | | | | | |
| | | | 1 | p.R2453P | | | | | | |
| | | | 1 | p.R2486G | 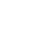 | | | | | |
| | | | 1 | p.R2713K | 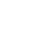 | | | | | |
| | | | 1 | p.R2832C | 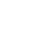 | | | | | |
| | | | 1 | p.R2871_H2872>S | 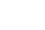 | | | | | |
| | | | 1 | p.R2912K | | | | | | 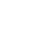 |
| | | | 4 | p.R3008C | 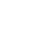 | | | | | |
| | | | 4 | p.R3008H | 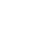 | | | | | |
| | | | 3 | p.R3047* | 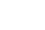 | | | | | |
| | | | 2 | p.R337C | | | | | | |
| | | | 1 | p.R337H | | | | | | |
| | | | 2 | p.R337S | 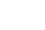 | | | | | |
| | | | 1 | p.R717W | 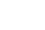 | | | | | |
| | | | 1 | p.S1179F | 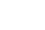 | | | | | |
| | | | 1 | p.S151fs*2 | | | | | | 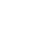 |
| | | | 1 | p.S1770* | 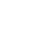 | | | | | |
| | | | 1 | p.S1905L | | | | | | |
| | | | 1 | p.S207C | 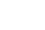 | | | | | |
| | | | 1 | p.S2375I | | | | | | |
| | | | 1 | p.S2394L | 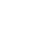 | | | | | |
| | | | 1 | p.S2408L | | | | | | |
| | | | 1 | p.S2546_I2548del | 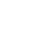 | | | | | |
| | | | 1 | p.S2859F | 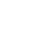 | | | | | |
| | | | 1 | p.S707fs*29 | 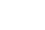 | | | | | |
| | | | 2 | p.S707P | | | | | | |
| | | | 1 | p.S853* | | | | | | |
| | | | 1 | p.S978P | 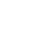 | | | | | |
| | | | 1 | p.T1735fs*11 | 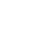 | | | | | |
| | | | 1 | p.T1743I | 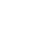 | | | | | |
| | | | 1 | p.T1953R | 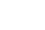 | | | | | |
| | | | 1 | p.T2396S | 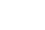 | | | | | |
| | | | 1 | p.T2438K | 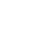 | | | | | |
| | | | 1 | p.T261fs*10 | 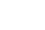 | | | | | |
| | | | 2 | p.T2666A | | | | | | |
| | | | 1 | p.T2911del | 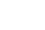 | | | | | |
| | | | 2 | p.T2947S | 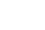 | | | | | |
| | | | 1 | p.T935T | | | | | | |
| | | | 1 | p.V1292_Q1331del | 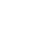 | | | | | |
| | | | 2 | p.V1941L | 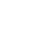 | | | | | |
| | | | 1 | p.V2424G | 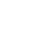 | | | | | |
| | | | 1 | p.V245A | | | | | | 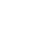 |
| | | | 2 | p.V410A | 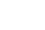 | | | | | |
| | | | 1 | p.W1221* | 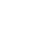 | | | | | |
| | | | 1 | p.W2845* | | | | | | 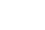 |
| | | | 1 | p.W308* | 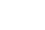 | | | | | |
| | | | 1 | p.W393* | 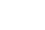 | | | | | |
| | | | 1 | p.W57* | 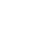 | | | | | |
| | | | 1 | p.Y1392fs*7 | 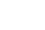 | | | | | |
| | | | 1 | p.Y1475C | 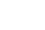 | | | | | |
| | | | 1 | p.Y1961C | 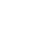 | | | | | |
| | | | 1 | p.Y2019S | 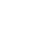 | | | | | |
| | | | 1 | p.Y2627fs*29 | 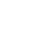 | | | | | |
| | | | 1 | p.Y2817* | 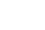 | | | | | |
| | | | 1 | p.Y2954C | | | | | | |
| | | | 1 | p.Y332C | 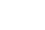 | | | | | |
| NOTCH1 | 5090 | 645 | 1 | p.1719_1720>QKGPLAAFLGA LASLGSLTIPYLI | 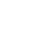 | | | | | |
| | | | 1 | p.1741_1742 >MKLVEPPPPAQL HFMYVA | 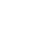 | | | | | |
| | | | 1 | p.A1611_A1636 > A | 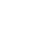 | | | | | |
| | | | 1 | p.A1611T | 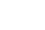 | | | | | |
| | | | 1 | p.A1635S | 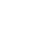 | | | | | |
| | | | 1 | p.A1651T | 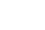 | | | | | |
| | | | 2 | p.A1697D | 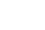 | | | | | |
| | | | 1 | p.A1701P | 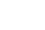 | | | | | |
| | | | 1 | p.A1701V | 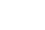 | | | | | |
| | | | 6 | p.A1702P | 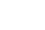 | | | | | |
| | | | 1 | p.A1721_V1722 > YG | 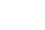 | | | | | |
| | | | 1 | p.A1741_A1742ins11 | 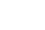 | | | | | |
| | | | 1 | p.A1741_A1742ins17 | 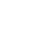 | | | | | |
| | | | 1 | p.A1741_A1742ins36 | 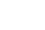 | | | | | |
| | | | 1 | p.A1742_A1743insGALHFMYV A | 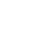 | | | | | |
| | | | 3 | p.A2280V | 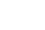 | | | | | 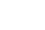 |
| | | | 1 | p.A2332T | 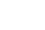 | | | | | |
| | | | 1 | p.A2340fs*15 | 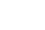 | | | | | |
| | | | 1 | p.A2357_S2358>TN | 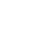 | | | | | |
| | | | 1 | p.A2425V | 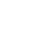 | | | | | |
| | | | 1 | p.A2426fs | 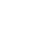 | | | | | |
| | | | 1 | p.A2426fs*15 | 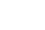 | | | | | |
| | | | 1 | p.A2442V | 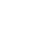 | | | | | |
| | | | 1 | p.A2444fs*39 | | | | | 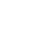 | |
| | | | 1 | p.A2453T | | | | | | 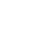 |
| | | | 5 | p.A2464fs*14 | 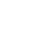 | | | | | |
| | | | 1 | p.A2554D | 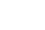 | | | | | |
| | | | 1 | p.C1117C | | | | | | |
| | | | 1 | p.C1686F | | | | | 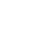 | |
| | | | 3 | p.C1693R | 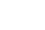 | | | | | |
| | | | 1 | p.D1547G | 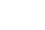 | | | | | |
| | | | 1 | p.D1610_A1611insPQP | 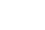 | | | | | |
| | | | 1 | p.D1610_R1634> | 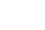 | | | | | |
| | | | 1 | p.D1610_R1634del | 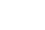 | | | | | |
| | | | 2 | p.D1610V | 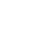 | | | | | |
| | | | 1 | p.D1643H | | | | | | |
| | | | 1 | p.D1682G | 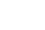 | | | | | |
| | | | 11 | p.D1699D | 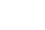 | | | | | |
| | | | 1 | p.D2443fs | 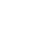 | | | | | |
| | | | 1 | p.D2443fs*2 | 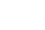 | | | | | |
| | | | 1 | p.D2443fs*35 | 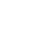 | | | | | |
| | | | 3 | p.D2443fs*39 | | | | | 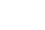 | |
| | | | 1 | p.D620Y | | | | | | |
| | | | 1 | p.E1584_Q1585insPVELMPPE | 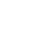 | | | | | |
| | | | 1 | p.E1584>AQ | | | | | 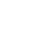 | |
| | | | 1 | p.E1584>GTHPKE | 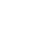 | | | | | |
| | | | 1 | p.E1584G | 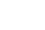 | | | | | |
| | | | 1 | p.E2268fs*31 | 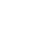 | | | | | |
| | | | 1 | p.E2268fs*89 | 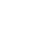 | | | | | |
| | | | 1 | p.E2507* | | | | | 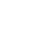 | |
| | | | 1 | p.E2507fs*6 | 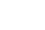 | | | | | |
| | | | 1 | p.E2516fs*1 | 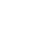 | | | | | |
| | | | 1 | p.E2516fs*3 | 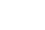 | | | | | |
| | | | 1 | p.E2516fs*71 | | | | | 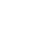 | |
| | | | 1 | p.F1541L | | | | | 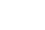 | |
| | | | 1 | p.F1591_E1596>LLGG | 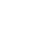 | | | | | |
| | | | 1 | p.F1591>SI | 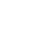 | | | | | |
| | | | 1 | p.F1593_F1594>TA | | | | | 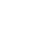 | |
| | | | 2 | p.F1593_L1594ins12 | 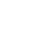 | | | | | |
| | | | 1 | p.F1593_L1594insC | 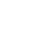 | | | | | |
| | | | 1 | p.F1593_L1594insDLS | 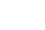 | | | | | |
| | | | 1 | p.F1593_L1594insGVN | 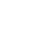 | | | | | |
| | | | 1 | p.F1593_L1594insSP | 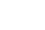 | | | | | |
| | | | 1 | p.F1593_R1595>HFDG | 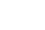 | | | | | |
| | | | 1 | p.F1593>KED | | | | | 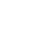 | |
| | | | 3 | p.F1593>LA | 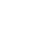 | | | | | |
| | | | 1 | p.F1593>LG | 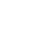 | | | | | |
| | | | 2 | p.F1593>LGA | 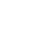 | | | | | |
| | | | 1 | p.F1593>LGP | | | | | 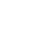 | |
| | | | 3 | p.F1593>LS | 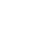 | | | | | |
| | | | 1 | p.F1593>LSP | | | | | 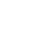 | |
| | | | 1 | p.F1593>PEH | 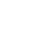 | | | | | |
| | | | 3 | p.F1593C | 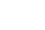 | | | | | |
| | | | 1 | p.F1593L | 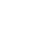 | | | | | |
| | | | 12 | p.F1593S | 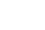 | | | | | |
| | | | 1 | p.F1607_A1611>LVPSK | 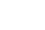 | | | | | |
| | | | 1 | p.F1607_F>LPL | | | | | 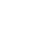 | |
| | | | 1 | p.F1607_K1608>LCPEM | | | | | 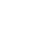 | |
| | | | 1 | p.F1607_K1608>LGLWRQ | 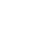 | | | | | |
| | | | 1 | p.F1607_K1608>RSE | 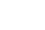 | | | | | |
| | | | 1 | p.F1607_K1608ins15 | 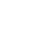 | | | | | |
| | | | 1 | p.F1607_K1608insGS | | | | | 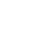 | |
| | | | 1 | p.F1607_K1608insLVGCGQ | | | | | 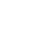 | |
| | | | 1 | p.F1607_K1608insNPNVVLFK | | | | | 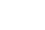 | |
| | | | 1 | p.F1607_K1608insVRVTHTK | 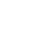 | | | | | |
| | | | 1 | p.F1607_R1609>LG | | | | | 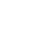 | |
| | | | 1 | p.F1607>FVA | 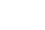 | | | | | |
| | | | 2 | p.F1607>LD | 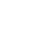 | | | | | |
| | | | 1 | p.F1607>LDP | 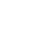 | | | | | |
| | | | 1 | p.F1607>LGM | 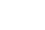 | | | | | |
| | | | 1 | p.F1607>LGT | 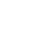 | | | | | |
| | | | 1 | p.F1607>LNPLS | 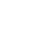 | | | | | |
| | | | 1 | p.F1607>LPPHP | 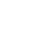 | | | | | |
| | | | 2 | p.F1607>LPRNED | 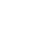 | | | | | |
| | | | 1 | p.F1607>LRFL | | | | | 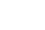 | |
| | | | 1 | p.F1607>LSMPP | | | | | 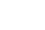 | |
| | | | 1 | p.F1607>WNS | | | | | 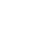 | |
| | | | 1 | p.F1618_P1619del | 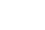 | | | | | |
| | | | 1 | p.F1618_P1619insEPP | 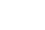 | | | | | |
| | | | 1 | p.F1618_Y1620>WSP | 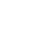 | | | | | |
| | | | 1 | p.F1618>FKN | 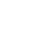 | | | | | |
| | | | 3 | p.F1618del | | | | | 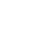 | |
| | | | 1 | p.F1694S | 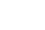 | | | | | |
| | | | 1 | p.F1737_M1738ins13 | | | | | 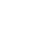 | |
| | | | 1 | p.F2267fs*87 | 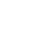 | | | | | |
| | | | 1 | p.F2482fs*5 | 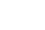 | | | | | |
| | | | 1 | p.F2510fs*1 | | | | | 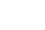 | |
| | | | 1 | p.G1137V | | | | | | |
| | | | 1 | p.G1216D | | | | | | |
| | | | 1 | p.G135W | | | | | | |
| | | | 1 | p.G1559V | | | | | | |
| | | | I | p.G1647S | 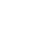 | | | | | |
| | | | 1 | p.G1657S | 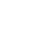 | | | | | |
| | | | 1 | p.G1660D | 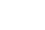 | | | | | |
| | | | 1 | p.G1705* | | | | | | |
| | | | 2 | p.G2153R | | | | | | 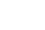 |
| | | | 1 | p.G2153S | | | | | | |
| | | | 1 | p.G2246R | 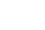 | | | | | |
| | | | 1 | p.G2263fs*6 | 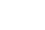 | | | | | |
| | | | 1 | p.G2334fs*21 | 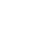 | | | | | |
| | | | 1 | p.G2421fs*3 | | | | | 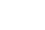 | |
| | | | 3 | p.H1592_F1593>QT | 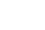 | | | | | |
| | | | 1 | p.H1592_F1593insY | | | | | 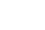 | |
| | | | 1 | p.H1592Y | 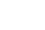 | | | | | |
| | | | 1 | p.H1602_T1603insQ | 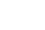 | | | | | |
| | | | 1 | p.H1602P | 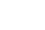 | | | | | |
| | | | 1 | p.H1612_G1613>QIVVFKRDA HG | 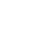 | | | | | |
| | | | 1 | p.H1612_P1619>RGT | 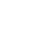 | | | | | |
| | | | 1 | p.H2276fs.79 | | | | | | |
| | | | 1 | p.H2419fs.16 | 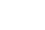 | | | | | |
| | | | 1 | p.H2429fs.8 | 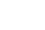 | | | | | |
| | | | 1 | p.H2508Y | | | | | 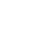 | |
| | | | 1 | p.I1617_R1623>G | 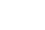 | | | | | |
| | | | 7 | p.I1617N | 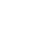 | | | | | |
| | | | 1 | p.I1632V | 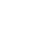 | | | | | |
| | | | 1 | p.I1633I | 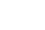 | | | | | |
| | | | 1 | p.I1676_V1677>I | 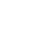 | | | | | |
| | | | 1 | p.I1676_V1677>MF | 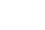 | | | | | |
| | | | 1 | p.I1676>TAFL | 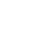 | | | | | |
| | | | 4 | p.I1681N | 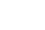 | | | | | |
| | | | 2 | p.I1681S | 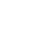 | | | | | |
| | | | 2 | p.I1719T | 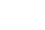 | | | | | |
| | | | 1 | p.I2457fs.21 | 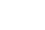 | | | | | |
| | | | 1 | p.K1608_R1609insPAK | 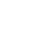 | | | | | |
| | | | 1 | p.K1608>GPPLQ | 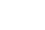 | | | | | |
| | | | 1 | p.K1608N | 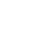 | | | | | |
| | | | 2 | p.K1783_R1784ins31 | 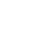 | | | | | |
| | | | 1 | p.L122fs.3 | | | | | | |
| | | | 31 | p.L1575P | 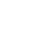 | | | | | |
| | | | 2 | p.L1575Q | 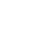 | | | | | |
| | | | 1 | p.L1586>PPEAV | 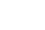 | | | | | |
| | | | 35 | p.L1586P | 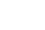 | | | | | |
| | | | 1 | p.L1594_R1595ins12 | 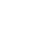 | | | | | |
| | | | 1 | p.L1594_R1595insA | 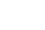 | | | | | |
| | | | 1 | p.L1594>NPM | 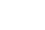 | | | | | |
| | | | 34 | p.L1594P | 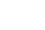 | | | | | |
| | | | 1 | p.L1597_S1598insG | 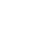 | | | | | |
| | | | 3 | p.L1597H | 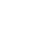 | | | | | |
| | | | 1 | p.L1601_H1602insA | 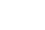 | | | | | |
| | | | 1 | p.L1601_H1602insL | 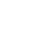 | | | | | |
| | | | 40 | p.L1601P | 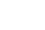 | | | | | |
| | | | 5 | p.L1601Q | 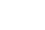 | | | | | |
| | | | 29 | p.L1679P | 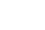 | | | | | |
| | | | 5 | p.L1679Q | 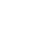 | | | | | |
| | | | 1 | p.L1707_A1708ins14 | 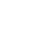 | | | | | |
| | | | 4 | p.L1710P | 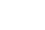 | | | | | |
| | | | 1 | p.L2327fs*5 | | | | | 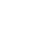 | |
| | | | 1 | p.L2336fs*19 | | | | | 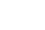 | |
| | | | 1 | p.L2336fs*20 | | | | | 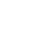 | |
| | | | 1 | p.L2343fs*12 | | | | | 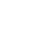 | |
| | | | 1 | p.L2391_Q2392>PFPF* | | | | | 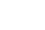 | |
| | | | 1 | p.L2430_G2431>CLVSR | | | | | 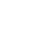 | |
| | | | 1 | p.L2435fs*1 | 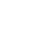 | | | | | |
| | | | 1 | p.L2435fs*2 | 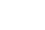 | | | | | |
| | | | 1 | p.L2447fs*33 | 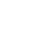 | | | | | |
| | | | 2 | p.L2458V | 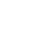 | | | | | |
| | | | 1 | p.L2465L | 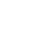 | | | | | |
| | | | 2 | p.L2469fs*10 | 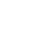 | | | | | |
| | | | 1 | p.L2469fs*11 | 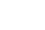 | | | | | |
| | | | 1 | p.L2473fs*1 | | | | | 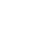 | |
| | | | 1 | p.L2473fs*7 | 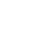 | | | | | |
| | | | 1 | p.L2511L | 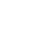 | | | | | |
| | | | 1 | p.M1581_P1582del | | | | | 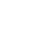 | |
| | | | 1 | p.M1581_P1582insLMHLAF | 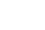 | | | | | |
| | | | 1 | p.M1581_P1582insPRYEL | 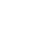 | | | | | |
| | | | 2 | p.M1581del | 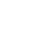 | | | | | |
| | | | 1 | p.M1616_F1618>L | 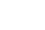 | | | | | |
| | | | 1 | p.M1738_Y1739ins35 | | | | | 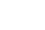 | |
| | | | 1 | p.M2057fs*211 | | | | | | 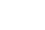 |
| | | | 1 | p.M2347fs*16 | 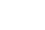 | | | | | |
| | | | 1 | p.M2347fs*9 | 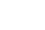 | | | | | |
| | | | 1 | p.N1900I | 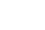 | | | | | |
| | | | 1 | p.N2296_F2297insWV | 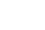 | | | | | |
| | | | 1 | p.N2390fs*33 | 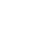 | | | | | |
| | | | 1 | p.N2402_I2403>GPSLNN | 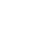 | | | | | |
| | | | 1 | p.N2402fs*21 | 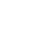 | | | | | |
| | | | 1 | p.P1582_E1584>Q | 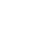 | | | | | |
| | | | 1 | p.P1583_E1584insP | 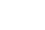 | | | | | |
| | | | 1 | p.P1583_L1586>IEA | 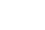 | | | | | |
| | | | 4 | p.P1583del | 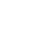 | | | | | |
| | | | 1 | p.P2272S | 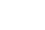 | | | | | |
| | | | 1 | p.P2333fs*22 | 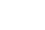 | | | | | |
| | | | 1 | p.P2411fs*12 | 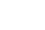 | | | | | |
| | | | 1 | p.P2412del | | | | | 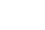 | |
| | | | 1 | p.P2412P | 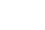 | | | | | |
| | | | 1 | p.P2413S | 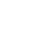 | | | | | |
| | | | 3 | p.P2413T | 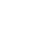 | | | | | |
| | | | 1 | p.P2414L | 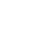 | | | | | |
| | | | 2 | p.P2416del | 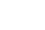 | | | | | |
| | | | 2 | p.P2418L | 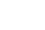 | | | | | |
| | | | 1 | p.P2439fs*40 | | | | | 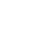 | |
| | | | 1 | p.P2439fs*41 | | | | | 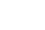 | |
| | | | 1 | p.P2439L | 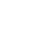 | | | | | |
| | | | 1 | p.P2459fs*21 | 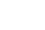 | | | | | |
| | | | 1 | p.P2459fs*61 | 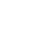 | | | | | |
| | | | 2 | p.P2463fs*15 | 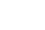 | | | | | |
| | | | 1 | p.P2475fs*1 | 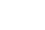 | | | | | |
| | | | 1 | p.P2475fs*3 | | | | | 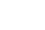 | |
| | | | 2 | p.P2475fs*34 | 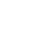 | | | | | |
| | | | 2 | p.P2475fs*5 | 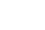 | | | | | |
| | | | 1 | p.P2476fs | 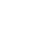 | | | | | |
| | | | 1 | p.P2476fs*2 | 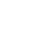 | | | | | |
| | | | 1 | p.P2494fs*13 | | | | | 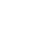 | |
| | | | 3 | p.P2494fs*3 | | | | | 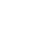 | |
| | | | 1 | p.P2506fs*6 | 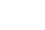 | | | | | |
| | | | 3 | p.P2506P | 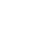 | | | | | |
| | | | 1 | p.P2509fs*8 | 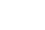 | | | | | |
| | | | 2 | p.P2513fs*3 | | | | | 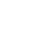 | |
| | | | 5 | p.P2513L | 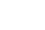 | | | | | |
| | | | 1 | p.P2515fs | 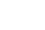 | | | | | |
| | | | 29 | p.P2515fs*4 | 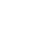 | | | | | |
| | | | 2 | p.P2518* | 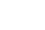 | | | | | |
| | | | 1 | p.P2518fs*6 | 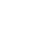 | | | | | |
| | | | 1 | p.Q1050L | | | | | | 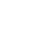 |
| | | | 2 | p.Q1585>PVELMPPE | 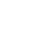 | | | | | |
| | | | 1 | p.Q1585del | 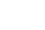 | | | | | |
| | | | 1 | p.Q1615_F1618>LCR | 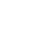 | | | | | |
| | | | 1 | p.Q1615_M1616>L | 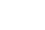 | | | | | |
| | | | 1 | p.Q1615K | | | | | 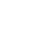 | |
| | | | 1 | p.Q1685_C1686insLEGQR | 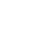 | | | | | |
| | | | 1 | p.Q2316* | | | | | 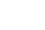 | |
| | | | 1 | p.Q2344fs*11 | 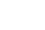 | | | | | |
| | | | 2 | p.Q2392* | 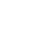 | | | | | |
| | | | 2 | p.Q2394* | 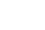 | | | | | |
| | | | 1 | p.Q2395* | | | | | 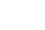 | |
| | | | 1 | p.Q2396* | | | | | 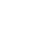 | |
| | | | 1 | p.Q2399* | | | | | 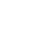 | |
| | | | 1 | p.Q2404* | | | | | 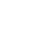 | |
| | | | 1 | p.Q2406* | 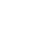 | | | | | |
| | | | 1 | p.02407* | 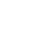 | | | | | |
| | | | 1 | p.Q2410* | | | | | 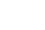 | |
| | | | 2 | p.Q2417* | 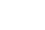 | | | | | |
| | | | 4 | p.Q2441* | 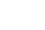 | | | | | |
| | | | 1 | p.Q2445* | 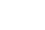 | | | | | |
| | | | 1 | p.Q2445fs*65 | 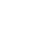 | | | | | |
| | | | 9 | p.Q2460* | 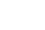 | | | | | |
| | | | 2 | p.Q2460fs*18 | 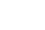 | | | | | |
| | | | 2 | p.Q2502* | 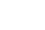 | | | | | |
| | | | 3 | p.Q2504* | 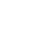 | | | | | |
| | | | 1 | p.Q2504fs | 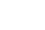 | | | | | |
| | | | 1 | p.Q2504fs*5 | 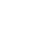 | | | | | |
| | | | 2 | p.Q2520* | 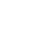 | | | | | |
| | | | 3 | p.R1587P | 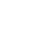 | | | | | |
| | | | 3 | p.R1595_E1596ins12 | 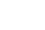 | | | | | |
| | | | 1 | p.R1595_L1597>L | | | | | 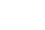 | |
| | | | 2 | p.R1595>PRLPHNSSFHFLR | 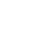 | | | | | |
| | | | 1 | p.R1595>PRLPHNSSSHFL | 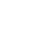 | | | | | |
| | | | 1 | p.R1599>QS | 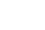 | | | | | |
| | | | 20 | p.R1599P | 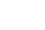 | | | | 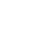 | |
| | | | 1 | p.R1609_A1611>T | 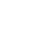 | | | | | |
| | | | 1 | p.R1609_D1610ins12 | 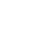 | | | | | |
| | | | 1 | p.R1609S | 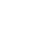 | | | | | |
| | | | 1 | p.R1628H | 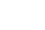 | | | | | |
| | | | 1 | p.R1628Q | 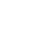 | | | | | |
| | | | 1 | p.R1634L | 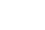 | | | | | |
| | | | 1 | p.R1663L | 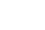 | | | | | |
| | | | 1 | p.R2160H | | | | | | |
| | | | 1 | p.R2273fs*78 | 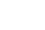 | | | | | |
| | | | 1 | p.R2328W | | | | | | |
| | | | 1 | p.S1598I | 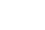 | | | | | |
| | | | 3 | p.S1675_I1676insG | 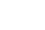 | | | | | |
| | | | 1 | p.S1675P | 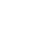 | | | | | |
| | | | 1 | p.S1709S | | | | | | 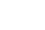 |
| | | | 1 | p.S2290R | 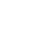 | | | | | |
| | | | 1 | p.S2291S | 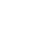 | | | | | |
| | | | 2 | p.S2330fs*25 | 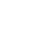 | | | | | |
| | | | 1 | p.S2330fs*7 | 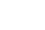 | | | | | |
| | | | 1 | p.S2337fs*18 | 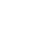 | | | | | |
| | | | 1 | p.S2342fs*1 | | | | | 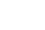 | |
| | | | 1 | p.S2342fs*13 | 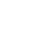 | | | | | |
| | | | 1 | p.S2342fs*7 | | | | | | |
| | | | 2 | p.S2408N | 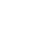 | | | | | |
| | | | 1 | p.S2423fs*1 | 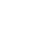 | | | | | |
| | | | 1 | p.S2424* | 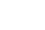 | | | | | |
| | | | 2 | p.S2427fs*4 | 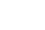 | | | | | |
| | | | 1 | p.S2433fs*5 | 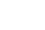 | | | | | |
| | | | 1 | p.S2436fs*2 | 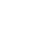 | | | | | |
| | | | 1 | p.S2440fs*1 | 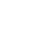 | | | | | |
| | | | 4 | p.S2440fs*4 | 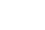 | | | | | |
| | | | 1 | p.S2440G | 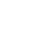 | | | | | |
| | | | 1 | p.S2450fs*28 | 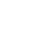 | | | | | |
| | | | 1 | p.S2468* | 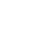 | | | | | |
| | | | 1 | p.S2468fs*1 | 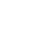 | | | | | |
| | | | 1 | p.S2468fs*10 | | | | | 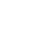 | |
| | | | 2 | p.S2468fs*11 | 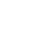 | | | | | |
| | | | 1 | p.S2468fs*15 | | | | | 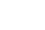 | |
| | | | 2 | p.S2487* | 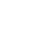 | | | | | |
| | | | 1 | p.S2487fs*7 | 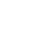 | | | | | |
| | | | 1 | p.S2492fs*67 | 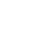 | | | | | |
| | | | 3 | p.S2493* | 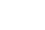 | | | | | |
| | | | 1 | p.S2493>S* | | | | | 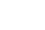 | |
| | | | 1 | p.S2493>SP* | 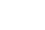 | | | | | |
| | | | 1 | p.S2493fs*100 | 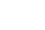 | | | | | |
| | | | 1 | p.S2493fs*3 | 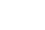 | | | | | |
| | | | 1 | p.S2514F | 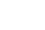 | | | | | |
| | | | 1 | p.S2514fs*4 | | | | | 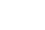 | |
| | | | 4 | p.S2524* | 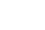 | | | | | |
| | | | 1 | p.S2528fs*80 | 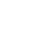 | | | | | |
| | | | 1 | p.S356del | | | | | | 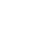 |
| | | | 1 | p.T1574_V1576del | 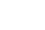 | | | | | |
| | | | 1 | p.T1603_N1604ins17 | 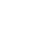 | | | | | |
| | | | 1 | p.T1997M | | | | | | |
| | | | 1 | p.T2467fs*11 | | | | | 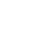 | |
| | | | 1 | p.T2467fs*12 | 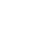 | | | | | |
| | | | 1 | p.T2467M | 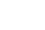 | | | | | |
| | | | 1 | p.T2484A | 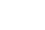 | | | | | |
| | | | 2 | p.T2484M | 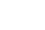 | | | | | |
| | | | 1 | p.T2512fs*1 | 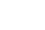 | | | | | |
| | | | 1 | p.T445T | | | | | | |
| | | | 1 | p.T971I | | | | | | |
| | | | 1 | p.V1576_V1578del | 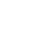 | | | | | |
| | | | 1 | p.V1577_V1578>FRP | 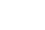 | | | | | |
| | | | 1 | p.V1577A | | | | | 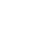 | |
| | | | 3 | p.V1577E | 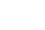 | | | | | |
| | | | 1 | p.V1578_V1579insA | | | | | 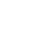 | |
| | | | 1 | p.V1578_V1579insGV | 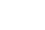 | | | | | |
| | | | 1 | p.V1578A | 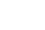 | | | | | |
| | | | 1 | p.V1579A | 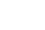 | | | | | |
| | | | 20 | p.V1579del | 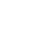 | | | | | |
| | | | 2 | p.V1579E | 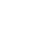 | | | | | |
| | | | 1 | p.V1579G | 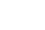 | | | | | |
| | | | 1 | p.V1605_R1609>LKGCD | 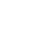 | | | | | |
| | | | 1 | p.V1605_V1606del | 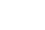 | | | | | |
| | | | 1 | p.V1605_V1606insN | 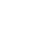 | | | | | |
| | | | 1 | p.V1605E | 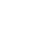 | | | | | |
| | | | 1 | p.V1605G | 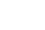 | | | | | |
| | | | 1 | p.V1606_F1607insLGR | 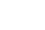 | | | | | |
| | | | 1 | p.V1606_F1607insLVY | 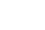 | | | | | |
| | | | 1 | p.V1606del | | | | | 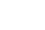 | |
| | | | 5 | p.V1672I | | | | | | |
| | | | 1 | p.V1677_Y1678insA | 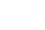 | | | | | |
| | | | 1 | p.V1677>GIV | 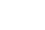 | | | | | |
| | | | 3 | p.V1677D | | | | | 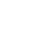 | |
| | | | 1 | p.V1677H | 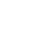 | | | | | |
| | | | 2 | p.V1722_V1722ins? | 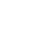 | | | | | |
| | | | 1 | p.V1722>ARWGSLNIPYLIEA | 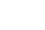 | | | | | |
| | | | 1 | p.V1722>PPGSL | 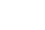 | | | | | |
| | | | 1 | p.V1722E | 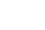 | | | | | |
| | | | 1 | p.V1722G | 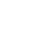 | | | | | |
| | | | 4 | p.V1722M | 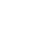 | | | | | |
| | | | 1 | p.V1740_A1741ins14 | | | | | 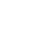 | |
| | | | 1 | p.V1740_A1741ins15 | | | | | 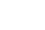 | |
| | | | 3 | p.V2286I | 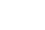 | | | | | |
| | | | 1 | p.V2331fs*23 | | | | | 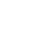 | |
| | | | 1 | p.V2422fs*2 | 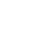 | | | | | |
| | | | 1 | p.V2422M | 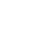 | | | | | |
| | | | 1 | p.V2444A | 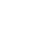 | | | | | |
| | | | 1 | p.V2444fs*27 | 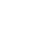 | | | | | |
| | | | 2 | p.V2444fs*3 | 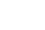 | | | | | |
| | | | 1 | p.V2444fs*34 | 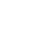 | | | | | |
| | | | 11 | p.V2444fs*35 | 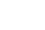 | | | | | |
| | | | 2 | p.V2444fs*36 | 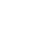 | | | | | |
| | | | 6 | p.V2444fs*37 | | | | | 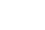 | |
| | | | 1 | p.V2444fs*39 | 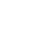 | | | | | |
| | | | 1 | p.V2444fs*69 | 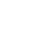 | | | | | |
| | | | 1 | p.V2444fs*73 | | | | | 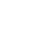 | |
| | | | 1 | p.V2454fs*25 | 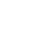 | | | | | |
| | | | 1 | p.V2474fs*4 | 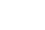 | | | | | |
| | | | 1 | p.V2474fs*5 | 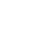 | | | | | |
| | | | 1 | p.V2537I | | | | | 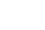 | |
| | | | 1 | p.W2521* | 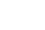 | | | | | |
| | | | 1 | p.Y1620_Y1621>PGG | | | | | 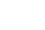 | |
| | | | 1 | p.Y1620N | | | | | 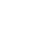 | |
| | | | 1 | p.Y1678>RAS | 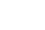 | | | | | |
| | | | 1 | p.Y1717F | 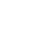 | | | | | |
| | | | 1 | p.Y1739_V1740ins11 | | | | | 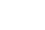 | |
| | | | 1 | p.Y2491* | 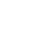 | | | | | |
| | | | 1 | p.Y2491fs*1 | 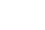 | | | | | |
| ZMYM3 | 122 | 1 | 1 | p.C883* | | | | | | |

TABLE 6
Comparison of the clinical characteristics of the discovery
(n = 91) vs extension (n = 101) samples.

| | Discovery | Extension | |
|---|---|---|---|
| | Cohort | Cohort | p-value |
| N | 91 | 101 | |
| Age at Diagnosis | 54 (34, 78) | 55 (30, 79) | 0.5 |
| (years) | | | |
| median (range) | | | |
| Age ³55 yrs. | 40 (44) | 52 (51) | 0.31 |
| Sex | | | |
| Female | 35 (38) | 51 (50) | 0.11 |
| Male | 56 (62) | 50 (50) | |
| Time from Dx to 1st | 30 (0.4, 154) | 32 (1.3, 234) | 0.7 |
| Therapy (months), | | | |
| median (range) | | | |
| # Patients initiating | 58 (64) | 38 (38) | <0.001 |
| first therapy | | | |
| IGHV | | | |
| Mutated | 38 (42) | 56 (55) | 0.04 |
| Unmutated | 40 (44) | 26 (26) | |
| Unknown | 13 (14) | 19 (19) | |
| ZAP-70 | | | |
| Positive | 38 (42) | 33 (33) | 0.17 |
| Negative | 44 (48) | 49 (49) | |
| Unknown | 9 (10) | 19 (19) | |
| FISH Cytogenetics | | | |
| del (13q-) het | 53 (58) | 59 (58) | 0.55 |
| del (13q-) homo | 12 (13) | 0 (0) | <0.001 |
| trisomy 12 | 13 (14) | 15 (15) | 0.84 |
| del (11q) | 22 (24) | 11 (11) | 0.03 |
| del(17p) | 15 (16) | 14 (14) | 0.84 |
| Unknown | 0 (0) | 8 (8) | |
| Somatic Mutations | | | |
| SF3B1-K700E | 7 (8) | 3 (3) | 0.2 |
| MYD88-P258L, | 7 (8) | 5 (5) | 0.55 |
| L265P | | | |
| NOTCH1-P2514fs | 4 (4) | 8 (8) | 0.38 |

TABLE 7
Additional mutations in the five core pathways.

| Pathway | Gene Name | Gene ID | Start_position | Variant_Classification | cDNA_Change | Protein_Change | Annotation | Patient ID |
|---|---|---|---|---|---|---|---|---|
| DNA damage | ANAPC4 | 29945 | 24993979 | Splice_Site_SNP | c.e4_splice_site | | uc003gro.1 | P19 |
| and Cell | CDC14B | 8555 | 98324609 | Missense | c.1795C>G | p.T448R | uc004awj.1 | P58 |
| cycle control | PTTG1 | 9232 | 159781905 | Missense | c.53C>A | p.T3N | uc003lyj.1 | P72 |
| | ESPL1 | 9700 | 51949811 | Missense | c.909G>A | p.S273N | uc001sck.2 | P73 |
| | HDAC4 | 9759 | 239701828 | Missense | c.2426C>T | p.P545L | uc002vyk.2 | P39 |
| | E2F3 | 1871 | 20595009 | Missense | c.1322T>C | p.I332T | uc003nda.2 | P34 |
| | CCNB3 | 85417 | 50107426 | Missense | c.4170A>C | p.Q1291P | uc004dox.2 | P69 |
| | SMC1A | 8243 | 53439984 | Missense | c.2819C>A | p.T917N | uc004dsg.1 | P57 |
| | ERCC4 | 2072 | 13933620 | Missense | c.1088A>G | p.K360R | uc002dce.2 | P52 |
| | BRCA1 | 672 | 38499191 | Missense | c.2083G>A | p.S628N | uc002ict1 | P48 |
| | FANCA | 2175 | 88385382 | Missense | c.1331C>T | p.A430V | uc002fou.1 | P31 |
| | MSH4 | 4438 | 76086496 | Missense | c.1218C>G | p.L393V | uc001dhd.1 | P14 |
| Inflammatory | CD14 | 929 | 139991681 | Missense | c.1426T>C | p.S358P | uc003lgi.1 | P9 |
| pathways | TLR8 | 51311 | 12848204 | Missense | c.1329G>T | p.R393I | uc004cvd.1 | P52 |
| | RIPK1 | 8737 | 3058352 | Missense | c.2028A>G | p.K599R | uc010jni.1 | P41 |
| | MAP3K14 | 9020 | 40723695 | Missense | c.309C>G | p.A67G | uc002iiw.1 | P19 |
| | MAPK8 | 5599 | 49303987 | Missense | c.963G>A | p.E247K | uc009xnz.1 | P1 |
| | IRAK4 | 51135 | 42466478 | Missense | c.1322A>G | p.K400E | uc001rnu.2 | P77 |
| | TRAF3 | 7187 | 102408006 | Splice_Site_SNP | c.e4_splice_site | | uc001ymc.1 | P15 |
| | PPM1A | 5494 | 59819255 | Missense | c.396C>A | p.S100R | uc001xew.2 | P27 |
| | NFKBIA | 4792 | 34943526 | Missense | c.186C>A | p.L26M | uc001wtf.2 | P89 |
| | IFNA8 | 3445 | 21399358 | Missense | c.213C>G | p.F61L | uc003zpc.1 | P39 |
| RNA | SPOP | 8405 | 45051434 | Missense | c.859G>A | p.D130N | uc002ipb.1 | P32 |
| processing | PRPF8 | 10594 | 1524616 | Missense | c.3283C>T | p.R1057W | uc002fte.1 | P63 |
| | RBM39 | 9584 | 33776456 | Missense | c.796A>T | p.D151V | uc002xeb.1 | P34 |
| | U2AF2 | 11338 | 60864312 | Missense | c.1486T>A | p.M144K | uc002qlu.1 | P39 |
| | CPSF2 | 53981 | 91678442 | Missense | c.1080G>T | p.K281N | uc001yah.1 | P2 |
| | XPO1 | 7514 | 61572976 | Missense | c.1840G>A | p.E571K | uc002sbi.1 | P84 |

TABLE 8
Clinical characteristics of CLL patients harboring the 9 driver mutations.

| | | | | | |
|---|---|---|---|---|---|
| | Protein | | | | |
| Pt: Treatment status | change | Mutation type | Cytogenetic abnormalities | ZAP70 | IGHV |

TP53

| | | | | | |
|---|---|---|---|---|---|
| Untreated | | | | | |
| P74 | L111R | Missense | del (17p) | No | Unmut |
| P62 | R273C | Missense | None | No | Mut |
| P76 | H193L | Missense | del(13q) | No | Mut |
| P49 | N131del | In frame del | del(13q); del(17p) | Yes | Un |
| P90 | H214R | Missense | del(17p) | N/A | N/A |
| Treated | | | | | |
| P3 | R248Q | Missense | del (13q); del (17p) | Yes | Unmut |
| P9 | I255F | Missense | Trisomy 12; del (13q); del | No | Unmut |
| | | | (17p) | | |
| P41 | C238S | Missense | del (13q); del (17p) | No | Unmut |
| P42 | D281N | Missense | Trisomy 12; del (13q); del | Yes | Mut |
| | | | (17p) | | |
| P91 | S215R | Missense | del (13q) | Yes | Unmut |
| | *394L | Read through | | | |
| P72 | G187_splice | Splice site | del (13q); del (11q); del | Yes | Unmut |
| | | | (17p) | | |
| P33 | R273H | Missense | del(13q); del(17p) | Yes | Unmut |
| P39 | C135Y | Missense | del(13q); del(17p) | Yes | Unmut |
| P65 | R273H | Missense | Tri (12), del (13q); del | Yes | Unmut |
| | | | (17p) | | |

ATM

| | | | | | |
|---|---|---|---|---|---|
| Untreated | | | | | |
| P8 | L2135fs | Frame shift | None | N/A | Unmut |
| P17 | Y1252F | Missense | del (13q) | No | Mut |
| P23 | H2038R | Missense | Trisomy 12 | Yes | N/A |
| Treated | | | | | |
| P5 | Y2954C | Missense | del (13q); del (11q) | Yes | Mut |
| P73 | Q2522H | Missense | Trisomy 12; del (13q) | Yes | N/A |
| | Y2817* | Stop | (13q); del (11q) | | |
| P48 | L546fs | Frame shift | Del (13q); del (11q) | Yes | Unmut |
| P85 | C1726_splice | Splice site | Del (13q); del (11q) | Yes | Unmut |
| P61 | K468fs | Frame shift | normal | No | N/A |

MYD88

| | | | | | |
|---|---|---|---|---|---|
| Untreated | | | | | |
| P17 | L265P | Missense | del (13q) | No | Mut |
| P18 | M232T | Missense | del (13q) | No | Mut |
| P20 | L265P | Missense | del (13q) | Yes | Mut |
| P25 | L265P | Missense | Trisomy 12; del (13q) | No | Mut |
| P67 | M232T | Missense | del (13q) | No | Mut |
| P31 | L265P | Missense | del (13q) | Yes | Mut |
| Treated | | | | | |
| P5 | L265P | Missense | del (13q); del (11q) | Yes | Mut |
| P46 | P258L | Missense | del (13q); del (17p) | No | Mut |
| P66 | L265P | Missense | del (13q) | No | Mut |

SF3B1

| | | | | | |
|---|---|---|---|---|---|
| Untreated | | | | | |
| P32 | K700E | Missense | del (13q); del (11q) | No | Unmut |
| P8 | G742D | Missense | None | N/A | Unmut |
| P37 | K700E | Missense | del (11q) | Yes | Mut |
| P43 | K700E | Missense | del (11q); del (17p) | Yes | Unmut |
| P51 | G742D | Missense | del (11q) | N/A | N/A |
| P58 | G740E | Missense | del (13q) | Yes | Unmut |
| P84 | K741N | Missense | normal | No | Unmut |
| Treated | | | | | |
| P6 | N626H | Missense | del (13q); del (11q) | No | Unmut |
| P40 | Q903R | Missense | del (13q); del (11q) | Yes | Unmut |
| P60 | R625L | Missense | del (13q); del (11q) | Yes | Unmut |
| P91 | K700E | Missense | del (13q) | Yes | Unmut |
| P59 | K700E | Missense | del (13q); del (17p) | Yes | Unmut |
| P61 | K700E | Missense | normal | N/A | N/A |
| P85 | K700E | Missense | Del (13q); del (11q) | Yes | Unmut |

FBXW7

| | | | | | |
|---|---|---|---|---|---|
| Treated | | | | | |
| P12 | R505C | Missense | del (13q) | No | Mut |
| P35 | G597E | Missense | del (11q) | Yes | Unmut |
| | F280L | Missense | | | |
| P42 | R465H | Missense | del (13q); del (17p) | Yes | Mut |

DDX3X

| | | | | | |
|---|---|---|---|---|---|
| Treated | | | | | |
| P3 | S24* | Nonsense | del (13q); del (17p) | Yes | Unmut |
| P6 | K342_splice | Splice site | del (13q); del (11q) | No | Unmut |
| P37 | S410fs | Frame shift | del (11q) | Yes | Mut |

MAPK1

| | | | | | |
|---|---|---|---|---|---|
| Treated | | | | | |
| P29 | Y316F | Missense | del (13q) | N/A | Mut |
| | D291G | Missense | | | |
| P47 | D162N | Missense | del (13q) | Yes | Unmut |

NOTCH1

| | | | | | |
|---|---|---|---|---|---|
| Untreated | | | | | |
| P27 | P2514fs | Frame shift | Tri (12) | No | N/A |
| P82 | P2514fs | Frame shift | Tri (12), del (13q); del | Yes | Unmut |
| | | | (17p) | | |
| Treated | | | | | |
| P65 | P2514fs | Frame shift | del (13q); del (17p) | Yes | Unmut |
| P87 | P2514fs | Frame shift | Tri (12), del (13q); del | yes | Unmut |
| | | | (11q) | | |

ZMYM3

| | | | | | |
|---|---|---|---|---|---|
| Untreated | | | | | |
| P13 | S1254T | Missense | del (13q) | N/A | Mut |
| P86 | F1302S | Missense | Normal | Yes | Unmut |
| P38 | S53fs | Frame shift | del (11q) | Yes | Unmut |
| Treated | | | | | |
| P35 | Q399* | Nonsense | del (13q) | Yes | Unmut |

TABLE 9
Associations of driver mutations and (A) clinical characteristics and (B) FISH cytogenetics.
A.

| | | | | | | | | | | ZAP70 | | |
|---|---|---|---|---|---|---|---|---|---|---|---|---|
| Gene | Gender | | | Age (years) | | | IGHV | | | Neg- | Pos- | |
| mutation | Female | Male | p-value | <55 | >=55 | p-value | Unmutated | Mutated | p-value | ative | itive | p-value |
| N | 35 | 56 | | 51 | 40 | | 40 | 38 | | 44 | 38 | |
| p53 | 5 (14) | 9 (16) | 0.99 | 8 (16) | 6 (15) | 0.99 | 10 (25) | 3 (8) | 0.07 | 5 (11) | 8 (21) | 0.36 |
| SF3B1 | 7 (20) | 7 (13) | 0.38 | 8 (16) | 6 (15) | 0.99 | 9 (23) | 2 (5) | 0.048 | 5 (11) | 8 (21) | 0.36 |
| MYD88 | 3 (9) | 6 (11) | 0.99 | 6 (12) | 3 (8) | 0.73 | 0 (0) | 9 (24) | <0.001 | 6 (14) | 3 (8) | 0.49 |
| ATM | 2 (6) | 6 (11) | 0.71 | 5 (10) | 3 (8) | 0.99 | 3 (8) | 2 (5) | 0.99 | 2 (5) | 6 (16) | 0.14 |
| NOTCH1 | 3 (9) | 1 (2) | 0.16 | 0 (0) | 4 (10) | 0.034 | 3 (8) | 0 (0) | 0.24 | 1 (2) | 3 (8) | 0.33 |
| ZMYM3 | 2 (6) | 2 (4) | 0.64 | 4 (8) | 0 (0) | 0.13 | 4 (10) | 0 (0) | 0.12 | 0 (0) | 4 (11) | 0.042 |
| DDX3X | 0 (0) | 3 (5) | 0.28 | 1 (2) | 2 (5) | 0.58 | 2 (5) | 1 (3) | 0.99 | 1 (2) | 2 (5) | 0.59 |
| FBXW7 | 2 (6) | 1 (2) | 0.56 | 1 (2) | 2 (5) | 0.58 | 1 (3) | 2 (5) | 0.61 | 1 (2) | 2 (5) | 0.59 |
| MAPK1 | 1 (3) | 1 (2) | 0.99 | 2 (4) | 0 (0) | 0.5 | 1 (3) | 1 (3) | 0.99 | 0 (0) | 1 (3) | 0.46 |

B.

| | del(13q) Het | | | del(13q) Homo | | | Trisomy 12 | | | del(11q) | | | del(17p) | | |
|---|---|---|---|---|---|---|---|---|---|---|---|---|---|---|---|
| Gene | Neg- | Pos- | | Neg- | Pos- | | Neg- | Pos- | | Neg- | Pos- | | Neg- | Pos- | |
| mutation | ative | itive | value | ative | itive | p-value | ative | itive | p-value | ative | itive | value | ative | itive | p-value |
| N | 38 | 53 | | 79 | 12 | | 78 | 13 | | 69 | 22 | | 74 | 17 | |
| p53 | 4 (11) | 10 (19) | 0.38 | 13 (16) | 1 (8) | 0.68 | 12 (15) | 2 (15) | 0.99 | 13 (19) | 1 (5) | 0.17 | 3 (4) | 11 (65) | <0.001 |
| SF3B1 | 7 (18) | 7 (13) | 0.56 | 13 (16) | 1 (8) | 0.68 | 14 (18) | 0 (0) | 0.21 | 6 (9) | 8 (36) | 0.004 | 13 (18) | 1 (6) | 0.45 |
| MYD88 | 0 (0) | 9 (17) | 0.009 | 8 (10) | 1 (8) | 0.99 | 8 (10) | 1 (8) | 0.99 | 8 (12) | 1 (5) | 0.45 | 8 (11) | 1 (6) | 0.99 |
| ATM | 3 (8) | 5 (9) | 0.99 | 8 (10) | 0 (0) | 0.59 | 6 (8) | 2 (15) | 0.32 | 4 (6) | 4 (18) | 0.09 | 8 (11) | 0 (0) | 0.34 |
| NOTCH1 | 1 (3) | 3 (6) | 0.64 | 4 (5) | 0 (0) | 0.99 | 1 (1) | 3 (23) | 0.009 | 3 (4) | 1 (5) | 0.99 | 2 (3) | 2 (12) | 0.16 |
| ZMYM3 | 3 (8) | 1 (2) | 0.3 | 4 (5) | 0 (0) | 0.99 | 4 (5) | 0 (0) | 0.99 | 2 (3) | 2 (9) | 0.25 | 4 (5) | 0 (0) | 0.99 |
| DDX3X | 1 (3) | 2 (4) | 0.99 | 2 (3) | 1 (8) | 0.35 | 3 (4) | 0 (0) | 0.99 | 1 (1) | 2 (9) | 0.14 | 2 (3) | 1 (6) | 0.47 |
| FBXW7 | 1 (3) | 2 (4) | 0.99 | 3 (4) | 0 (0) | 0.99 | 1 (1) | 2 (15) | 0.052 | 2 (3) | 1 (5) | 0.57 | 2 (3) | 1 (6) | 0.47 |
| MAPK1 | 0 (0) | 2 (4) | 0.51 | 2 (3) | 0 (0) | 0.99 | 2 (3) | 0 (0) | 0.99 | 2 (3) | 0 (0) | 0.99 | 2 (3) | 0 (0) | 0.99 |

Note on multiple-hypothesis corrections:
q-valeu (1) = corrected for 9 hypotheses (the 9 possible genes being considered)
q-value (2) = corrected for 45 hypotheses (all combinations of genes × cytogentic abnormalities)

TABLE 10
% Tumor cells harboring cytogenetic abnormalities.

| | del(13q) | del(13q) | trisomy | | |
|---|---|---|---|---|---|
| Patient ID | het | homo | 12 | del(11q) | del(17p) |
| P1 | 86 | 0 | 0 | 90 | 0 |
| P2 | 0 | 0 | 0 | 0 | 0 |
| P3 | 80 | 0 | 0 | 0 | 28 |
| P4 | 0 | 46 | 0 | 0 | 0 |
| P5 | 73 | 0 | 0 | 86 | 0 |
| P6 | 40 | 10 | 0 | 15 | 0 |
| P7 | 17 | 0 | 0 | 32 | 0 |
| P8 | 0 | 0 | 0 | 0 | 0 |
| P9 | 16 | 0 | 75 | 0 | 14 |
| P10 | 10 | 0 | 0 | 0 | 0 |
| P11 | 63 | 26 | 0 | 0 | 0 |
| P12 | 16 | 0 | 35 | 0 | 8 |
| P13 | 39 | 0 | 0 | 0 | 7 |
| P14 | 88 | 0 | 0 | 0 | 0 |
| P15 | 0 | 0 | 38 | 0 | 0 |
| P16 | 0 | 89 | 0 | 0 | 0 |
| P17 | 77 | 0 | 0 | 0 | 0 |
| P18 | 30 | 0 | 0 | 0 | 0 |
| P19 | 65 | 0 | 0 | 0 | 0 |
| P20 | 61 | 0 | 0 | 0 | 0 |
| P21 | 61 | 0 | 0 | 0 | 0 |
| P22 | 10 | 0 | 0 | 0 | 6 |
| P23 | 0 | 0 | 85 | 0 | 0 |
| P24 | 0 | 90 | 0 | 0 | 0 |
| P25 | 10 | 0 | 50 | 0 | 0 |
| P26 | 0 | 27 | 0 | 0 | 0 |
| P27 | 0 | 0 | 27 | 0 | 6 |
| P28 | 83 | 0 | 0 | 0 | 0 |
| P29 | 20 | 0 | 0 | 0 | 6 |
| P30 | 20 | 0 | 0 | 0 | 0 |
| P31 | 11 | 0 | 0 | 0 | 7 |
| P32 | 24 | 0 | 0 | 89 | 0 |
| P33 | 62 | 0 | 0 | 0 | 97 |
| P34 | 20 | 0 | 0 | 33 | 0 |
| P35 | 7 | 0 | 0 | 81 | 0 |
| P36 | 30 | 0 | 0 | 43 | 0 |
| P37 | 0 | 0 | 0 | 50 | 0 |
| P38 | 0 | 0 | 0 | 72 | 0 |
| P39 | 10 | 0 | 0 | 0 | 15 |
| P40 | 16 | 0 | 0 | 27 | 0 |
| P41 | 72 | 0 | 0 | 0 | 47 |
| P42 | 72 | 0 | 18 | 0 | 86 |
| P43 | 0 | 0 | 0 | 67 | 9 |
| P44 | 0 | 0 | 0 | 0 | 46 |
| P45 | 87 | 0 | 0 | 94 | 0 |
| P46 | 26 | 51 | 0 | 0 | 11 |
| P47 | 52 | 0 | 0 | 0 | 0 |
| P48 | 96 | 0 | 0 | 91 | 0 |
| P49 | 15 | 0 | 0 | 0 | 61 |
| P50 | 0 | 0 | 0 | 0 | 0 |
| P51 | 3 | 0 | 0 | 13 | 5 |
| P52 | 6 | 91 | 0 | 0 | 0 |
| P53 | 0 | 0 | 0 | 0 | 0 |
| P54 | 36 | 7 | 0 | 0 | 0 |
| P55 | 0 | 0 | 73 | 0 | 3 |
| P56 | 0 | 0 | 0 | 0 | 0 |
| P57 | 4 | 0 | 56 | 0 | 0 |
| P58 | 24 | 0 | 0 | 0 | 0 |
| P59 | 0 | 0 | 0 | 0 | 0 |
| P60 | 93 | 0 | 0 | 34 | 0 |
| P61 | 0 | 0 | 0 | 0 | 0 |
| P62 | 0 | 0 | 0 | 0 | 0 |
| P63 | 0 | 0 | 0 | 0 | 0 |
| P64 | 0 | 82 | 0 | 0 | 9 |
| P65 | 23 | 0 | 0 | 0 | 43 |
| P66 | 24 | 0 | 0 | 0 | 0 |
| P67 | 31 | 0 | 0 | 0 | 6 |
| P68 | 61 | 0 | 0 | 0 | 0 |
| P69 | 4 | 0 | 0 | 0 | 0 |
| P70 | 0 | 61 | 0 | 0 | 0 |
| P71 | 64 | 0 | 0 | 7 | 0 |
| P72 | 97 | 0 | 0 | 19 | 46 |
| P73 | 100 | 0 | 35 | 94 | 0 |
| P74 | 0 | 0 | 0 | 0 | 45 |
| P75 | 6 | 0 | 0 | 0 | 0 |
| P76 | 6 | 40 | 0 | 0 | 0 |
| P77 | 71 | 0 | 0 | 0 | 0 |
| P78 | 25 | 0 | 0 | 29 | 29 |
| P79 | 0 | 0 | 0 | 0 | 0 |
| P80 | 81 | 0 | 0 | 0 | 0 |
| P81 | 0 | 0 | 0 | 0 | 0 |
| P82 | 9 | 0 | 32 | 0 | 12 |
| P83 | 72 | 0 | 0 | 0 | 0 |
| P84 | 5 | 0 | 0 | 0 | 0 |
| P85 | 87 | 0 | 0 | 93 | 0 |
| P86 | 0 | 0 | 0 | 0 | 0 |
| P87 | 51 | 0 | 73 | 89 | 0 |
| P88 | 0 | 0 | 76 | 0 | 0 |
| P89 | 0 | 0 | 0 | 4 | 0 |
| P90 | 0 | 0 | 0 | 0 | 47 |
| P91 | 44 | 0 | 0 | 0 | 0 |

TABLE 11
Primers for the quantitative PCR of
BRD2 and RIOK3 transcripts.

| Target | Splicing | | |
|---|---|---|---|
| gene | status | Primers | |
| BRD2 | Spliced | Applied Biosystems | |
| | | (Hs01121991_g1) | |
| | Unspliced | Forward | GCAAGATTTTATACCATGTTC |
| | | | ACCAACT |
| | | | (SEQ ID NO: 1) |
| | | Reverse | CCCACCTACTAAATGAACACACAGA |
| | | | (SEQ ID NO: 2) |
| | | Probe | CTCACCTTGTTGTAAATGT |
| | | | (SEQ ID NO: 3) |
| RIOK3 | Spliced | Forward | CACAGCTTAGGCGTGAAGAAAA |
| | | | (SEQ ID NO: 4) |
| | | Reverse | GCTGTCTTCATAAGGATGCACTTTT |
| | | | (SEQ ID NO: 5) |
| | | Probe | AAGGAAATGGAAACTTTG |
| | | | (SEQ ID NO: 6) |
| | Unspliced | Forward | CACAGCTTAGGCGTGAAGAAAA |
| | | | (SEQ ID NO: 7) |
| | | Reverse | CCACTCAATGAAGTTGTCACAA |
| | | | TAAGG |
| | | | (SEQ ID NO: 8) |
| | | Probe | CAATGGAGATAGCAAAGGTATT |
| | | | (SEQ ID NO: 9) |

### REFERENCES

Alami, R., Fan, Y., Pack, S., Sonbuchner, T. M., Besse, A., Lin, Q., Greally, J. M., Skoultchi, A. I., and Bouhassira, E. E. (2003). Mammalian linker-histone subtypes differentially affect gene expression in vivo. Proc Natl Acad Sci USA. 100, 5920-5925.
Antoniak, C. (1974). Mixtures of Dirichlet processes with applications to Bayesian nonparametric problems. The Annals of Statistics. 2, 1152-1174.
Armistead, P., Mohseni, M., Gerwin, R., Walsh, E., Iravani, M., Chahardouli, B., Rostami, S., Zhang, W., Neuberg, D., Rioux, J., et al. (2008). Erythroid-lineage-specific engraftment in patients with severe hemoglobinopathy following allogeneic hematopoietic stem cell transplantation. Exp Hematol. 36, 1205-1215.
Austen B, Powell J E, Alvi A, et al. Mutations in the ATM gene lead to impaired overall and treatment-free survival that is independent of IGVH mutation status in patients with B-CLL. Blood 2005; 106:3175-82.
Babaei-Jadidi R, Li N, Saadeddin A, et al. FBXW7 influences murine intestinal homeostasis and cancer, targeting Notch, Jun, and DEK for degradation. J Exp Med 2011; 208:295-312.
Benjamini, Y., and Hochberg, Y. (1995). Controlling the false discovery rate: a practical and powerful approach to multiple testing. Journal of the Royal Statistical Society. 57, 289-300.
Berger M F, Lawrence M S, Demichelis F, et al. The genomic complexity of primary human prostate cancer. Nature 2011; 470:214-20.
Berger M F, Lawrence M S, Demichelis F, et al. The genomic complexity of primary human prostate cancer. Nature 2011; 470:214-20.
Beroukhim, R., Mermel, C. H., Porter, D., Wei, G., Raychaudhuri, S., Donovan, J., Barretina, J., Boehm, J. S., Dobson, J., Urashima, M., et al. (2010). The landscape of somatic copy-number alteration across human cancers. Nature. 463, 899-905.
Bos, J. L. (1989). ras oncogenes in human cancer: a review. Cancer Res. 49, 4682-4689.
Brown, J. R., Hanna, M., Tesar, B., Werner, L., Pochet, N., Asara, J. M., Wang, Y. E., Dal Cin, P., Fernandes, S. M., Thompson, C., et al. (2012). Integrative genomic analysis implicates gain of PIK3CA at 3q26 and MYC at 8q24 in chronic lymphocytic leukemia. Clin Cancer Res. 18, 3791-3802.
Cancer Genome Atlas Research Network. Comprehensive genomic characterization defines human glioblastoma genes and core pathways. Nature 2008; 455:1061-8.
Cancer Genome Atlas Research Network. Integrated genomic analyses of ovarian carcinoma. Nature 2011; 474:609-15.
Carter, S. L., Cibulskis, K., Helman, E., McKenna, A., Shen, H., Zack, T., Laird, P. W., Onofrio, R. C., Winckler, W., Weir, B. A., et al. (2012). Absolute quantification of somatic DNA alterations in human cancer. Nat. Biotechnol. 30, 413-421.
Carter, S. L., Meyerson, M., & Getz, G. (2011). Accurate estimation of homologue-specific DNA concentration ratios in cancer samples allows long-range haplotyping. Available from Nature Precedings online.
Chapman M A, Lawrence M S, Keats J J, et al. Initial genome sequencing and analysis of multiple myeloma. Nature 2011; 471:467-72.
Cheson, B. D., Pfistner, B., Juweid, M. E., Gascoyne, R. D., Specht, L., Horning, S. J., Coiffier, B., Fisher, R. I., Hagenbeek, A., Zucca, E., et al. (2007). Revised response criteria for malignant lymphoma. J Clin Oncol. 25, 579-586.
Cibulskis, K., McKenna, A., Fennell, T., Banks, E., DePristo, M., and Getz, G. (2011). ContEst: estimating cross-contamination of human samples in next-generation sequencing data. Bioinformatics. 27, 2601-2602.
CLL Trialists Collaborative Group (1999). Chemotherapeutic options in chronic lymphocytic leukemia: a meta-analysis of the randomized trials. J Natl Cancer Inst. 91, 861-868.
Corrionero A, Minana B, Valcarcel J. Reduced fidelity of branch point recognition and alternative splicing induced by the anti-tumor drug spliceostatin A. Genes Dev 2011; 25:445-59.
Damle, R. N., Wasil, T., Fais, F., Ghiotto, F., Valetto, A., Allen, S. L., Buchbinder, A., Budman, D., Dittmar, K., Kolitz, J., et al. (1999). Ig V gene mutation status and CD38 expression as novel prognostic indicators in chronic lymphocytic leukemia. Blood. 94, 1840-1847.
DePristo M A, Banks E, Poplin R, et al. A framework for variation discovery and genotyping using next-generation DNA sequencing data. Nat Genet. 2011; 43:491-8.
Deutsch, A. J., Aigelsreiter, A., Staber, P. B., Beham, A., Linkesch, W., Guelly, C., Brezinschek, R. I., Fruhwirth, M., Emberger, W., Buettner, M., et al. (2007). MALT lymphoma and extranodal diffuse large B-cell lymphoma are targeted by aberrant somatic hypermutation. Blood. 109, 3500-3504.
Ding L, Getz G, Wheeler D A, et al. Somatic mutations affect key pathways in lung adenocarcinoma. Nature 2008; 455:1069-75.
Ding, L., Ley, T. J., Larson, D. E., Miller, C. A., Koboldt, D. C., Welch, J. S., Ritchey, J. K., Young, M. A., Lamprecht, T., McLellan, M. D., et al. (2012). Clonal evolution in relapsed acute myeloid leukaemia revealed by whole-genome sequencing. Nature. 481, 506-510.
Dohner H, Stilgenbauer S, Benner A, et al. Genomic aberrations and survival in chronic lymphocytic leukemia. N Engl J Med 2000; 343:1910-6.
Döhner, H. (2005). The use of molecular markers in selecting therapy for CLL. Clin Adv Hematol Oncol. 3, 103-104.
Edelmann, J., Holzmann, K., Miller, F., Winkler, D., Buhler, A., Zenz, T., Bullinger, L., Kuhn, M. W., Gerhardinger, A., Bloehdorn, J., et al. (2012). High-resolution genomic profiling of chronic lymphocytic leukemia reveals new recurrent genomic alterations. Blood. [Epub ahead of print].
Egan, J. B., Shi, C. X., Tembe, W., Christoforides, A., Kurdoglu, A., Sinari, S., Middha, S., Asmann, Y., Schmidt, J., Braggio, E., et al. (2012). Whole-genome sequencing of multiple myeloma from diagnosis to plasma cell leukemia reveals genomic initiating events, evolution, and clonal tides. Blood. 120, 1060-1066.
Escobar, M., and West, M. (1995). Bayesian density estimation and inference using mixtures. Journal of the American Statistical Association. 90, 577-588.
Eskandarpour, M., Huang, F., Reeves, K. A., Clark, E., and Hansson, J. (2009). Oncogenic NRAS has multiple effects on the malignant phenotype of human melanoma cells cultured in vitro. Int J Cancer. 124, 16-26.
Fabbri G, Rasi S, Rossi D, et al. Analysis of the chronic lymphocytic leukemia coding genome: role of NOTCH1 mutational activation. J Exp Med 2011.
Fan L, Lagisetti C, Edwards C C, Webb T R, Potter P M. Sudemycins, Novel Small Molecule Analogues of FR901464, Induce Alternative Gene Splicing. ACS Chem Biol 2011.
Fisher, R. A. (1932). Statistical methods for research workers, 4th edn (Oliver and Boyd).
Fisher, S., Barry, A., Abreu, J., Minie, B., Nolan, J., Delorey, T. M., Young, G., Fennell, T. J., Allen, A., Ambrogio, L., et al. (2011). A scalable, fully automated process for construction of sequence-ready human exome targeted capture libraries. Genome Biol. 12, R1.
Forbes, S. A., Bhamra, G., Bamford, S., Dawson, E., Kok, C., Clements, J., Menzies, A., Teague, J. W., Futreal, P. A., and Stratton, M. R. (2008). The Catalogue of SomaticMutations in Cancer (COSMIC). Curr Protoc Hum Genet. Chapter 10, Unit 10 11.
Forbes, S. A., Tang, G., Bindal, N., Bamford, S., Dawson, E., Cole, C., Kok, C. Y., Jia, M., Ewing, R., Menzies, A., et al. (2010). COSMIC (the Catalogue of Somatic Mutations in Cancer): a resource to investigate acquired mutations in human cancer. Nucleic Acids Res. 38, D652-657.
Futreal, P. A., Coin, L., Marshall, M., Down, T., Hubbard, T., Wooster, R., Rahman, N., and Stratton, M. R. (2004). A census of human cancer genes. Nat Rev Cancer. 4, 177-183.
GenePattern 2.0. Nat. Genet. 38, 500-501.
Gerlinger, M., and Swanton, C. (2010). How Darwinian models inform therapeutic failure initiated by clonal heterogeneity in cancer medicine. Br J Cancer. 103, 1139-1143.
Gerlinger, M., Rowan, A. J., Horswell, S., Larkin, J., Endesfelder, D., Gronroos, E., Martinez, P., Matthews, N., Stewart, A., Tarpey, P., et al. (2012). Intratumor heterogeneity and branched evolution revealed by multiregion sequencing. N Engl J. Med. 366, 883-892.
Gerstung, M., Beisel, C., Rechsteiner, M., Wild, P., Schraml, P., Moch, H., and Beerenwinkel, N. (2012). Reliable detection of subclonal single-nucleotide variants in tumour cell populations. Nat. Commun. 3, 811.
Getz G, Hofling H, Mesirov J P, et al. Comment on “The consensus coding sequences of human breast and colorectal cancers”. Science 2007; 317:1500.
Gnirke A, Melnikov A, Maguire J, et al. Solution hybrid selection with ultra-long oligonucleotides for massively parallel targeted sequencing. Nat Biotechnol 2009; 27:182-9.
Greaves, M., and Maley, C. C. (2012). Clonal evolution in cancer. Nature. 481, 306-313.
Grossmann, V., Tiacci, E., Holmes, A. B., Kohlmann, A., Martelli, M. P., Kern, W., Spanhol-Rosseto, A., Klein, H. U., Dugas, M., Schindela, S., et al. (2011). Whole-exome sequencing identifies somatic mutations of BCOR in acute myeloid leukemia with normal karyotype. Blood. 118, 6153-6163.
Grubor, V., Krasnitz, A., Troge, J., Meth, J., Lakshmi, B., Kendall, J., Yamrom, B., Alex, G., Pai, D., Navin, N., et al. (2009). Novel genomic alterations and clonal evolution in chronic lymphocytic leukemia revealed by representational oligonucleotide microarray analysis (ROMA). Blood. 113, 1294-1303. Gutierrez A, Jr., Tschumper R C, Wu X, et al. LEF-1 is a prosurvival factor in chronic lymphocytic leukemia and is expressed in the preleukemic state of monoclonal B cell lymphocytosis. Blood 2010.
Hallek, M., Cheson, B., Catovsky, D., Caligaris-Cappio, F., Dighiero, G., Döhner, H., Hillmen, P., Keating, M., Montserrat, E., Rai, K., et al. (2008). Guidelines for the diagnosis and treatment of chronic lymphocytic leukemia: a report from the International Workshop on Chronic Lymphocytic Leukemia updating the National Cancer Institute-Working Group 1996 guidelines. Blood. 111, 5446-5456.
Hosgood, H. D., 3rd, Baris, D., Zhang, Y., Berndt, S. I., Menashe, I., Morton, L. M., Lee, K. M., Yeager, M., Zahm, S. H., Chanock, S., et al. (2009). Genetic variation in cell cycle and apoptosis related genes and multiple myeloma risk. Leuk Res. 33, 1609-1614.
Huber, W., von Heydebreck, A., Sultmann, H., Poustka, A., and Vingron, M. (2002). Variance stabilization applied to microarray data calibration and to the quantification of differential expression. Bioinformatics. 18 Suppl 1, S96-104.
Irizarry, R. A., Hobbs, B., Collin, F., Beazer-Barclay, Y. D., Antonellis, K. J., Scherf, U., and Speed, T. P. (2003). Exploration, normalization, and summaries of high density oligonucleotide array probe level data. Biostatistics. 4, 249-264.
Jablonski, D. (2001). Lessons from the past: evolutionary impacts of mass extinctions. Proc Natl Acad Sci USA. 98, 5393-5398.
Johnson, W. E., Li, C., and Rabinovic, A. (2007). Adjusting batch effects in microarrayexpression data using empirical Bayes methods. Biostatistics. 8, 118-127.
Kaida D, Motoyoshi H, Tashiro E, et al. Spliceostatin A targets SF3b and inhibits both splicing and nuclear retention of pre-mRNA. Nat Chem Biol 2007; 3:576-83.
Klein U, Tu Y, Stolovitzky G A, et al. Gene expression profiling of B cell chronic lymphocytic leukemia reveals a homogeneous phenotype related to memory B cells. J Exp Med 2001; 194:1625-38.
Klein, U., Lia, M., Crespo, M., Siegel, R., Shen, Q., Mo, T., Ambesi-Impiombato, A., Califano, A., Migliazza, A., Bhagat, G., et al. (2010). The DLEU2/miR-15a/16-1 cluster controls B cell proliferation and its deletion leads to chronic lymphocytic leukemia. Cancer Cell. 17, 28-40.
Kotake Y, Sagane K, Owa T, et al. Splicing factor SF3b as a target of the antitumor natural product pladienolide. Nat Chem Biol 2007; 3:570-5.
Lee M G, Wynder C, Cooch N, Shiekhattar R. An essential role for CoREST in nucleosomal histone 3 lysine 4 demethylation. Nature 2005; 437:432-5.
Ley T J, Mardis E R, Ding L, et al. DNA sequencing of a cytogenetically normal acute myeloid leukaemia genome. Nature 2008; 456:66-72.
Li H, Ruan J, Durbin R. Mapping short DNA sequencing reads and calling variants using mapping quality scores. Genome Res 2008; 18:1851-8.
Lin, K., Tam, C., Keating, M., Wierda, W., O'Brien, S., Lerner, S., Coombes, K., Schlette, E., Ferrajoli, A., Barron, L., et al. (2009). Relevance of the immunoglobulin VH somatic mutation status in patients with chronic lymphocytic leukemia treated with fludarabine, cyclophosphamide, and rituximab (FCR) or related chemoimmunotherapy regimens. Blood. 113, 3168-3171.
Liu, W., Laitinen, S., Khan, S., Vihinen, M., Kowalski, J., Yu, G., Chen, L., Ewing, C. M., Eisenberger, M. A., Carducci, M. A., et al. (2009). Copy number analysis indicates monoclonal origin of lethal metastatic prostate cancer. Nat. Med. 15, 559-565.
Lohr, J. G., Stojanov, P., Lawrence, M. S., Auclair, D., Chapuy, B., Sougnez, C., Cruz-Gordillo, P., Knoechel, B., Asmann, Y. W., Slager, S. L., et al. (2012). Discovery and prioritization of somatic mutations in diffuse large B-cell lymphoma (DLBCL) by whole-exome sequencing. Proc Natl Acad Sci USA. 109, 3879-3884.
Makinen, N., Mehine, M., Tolvanen, J., Kaasinen, E., Li, Y., Lehtonen, H. J., Gentile, M., Yan, J., Enge, M., Taipale, M., et al. (2011). MED12, the mediator complex subunit 12 gene, is mutated at high frequency in uterine leiomyomas. Science. 334, 252-255.
Maley, C. C., Galipeau, P. C., Finley, J. C., Wongsurawat, V. J., Li, X., Sanchez, C. A., Paulson, T. G., Blount, P. L., Risques, R. A., Rabinovitch, P. S., et al. (2006). Genetic clonal diversity predicts progression to esophageal adenocarcinoma. Nat. Genet. 38, 468-473.
Mardis E R, Ding L, Dooling D J, et al. Recurring mutations found by sequencing an acute myeloid leukemia genome. N Engl J Med 2009; 361:1058-66.
Marechal, Y., Queant, S., Polizzi, S., Pouillon, V., and Schurmans, S. (2011). Inositol 1,4,5-trisphosphate 3-kinase B controls survival and prevents anergy in B cells. Immunobiology. 216, 103-109.
Masica, D. L., and Karchin, R. (2011). Correlation of somatic mutation and expression identifies genes important in human glioblastoma progression and survival. Cancer Res. 71, 4550-4561.
Massiello A, Roesser J R, Chalfant C E. SAP155 Binds to ceramide-responsive RNA cis-element 1 and regulates the alternative 5′ splice site selection of Bcl-x pre-mRNA. FASEB J 2006; 20:1680-2.
McCarthy, H., Wierda, W. G., Barron, L. L., Cromwell, C. C., Wang, J., Coombes, K. R., Rangel, R., Elenitoba-Johnson, K. S., Keating, M. J., and Abruzzo, L. V. (2003). High expression of activation-induced cytidine deaminase (AID) and splice variants is a distinctive feature of poor-prognosis chronic lymphocytic leukemia. Blood. 101, 4903-4908.
Mermel, C. H., Schumacher, S. E., Hill, B., Meyerson, M. L., Beroukhim, R., and Getz, G. (2011). GISTIC2.0 facilitates sensitive and confident localization of the targets of focal somatic copy-number alteration in human cancers. Genome Biol. 12, R41.
Morin R D, Mendez-Lago M, Mungall A J, et al. Frequent mutation of histone-modifying genes in non-Hodgkin lymphoma. Nature 2011.
Mullighan, C. G., Phillips, L. A., Su, X., Ma, J., Miller, C. B., Shurtleff, S. A., and Downing, J. R. (2008). Genomic analysis of the clonal origins of relapsed acute lymphoblastic leukemia. Science. 322, 1377-1380.
Muzio M, Apollonio B, Scielzo C, et al. Constitutive activation of distinct BCR-signaling pathways in a subset of CLL patients: a molecular signature of anergy. Blood 2008; 112:188-95.
Myllykangas S, Ji HP. Targeted deep resequencing of the human cancer genome using next-generation technologies. Biotechnol Genet Eng Rev 2010; 27:135-58
Navin, N., Kendall, J., Troge, J., Andrews, P., Rodgers, L., Mclndoo, J., Cook, K., Stepansky, A., Levy, D., Esposito, D., et al. (2011). Tumour evolution inferred by single-cell sequencing. Nature. 472, 90-94.
Navin, N., Krasnitz, A., Rodgers, L., Cook, K., Meth, J., Kendall, J., Riggs, M., Eberling, Y., Troge, J., Grubor, V., et al. (2010). Inferring tumor progression from genomic heterogeneity. Genome Res. 20, 68-80.
Network CGAR. Comprehensive genomic characterization defines human glioblastoma genes and core pathways. Nature 2008; 455:1061-8.
Ng P C, Kirkness E F, Whole genome sequencing. Methods Mol Biol 2010; 628:215-26.
Ngo V N, Young R M, Schmitz R, et al. Oncogenically active MYD88 mutations in human lymphoma. Nature 2011; 470:115-9.
Nik-Zainal, S., Van Loo, P., Wedge, D. C., Alexandrov, L. B., Greenman, C. D., Lau, K. W., Raine, K., Jones, D., Marshall, J., Ramakrishna, M., et al. (2012). The life history of 21 breast cancers. Cell. 149, 994-1007.
Nowak, M. A., and Sigmund, K. (2004). Evolutionary dynamics of biological games. Science. 303, 793-799.
O'Neil J, Grim J, Strack P, et al. FBW7 mutations in leukemic cells mediate NOTCH pathway activation and resistance to gamma-secretase inhibitors. J Exp Med 2007; 204:1813-24.
Pepper C, Thomas A, Hoy T, Milligan D, Bentley P, Fegan C. The vitamin D3 analog EB1089 induces apoptosis via a p53-independent mechanism involving p38 MAP kinase activation and suppression of ERK activity in B-cell chronic lymphocytic leukemia cells in vitro. Blood 2003; 101:2454-60.
Puente X S, Pinyol M, Quesada V, et al. Whole-genome sequencing identifies recurrent mutations in chronic lymphocytic leukaemia. Nature 2011.
Quesada, V., Conde, L., Villamor, N., Ordonez, G. R., Jares, P., Bassaganyas, L., Ramsay, A. J., Bea, S., Pinyol, M., Martinez-Trillos, A., et al. (2012). Exome sequencing identifies recurrent mutations of the splicing factor SF3B1 gene in chronic lymphocytic leukemia. Nat. Genet. 44, 47-52.
Rassenti L Z, Huynh L, Toy T L, et al. ZAP-70 compared with immunoglobulin heavy-chain gene mutation status as a predictor of disease progression in chronic lymphocytic leukemia. N Engl J Med 2004; 351:893-901.
Rassenti, L., Jain, S., Keating, M., Wierda, W., Greyer, M., Byrd, J., Kay, N., Brown, J., Gribben, J., Neuberg, D., et al. (2008). Relative value of ZAP-70, CD38, and immunoglobulin mutation status in predicting aggressive disease in chronic lymphocytic leukemia. Blood. 112, 1923-1930.
Reich, M., Liefeld, T., Gould, J., Lerner, J., Tamayo, P., and Mesirov, J. P. (2006). Rice, G. I., Bond, J., Asipu, A., Brunette, R. L., Manfield, I. W., Carr, I. M., Fuller, J. C., Jackson, R. M., Lamb, T., Briggs, T. A., et al. (2009). Mutations involved in Aicardi-Goutieres syndrome implicate SAMHD1 as regulator of the innate immune response. Nat Genet. 41, 829-832.
Robinson J T, Thorvaldsdottir H, Winckler W, et al. Integrative genomics viewer. Nat Biotechnol 2011; 29:24-6.
Rosner A, Rinkevich B. The DDX3 subfamily of the DEAD box helicases: divergent roles as unveiled by studying different organisms and in vitro assays. Curr Med Chem 2007; 14:2517-25.
Schuh, A., Becq, J., Humphray, S., Alexa, A., Burns, A., Clifford, R., Feller, S. M., Grocock, R., Henderson, S., Khrebtukova, I., et al. (2012). Monitoring chronic lymphocytic leukemia progression by whole genome sequencing reveals heterogeneous clonal evolution patterns. Blood. [Epub ahead of print].
Shah, S. P., Roth, A., Goya, R., Oloumi, A., Ha, G., Zhao, Y., Turashvili, G., Ding, J., Tse, K., Haffari, G., et al. (2012). The clonal and mutational evolution spectrum of primary triple-negative breast cancers. Nature. 486, 395-399.
Shanafelt, T. D., Hanson, C., Dewald, G. W., Witzig, T. E., LaPlant, B., Abrahamzon, J., Jelinek, D. F., and Kay, N. E. (2008). Karyotype evolution on fluorescent in situ hybridization analysis is associated with short survival in patients with chronic lymphocytic leukemia and is related to CD49d expression. J Clin Oncol. 26, e5-6.
Siepel, A., Bejerano, G., Pedersen, J. S., Hinrichs, A. S., Hou, M., Rosenbloom, K., Clawson, H., Spieth, J., Hillier, L. W., Richards, S., et al. (2005). Evolutionarily conserved elements in vertebrate, insect, worm, and yeast genomes. Genome Res. 15, 1034-1050.
Smoley S A, Van Dyke D L, Kay N E, et al. Standardization of fluorescence in situ hybridization studies on chronic lymphocytic leukemia (CLL) blood and marrow cells by the CLL Research Consortium. Cancer Genet Cytogenet 2010; 203:141-8.
Snuderl, M., Fazlollahi, L., Le, L. P., Nitta, M., Zhelyazkova, B. H., Davidson, C. J., Akhavanfard, S., Cahill, D. P., Aldape, K. D., Betensky, R. A., et al. (2011). Mosaic amplification of multiple receptor tyrosine kinase genes in glioblastoma. Cancer Cell. 20, 810-817.
Stephens, P. J., Tarpey, P. S., Davies, H., Van Loo, P., Greenman, C., Wedge, D. C., Nik-Zainal, S., Martin, S., Varela, I., Bignell, G. R., et al. (2012). The landscape of cancer genes and mutational processes in breast cancer. Nature. 486, 400-404.
Stilgenbauer, S., Sander, S., Bullinger, L., Benner, A., Leupolt, E., Winkler, D., Krober, A., Kienle, D., Lichter, P., and Dohner, H. (2007). Clonal evolution in chronic lymphocytic leukemia: acquisition of high-risk genomic aberrations associated with unmutated V H, resistance to therapy, and short survival. Haematologica. 92, 1242-1245.
Subramanian A, Tamayo P, Mootha V K, et al. Gene set enrichment analysis: a knowledge-based approach for interpreting genome-wide expression profiles. Proc Natl Acad Sci USA 2005; 102:15545-50.
Tiacci E, Trifonov V, Schiavoni G, et al. BRAF mutations in hairy-cell leukemia. N Engl J Med 2011; 364:2305-15.
Trbusek M, Smardova J, Malcikova J, et al. Missense Mutations Located in Structural p53 DNA-Binding Motifs Are Associated With Extremely Poor Survival in Chronic Lymphocytic Leukemia. J Clin Oncol 2011; 29:2703-8.
Unoki, M., and Nakamura, Y. (2003). EGR2 induces apoptosis in various cancer cell lines by direct transactivation of BNIP3L and BAK. Oncogene. 22, 2172-2185.
Vincent, T. L., and Gatenby, R. A. (2008). An evolutionary model for initiation, promotion, and progression in carcinogenesis. Int J Oncol. 32, 729-737.
Wahl M C, Will C L, Luhrmann R. The spliceosome: design principles of a dynamic RNP machine. Cell 2009; 136:701-18.
Walter, M. J., Shen, D., Ding, L., Shao, J., Koboldt, D. C., Chen, K., Larson, D. E., McLellan, M. D., Dooling, D., Abbott, R., et al. (2012). Clonal architecture of secondary acute myeloid leukemia. N Engl J. Med. 366, 1090-1098.
Wang, L., Lawrence, M. S., Wan, Y., Stojanov, P., Sougnez, C., Stevenson, K., Werner, L., Sivachenko, A., DeLuca, D. S., Zhang, L., et al. (2011). SF3B1 and other novel cancer genes in chronic lymphocytic leukemia. N Engl J. Med. 365, 2497-2506.
Welch, J. S., Ley, T. J., Link, D. C., Miller, C. A., Larson, D. E., Koboldt, D. C., Wartman, L. D., Lamprecht, T. L., Liu, F., Xia, J., et al. (2012). The origin and evolution of mutations in acute myeloid leukemia. Cell. 150, 264-278.
Yada M, Hatakeyama S, Kamura T, et al. Phosphorylation-dependent degradation of c-Myc is mediated by the F-box protein Fbw7. EMBO J. 2004; 23:2116-25.
Yoshida, K., Sanada, M., Shiraishi, Y., Nowak, D., Nagata, Y., Yamamoto, R., Sato, Y., Sato-Otsubo, A., Kon, A., Nagasaki, M., et al. (2011). Frequent pathway mutations of splicing machinery in myelodysplasia. Nature. 478, 64-69.
Zenz T, Eichhorst B, Busch R, et al. TP53 mutation and survival in chronic lymphocytic leukemia. J Clin Oncol 2010; 28:4473-9.
Zenz T, Mertens D, Kuppers R, Dohner H, Stilgenbauer S. From pathogenesis to treatment of chronic lymphocytic leukaemia. Nat Rev Cancer 2010; 10:37-50.
Zhang W, Choi J, Zeng W, et al. Graft-versus-leukemia antigen CML66 elicits coordinated B-cell and T-cell immunity after donor lymphocyte infusion. Clin Cancer Res 2010; 16:2729-39.
Zhang, L., Znoyko, I., Costa, L. J., Conlin, L. K., Daber, R. D., Self, S. E., and Wolff, D. J. (2011). Clonal diversity analysis using SNP microarray: a new prognostic tool for chronic lymphocytic leukemia. Cancer Genet. 204, 654-665.

### Other Embodiments

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present invention.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed is:

1. A method of determining a treatment regimen for a subject having chronic lymphocytic leukemia (CLL) comprising identifying a mutation in the SF3B1 gene in a subject sample, wherein the presence of one or more mutations in the SF3B1 gene indicates that the subject should receive an alternative treatment regimen.

2. A method of determining whether a subject having chronic lymphocytic leukemia (CLL) would derive a clinical benefit of early treatment comprising identifying a mutation in the SF3B1 gene in a subject sample, wherein the presence of one or more mutations in the SF3B1 gene indicates that the subject would derive a clinical benefit of early treatment.

3. A method of predicting survivability of a subject having chronic lymphocytic leukemia (CLL) comprising identifying a mutation in the SF3B1 gene in a subject sample, wherein the presence of one or more mutations in the SF3B1 gene indicates that the subject is less likely to survive.

4. A method of identifying a candidate subject for a clinical trial for a treatment protocol for chronic lymphocytic leukemia (CLL) comprising identifying a mutation in the SF3B1 gene in a subject sample, wherein the presence of one or more mutations in the SF3B1 gene indicates that the subject is a candidate for the clinical trial.

5. The method of any one of claims 1-4, wherein the mutation is a missense mutation.

6. The method of any one of claims 1-5, wherein the mutation is a R625L, a N626H, a K700E, a G740E, a K741N or a Q903R, a E622D, a R625G, a Q659R, a K666Q, a K666E, or a G742D mutation in the SF3B1 polypeptide.

7. The method of any one of claims 1-5, wherein the mutation in the SF3B1 gene is within exons 14-17 of the SF3B1 gene.

8. The method of any one of claims 1-7, further comprising detecting at least one other CLL-associated marker.

9. The method of claim 8, wherein the at least one other CLL-associated marker is mutated IGVH or ZAP70 expression status.

10. The method of claim 8, wherein the at least one other CLL-associated marker is a mutation is a risk allele selected from the group consisting of HIST1H1E, NRAS, BCOR, RIPK1, SAMHD1, KRAS, MED12, ITPKB, and EGR2.

11. The method of any one of claims 1-10, further comprising identifying at least one CLL-associated chromosomal abnormality.

12. The method of claim 11, wherein the at least one CLL-associated chromosomal abnormality is selected from the group consisting of 8p deletion, 11q deletion, 17p deletion, Trisomy 12, 13q deletion, monosomy 13, and rearrangements of chromosome 14.

13. A method of treating or alleviating a symptom of chronic lymphocytic leukemia (CLL) comprising administering to a subject a compound that modulates SF3B1.

14. The method of claim 13, wherein said compound is spliceostatin, E7107, or pladienolide.

15. A kit comprising:
(i) a first reagent that detects a mutation in the SF3B1 gene;
(ii) optionally, a second reagent that detects at least one other CLL-associated marker;
(iii) optionally, a third reagent that detects at least one CLL-associated chromosomal abnormality; and
(iv) instructions for their use.

16. The kit of claim 15, wherein the mutation in the SF3B1 gene is a R625L, a N626H, a K700E, a G740E, a K741N or a Q903R, a E622D, a R625G, a Q659R, a K666Q, a K666E, or a G742D mutation in the SF3B1 polypeptide.

17. The kit of claim 15, wherein the mutation in the SF3B1 gene is within exons 14-17 of the SF3B1 gene.

18. The kit of any of claim 15-17, wherein the at least one other CLL-associated marker is ZAP70 expression or mutated IGVH status.

19. The kit of any of claim 15-18, wherein the at least one other CLL-associated marker is a mutation in a risk allele selected from the group consisting of HIST1H1E, NRAS, BCOR, RIPK1, SAMHD1, KRAS, MED12, ITPKB, and EGR2.

20. The kit of any of claim 15-19, wherein the at least one other CLL-associated marker is a mutation in a risk allele selected from the group consisting of TP53, ATM, MYD88, NOTCH1, DDX3X, ZMYM3, FBXW7, XPO1, CHD2, or POT1.

21. The kit of any of claims 15-20, wherein the at least one CLL-associated chromosomal abnormality is selected from the group consisting of 8p deletion 11q deletion, 17p deletion, Trisomy 12, 13q deletion, monosomy 13, and rearrangements of chromosome 14.

22. The kit of any of claims 15-21, wherein the first, second and third reagents are polynucleotides that are capable of hybridizing to the genes or chromosomes of (i), (ii) and/or (iii), wherein said polynucleotides are optionally linked to a detection label.

23. A method comprising
(a) analyzing genomic DNA in a sample obtained from a subject having or suspected of having chronic lymphocytic leukemia (CLL) for the presence of mutation in a risk allele,
(b) determining whether the mutation is clonal or subclonal, and
(c) identifying the subject as a subject at elevated risk of having CLL with rapid disease progression if the mutation is a driver event and subclonal.

24. The method of claim 23, wherein the risk allele is selected from SF3B1, HIST1H1E, NRAS, BCOR, RIPK1, SAMHD1, KRAS, MED12, ITPKB, EGR2, DDX3X, ZMYM3, and FBXW7.

25. The method of claim 23, wherein the risk allele is selected from HIST1H1E, NRAS, BCOR, RIPK1, SAMHD1, KRAS, MED12, ITPKB, and EGR2.

26. The method of claim 23, wherein the risk allele is selected from TP53, MYD88, NOTCH1, XPO1, CHD2, POT1, and ATM, or wherein the mutation is del(8p), del(13q), del(11q), del(17p), or trisomy 12.

27. A method comprising
(a) analyzing genomic DNA in a sample obtained from a subject having or suspected of having chronic lymphocytic leukemia (CLL) for presence of a mutation in a risk allele selected from the group consisting of SF3B1, HIST1H1E, NRAS, BCOR, RIPK1, SAMHD1, KRAS, MED12, ITPKB, EGR2, DDX3X, ZMYM3, and FBXW7, and
(b) determining whether the mutation is clonal or subclonal, and
(c) identifying the subject as a subject at elevated risk of having CLL with rapid disease progression if the mutation is subclonal.

28. The method of 27, further comprising detecting a mutation in a risk allele selected from the group consisting of TP53, MYD88, NOTCH1, XPO1, CHD2, POT1, ATM, and/or for a mutation selected from the group consisting of del(8p), del(13q), del(11q), del(17p), and trisomy 12.

29. A method comprising
detecting, in genomic DNA of a sample from a subject having or suspected of having chronic lymphocytic leukemia (CLL), presence or absence of a mutation in a risk allele selected from the group consisting of SF3B1, HIST1H1E, NRAS, BCOR, RIPK1, SAMHD1, KRAS, MED12, ITPKB, EGR2, DDX3X, ZMYM3, and FBXW7, in a subclonal population of the CLL sample.

30. A method comprising
(a) analyzing genomic DNA in a sample obtained from a subject having or suspected of having chronic lymphocytic leukemia (CLL) for the presence of a subclonal mutation in a risk allele selected from the group consisting of SF3B1, HIST1H1E, NRAS, BCOR, RIPK1, SAMHD1, KRAS, MED12, ITPKB, EGR2, DDX3X, ZMYM3, and FBXW7, and
(b) identifying the subject as having an elevated risk of rapid disease progression if the sample is positive for the subclonal mutation.

31. The method of 30, further comprising analyzing the genomic DNA for a mutation in a risk allele selected from the group consisting of TP53, MYD88, NOTCH1, XPO1, CHD2, POT1, and ATM, and/or for a mutation selected from the group consisting of del(8p), del(13q), del(11q), del(17p), and trisomy 12.

32. A kit for determining a prognosis of a patient with chronic lymphocytic leukemia (CLL) comprising
reagents for detecting subclonal mutations in one or more risk alleles selected from the group consisting of SF3B1, HIST1H1E, NRAS, BCOR, RIPK1, SAMHD1, KRAS, MED12, ITPKB, EGR2, DDX3X, ZMYM3, and FBXW7, in a sample from a patient, and
instructions for determining the prognosis of the patient based on presence or absence of said subclonal mutations, wherein the presence of a subclonal mutation indicates the patient has an elevated risk of rapid CLL disease progression, thereby determining the prognosis of the patient with CLL.

33. The kit of 32, further comprising reagents for detecting mutations in one more risk alleles selected from the group consisting of TP53, MYD88, NOTCH1, XPO1, CHD2, POT1, and ATM, or for detecting mutations that are selected from the group consisting of del(8p), del(13q), del(11q), del(17p), or trisomy 12.

34. A method comprising
(a) detecting a mutation in genomic DNA from a sample obtained from a subject having or suspected of having chronic lymphocytic leukemia (CLL),
(b) detecting clonal and subclonal populations of cells carrying the mutation, and
(c) identifying the subject as a subject at elevated risk of having CLL with rapid disease progression if the mutation is a driver event present in a subclonal population of cells.

35. A method comprising
(a) analyzing genomic DNA in a sample obtained from a subject having or suspected of having chronic lymphocytic leukemia (CLL) for the presence of a mutation in one or more of at least 2 risk alleles chosen from the group consisting of SF3B1, HIST1H1E, NRAS, BCOR, RIPK1, SAMHD1, KRAS, MED12, ITPKB, EGR2, DDX3X, ZMYM3, FBXW7, ATM, TP53, MYD88, NOTCH1, XPO1, CHD2, POT1, del(8p), del(13q), del(11q), del(17p), and trisomy 12, and
(b) determining whether the mutation is clonal or subclonal, and
(c) identifying the subject as a subject at elevated risk of having CLL with rapid disease progression if the mutation is subclonal.

36. The method of claim 35, wherein the genomic DNA is analyzed for the presence of a mutation in one or more of at least 5 or at least 10 of the risk alleles.

37. The method of any one of claims 23-31 and 34-36, wherein the sample is obtained from peripheral blood, bone marrow, or lymph node tissue.

38. The method of any one of claims 23-31 and 34-36, wherein the genomic DNA is analyzed using whole genome sequencing (WGS), whole exome sequencing (WES), single nucleotide polymorphism (SNP) analysis, deep sequencing, targeted gene sequencing, or any combination thereof.

39. The method of any one of claims 23-31 and 34-36, wherein mutations in more than one risk allele are analyzed.

40. The method of any one of claims 23-31 and 34-36, further comprising treating a subject identified as a subject at elevated risk of having CLL with rapid disease progression.

41. The method of any one of claims 23-31 and 34-36, wherein the method is performed before and after treatment.

42. The method of any one of claims 23-31 and 34-36, further comprising repeating the method every 6 months or if there is a change in clinical status.

43. The method of any one of claims 23-31 and 34-36, wherein clonal or subclonal mutations and/or populations of cells are detected using whole genome sequencing (WGS), whole exome sequencing (WES), single nucleotide polymorphism (SNP) analysis, deep sequencing, targeted gene sequencing, or any combination thereof.
